(19) United States
(12) Patent Application Publication
(10) Pub. No.: US 2024/0309412 A1
(43) Pub. Date: Sep. 19, 2024

(54) METHODS FOR PRODUCING BETALAINS IN YEAST

(71) Applicant: Danmarks Tekniske Universitet, Kongens Lyngby (DK)

(72) Inventors: Irina Borodina, Kongens Lyngby (DK); Mahsa Babaei, Kongens Lyngby (DK); Jane Dannow Dyekjær, Kongens Lyngby (DK); Johann Dietmar Körner, Kongens Lyngby (DK); Philip Tinggaard Thomsen, Kongens Lyngby (DK); Christiane Ursula Glitz, Kongens Lyngby (DK); Marc Cernuda Pastor, Kongens Lyngby (DK)

(21) Appl. No.: 18/564,150

(22) PCT Filed: May 31, 2022
(86) PCT No.: PCT/EP2022/064722

§ 371 Date: Nov. 27, 2023

(30) Foreign Application Priority Data
May 31, 2021 (EP) 21176783.5

(51) Int. Cl. C12P 17/18; C12N 9/02; C12N 9/10

(52) U.S. Cl. CPC C12P 17/182; C12N 9/0069; C12N 9/0071; C12N 9/1051; C12Y 113/11029; C12Y 114/16002; C12Y 204/01128

(57) ABSTRACT

The invention presented herein relates to yeast cells and methods for production of betalains.

### TECHNICAL FIELD

The present invention relates to microbial cell factories, in particular yeast factories, for production of betalains.

### BACKGROUND

Betalains are natural water-soluble colours that are characterized with vibrant red-violet shades in food matrix. Compared to other plant-derived colours in nature, particularly the abundantly found anthocyanins, betalains have special features that excels them as food colorants. These features include higher solubility in water, up to three times higher tinctorial strength, and pH stability in the range of 3-7. Betalains have applications in desserts, confectioneries, dry mixes, dairy and meat products. Betalains are not only useful as natural pigments, but some of them also have health-beneficial properties. For example, the betalain betanin was shown to induce apoptosis in human chronic myeloid leukemia, and some betalains showed in vitro cytotoxicity to HepG2 cancer cells. The inhibition of induced tumours by supplying the drinking water of rats with 78 μg/mL of betanin has also been reported. In humans, daily uptake of 100 mg betalain-rich red beet concentrate was shown to significantly promote anti-inflammatory response.

Betalains are tyrosine-derived pigments found in for example plants of the order Caryophyllales; in the fungi Amanita; in Hygrocybe; and, recently discovered, in the bacterium Gluconacetobacter diazotrophicus. Among the Caryophyllales plants, the red beet Beta vulgaris, the prickly pear cactus Opuntia ficus-indica, the garden four-o'clock Mirabilis jalapa, Portulaca grandiflora, and the paperflower Bougainvillea glabra are the most well-known betalain-producing plants (Khan and Giridhar 2015). The betalains are most commonly classified based on the colour spectrum of the compounds; the yellow-orange betalains are called betaxanthins (FIG. 1a-b), while the red-violet betalains are called betacyanins (FIG. 2a-e). The colour of betalains is related to the formation of a conjugated π-electron system of the 1,7-diazaheptamethin system, which is created by Schiff-base condensation of a nucleophilic amine and betalamic acid aldehyde group.

Currently, the majority of the betalains are obtained by extraction from plants, such as red beet or cactus pear. The content of betalains in plants is rather low, e.g. betanin is present at 300-600 mg/kg of red beet roots. Moreover, the most commonly used extraction methods lead to the presence of pyrazine and geosmin in the red beet root extract, giving it an undesirable earthy flavor (Dos Santos et al. 2018).

Recombinant production of betalains by fermentation of recombinant micrograms will result in lower cost and higher purity.

### SUMMARY

The invention is as defined in the claims.

The invention presented herein relates to a yeast cell capable of producing betalains. Betalains are a class of yellow to violet pigments which can be used as natural food dyes. Thus, the invention presented herein discloses a yeast cell platform for environment-friendly production of natural food dyes.

Provided herein is a yeast cell capable of producing one or more betalains, said yeast cell expressing:
a. a first heterologous enzyme (TYH) capable of hydroxylating L-tyrosine and oxidizing L-DOPA, such as CYP76ADα;
b. a second heterologous enzyme which is a 4,5-DOPA extradiol dioxygenase (DOD); and
c. a third heterologous enzyme having glycosyltransferase activity, wherein said enzyme is selected from:
   i. Chenopodium quinoa glycosyltransferase CqSGT2 set forth in SEQ ID NO: 65, or a functional variant thereof having at least 70% sequence identity thereto;
   ii. Beta vulgaris glycosyltransferase BvSGT2 set forth in SEQ ID NO: 53, or a functional variant thereof having at least 70% identity thereto;
   iii. Bougainvillea glabra glycosyltransferase BgGT2 set forth in SEQ ID NO: 67, or a functional variant thereof having at least 70% identity thereto; and
   iv. Beta vulgaris glycosyltransferase BvSGT4 set forth in SEQ ID NO: 57, or a functional variant thereof having at least 70% identity thereto;
whereby said cell is capable of producing one or more betalains, wherein said one or more betalains comprise one or more glycosylated betalains such as betanin and/or isobetanin.

Also provided herein is a yeast cell capable of producing on or more betalains, said yeast cell expressing:
a. a first heterologous enzyme (TYH) capable of hydroxylating L-tyrosine and oxidizing L-DOPA, such as CYP76ADα; a second heterologous enzyme which is a 4,5-DOPA extradiol dioxygenase (DOD); and a third heterologous enzyme, having glycosyltransferase activity, such as an activity selected from a betanidin-5-O-glucosyltransferase (B50G) activity and a cyclo-DOPA-5-O-glucosyltransferase (cDOPA50GT) activity, such as a glycosyltransferase, such as a scopoletin glucosyltransferase (SGT), whereby said cell is capable of producing one or more betalains, wherein said one or more betalains comprise one or more glycosylated betalains such as betanin and/or isobetanin; and/or
b. a first heterologous enzyme (TYH) capable of hydroxylating L-tyrosine and oxidizing L-DOPA, such as CYP76ADα; and a second heterologous enzyme which is a DOD having a truncation in its C-terminal end (DOD*), whereby said cell is capable of producing one or more betalains, wherein said one or more betalains comprise one or more betaxanthins.

Further provided herein is a method for production of one or more betalains in a yeast cell, said method comprising the steps of incubating a yeast cell in a medium, wherein said yeast cell expresses:
a. a first heterologous enzyme (TYH) capable of hydroxylating L-tyrosine and oxidizing L-DOPA, such as CYP76ADα;
b. a second heterologous enzyme which is a 4,5-DOPA extradiol dioxygenase (DOD); and
a. a third heterologous enzyme, having glycosyltransferase activity, wherein said enzyme is selected from:
   i. Chenopodium quinoa glycosyltransferase CqSGT2 set forth in SEQ ID NO: 65, or a functional variant thereof having at least 70% sequence identity thereto;
   ii. Beta vulgaris glycosyltransferase BvSGT2 set forth in SEQ ID NO: 53, or a functional variant thereof having at least 70% identity thereto;
   iii. Bougainvillea glabra glycosyltransferase BgGT2 set forth in SEQ ID NO: 67, or a functional variant thereof having at least 70% identity thereto; and
   iv. Beta vulgaris glycosyltransferase BvSGT4 set forth in SEQ ID NO: 57, or a functional variant thereof having at least 70% identity thereto;
      whereby said cell is capable of producing one or more betalains, wherein said one or more betalains comprise one or more glycosylated betalains such as betanin and/or isobetanin.

Also provided herein is a method for production of one or more betalains in a yeast cell, said method comprising the steps of incubating a yeast cell in a medium, wherein said yeast cell expresses:
a. a first heterologous enzyme (TYH) capable of hydroxylating L-tyrosine and oxidizing L-DOPA, such as CYP76ADα; a second heterologous which is a 4,5-DOPA extradiol dioxygenase (DOD); and a third heterologous enzyme, having glycosyltransferase activity, such as an activity selected from a betanidin-5-O-glucosyltransferase (B50G) activity and a cyclo-DOPA-5-O-glucosyltransferase (cDOPA50GT) activity, such as a glycosyltransferase, such as a scopoletin glucosyltransferase (SGT), whereby said cell is capable of producing one or more betalains, wherein said one or more betalains comprise one or more glycosylated betalains such as betanin and/or isobetanin; and/or
b. a first heterologous enzyme (TYH) capable of hydroxylating L-tyrosine and oxidizing L-DOPA, such as CYP76ADα; and a second heterologous enzyme, which is a DOD having a truncation in its C-terminal end (DOD*), whereby said cell is capable of producing one or more betalains, wherein said one or more betalains comprise one or more betaxanthins.

Also provided herein is a system of nucleic acid constructs comprising polynucleotides encoding:
a. a TYH, such as CYP76ADα capable of:
   i. hydroxylating L-tyrosine; and/or
   ii. oxidizing L-DOPA; and
b. a DOD capable of oxygenating L-DOPA; and
c. a glycosyltransferase, such as an SGT, capable of:
   i. glycosylating cyclo-DOPA; and/or
   ii. glycosylating betanidin.

Also provided herein is a system of nucleic acid constructs comprising polynucleotides encoding:
a. a TYH, such as CYP76ADα capable of:
   i. hydroxylating L-tyrosine; and/or
   ii. oxidizing L-DOPA; and
b. a DOD capable of oxygenating L-DOPA; and
c. an enzyme having glycosyltransferase activity, wherein said enzyme is selected from:
   i. Chenopodium quinoa glycosyltransferase CqSGT2 set forth in SEQ ID NO: 65, or a functional variant thereof having at least 70% sequence identity thereto;
   ii. Beta vulgaris glycosyltransferase BvSGT2 set forth in SEQ ID NO: 54, or a functional variant thereof having at least 70% identity thereto;
   iii. Bougainvillea glabra glycosyltransferase BgGT2 set forth in SEQ ID NO: 67, or a functional variant thereof having at least 70% identity thereto; and
   iv. Beta vulgaris glycosyltransferase BvSGT4 set forth in SEQ ID NO: 58, or a functional variant thereof having at least 70% identity thereto.

Further provided herein is the use of a polynucleotide as set forth in SEQ ID NO: 54, SEQ ID NO: 65, SEQ ID NO: 67 or SEQ ID NO: 58 for obtaining a protein capable of glycosylating a betalain and/or a betalain precursor, such as a protein capable of glycosylating betanidin and/or cyclo-DOPA, such as a protein with betanidin-5-O-glucosyltransferase activity and/or a protein with cyclo-DOPA 5-O-glucosyltransferase activity.

Also provided herein is the use of and enzyme having glycosyltransferase activity, such as a glycosyltransferase, such as an SGT, as a betanidin-5-O-glucosyltransferase (B50G) and/or a cyclo-DOPA 5-O-glucosyltransferase (cDOPA5OGT), preferably wherein said enzyme having glycosyltransferase activity is selected from the glycosyltransferase from Beta vulgaris set forth in SEQ ID NO: 53 (BvSGT2), the glycosyltransferase from Beta vulgaris set forth in SEQ ID NO: 57 (BvSGT4), the glycosyltransferase from Chenopodium quinoa as set forth in SEQ ID NO: 65 (CqSGT2) and the glycosyltransferase from Bougainvillea glabra set forth in SEQ ID NO: 67 (BgGT2), or functional variants having at least 80% identity thereto.

Further provided herein is the use of:
i. Chenopodium quinoa glycosyltransferase CqSGT2 set forth in SEQ ID NO: 65, or a functional variant thereof having at least 70% sequence identity thereto;
ii. Beta vulgaris glycosyltransferase BvSGT2 set forth in SEQ ID NO: 53, or a functional variant thereof having at least 70% identity thereto;
iii. Bougainvillea glabra glycosyltransferase BgGT2 set forth in SEQ ID NO: 67, or a functional variant thereof having at least 70% identity thereto; and/or
iv. Beta vulgaris glycosyltransferase BvSGT4 set forth in SEQ ID NO: 57, or a functional variant thereof having at least 70% identity thereto;
to catalyse the conversion of cyclo-DOPA to cyclo-DOPA-5-O-glucoside and/or glycosylating betanidin and/or to catalyse the glycosylation of betanidin.

Also provided herein is the use of a DOD variant (DOD*) to catalyse the conversion of L-DOPA to 4,5-seco-DOPA, which is a DOD truncation mutant of having a truncation of at least 5 amino acids at the C-terminal end, such as at least 6, such as at least 7, such as at least 8, such as at least 9, such as at least 10, such as at least 12, such as at least 14, such as at least 16, such as at least 18, such as at least 20, such as at least 25, such as at least 30, such as at least 35, such as at least 40, such as at least 45, such as at least 50 amino acids at the C-terminal end.

Further provided herein is a betalain, such as a betacyanin such as betanidin, betanin or isobetanin, obtainable by the methods presented herein.

Also provided herein is the use of a betalain, such as a betacyanin such as betanidin, betanin or isobetanin, obtainable by the methods presented herein.

Further provided herein is the use of a heterologous TYH, DOD, DOD*, and/or enzyme having glycosyltransferase activity, such as a glycosyltransferase, such as an SGT, as defined herein in a method of production of one or more betalains.

Further provided herein is the use of a heterologous TYH, DOD and enzyme having glycosyltransferase activity, such as a glycosyltransferase, as defined herein, in a method of production of one or more betalains.

Also provided herein is a kit of parts comprising:
a. the yeast cell described herein; and/or
b. the nucleic acid system described herein, wherein said system is for modifying a yeast cell; and
c. instructions for use; and
d. optionally, the yeast cell to be modified.

Also provided herein is a method for producing at least 0.5 mg/L of one or more betalains, wherein said one or more betalains comprise a glycosylated betalain such as betanin and/or isobetanin, such as at least 1 mg/L, such as at least 1.5 mg/L, such as at least 5 mg/L, such as at least 10 mg/L, such as at least 25 mg/L, such as at least 50 mg/L, such as at least 100 mg/L, such as at least 250 mg/L, such as at least 500 mg/L, such as at least 750 mg/L, such as at least 1 g/L, such as at least 2 g/L, such as at least 3 g/L, such as at least 4 g/L, such as at least 5 g/L, such as at least 6 g/L, such as at least 7 g/L, such as at least 8 g/L, such as at least 9 g/L, such as at least 10 g/L, such as at least 11 g/L, such as at least 12 g/L, such as at least 13 g/L, such as at least 14 g/L, such as at least 15 g/L, such as at least 16 g/L, such as at least 17 g/L, such as at least 18 g/L, such as at least 19 g/L, such as at least 20 g/L, such as at least 25 g/L, such as at least 30 g/L, such as at least 35 g/L, such as at least 40 g/L, such as at least 45 g/L, such as at least 50 g/L, or more, wherein said method is according to the method presented herein.

### DESCRIPTION OF DRAWINGS

FIG. 1. a)-b) Examples of betaxanthin (Bx) compounds: 1) Betalamic acid, 2) Proline-Bx (Indicaxanthin), 3) 4-hydroxy-proline-Bx (Portulacaxanthin I), 4) tyrosine-Bx (Portulacaxanthin II), 5) Glycine-Bx (Portulacaxanthin III), 6) Glutamine-Bx (Vulgaxanthin I), 7) Glutamic acid-Bx (Vulgaxanthin II), 8) Asparagine-Bx (Vulgaxanthin III), 9) Leucine-Bx (Vulgaxanthin IV), 10) Methionine sulfoxide-Bx (Miraxanthin I), 11) Aspartic acid-Bx (Miraxanthin II), 12) Tyramine-Bx (Miraxanthin III), 13) Dopamine-Bx (Miraxanthin IV), 14) 3-methoxytyramine-Bx (Miraxanthin V), 15) Histamine-Bx, 16) DOPA-Bx (Dopaxanthin), 17) pentahomoserine-Bx, 2-amino-5-hydroxypentanoic acid-Bx (Humilixanthin), 18) GABA-Bx, 19) Methylated arginine-Bx, 20) Serine-Bx, 21) Tryptophan-Bx, 22) Valine-Bx, 23) Phenylalanine-Bx, 24) Isoleucine-Bx, 25) Alanine-Bx, 26) Histidine-Bx (Muscaaurin VII), 27) Methionine-Bx, 28) Threonine-Bx, 29) Arginine-Bx, 30) Lysine-Bx, 31) Ethanolamine-Bx, 32) Putrescine-Bx.

FIG. 2. a)-f) Chemical structure of examples of betacyanins. The name of the molecule together with the original source of this compound in plants and also part of the plant (in symbols) is stated.

FIG. 3. Biosynthetic pathway towards betalains.

FIG. 4. Experimental workflow for constructing and screening yeast libraries that allow identification of suitable combinations of DOD-TYH orthologues for betaxanthins production.

FIG. 5. a) Calibration curve and HPLC-chromatograms of betanin for yeast cultures. b) The 3D contour plot for the betanin molecule in a standard sample and in ST10614 shows an absorption maxima at around A=530 nm, while this peak is absent in betaxanthin-producing strain (ST10529).

FIG. 6. Betaxanthins measured as mean specific fluorescence of several yeast isolates from the sorted library 3 (as of FIG. 4) at 24 and 48 hours. The measurements were performed in biological triplicates. In each isolate, the DOD and TYH variants were determined by Sanger sequencing and are indicated on the graph.

FIG. 7. Betaxanthins production in yeast strains expressing three different DOD-TYH combinations.

FIG. 8. Expression of scopoletin glucosyltransferases from Beta vulgaris (BvSGTs) in betaxanthin-producing yeasts results in betalains production (red color wavelength). The bathochromic shift is observed from betaxanthins to betacyanins in strains expressing BvSGT2, BvSGT4, and DbB5GT.

FIG. 9. Expression of scopoletin glucosyltransferases from Beta vulgaris (BvSGTs) in betaxanthin-producing yeasts results in production of glycosylated betalains. Betanin content (mg/L) in cell cultures was measured by HPLC.

FIG. 10. Targeted genes with up/down-regulation in single strains for betaxanthin titer improvement with fold change in fluorescence/OD₆₀₀ (betaxanthin specific titer). a) The perturbation of these targets was done by activation (VPR₋₅₀₀, VPR₋₃₅₀ and VPR₋₂₀₀) or repression (VPR₊₃₀, Mxi1₋₃₅₀ and Mxi₋₂₀₀). b) The corresponding change in betaxanthin titers for cells with perturbations reported in FIG. 8. The measurements were performed in biological duplicates.

FIG. 11. a) Betalain production in yeast Y. lipolytica. All the genes are in codon-optimized versions for Y. lipolytica. By expressing only DOD and TYH enzymes (ST11022), the yeast produces pink/red betalains with absorption maxima of λ=510 nm (different from betacyanins with λₘₐₓ=530 nm). b) After integrating the glycosyltransferases, a bathochromic shift was observed for betacyanins (λₘₐₓ=530 nm) in strains ST11195 and ST11193. c) The 3D contour plot shows the presence of both compounds at λₘₐₓ=530 nm (betacyanins) and also λₘₐₓ=510 nm for strain ST11193. d) The betanin content (mg/L) was measured by HPLC.

FIG. 12. Comparison of the betacyanin production of DbB5GT and the glycosyltransferases BvSGT2, BvSGT4, CqSGT2 and BgGT2 expressed in yeast strain ST10529 in a small-scale cultivation. The betanin and isobetanin content (mg/L) was measured by HPLC and normalized to an OD₆₀₀ of 1 of the cell cultures.

FIG. 13. Betanin (a) and betaxanthin (b) production in Y. lipolytica strains engineered for enhanced L-tyrosine precursor supply and/or heterologous pathway flux. Betanin production was quantified from the supernatant's absorbance measured at 535 nm, relative to a dilution row of red beet extract diluted with dextrin. Quantification of betaxanthin production was fluorescence-based. Strains were inoculated from precultures into MM to an approximate OD₆₀₀ of 0.1. Cultivations were carried out in biological triplicates.

FIG. 14. Betanin production in Y. lipolytica strain ST11942 with increasing L-tyrosine supplementation. Betanin production was quantified from the supernatant's absorbance measured at 535 nm, relative to a dilution row of red beet extract diluted with dextrin. Strains were inoculated from precultures into MM to approximately an OD₆₀₀ of 0.1. Cultivations were carried out in biological duplicates.

FIG. 15. HPLC chromatogram of the commercial betanin standard from Sigma (1 g/L in H₂O) and ST11825 (ST10529 with BvSGT2). In the standard, betanin and its isomer isobetanin are present in almost equal amount. The yeast strain mainly produces betanin but also produces isobetanin.

FIG. 16. Betanin and isobetanin production from Y. lipolytica strain ST11942 and ST12309 (ST11942 Δ4-hppd) as measured in the supernatant by HPLC. Betanin and isobetanin production was quantified using the red beet extracted diluted with dextrin as an internal standard. Cultivations were inoculated from precultures into 50 mL MM in 500 mL shakeflasks to approximately an OD₆₀₀ of 0.1. Cultivations were carried out in biological duplicates.

FIG. 17. Synthesis of pyomelanin through the homogentisic acid (HGA) metabolic pathway in Yarrowia lipolytica. Abbreviations; TAT: Tyrosine aminotransferase, HppD: 4-hydroxyphenylpyruvate dioxygenase FIG. 18. HPLC chromatogram of the extracellular metabolites comparatively in ST11942 (top) and ST12309 (bottom). It is clearly seen that in addition to producing more of the betalain pathway-specific compounds; betanin (˜535 nm), isobetanin (˜535 nm), and betalamic acid (˜410 nm), much less of other metabolites are produced—here among compounds with absorptions maxima fitting HGA (˜290 nm).

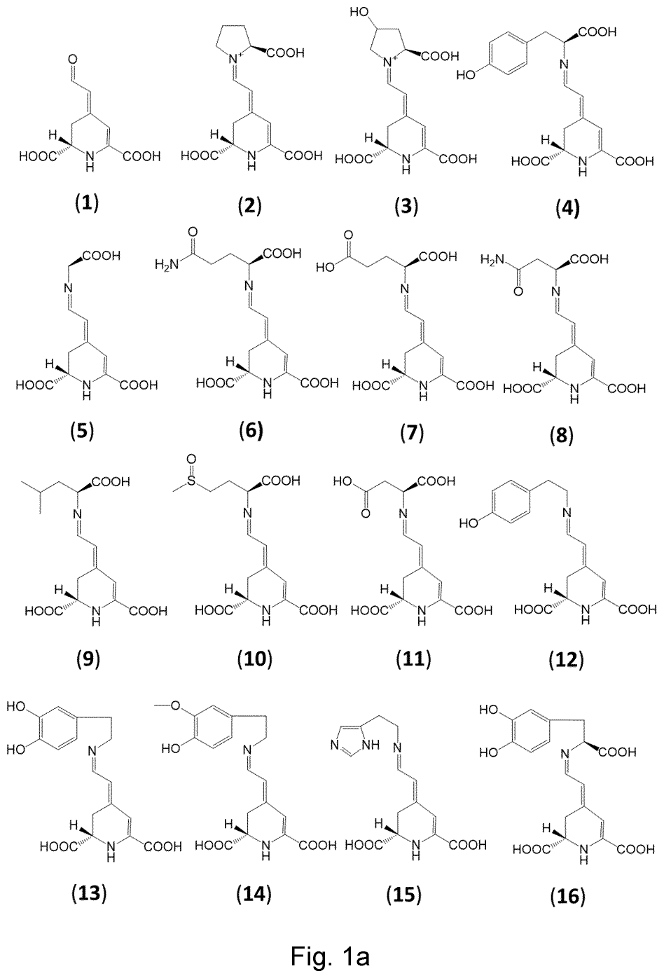

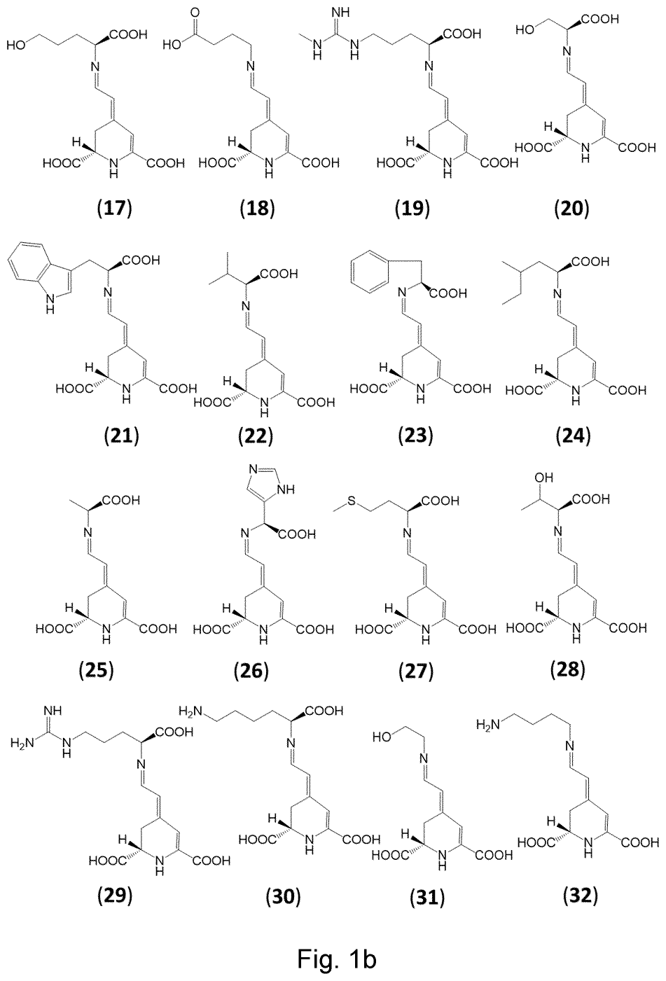

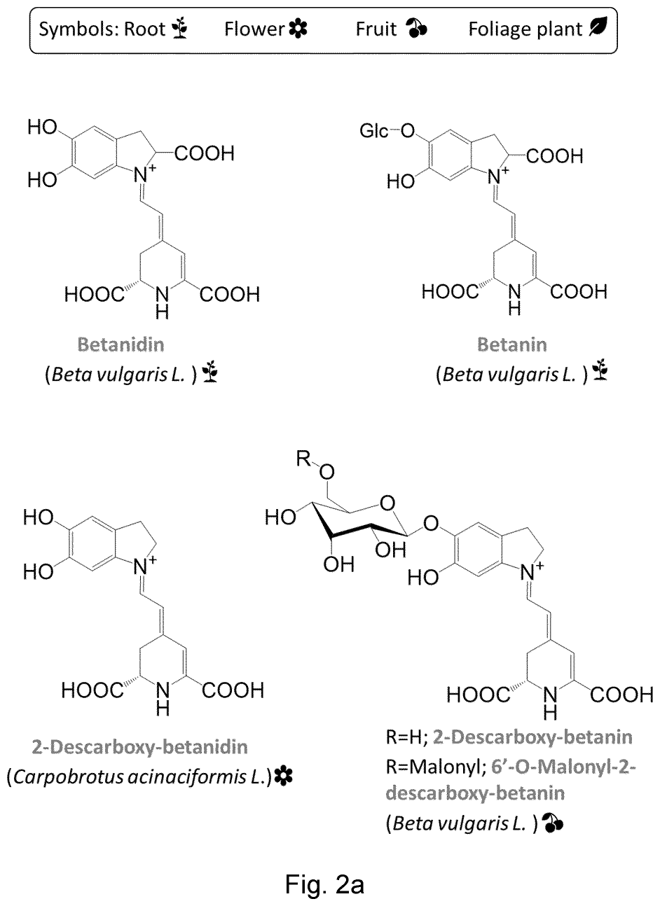

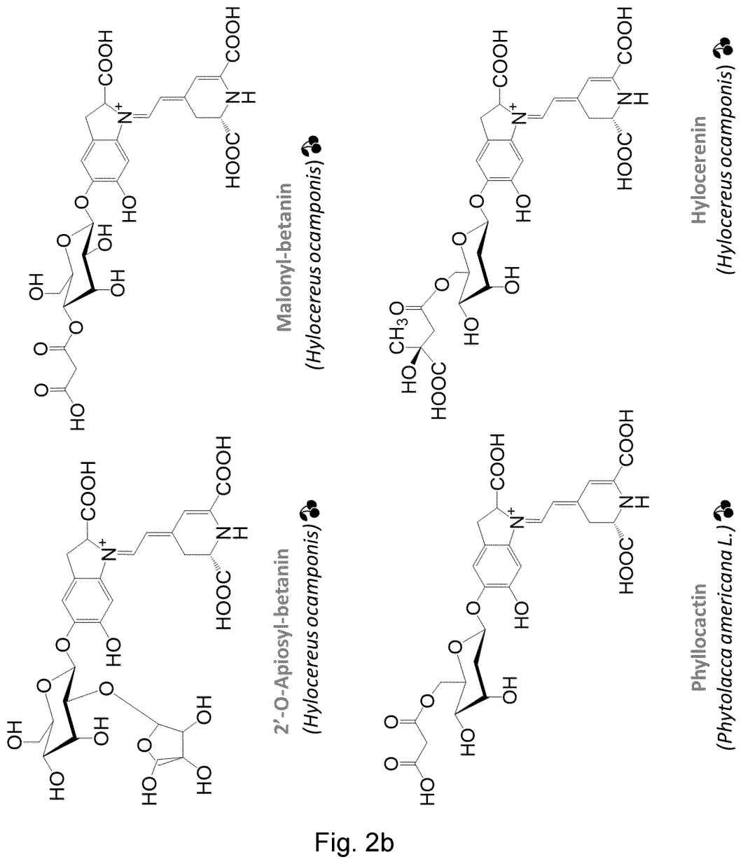

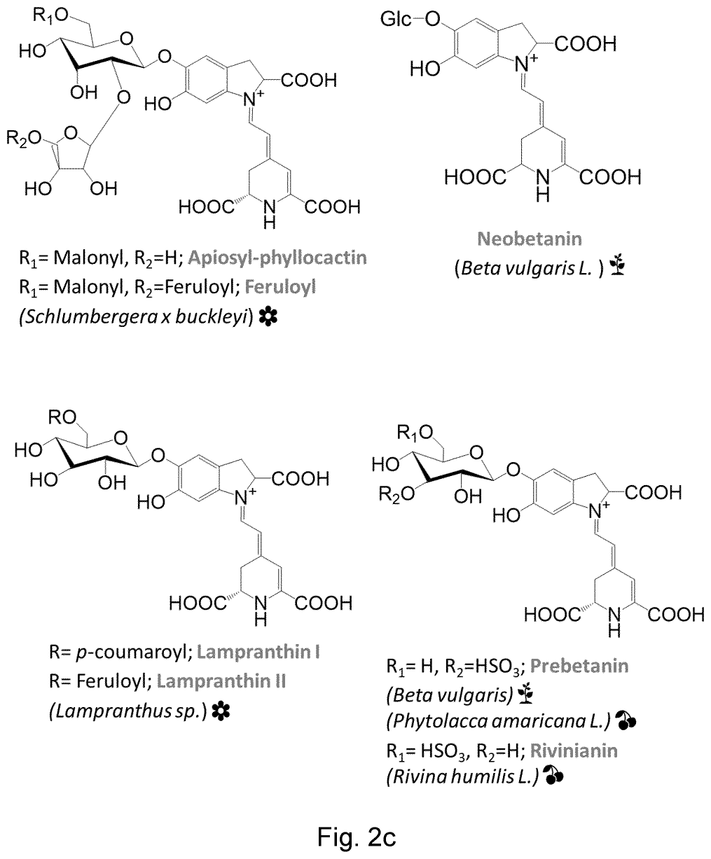

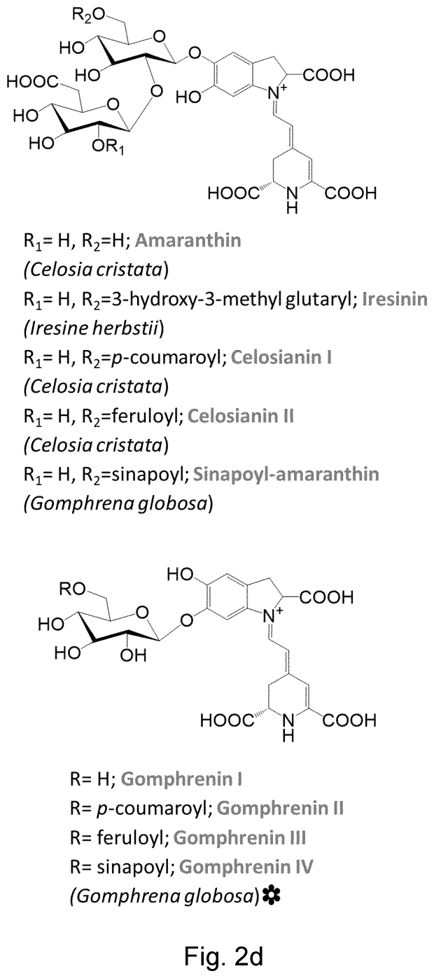

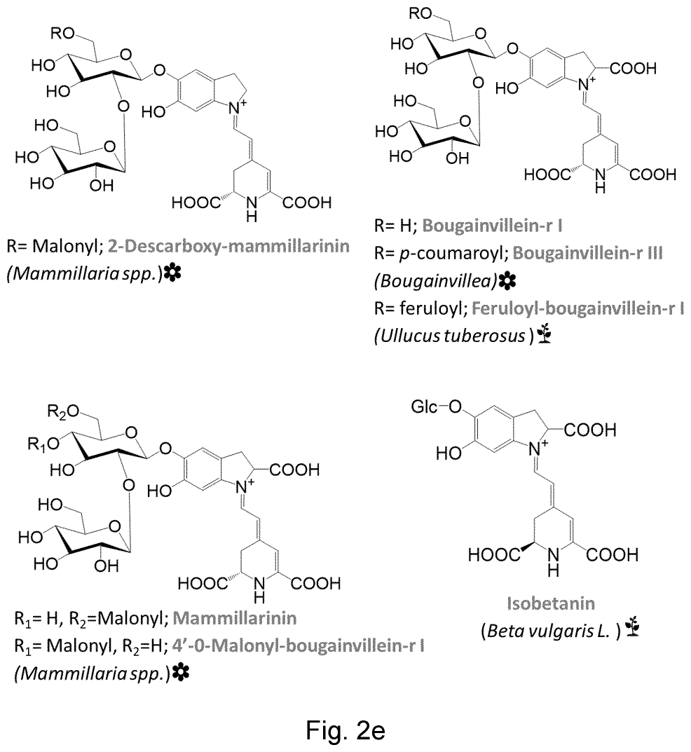

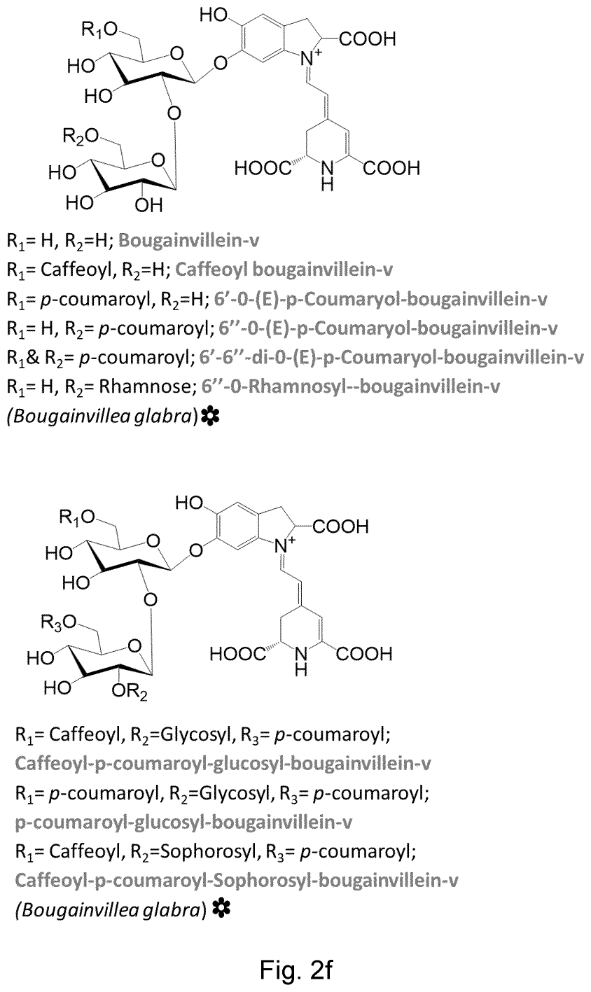

### DETAILED DESCRIPTION

### Definitions

Betacyanin: the term “betacyanin” refers herein to a category of betalains. Betacyanins include red to violet betalain pigments, such as for example betanin and isobetanin.

Betalain: the term “betalain” refers herein to a class of tyrosine-derived pigments found for example in plants of the Caryophyllales. There are two categories of betalains; betaxanthins and betacyanins.

Betaxanthin: the term “betaxanthin” refers herein to a category of betalains. Betaxanthins include yellow to orange betalain pigments.

Functional variant: the term “functional variant” refers herein to functional variants of an enzyme, which retain at least some of the activity of the parent enzyme. Thus, a functional variant of a TYH, a DOD and/or an enzyme having glycosyltransferase activity, such as a glycosyltransferase, such as an SGT, can catalyse the same conversion as the TYH, the DOD, and/or the enzyme having glycosyltransferase activity, such as the glycosyltransferase, such as the SGT, respectively, from which they are derived, although the efficiency of the reaction may be different, e.g. the efficiency is decreased or increased compared to the parent enzyme or the substrate specificity is modified.

Glycosylated betalain: the term “glycosylated betalain” refers herein to a betalain that has been glycosylated, i.e. a betalain on which a carbohydrate, i.e. a glycosyl donor, has been attached. In particular, a glycosylated betalain herein refers to a glycosylated betacyanin, such as a betanin and/or an isobetanin.

Heterologous: the term “heterologous” when referring to a polypeptide, such as a protein or an enzyme, or to a polynucleotide, shall herein be construed to refer to a polypeptide or a polynucleotide which is not naturally present in a wild type cell. For example, the term “heterologous DOD” when applied to Saccharomyces cerevisiae refers to a DOD which is not naturally present in a wild type S. cerevisiae cell, e.g. a DOD derived from Portulaca grandiflora.

Identity/homology: the terms “identity and homology”, with respect to a polynucleotide (or polypeptide), are defined herein as the percentage of nucleic acids (or amino acids) in the candidate sequence that are identical or homologous, respectively, to the residues of a corresponding native nucleic acids (or amino acids), after aligning the sequences and introducing gaps, if necessary, to achieve the maximum percent identity/similarity/homology, and considering any conservative substitutions according to the NCIUB rules (hftp://www.chem.qmul.ac.uk/iubmb/misc/naseq.html; NC-IUB, Eur J Biochem (1985)) as part of the sequence identity. Neither 5′ or 3′ extensions nor insertions (for nucleic acids) or N′ or C′ extensions nor insertions (for polypeptides) result in a reduction of identity, similarity or homology. Methods and computer programs for the alignments are well known in the art. Generally, a given homology between two sequences implies that the identity between these sequences is at least equal to the homology; for example, if two sequences are 70% homologous to one another, they cannot be less than 70% identical to one another—but could be sharing 80% identity.

Native to: the term when referring to a polypeptide or a polynucleotide native to an organism means that said polypeptide or polynucleotide is naturally found in said organism.

Titer: the titer of a compound refers herein to the produced concentration of a compound. When the compound is produced by a cell, the term refers to the total concentration produced by the cell, i.e. the total amount of the compound divided by the volume of the culture medium. This means that, particularly for volatile compounds, the titer includes the portion of the compound which may have evaporated from the culture medium, and it is thus determined by collecting the produced compound from the fermentation broth and from potential off-gas from the fermenter.

### Betalain Production

The inventors of the present invention have discovered that the expression of certain enzymes enables and/or improves production of betalains in yeast cells. Betalains are a class of red to violet (betacyanins) and yellow to orange (betaxanthins) pigments which can be used as natural food dyes.

Currently, the majority of the betalains are obtained by extraction from plants, such as red beet or cactus pear. The content of betalains in plants is rather low, betanin is for example only present at 300-600 mg/kg of red beet roots. Moreover, the most commonly used extraction methods lead to the presence of pyrazine and geosmin in the red beet root extract, giving it an undesirable earthy flavour.

Production of betalains in yeast cells is less expensive and yields betalains of higher purity and yield, without undesirable flavours. Thus, the yeast cells disclosed herein provides a platform for improved and environment-friendly production of natural food dyes well suitable for food coloring.

In particular, the inventors have surprisingly discovered that certain glycosyltransferases, such as scopoletin glycosyltransferases (SGTs) and other glycosyltransferases, can be used to glycosylate betalains in order to generate glycosylated betalains, such as betanin and isobetanin.

The inventors have also surprisingly discovered that the titer and/or the purity of glycosylated betalains, such as betanin and/or isobetanin, produced in yeast cells such as Yarrowia lipolytica, can be improved by mutating a mutation in 4-hydroxyphenylpyruvate dioxygenase (4-HPPD), such as by a mutation decreasing the activity of 4-HPPD.

The inventors have further shown that expression of a truncated variant of the 4,5-DOPA extradiol dioxygenase (DOD*) can improve the titer of both betaxanthins and glycosylated betalains.

Thus, provided herein is a yeast cell capable of producing one or more betalains, said yeast cell expressing:
a. a first heterologous enzyme (TYH) capable of hydroxylating L-tyrosine and oxidizing L-DOPA, such as CYP76ADα;
b. a second heterologous enzyme which is a 4,5-DOPA extradiol dioxygenase (DOD); and
c. a third heterologous enzyme having glycosyltransferase activity, wherein said enzyme is selected from:
   i. Chenopodium quinoa glycosyltransferase CqSGT2 set forth in SEQ ID NO: 65, or a functional variant thereof having at least 70% sequence identity thereto;
   ii. Beta vulgaris glycosyltransferase BvSGT2 set forth in SEQ ID NO: 53, or a functional variant thereof having at least 70% identity thereto;
   iii. Bougainvillea glabra glycosyltransferase BgGT2 set forth in SEQ ID NO: 67, or a functional variant thereof having at least 70% identity thereto; and
   iv. Beta vulgaris glycosyltransferase BvSGT4 set forth in SEQ ID NO: 57, or a functional variant thereof having at least 70% identity thereto;
whereby said cell is capable of producing one or more betalains, wherein said one or more betalains comprise one or more glycosylated betalains such as betanin and/or isobetanin.

Also provided herein is a yeast cell capable of producing one or more betalains, said yeast cell expressing:
a. a first heterologous enzyme (TYH) capable of hydroxylating L-tyrosine and oxidizing L-DOPA, such as CYP76ADα; a second heterologous enzyme which is a 4,5-DOPA extradiol dioxygenase (DOD); and a third heterologous enzyme having glycosyltransferase activity, such as an activity selected from a betanidin-5-O-glucosyltransferase (B50G) activity and a cyclo-DOPA-5-O-glucosyltransferase (cDOPA50GT) activity, such as a glycosyltransferase, such as a scopoletin glucosyltransferase (SGT), whereby said cell is capable of producing one or more betalains, wherein said one or more betalains comprise one or more glycosylated betalains such as betanin and/or isobetanin; and/or
b. a first heterologous enzyme (TYH) capable of hydroxylating L-tyrosine and oxidizing L-DOPA, such as CYP76ADα; and a second heterologous enzyme which is a DOD having a truncation in its C-terminal end (DOD*), whereby said cell is capable of producing one or more betalains, wherein said one or more betalains comprise one or more betaxanthins.

In some embodiments, the yeast cell of step a) is capable of producing one or more betalains, wherein said one or more betalains comprise one or more betaxanthins.

In some embodiments, the yeast cell is capable of expressing both the enzymes of step a) and the enzymes of step b) at the same time. Thus, in some embodiments, the yeast cell expresses a first heterologous enzyme (TYH) capable of hydroxylating L-tyrosine and oxidizing L-DOPA, such as CYP76ADα; a second heterologous enzyme which is a DOD having a truncation in its C-terminal end (DOD*); and a third heterologous enzyme, having glycosyltransferase activity, such as an activity selected from a betanidin-5-O-glucosyltransferase (B50G) activity and a cyclo-DOPA-5-O-glucosyltransferase (cDOPA50GT) activity, such as a glycosyltransferase, such as a scopoletin glucosyltransferase (SGT), whereby said cell is capable of producing one or more betalains, wherein said one or more betalains comprise one or more glycosylated betalains such as betanin and/or isobetanin. In some embodiments, said cell is further capable of producing one or more betalains, wherein said one or more betalains comprise one or more betaxanthins.

In some embodiments, the yeast cell presented herein is capable of producing one or more glycosylated betalains, such as betanin and/or isobetanin, and one or more betaxanthins. In other words, the yeast cell produces a mixture of one or more glycosylated betalains, such as betanin and/or isobetanin, and one or more betaxanthins. In some embodiments, said mixture further comprises betanidin and/or other compounds which are precursors, i.e. upstream in the production pathway, of glycosylated betalains and betaxanthins.

In one embodiment of the invention:
the TYH is capable of converting L-tyrosine to L-3,4-dihydroxyphenylalanine (L-DOPA) and/or converting L-DOPA to L-Dopaquinone;
the DOD and/or the DOD* is capable of converting L-DOPA to 4,5-seco-DOPA;
the enzyme having glycosyltransferase activity, such as the glycosyltransferase, such as the SGT, is capable of converting cyclo-DOPA to cyclo-DOPA-5-O-glucoside and/or glycosylating betanidin, thereby converting betanidin to a glycosylated betalain such as betanin and/or isobetanin;
   and wherein one or more of the following reactions are spontaneous reactions:
conversion of 4,5-seco-DOPA to betalamic acid;
conversion of betalamic acid to one or more of a betaxanthin, betanidin, betanin or isobetanin;
conversion of L-dopaquinone to cyclo-DOPA;
conversion of cyclo-DOPA to betanidin.

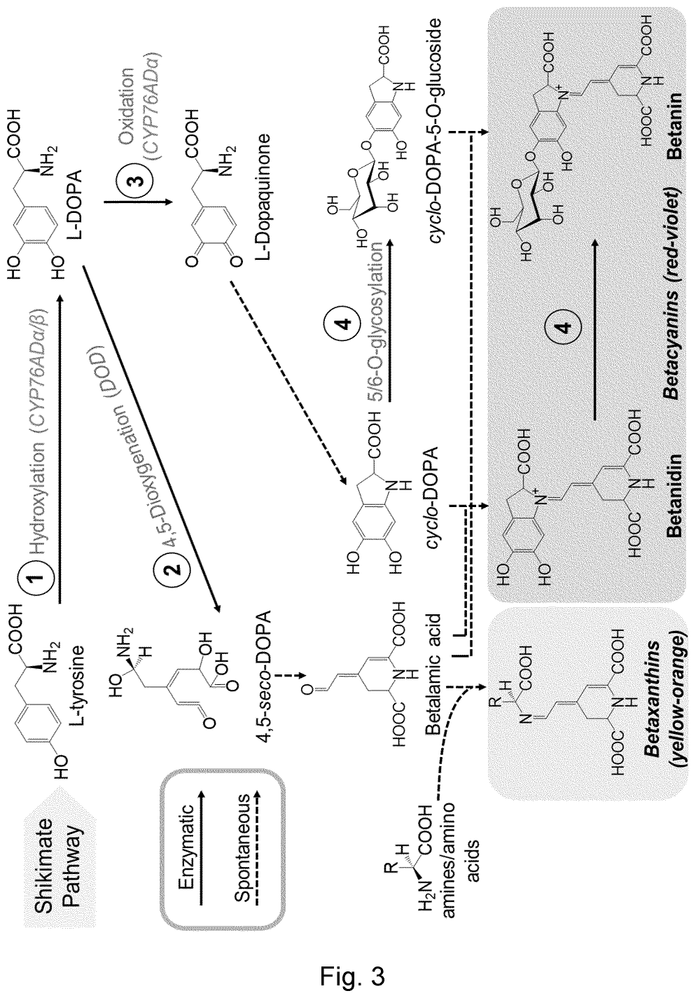

An overview of the pathway is presented in FIG. 3. The yeast cell of the present disclosure expresses a TYH which is capable of converting L-tyrosine to L-DOPA. The L-DOPA may be converted into L-dopaquinone by the action of the TYH, and/or into 4,5-seco-DOPA by the action of the DOD and/or the DOD* also expressed in the cell.

L-dopaquinone can be converted to cyclo-DOPA in a spontaneous reaction. Cyclo-DOPA can then be converted to cyclo-DOPA-5-O-glucoside by the action of an enzyme with glycosyltransferase activity, such as by the action of an enzyme with cyclo-DOPA-5-O-glucosyltransferase (cDOPA5OGT) activity, such as by a glycosyltransferase, such as by an SGT, or to betanidin by a spontaneous reaction with betalamic acid.

Cyclo-DOPA-5-O-glucoside can be converted to betanidin in a spontaneous reaction with betalamic acid. Betanidin can be converted to betanin and/or isobetanin by an enzyme with glycosyltransferase activity, such as by the action of an enzyme with betanidin-5-O-glucosyltransferase (B50G) activity, such as by a glycosyltransferase, such as by an SGT.

4,5-seco-DOPA can be converted to betalamic acid in a spontaneous reaction. Betalamic acid can be converted to betaxanthins by spontaneous reaction with an amine or amino acid.

In some embodiments, L-tyrosine is supplied in the growth medium. In some embodiments, the growth medium is supplemented with at least 100 mg/L L-tyrosine, such as at least 200 mg/L L-tyrosine, such as at least 400 mg/L L-tyrosine, such as at least 600 mg/L L-tyrosine, such as at least 800 mg/L L-tyrosine, such as at least 1.2 g/L L-tyrosine, such as at least 1.4 L-tyrosine, such as at least 1.6 g/L L-tyrosine, such as at least 1.8 g/L L-tyrosine, such as at least 2 g/L L-tyrosine, such as at least 3 g/L L-tyrosine, such as at least 4 g/L L-tyrosine, such as at least 6 g/L L-tyrosine, such as at least 8 g/L L-tyrosine.

In other embodiments, L-tyrosine is produced by the yeast cell. In some embodiments, the yeast cell is engineered to improve its production of L-tyrosine, such as defined herein in the section “Yeast cell”.

Further provided herein is the use of a heterologous TYH, DOD, DOD*, and/or enzyme having glycosyltransferase activity, such as a glycosyltransferase, such as an SGT, as defined in herein in a method of production of one or more betalains.

In some embodiments, the heterologous TYH, DOD, DOD*, and/or enzyme having glycosyltransferase activity, such as the glycosyltransferase, such as the SGT, as defined herein are used in a method of production of one or more betalains, wherein said one or more betalains comprise one or more glycosylated betalains, such as betanin and/or isobetanin.

In some embodiments, the heterologous TYH, DOD, DOD*, and/or enzyme having glycosyltransferase activity, such as the glycosyltransferase, such as the SGT, as defined herein are used in a method of production of one or more betalains, wherein said one or more betalains comprise one or more betaxanthins.

In some embodiments, the method is performed in vivo, such as in a cell, such as in a prokaryotic or a eukaryotic cell. In some embodiments, the TYH, DOD, DOD*, and/or enzyme having glycosyltransferase activity, such as the glycosyltransferase, such as the SGT, may be purified or obtained from cell extract, and may further be contacted with the substrate or substrates to obtain the betalains, such as the betaxanthins and/or the glycosylated betalains. Alternatively, the betalains, such as the betaxanthins and/or the glycosylated betalains may be obtained by contacting the enzyme or enzymes with cells secreting the substrates. In some embodiment, the method is performed in vivo.

The first heterologous enzyme (TYH) may be as defined in the section “CYP76AD (TYH)”. The second heterologous enzyme (DOD or DOD*) may be as defined in the section “4,5-DOPA extradiol dioxygenase”. The third heterologous enzyme (Enzyme having glycosyltransferase activity) may be as defined in the section “Enzyme having glycosyltransferase activity”.

Also provided herein is a kit of parts comprising:
a. the yeast cell as presented herein; and/or
b. the nucleic acid system as presented herein, wherein said construct is for modifying a yeast cell; and
c. instructions for use; and
d. optionally, the yeast cell to be modified.

Further provided herein is the use of betalains obtained by the methods disclosed herein as natural food dyes.

Also provided herein is a method for colouring foodstuff, comprising producing betalains according to the methods presented herein, and adding or mixing them with the foodstuff to be coloured.

Betalains Betalains are water-soluble, tyrosine-derived pigments, in which betalamic acid is the central chromophore. Betalains can be divided into two groups of compounds; betacyanins, which are red to violet pigments derived by condensation of betalamic acid with cyclo-dihydroxyphenylalanine (cyclo-DOPA); and betaxanthins, which are yellow to orange pigments derived from betalamic acid via conjugation with different amines and amino acids. Betacyanins have an absorbance spectrum with a maximal wavelength centred at 536 nm, while betaxanthins have a maximal wavelength centred at 480 nm.

The first committed step in the betalain biosynthesis pathway is a tyrosine hydroxylase reaction, where L-tyrosine is converted to L-3,4-dehydroxyphenylalanine (L-DOPA). L-DOPA may be converted to L-dopaquinone via oxidation, and further converted into cyclo-DOPA through spontaneous cyclization. All these reactions may be catalysed by the P450 cytochrome enzyme CYP76AD (cytochrome P450 76AD), such as by CYP76ADαβ. Throughout this document, CYP76ADs, hereunder CYP76Adαβs, are termed as TYHs. cyclo-DOPA may be glycosylated by an enzyme with cDOPA50GT activity to form cyclo-DOPA-5-O-glucoside (cDOPA50G) cDOPA50G may spontaneously react with betalamic acid to form betanin. Alternatively, cyclo-DOPA may converted into betanidin via spontaneous reaction with betalamic acid.

L-DOPA may alternatively be converted into 4,5-seco-DOPA in a reaction catalysed by a 4,5-DOPA extradiol dioxygenase (DOD). 4,5-seco-DOPA may be further converted into betalamic acid through spontaneous cyclization. Betalamic acid may be further converted into a betaxanthin though spontaneous reaction with an amino or an amine group. Alternatively, betalamic acid may spontaneously be converted into betanidin via spontaneous reaction with cyclo-DOPA.

Betanidin may be converted into betanin and/or isobetanin in a reaction catalysed by an enzyme with B50G activity.

Thus, provided herein is a betalain, such as a betacyanin such as betanidin, betanin or isobetanin, or a betaxanthin obtainable by the methods presented herein.

Further provided herein is the use of a betalain, such as a betacyanin such as betanidin, betanin or isobetanin, or a betaxanthin obtainable by the methods presented herein.

In some embodiments, the betalain is a glycosylated betalain, such as a glycosylated betacyanin. In some embodiments, the glycosylated betalain is selected from the group of glycosylated betalains consisting of betanin, isobetanin, 2-descarboxy-betanin, 6′-O-malonyl-2-descarboxy-betanin, 2′-O-aposyl-betanin, phyllocactin, apiosyl-phyllocactin, feruloyl, malonyl-betanin, hylocerenin, lampranthin I, lampranthin II, prebetanin, rivinianin, neobetanin, amaranthin, iresinin, celosianin I, celosianin II, sinapoyl-amaranthin, gomphrenin I, gomphrenin II, gomphreninn III, gomphrenin IV, bougainvillein-r I, bougainvillein-r II, feruloyl-bougainvillein-r I, bougainvillein-v, mammillarinin, 4′-O-malonyl-bougainvillein-r I, 2-descarbody-mammillarinin, p-coumaroyl-glucosyl-bougainvillein-v, caffeoyl-p-coumaroyl-glucosyl-bougainvillein-v, and caffeoyl-p-coumaroyl-sophorosyl-bougainvillein-v. In a preferred embodiment, the betacyanin is betanin and/or isobetanin.

In some embodiments, the betalain is a betaxanthin. In some embodiments, the betaxanthin is selected from the group of betaxanthins consisting of betalamic acid, indicaxanthin, portulacaxanthin I, portulacaxanthin II, portulacaxanthin III, vulgaxanthin I, vulgaxanthin II, vulgaxanthin III, vulgaxanthin IV, miraxanthin I, miraxanthin II, miraxanthin III, miraxanthin IV, miraxanthin V, histamine-betaxanthin, dopaxanthin, humilixanthin, γ-aminobutyric acid (GABA)-betaxanthin, methylated arginine-betaxanthin, seine-betaxanthin, tryptophan-betaxanthin, valine-betaxanthin, phenylalanine-betaxanthin, isoleucine-betaxanthin, alanine-betaxanthin, histidine-betaxanthin, methionine-betaxanthin, threonine-betaxanthin, arginine-betaxanthin, lysine-betaxanthin, ethanolamine-betaxanthin and putrescine-betaxanthin.

In other words, in some embodiments of the present inventions, the betalain is a glycosylated betalain, wherein said betalain is glycosylated at at least one position, such as at least two positions, such as at least three positions, such as at least four positions, such as at least five positions.

### Yeast Cell

The yeast cell may be any type of yeast cell. In some embodiments, the genus of the yeast cell is selected from the group consisting of Saccharomyces, Pichia, Yarrowia, Kluyveromyces, Candida, Rhodotorula, Rhodosporidium, Cryptococcus, Trichosporon and Lipomyces, such as Saccharomyces cerevisiae, Saccharomyces boulardi, Candida tropicalis, Pichia pastoris, Kluyveromyces marxianus, Cryptococcus albidus, Lipomyces lipofera, Lipomyces starkeyi, Rhodosporidium toruloides, Rhodotorula glutinis, Trichosporon pullulan and Yarrowia lipolytica. In a preferred embodiment, the genus is Saccharomyces or Yarrowia, most preferably the genus is Yarrowia.

The yeast cell to be modified, which will also be referred to as the host cell, may express native enzymes which are of the same or of a different class as the enzymes which are necessary for the production of betalains. In some cases, however, such native enzymes may have a negative impact on the titer of betalains which can be obtained; the native enzymes may thus be inactivated by methods known in the art, such as gene editing. For example, the genes encoding the native enzymes having a negative impact on the titer may be deleted or mutated so as to lead to total or partial loss of activity of the native enzyme.

During production of betalains in the yeast cell, byproducts, i.e. side-products, may form. Such byproducts may for example include other pigments, such as brown pigments. For example, during prolonged cultivation of Yarrowia lipolytica, melanins, such as allomelanins—and hereunder pyomelanin, may be accumulated in the growth medium.

The inventors have discovered that production of melanins in the yeast cell interefers with the production and/or extraction of betalains, such as the production and/or extraction of betanin and/or isobetanin. In particular, the inventors have discovered that decreasing the biosynthesis of melanins may have a positive impact on the betalain titer.

Thus, in some embodiments, the yeast cell has been modified for decreased production of byproducts i.e. decreased formation of side-products. In other words, the yeast cell has one or more mutations in genes involved in byproduct formation, such as in one or more genes encoding for one or more proteins which are involved in catalysing the formation of byproducts, wherein such mutations lead to partial or total loss of activity of said protein(s). In other words, said yeast cell having said mutation produces less or no byproducts. Thus, in said yeast cell, less or no products are produced in processes which are competitive with that of betalain production.

In some embodiments, the yeast cell has a mutation resulting in reduced activity of one or more genes involved in the biosynthesis of melanins. In a preferred embodiment, the yeast cell has a mutation resulting in reduced activity of 4-hydroxyphenylpyruvate dioxygenase (4-HPPD). Preferably, the yeast cell has a mutation in the gene encoding for 4-HPPD, such as a mutation leading to partial or total loss of activity of 4-HPPD. In one embodiment, the yeast cell is a Yarrowia lipolytica yeast cell and the 4-HPPD is a Yarrowia lipolytica 4-HPPD (SEQ ID NO: 69). A mutation resulting in reduced activity of 4-HPPD could be an insertion, for example an insertion resulting in a frameshift; a deletion, whether partial or total; a substitution, which could for instance disrupt the tertiary structure of the enzyme; whereby 4-HPPD is no longer expressed or is no longer functional. The non-functionality or reduced activity of 4-HPPD may for example be confirmed by the reduced formation of melanins or melanins' precursors, such as homogentisic acid.

In some embodiments, the yeast cell has been modified to express the TYH, DOD and/or DOD* and/or the enzyme having glycosyltransferase activity, such as the glycosyltransferase, such as the SGT, at the genomic level, e.g. by gene editing in the genome. The yeast cell may also be modified by insertion of at least one nucleic acid construct such as at least one vector, for example a plasmid, or by introduction in the cell of a system comprising several nucleic acids as detailed herein below. The vector may be designed as is known to the skilled person to either enable integration of nucleic acid sequences in the genome, or to enable expression of a polypeptide encoded by a nucleic acid sequence comprised in the vector without genome integration.

In some embodiments, the genes encoding said TYH, DOD and/or enzyme having glycosyltransferase activity, such as the glycosyltransferse, such as the SGT, have been codon optimized for said yeast cell. In other embodiments, the genes encoding said TYH, DOD and/or enzyme having glycosyltransferase activity, such as the glycosyltransferse, such as the SGT, are under control of an inducible promoter.

In some embodiments, the genes encoding said TYH, DOD and/or enzyme having glycosyltransferase activity, such as the glycosyltransferse, such as the SGT, are present in high copy number, and/or they are each independently comprised within the genome of the yeast cell or within a vector comprised in the yeast cell.

In some embodiments, at least one of the genes encoding the TYH, the DOD and/or the enzyme having glycosyltransferase activity is present in at least two copies, such as at least three copies, such as at least four copies, such as at least five copies. In one embodiment, the gene encoding the TYH is present in at least two copies, such as at least three copies, such as at least four copies, such as at least five copies. In one embodiment, the gene encoding the DOD is present in at least two copies, such as at least three copies, such as at least four copies, such as at least five copies. In one embodiment, the gene encoding the enzyme having glycosyltransferase activity is present in at least two copies, such as at least three copies, such as at least four copies, such as at least five copies.

In other words, in some embodiments, the yeast cell expresses at least two different enzymes having glycosyltransferase activity, such as at least three different enzymes having glycosyltransferase activity, such as at least four different enzymes having glycosyltransferase activity. Thus, the yeast cell may for example express a Chenopodium quinoa glycosyltransferase, such as the Chenopodium quinoa glycosyltransferase set forth in SEQ ID NO: 65 (CqSGT2), and a Beta vulgaris glycosyltransferase, such as the Beta vulgaris glycosyltransferase set forth in SEQ ID NO: 53 (BvSGT2).

In some embodiments, the yeast cell has been modified to produce high amounts of L-tyrosine. In some embodiments, the yeast cell has a mutation in at least one of the genes involved in L-tyrosine biosynthesis. For example, the yeast cell has one or more point mutation(s) in one or more enzyme(s) involved in L-tyrosine biosynthesis. In one embodiment, said one or more point mutation(s) results in said enzyme(s) being less sensitive to feedback inhibition by aromatic amino acids. In other words, said one or more enzyme(s) with said one or more point mutation(s) are not inhibited, or inhibited to a lesser degree than its native counterpart having no point mutation(s), by aromatic amino acids, such as by amino acids L-tyrosine, L-phenylalanine and/or L-tryptophan.

In one embodiment, the yeast cell has a point mutation in 3-deoxy-7-phosphoheptulonate synthase (Aro4). In one embodiment, the yeast cell has a point mutation in Yarrowia lipolytica Aro4 (SEQ ID NO: 71), such as a point mutation in the wild-type Aro4 from Yarrowia lipolytica, such as wherein amino acid no. 221 of Yarrowia lipolytica Aro4 is substituted with leucine. In another embodiment, the yeast cell has a point mutation in Saccharomyces cerevisiae Aro4 (SEQ ID NO: 75), such as a point mutation in the wild-type Aro4 from Saccharomyces cerevisiae, such wherein amino acid no. 229 of Saccharomyces cerevisiae Aro4 is substituted with leucine.

In yet another embodiment, the yeast cell has a point mutation in chorismate mutase (Aro7). In one embodiment, the yeast cell has a point mutation in Yarrowia lipolytica Aro7 (SEQ ID NO: 73), such as a point mutation in the wild-type Aro4 from Yarrowia lipolytica, such wherein amino acid no. 139 of Yarrowia lipolytica Aro4 is substituted with serine. In another embodiment, the yeast cell has a point mutation in Saccharomyces cerevisiae Aro7 (SEQ ID NO: 77), such as a point mutation in the wild-type Aro7 from Saccharomyces cerevisiae, such wherein amino acid no. 141 of Saccharomyces cerevisiae Aro7 is substituted with serine.

In some embodiments, the yeast cell comprises a system of vectors, as described in the section “Nucleic acid”.

### CYP76AD (TYH)

In the present invention, TYHs refers to CYP76AD enzymes with tyrosine hydroxylase activity. The term ‘CYP76AD’ and ‘TYH’ will be used herein interchangeably. The term ‘heterologous TYH’ refers to a TYH which is not naturally expressed by the organism, such as by the yeast cell.

The TYHs presented herein catalyse the following reactions:
L-tyrosine→L-DOPA
L-DOPA→L-dopaquinone

The EC number for the overall reaction is EC 1.14.18.1.

L-dopaquinone subsequentially cyclizes to form cyclo-DOPA in a spontaneous reaction.

In some embodiments of the present invention the TYH is native to a plant, such as of the genus Abronia, Acleisanthes, Basella, Beta, Cleretum, Ercilla, Mirabilis, Optunia, or Phytolacca, such as Abronia nealleyi, Acleisanthes obtusa, Basella alba, Beta vulgaris, Cleretum bellidiforme, Ercilla volubis, Mirabilis multiflora, Optunia ficus-indica, or Phytolacca dioica, or a functional variant thereof having at least 80% identity thereto, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% identity to said TYH.

In some embodiments, the TYH is a TYH selected from the group of TYH set forth in SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, and SEQ ID NO: 47, or functional variants thereof having at least 60% identity thereto, such as at least 61% identity, such as at least 62% identity, such as at least 63% identity, such as at least 64% identity, such as at least 65% identity, such as at least 66% identity, such as at least 67% identity, such as at least 68% identity, such as at least 69% identity, such as at least 70% identity, such as at least 71% identity, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% identity to a TYH selected from the group of TYHs as set forth in SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, and SEQ ID NO: 47.

In one embodiment, the heterologous TYH is an Abronia TYH. In one embodiment, the TYH is an Abronia nealleyi TYH, such as the TYH as set forth in SEQ ID NO: 37 (AnTYH). In some embodiments, the TYH is a functional variant of an Abronia TYH, a functional variant of an Abronia nealleyi TYH or a functional variant of the TYH as set forth in SEQ ID NO: 37 (AnTYH), having at least 60% identity thereto.

In one embodiment, the heterologous TYH is an Acleisanthes TYH. In one embodiment, the TYH is an Acleisanthes obtusa TYH, such as the TYH as set forth in SEQ ID NO: 39 (AoTYH). In some embodiments, the TYH is a functional variant of an Acleisanthes TYH, a functional variant of an Acleisanthes obtusa TYH or a functional variant of the TYH as set forth in SEQ ID NO: 39 (AoTYH), having at least 60% identity thereto.

In one embodiment, the heterologous TYH is a Basella TYH. In one embodiment, the TYH is a Basella alba TYH, such as the TYH as set forth in SEQ ID NO: 29 (BaTYH). In some embodiments, the TYH is a functional variant of a Basella TYH, a functional variant of a Basella alba TYH or a functional variant of the TYH as set forth in SEQ ID NO: 29 (BaTYH), having at least 60% identity thereto.

In one embodiment, the heterologous TYH is a Beta TYH. In one embodiment, the TYH is a Beta vulgaris TYH, such as the TYH as set forth in SEQ ID NO: 27 (BvCYP76AD^{W13L}). In some embodiments, the TYH is a functional variant of a Beta TYH, a functional variant of a Beta vulgaris TYH or a functional variant of the TYH as set forth in SEQ ID NO: 27 (BvCYP76AD^{W13L}), having at least 60% identity thereto.

In one embodiment, the heterologous TYH is a Cleretum TYH. In one embodiment, the TYH is a Cleretum bellidiforme TYH, such as the TYH as set forth in SEQ ID NO: 31 (CbTYH). In some embodiments, the TYH is a functional variant of a Cleretum TYH, a functional variant of a Cleretum bellidiforme TYH or a functional variant of the TYH as set forth in SEQ ID NO: 31 (CbTYH), having at least 60% identity thereto.

In one embodiment, the heterologous TYH is an Ercilla TYH. In one embodiment, the TYH is an Ercilla volubis TYH, such as the TYH as set forth in SEQ ID NO: 43 (EvTYH). In some embodiments, the TYH is a functional variant of an Ercilla TYH, a functional variant of an Ercilla volubis TYH or a functional variant of the TYH as set forth in SEQ ID NO: 43 (EvTYH), having at least 60% identity thereto.

In one embodiment, the heterologous TYH is a Mirabilis TYH. In one embodiment, the TYH is a Mirabilis multiflora TYH, such as the TYH as set forth in SEQ ID NO: 41 (MmTYH1) or the TYH as set forth in SEQ ID NO: 47 (MmTYH2). In some embodiments, the TYH is a functional variant of a Mirabilis multiflora TYH, a functional variant of a Mirabilis TYH or a functional variant of the TYH as set forth in SEQ ID NO: 41 (MmTYH1) or SEQ ID NO: 47 (MmTYH2), having at least 60% identity thereto.

In one embodiment, the heterologous TYH is an Opuntia TYH. In one embodiment, the TYH is an Opuntia ficus-indica TYH, such as the TYH as set forth in SEQ ID NO: 35 (OfTYH). In some embodiments, the TYH is a functional variant of an Opuntia TYH, a functional variant of an Opuntia ficus-indica TYH or a functional variant of the TYH as set forth in SEQ ID NO: 35 (OfTYH), having at least 60% identity thereto.

In one embodiment, the heterologous TYH is a Phytolacca TYH. In one embodiment, the TYH is a Phytolacca americana TYH, such as the TYH as set forth in SEQ ID NO: 33 (PaTYH), or a Phytolacca dioica TYH, such as the TYH set forth in SEQ ID NO: 45 (PdTYH). In some embodiments, the TYH is a functional variant of a Phytolacca TYH, a functional variant of a Phytolacca americana TYH, a functional variant of a Phytolacca dioica TYH, or a functional variant of the TYH as set forth in SEQ ID NO: 33 (PaTYH) or SEQ ID NO: 45 (PdTYH), having at least 60% identity thereto.

A functional variant of a TYH refers to a variant of a TYH, which retains at least some or all of the TYH activity, and which has at least 60% identity, such as at least 61% identity, such as at least 62% identity, such as at least 63% identity, such as at least 64% identity, such as at least 65% identity, such as at least 66% identity, such as at least 67% identity, such as at least 68% identity, such as at least 69% identity, such as at least 70% identity, such as at least 71% identity, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% identity thereto.

A functional variant of a TYH refers to a variant of TYH which retains at least some of the activity of the parent enzyme. Thus, a functional variant of a TYH can catalyze the same conversion as the TYH from which it is derived, although the efficiency of the reaction may be different, e.g. the efficiency may be decreased or increased compared to the parent enzyme. Testing whether or not an enzyme is a functional variant of a TYH can be tested using methods known in the art. For example, the TYH variant can be expressed in a cell, wherein the cell medium contains the substrate of TYH, i.e. L-tyrosine, or said substrate is produced in said cell. After incubating the cell for 24 hours, the amount of product, i.e. the amount of L-DOPA and/or L-dopaquinone, generated by the cell, i.e. by the TYH variant, can be measured. If the TYH variant generates the same product, i.e. L-DOPA and/or L-dopaquinone, as the TYH does, if said TYH is tested under the same conditions, i.e. expressed in a cell and incubated for 24 h, the TYH variant is a functional variant of said TYH.

### 4,5-DOPA Extradiol Dioxygenase (DOD)

The term ‘4,5-DOPA extradiol dioxygenase’ and ‘DOD’ will be used herein interchangeably. The term ‘heterologous DOD’ refers to a DOD which is not naturally expressed by the yeast cell. A DOD as presented herein is an enzyme catalysing the following reaction:

L-DOPA→4,5-seco-DOPA

The EC number for the reaction is EC 1.13.11.29.

4,5-seco-DOPA subsequentially spontaneously cyclizes to form betalamic acid.

In some embodiments of the present invention, the DOD is native to a plant, such as of the genus Amaranthus, Beta, Bougainvillea, Mirabilis Phytolacca, Portulaca, Spinacia, or Suaeda, such as Amaranthus hypochondriacus, Amaranthus tricolour, Beta vulgaris, Bougainvillea glabra, Mirabilis jalapa, Phytolacca americana, Portulaca grandiflora, Spinacia oleracea, or Suaeda salsa, or a functional variant thereof having at least 80% identity thereto.

In some embodiments, the DOD is a DOD selected from the group of DOD set forth in SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, and SEQ ID NO: 25, or functional variants thereof having at least 60% identity thereto, such as at least 61% identity, such as at least 62% identity, such as at least 63% identity, such as at least 64% identity, such as at least 65% identity, such as at least 66% identity, such as at least 67% identity, such as at least 68% identity, such as at least 69% identity, such as at least 70% identity, such as at least 71% identity, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% identity a desaturase selected from the group of desaturases set forth in SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, and SEQ ID NO: 25.

In one embodiment, the heterologous DOD is an Amaranthus DOD. In one embodiment, the DOD is an Amaranthus tricolour DOD, such as the DOD as set forth in SEQ ID NO: 13 (AtDOD). In one embodiment, the DOD is an Amaranthus hypochondriacus DOD, such as the DOD as set forth in SEQ ID NO: 15 (AhDOD). In some embodiments, the DOD is a functional variant of an Amaranthus DOD, a functional variant of an Amaranthus tricolour DOD, a functional variant of an Amaranthus hypochondriacus DOD, a functional variant of the DOD as set forth in SEQ ID NO: 13 (AtDOD), or a functional variant of the DOD as set forth in SEQ ID NO: 15 (AhDOD), having at least 60% identity thereto.

In one embodiment, the heterologous DOD is a Beta DOD. In one embodiment, the DOD is a Beta vulgaris DOD, such as the DOD as set forth in SEQ ID NO: 3 (BvDOD), the DOD as set forth in SEQ ID NO: 23 (BvDOD2), or the DOD as set forth in SEQ ID NO: 25 (BvDOD3). In some embodiments, the DOD is a functional variant of a Beta DOD, a functional variant of a Beta vulgaris DOD, a v functional variant of the DOD as set forth in SEQ ID NO: 3 (BvDOD), a functional variant of the DOD as set forth in SEQ ID NO: 23 (BvDOD2) or a functional variant of the DOD as set forth in SEQ ID NO: 25 (BvDOD3), having at least 60% identity thereto.

In one embodiment, the heterologous DOD is a Bougainvillea DOD. In one embodiment, the DOD is a Bougainvillea glabra DOD, such as the DOD as set forth in SEQ ID NO: 5 (BgDOD1) or the DOD as set forth in SEQ ID NO: 21 (BgDOD2). In some embodiments, the DOD is a functional variant of a Bougainvillea DOD, a functional variant of a Bougainvillea glabra DOD, a functional variant of the DOD as set forth in SEQ ID NO: 5 (BgDOD1), or a functional variant of the DOD as set forth in SEQ ID NO: 21 (BgDOD2), having at least 60% identity thereto

In one embodiment, the heterologous DOD is a Mirabilis DOD. In one embodiment, the DOD is a Mirabilis jalapa DOD, such as the DOD as set forth in SEQ ID NO: 1 (MjDOD). In some embodiments, the DOD is a functional variant of a Mirabilis DOD, a functional variant of a Mirabilis jalapa DOD or a functional variant of the DOD as set forth in SEQ ID NO: 1 (MjDOD), having at least 60% identity thereto.

In one embodiment, the heterologous DOD is a Phytolacca DOD. In one embodiment, the DOD is a Phytolacca americana DOD, such as the DOD as set forth in SEQ ID NO: 17 (PaDOD). In some embodiments, the DOD is a functional variant of a Phytolacca DOD, a functional variant of a Phytolacca americana DOD or a functional variant of the DOD as set forth in SEQ ID NO: 17 (PaDOD), having at least 60% identity thereto.

In one embodiment, the heterologous DOD is a Portulaca DOD. In one embodiment, the DOD is a Portulaca grandiflora DOD, such as the DOD as set forth in SEQ ID NO: 7 (PgDOD). In some embodiments, the DOD is a functional variant of a Portulaca DOD, a functional variant of a Portulaca grandiflora DOD or a functional variant of the DOD as set forth in SEQ ID NO: 7 (PgDOD), having at least 60% identity thereto.

In one embodiment, the heterologous DOD is a Spinacia DOD. In one embodiment, the DOD is a Spinacia oleracea DOD, such as the DOD as set forth in SEQ ID NO: 11 (SoDOD). In some embodiments, the DOD is a functional variant of a Spinacia DOD, a functional variant of a Spinacia oleracea DOD or a functional variant of the DOD as set forth in SEQ ID NO: 11 (SoDOD), having at least 60% identity thereto.

In one embodiment, the heterologous DOD is a Suaeda DOD. In one embodiment, the DOD is a Suaeda salsa DOD, such as the DOD as set forth in SEQ ID NO: 19 (SsDOD). In some embodiments, the DOD is a v functional variant of a Suaeda DOD, a functional variant of a Suaeda salsa DOD or a functional variant of the DOD as set forth in SEQ ID NO: 19 (SsDOD), having at least 60% identity thereto.

A functional variant of a DOD refers to a variant of a DOD, which retains at least some or all of the DOD activity, and which has at least 60% identity, such as at least 61% identity, such as at least 62% identity, such as at least 63% identity, such as at least 64% identity, such as at least 65% identity, such as at least 66% identity, such as at least 67% identity, such as at least 68% identity, such as at least 69% identity, such as at least 70% identity, such as at least 71% identity, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% identity thereto.

Further provided herein is a DOD variant (DOD*) which is a DOD truncation mutant having a truncation of at least 5 amino acids at the C-terminal end, such as at least 6, such as at least 7, such as at least 8, such as at least 9, such as at least 10, such as at least 12, such as at least 14, such as at least 16, such as at least 18, such as at least 20, such as at least 25, such as at least 30, such as at least 35, such as at least 40, such as at least 45, such as at least 50 amino acids at the C-terminal end. In one embodiment, the DOD* has a mutation resulting in an early stop codon.

Also provided herein is the use of a DOD variant (DOD*) to catalyse the conversion of L-DOPA to 4,5-seco-DOPA.

In some embodiments, the DOD* is derived from a DOD which is native to a plant, such as of the genus Amaranthus, Beta, Bougainvillea, Mirabilis Phytolacca, Portulaca, Spinacia, or Suaeda, such as Amaranthus hypochondriacus, Amaranthus tricolour, Beta vulgaris, Bougainvillea glabra, Mirabilis jalapa, Phytolacca americana, Portulaca grandiflora, Spinacia oleracea, or Suaeda salsa, or a functional variant thereof having at least 80% identity thereto.

In one embodiment, the DOD* is a truncation of an Amaranthus DOD. In one embodiment, the DOD* is a truncation of an Amaranthus tricolour DOD, such a truncation of as the DOD as set forth in SEQ ID NO: 13 (AtDOD). In one embodiment, the DOD* is a truncation of an Amaranthus hypochondriacus DOD, such as a truncation of the DOD as set forth in SEQ ID NO: 15 (AhDOD). In some embodiments, the DOD* is a truncation of a functional variant of an Amaranthus DOD, a truncation of a functional variant of an Amaranthus tricolour DOD, a truncation of a functional variant of an Amaranthus hypochondriacus DOD, a truncation of a functional variant of the DOD as set forth in SEQ ID NO: 13 (AtDOD), or a truncation of a functional variant of the DOD as set forth in SEQ ID NO: 15 (AhDOD), having at least 60% identity thereto.

In one embodiment, the DOD* is a truncation of a Beta DOD. In one embodiment, the DOD* is a truncation of a Beta vulgaris DOD, such as a truncation of the DOD as set forth in SEQ ID NO: 3 (BvDOD), a truncation of the DOD as set forth in SEQ ID NO: 23 (BvDOD2), or a truncation of the DOD as set forth in SEQ ID NO: 25 (BvDOD3). In some embodiments, the DOD* is a truncation of a functional variant of a Beta DOD, a truncation of a functional variant of a Beta vulgaris DOD, a truncation of a functional variant of the DOD as set forth in SEQ ID NO: 3 (BvDOD), a truncation of a functional variant of the DOD as set forth in SEQ ID NO: 23 (BvDOD2), or a truncation of a functional variant of the DOD as set forth in SEQ ID NO: 25 (BvDOD3), having at least 60% identity thereto.

In one embodiment, the DOD* is truncation of a Bougainvillea DOD. In one embodiment, the DOD* is a truncation of a Bougainvillea glabra DOD, such as a truncation of the DOD as set forth in SEQ ID NO: 5 (BgDOD1) or a truncation of the DOD as set forth in SEQ ID NO: 21 (BgDOD2). In some embodiments, the DOD* is a truncation of a functional variant of a Bougainvillea DOD, a truncation of a functional variant of a Bougainvillea glabra DOD, a truncation of a functional variant of the DOD as set forth in SEQ ID NO: 5 (BgDOD1), or a truncation of a functional variant of the DOD as set forth in SEQ ID NO: 21 (BgDOD2), having at least 60% identity thereto.

In one embodiment, the DOD* is a truncation of a Mirabilis DOD. In one embodiment, the DOD* is a truncation of a Mirabilis jalapa DOD, such as a truncation of the DOD as set forth in SEQ ID NO: 1 (MjDOD). In some embodiments, the DOD* is a truncation of a functional variant of a Mirabilis DOD*, a truncation of a functional variant of a Mirabilis jalapa DOD or a truncation of a functional variant of the DOD as set forth in SEQ ID NO: 1 (MjDOD), having at least 60% identity thereto.

In one embodiment, the DOD* is a truncation of a Phytolacca DOD. In one embodiment, the DOD* is a truncation of a Phytolacca americana DOD, such as a functional truncation of the DOD as set forth in SEQ ID NO: 17 (PaDOD). In some embodiments, the DOD* is a truncation of a functional variant of a Phytolacca DOD, a truncation of a functional variant of a Phytolacca americana DOD or a truncation of a functional variant of the DOD as set forth in SEQ ID NO: 17 (PaDOD), having at least 60% identity thereto.

In one embodiment, the DOD* is a truncation of a Portulaca DOD. In one embodiment, the DOD* is a truncation of a Portulaca grandiflora DOD, such as a truncation of the DOD as set forth in SEQ ID NO: 7 (PgDOD), such as the DOD* as set forth in SEQ ID NO: 9 (PgDOD*). In some embodiments, the DOD* is a truncation of a functional variant of a Portulaca DOD, a truncation of a functional variant of a Portulaca grandiflora DOD, a truncation of a functional variant of the DOD as set forth in SEQ ID NO: 7 (PgDOD), or a truncated DOD as set forth in SEQ ID NO: 9 (PgDOD*), having at least 60% identity thereto.

In one embodiment, the DOD* is a truncation of a Spinacia DOD. In one embodiment, the DOD* is a truncation of a Spinacia oleracea DOD, such as a truncation of the DOD as set forth in SEQ ID NO: 11 (SoDOD). In some embodiments, the DOD* is a truncation of a functional variant of a Spinacia DOD, a truncation of a functional variant of a Spinacia oleracea DOD or a truncation of a functional variant of the DOD as set forth in SEQ ID NO: 11 (SoDOD), having at least 60% identity thereto.

In one embodiment, the DOD* is truncation of a Suaeda DOD. In one embodiment, the DOD* is a truncation of a Suaeda salsa DOD, such as a truncation of the DOD as set forth in SEQ ID NO: 19 (SsDOD). In some embodiments, the DOD* is a truncation of a functional variant of a Suaeda DOD, a truncation of a functional variant of a Suaeda salsa DOD or truncation of a functional variant of the DOD as set forth in SEQ ID NO: 19 (SsDOD), having at least 60% identity thereto.

The truncated DOD, or DOD*, described herein, retain at least some of the activity of the parent DOD from which they are derived.

A functional variant of a DOD* refers to a variant of a DOD*, which retains at least some or all of the DOD* activity, and which has at least 60% identity, such as at least 61% identity, such as at least 62% identity, such as at least 63% identity, such as at least 64% identity, such as at least 65% identity, such as at least 66% identity, such as at least 67% identity, such as at least 68% identity, such as at least 69% identity, such as at least 70% identity, such as at least 71% identity, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% identity thereto.

Any of the above DOD*s can be expressed in the cell together with any combination of TYHs and/or SGTs described herein.

In some embodiments, the cell expresses a DOD*, for example PgDOD* (SEQ ID NO: 9); and:
a TYH selected from: a Beta TYH, such as an Beta vulgaris TYH, for example BvCYP76AD^{W13L} as set forth in SEQ ID NO: 27; a Basella TYH, such as an Basella alba TYH, for example BaTYH as set forth in SEQ ID NO: 29; a Cleretum TYH, such as a Cleretum bellidiforme TYH, for example CbTYH as set forth in SEQ ID NO: 31; a Phytolacca TYH, such as a Phytolacca americana TYH, for example PaTYH as set forth in SEQ ID NO: 33, or a Phytolacca dioica TYH, for example PdTYH as set forth in SEQ ID NO: 45; an Optunia TYH, such as an Optunia ficus-indicaTYH, for example OfTYH as set forth in SEQ ID NO: 35; an Abronia TYH, such as an Abronia nealleyi TYH, for example AnTYH as set forth in SEQ ID NO: 37; a Acleisanthes TYH, such as a Acleisanthes obtusa TYH, for example AoTYH as set forth in SEQ ID NO: 39; a Mirabilis TYH, such as a Mirabilis multiflora TYH, for example MmTYH1 as set forth in SEQ ID NO: 41 or MmTYH2 as set forth in SEQ ID NO: 47; or an Ercilla TYH, such as an Ercilla volubilis TYH, for example EvTYH as set forth in SEQ ID NO: 43;
   or functional variants thereof having at least 60% identity thereto.

In some embodiments, the cell expresses a DOD*, for example PgDOD* (SEQ ID NO: 9); and:
a TYH selected from: a Beta TYH, such as an Beta vulgaris TYH, for example BvCYP76AD^{W13L} as set forth in SEQ ID NO: 27; a Basella TYH, such as an Basella alba TYH, for example BaTYH as set forth in SEQ ID NO: 29; a Cleretum TYH, such as a Cleretum bellidiforme TYH, for example CbTYH as set forth in SEQ ID NO: 31; a Phytolacca TYH, such as a Phytolacca americana TYH, for example PaTYH as set forth in SEQ ID NO: 33, or a Phytolacca dioica TYH, for example PdTYH as set forth in SEQ ID NO: 45; an Optunia TYH, such as an Optunia ficus-indicaTYH, for example OfTYH as set forth in SEQ ID NO: 35; an Abronia TYH, such as an Abronia nealleyi TYH, for example AnTYH as set forth in SEQ ID NO: 37; a Acleisanthes TYH, such as a Acleisanthes obtusa TYH, for example AoTYH as set forth in SEQ ID NO: 39; a Mirabilis TYH, such as a Mirabilis multiflora TYH, for example MmTYH1 as set forth in SEQ ID NO: 41 or MmTYH2 as set forth in SEQ ID NO: 47; or an Ercilla TYH, such as an Ercilla volubilis TYH, for example EvTYH as set forth in SEQ ID NO: 43; and
an SGT selected from: a Beta SGT, such as a Beta vulgaris SGT, for example BvSGT1 as set forth in SEQ ID NO: 51, BvSGT2 as set forth in SEQ ID NO: 53, BvSGT3 as set forth in SEQ ID NO: 55, or BvSGT4 as set forth in SEQ ID NO: 57;
   or functional variants thereof having at least 60% identity thereto.

The term “functional variant having at least 60% identity” in relation to a given enzyme shall be understood to refer to functional variants having 60% identity or more to said enzyme, such as at least 61% identity, such as at least 62% identity, such as at least 63% identity, such as at least 64% identity, such as at least 65% identity, such as at least 66% identity, such as at least 67% identity, such as at least 68% identity, such as at least 69% identity, such as at least 70% identity, such as at least 71% identity, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% identity to the enzyme, or more.

A functional variant of a DOD refers to a variant of DOD which retains at least some of the activity of the parent enzyme. Thus, a functional variant of a DOD can catalyze the same conversion as the DOD from which it is derived, although the efficiency of the reaction may be different, e.g. the efficiency may be decreased or increased compared to the parent enzyme. Testing whether or not an enzyme is a functional variant of a DOD can be tested using methods known in the art. For example, the DOD variant can be expressed in a cell, wherein the cell medium contains the substrate of DOD, i.e. L-tyrosine, or said substrate is produced in said cell. After incubating the cell for 24 hours, the amount of product, i.e. the amount of L-DOPA and/or L-dopaquinone, generated by the cell, i.e. by the DOD variant, can be measured. If the DOD variant generates the same product, i.e. L-DOPA and/or L-dopaquinone, as the DOD does, if said DOD is tested under the same conditions, i.e. expressed in a cell and incubated for 24 h, the DOD variant is a functional variant of said DOD.

The cell may further express an enzyme having glycosyltransferase activity as described herein below.

### Enzyme Having Glycosyltransferase Activity

The term ‘enzyme having glycosyltransferase activity’, ‘glycosyltransferase’, ‘scopoletin glucosyltransferase’ and ‘SGT’ will be used herein interchangeably. The term ‘heterologous enzyme having glycosyltransferase activity’ refers to an enzyme having glycosyltransferase activity, such as a glycosyltransferase, which is not naturally expressed by the organism, such as by the yeast cell. Glycosyltransferases (EC 2.4) are enzymes that establish natural glycosidic linkages. They catalyze the transfer of saccharide moieties from an activated nucleotide sugar (“the glycosyl donor”) to a glycosyl acceptor molecule:
UDP-sugar substrate (glycosyl donor)+glycosyltransferase substrate (glycosyl acceptor)→UDP+glycosylated glycosyltransferase substrate (EC. 2.4)

In some embodiments, the enzyme having glycosyltransferase activity is a scopoletin glucosyltransferase (SGT), which is an enzyme that catalyses the reaction:
UDP-glucose+scopoletin→UDP+scopolin (EC 2.4.1.128)

The enzyme belongs to the family of glycosyltransferases, specifically hexosyltransferases. The systematic name of this enzyme class is UDP-glucose:scopoletin O-beta-D-glucosyltransferase.

The inventors have surprisingly discovered that certain glycosyltransferases, hereamong certain SGTs, can catalyse glycosylation of betalains and betalain precursors, such as betanidin and cyclo-DOPA, respectively. In other words, the inventors have discovered that certain glycosyltransferases, hereamong certain SGTs, have betanidin-5-O-glucosyltransferase (B50G) activity and cyclo-DOPA-5-O-glucosyltransferase (cDOPA50GT) activity, and that such certain glycosyltransferases, hereamong such certain SGTs can be used for the production of glycosylated betalains.

Hence, provided herein is the use of:
i. Chenopodium quinoa glycosyltransferase CqSGT2 set forth in SEQ ID NO: 65, or a functional variant thereof having at least 70% sequence identity thereto;
ii. Beta vulgaris glycosyltransferase BvSGT2 set forth in SEQ ID NO: 53, or a functional variant thereof having at least 70% identity thereto;
iii. Bougainvillea glabra glycosyltransferase BgGT2 set forth in SEQ ID NO: 67, or a functional variant thereof having at least 70% identity thereto; and/or
iv. Beta vulgaris glycosyltransferase BvSGT4 set forth in SEQ ID NO: 57, or a functional variant thereof having at least 70% identity thereto;
   as a betanidin-5-O-glucosyltransferase (B50G) and/or a cyclo-DOPA 5-O-glucosyltransferase (cDOPA5OGT).

Further provided herein is the use of:
i. Chenopodium quinoa glycosyltransferase CqSGT2 set forth in SEQ ID NO: 65, or a functional variant thereof having at least 70% sequence identity thereto;
ii. Beta vulgaris glycosyltransferase BvSGT2 set forth in SEQ ID NO: 53, or a functional variant thereof having at least 70% identity thereto;
iii. Bougainvillea glabra glycosyltransferase BgGT2 set forth in SEQ ID NO: 67, or a functional variant thereof having at least 70% identity thereto; and/or
iv. Beta vulgaris glycosyltransferase BvSGT4 set forth in SEQ ID NO: 57, or a functional variant thereof having at least 70% identity thereto;
   to catalyse the conversion of cyclo-DOPA to cyclo-DOPA-5-O-glucoside and/or glycosylating betanidin and/or to catalyse the glycosylation of betanidin.

In some embodiments of the present invention the enzyme having glycosyltransferase activity, such as the glycosyltransferase is native to a plant, such as of the genus Abronia, Beta, Bougainvillea, Chenopodium, Ercilla, or Portacula, such as Abronia nealleyi, Beta vulgaris, Bougainvillea glabra, Chenopodium quinoa, Ercilla volubilis, or Portacula grandiflora, or a functional variant thereof having at least 80% identity thereto.

In one embodiment, the heterologous enzyme having glycosyltransferase activity is a Beta glycosyltransferase. In one embodiment, the glycosyltransferase is a Beta vulgaris glycosyltransferase, such as the glycosyltransferase as set forth in SEQ ID NO: 51 (BvSGT1), the glycosyltransferase as set forth in SEQ ID NO: 53 (BvSGT2), the SGT as set forth in SEQ ID NO: 55 (BvSGT3), or the glycosyltransferase as set forth in SEQ ID NO: 57 (BvSGT4). In some embodiments, the glycosyltransferase is a functional variant of a Beta glycosyltransferase, a functional variant of a Beta vulgaris glycosyltransferase or a functional variant of the glycosyltransferase as set forth in SEQ ID NO: 51 (BvSGT1), the glycosyltransferase as set forth in SEQ ID NO: 53 (BvSGT2), the glycosyltransferase as set forth in SEQ ID NO: 55 (BvSGT3), or the glycosyltransferase as set forth in SEQ ID NO: 57 (BvSGT4), having at least 60% identity thereto.

In one embodiment, the heterologous enzyme having glycosyltransferase activity is a Chenopodium glycosyltransferase. In one embodiment, the glycosyltransferase is a Chenopodium quinoa glycosyltransferase, such as the glycosyltransferase as set forth in SEQ ID NO: 65 (CqSGT2). In some embodiments, the glycosyltransferase is a functional variant of a Chenopodium glycosyltransferase, a functional variant of a Chenopodium quinoa glycosyltransferase or a functional variant of the glycosyltransferase as set forth in SEQ ID NO: 67 (CqSGT2), having at least 60% identity thereto.

In one embodiment, the heterologous enzyme having glycosyltransferase activity is a Bougainvillea glycosyltransferase. In one embodiment, the glycosyltransferase is a Bougainvillea glabra glycosyltransferase, such as the glycosyltransferase as set forth in SEQ ID NO: 65 (BgGT2). In some embodiments, the glycosyltransferase is a functional variant of a Bougainvillea glycosyltransferase, a functional variant of a Bougainvillea glabra glycosyltransferase or a functional variant of the glycosyltransferase as set forth in SEQ ID NO: 67 (BgGT2), having at least 60% identity thereto.

A functional variant of a glycosyltransferase refers to a variant of a glycosyltransferase, which retains at least some or all of the glycosyltransferase activity, and which has at least 60% identity, such as at least 61% identity, such as at least 62% identity, such as at least 63% identity, such as at least 64% identity, such as at least 65% identity, such as at least 66% identity, such as at least 67% identity, such as at least 68% identity, such as at least 69% identity, such as at least 70% identity, such as at least 71% identity, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% identity thereto.

Any of the above enzymes having glycosyltransferase activity can be expressed in the cell together with any combination of TYHs and DODs described herein.

In some embodiments, the cell expresses a Beta glycosyltransferase, such as a Beta vulgaris glycosyltransferase, for example BvSGT1 (SEQ ID NO: 51); and one or both of:
a TYH selected from: a Beta TYH, such as an Beta vulgaris TYH, for example BvCYP76AD^{W13L} as set forth in SEQ ID NO: 27; a Basella TYH, such as an Basella alba TYH, for example BaTYH as set forth in SEQ ID NO: 29; a Cleretum TYH, such as a Cleretum bellidiforme TYH, for example CbTYH as set forth in SEQ ID NO: 31; a Phytolacca TYH, such as a Phytolacca americana TYH, for example PaTYH as set forth in SEQ ID NO: 33, or a Phytolacca dioica TYH, for example PdTYH as set forth in SEQ ID NO: 45; an Optunia TYH, such as an Optunia ficus-indicaTYH, for example OfTYH as set forth in SEQ ID NO: 35; an Abronia TYH, such as an Abronia nealleyi TYH, for example AnTYH as set forth in SEQ ID NO: 37; a Acleisanthes TYH, such as a Acleisanthes obtusa TYH, for example AoTYH as set forth in SEQ ID NO: 39; a Mirabilis TYH, such as a Mirabilis multiflora TYH, for example MmTYH1 as set forth in SEQ ID NO: 41 or MmTYH2 as set forth in SEQ ID NO: 47; or an Ercilla TYH, such as an Ercilla volubilis TYH, for example EvTYH as set forth in SEQ ID NO: 43; and
a DOD selected from: an Mirabilis DOD, such as an Mirabilis jalapa DOD, for example MjDOD as set forth in SEQ ID NO: 1; a Beta DOD, such as a Beta vulgaris DOD, for example BvDOD1 as set forth in SEQ ID NO: 3, BvDOD2 as set forth in SEQ ID NO: 23, or BvDOD3 as set forth in SEQ ID NO 25; a Bougainvillea DOD, such as a Bougainvillea glabra DOD, for example BgDOD1 as set forth in SEQ ID NO: 5 or BgDOD2 as set forth in SEQ ID NO: 21; a Portulaca DOD, such as a Portulaca grandiflora DOD, for example PgDOD as set forth in SEQ ID NO: 7; a truncated DOD (DOD*), such as for example PgDOD* as set forth in SEQ ID NO: 9; a Spinacia DOD, such as a Spinacia oleracea DOD, for example SoDOD as set forth in SEQ ID NO: 11; an Amaranthus DOD, such as an Amaranthus tricolour DOD, for example AtDOD as set forth in SEQ ID NO: 13, or such as an Amaranthus hypochondriacus DOD, for example AhDOD as set forth in SEQ ID NO: 15; a Phytolacca DOD, such as a Phytolacca americana DOD, for example PaDOD as set forth in SEQ ID NO: 17; or a Suaeda DOD, such as a Suaeda salsa DOD, for example SsDOD as set forth in SEQ ID NO: 19;
   or functional variants thereof having at least 60% identity thereto.

In some embodiments, the cell expresses a Beta glycosyltransferase, such as a Beta vulgaris glycosyltransferase, for example BvSGT2 (SEQ ID NO: 53); and one or both of:
a TYH selected from: a Beta TYH, such as an Beta vulgaris TYH, for example BvCYP76AD^{W13L} as set forth in SEQ ID NO: 27; a Basella TYH, such as an Basella alba TYH, for example BaTYH as set forth in SEQ ID NO: 29; a Cleretum TYH, such as a Cleretum bellidiforme TYH, for example CbTYH as set forth in SEQ ID NO: 31; a Phytolacca TYH, such as a Phytolacca americana TYH, for example PaTYH as set forth in SEQ ID NO: 33, or a Phytolacca dioica TYH, for example PdTYH as set forth in SEQ ID NO: 45; an Optunia TYH, such as an Optunia ficus-indicaTYH, for example OfTYH as set forth in SEQ ID NO: 35; an Abronia TYH, such as an Abronia nealleyi TYH, for example AnTYH as set forth in SEQ ID NO: 37; a Acleisanthes TYH, such as a Acleisanthes obtusa TYH, for example AoTYH as set forth in SEQ ID NO: 39; a Mirabilis TYH, such as a Mirabilis multiflora TYH, for example MmTYH1 as set forth in SEQ ID NO: 41 or MmTYH2 as set forth in SEQ ID NO: 47; or an Ercilla TYH, such as an Ercilla volubilis TYH, for example EvTYH as set forth in SEQ ID NO: 43; and
a DOD selected from: an Mirabilis DOD, such as an Mirabilis jalapa DOD, for example MjDOD as set forth in SEQ ID NO: 1; a Beta DOD, such as a Beta vulgaris DOD, for example BvDOD1 as set forth in SEQ ID NO: 3, BvDOD2 as set forth in SEQ ID NO: 23, or BvDOD3 as set forth in SEQ ID NO 25; a Bougainvillea DOD, such as a Bougainvillea glabra DOD, for example BgDOD1 as set forth in SEQ ID NO: 5 or BgDOD2 as set forth in SEQ ID NO: 21; a Portulaca DOD, such as a Portulaca grandiflora DOD, for example PgDOD as set forth in SEQ ID NO: 7; a truncated DOD (DOD*), such as for example PgDOD* as set forth in SEQ ID NO: 9; a Spinacia DOD, such as a Spinacia oleracea DOD, for example SoDOD as set forth in SEQ ID NO: 11; an Amaranthus DOD, such as an Amaranthus tricolour DOD, for example AtDOD as set forth in SEQ ID NO: 13, or such as an Amaranthus hypochondriacus DOD, for example AhDOD as set forth in SEQ ID NO: 15; a Phytolacca DOD, such as a Phytolacca americana DOD, for example PaDOD as set forth in SEQ ID NO: 17; or a Suaeda DOD, such as a Suaeda salsa DOD, for example SsDOD as set forth in SEQ ID NO: 19;
   or functional variants thereof having at least 60% identity thereto.

In some embodiments, the cell expresses a Beta glycosyltransferase, such as a Beta vulgaris glycosyltransferase, for example BvSGT3 (SEQ ID NO: 55); and one or both of:
a TYH selected from: a Beta TYH, such as an Beta vulgaris TYH, for example BvCYP76AD^{W13L} as set forth in SEQ ID NO: 27; a Basella TYH, such as an Basella alba TYH, for example BaTYH as set forth in SEQ ID NO: 29; a Cleretum TYH, such as a Cleretum bellidiforme TYH, for example CbTYH as set forth in SEQ ID NO: 31; a Phytolacca TYH, such as a Phytolacca americana TYH, for example PaTYH as set forth in SEQ ID NO: 33, or a Phytolacca dioica TYH, for example PdTYH as set forth in SEQ ID NO: 45; an Optunia TYH, such as an Optunia ficus-indicaTYH, for example OfTYH as set forth in SEQ ID NO: 35; an Abronia TYH, such as an Abronia nealleyi TYH, for example AnTYH as set forth in SEQ ID NO: 37; a Acleisanthes TYH, such as a Acleisanthes obtusa TYH, for example AoTYH as set forth in SEQ ID NO: 39; a Mirabilis TYH, such as a Mirabilis multiflora TYH, for example MmTYH1 as set forth in SEQ ID NO: 41 or MmTYH2 as set forth in SEQ ID NO: 47; or an Ercilla TYH, such as an Ercilla volubilis TYH, for example EvTYH as set forth in SEQ ID NO: 43; and
a DOD selected from: an Mirabilis DOD, such as an Mirabilis jalapa DOD, for example MjDOD as set forth in SEQ ID NO: 1; a Beta DOD, such as a Beta vulgaris DOD, for example BvDOD1 as set forth in SEQ ID NO: 3, BvDOD2 as set forth in SEQ ID NO: 23, or BvDOD3 as set forth in SEQ ID NO 25; a Bougainvillea DOD, such as a Bougainvillea glabra DOD, for example BgDOD1 as set forth in SEQ ID NO: 5 or BgDOD2 as set forth in SEQ ID NO: 21; a Portulaca DOD, such as a Portulaca grandiflora DOD, for example PgDOD as set forth in SEQ ID NO: 7; a truncated DOD (DOD*), such as for example PgDOD* as set forth in SEQ ID NO: 9; a Spinacia DOD, such as a Spinacia oleracea DOD, for example SoDOD as set forth in SEQ ID NO: 11; an Amaranthus DOD, such as an Amaranthus tricolour DOD, for example AtDOD as set forth in SEQ ID NO: 13, or such as an Amaranthus hypochondriacus DOD, for example AhDOD as set forth in SEQ ID NO: 15; a Phytolacca DOD, such as a Phytolacca americana DOD, for example PaDOD as set forth in SEQ ID NO: 17; or a Suaeda DOD, such as a Suaeda salsa DOD, for example SsDOD as set forth in SEQ ID NO: 19;
   or functional variants thereof having at least 60% identity thereto.

In some embodiments, the cell expresses a Beta glycosyltransferase, such as a Beta vulgaris glycosyltransferase, for example BvSGT4 (SEQ ID NO: 57); and one or both of:
a TYH selected from: a Beta TYH, such as an Beta vulgaris TYH, for example BvCYP76AD^{W13L} as set forth in SEQ ID NO: 27; a Basella TYH, such as an Basella alba TYH, for example BaTYH as set forth in SEQ ID NO: 29; a Cleretum TYH, such as a Cleretum bellidiforme TYH, for example CbTYH as set forth in SEQ ID NO: 31; a Phytolacca TYH, such as a Phytolacca americana TYH, for example PaTYH as set forth in SEQ ID NO: 33, or a Phytolacca dioica TYH, for example PdTYH as set forth in SEQ ID NO: 45; an Optunia TYH, such as an Optunia ficus-indicaTYH, for example OfTYH as set forth in SEQ ID NO: 35; an Abronia TYH, such as an Abronia nealleyi TYH, for example AnTYH as set forth in SEQ ID NO: 37; a Acleisanthes TYH, such as a Acleisanthes obtusa TYH, for example AoTYH as set forth in SEQ ID NO: 39; a Mirabilis TYH, such as a Mirabilis multiflora TYH, for example MmTYH1 as set forth in SEQ ID NO: 41 or MmTYH2 as set forth in SEQ ID NO: 47; or an Ercilla TYH, such as an Ercilla volubilis TYH, for example EvTYH as set forth in SEQ ID NO: 43; and
a DOD selected from: an Mirabilis DOD, such as an Mirabilis jalapa DOD, for example MjDOD as set forth in SEQ ID NO: 1; a Beta DOD, such as a Beta vulgaris DOD, for example BvDOD1 as set forth in SEQ ID NO: 3, BvDOD2 as set forth in SEQ ID NO: 23, or BvDOD3 as set forth in SEQ ID NO 25; a Bougainvillea DOD, such as a Bougainvillea glabra DOD, for example BgDOD1 as set forth in SEQ ID NO: 5 or BgDOD2 as set forth in SEQ ID NO: 21; a Portulaca DOD, such as a Portulaca grandiflora DOD, for example PgDOD as set forth in SEQ ID NO: 7; a truncated DOD (DOD*), such as for example PgDOD* as set forth in SEQ ID NO: 9; a Spinacia DOD, such as a Spinacia oleracea DOD, for example SoDOD as set forth in SEQ ID NO: 11; an Amaranthus DOD, such as an Amaranthus tricolour DOD, for example AtDOD as set forth in SEQ ID NO: 13, or such as an Amaranthus hypochondriacus DOD, for example AhDOD as set forth in SEQ ID NO: 15; a Phytolacca DOD, such as a Phytolacca americana DOD, for example PaDOD as set forth in SEQ ID NO: 17; or a Suaeda DOD, such as a Suaeda salsa DOD, for example SsDOD as set forth in SEQ ID NO: 19;
   or functional variants thereof having at least 60% identity thereto.

In some embodiments, the cell expresses a Chenopodium glycosyltransferase, such as a Chenopodium quinoa glycosyltransferase, for example CqSGT2 (SEQ ID NO: 65); and one or both of:
a TYH selected from: a Beta TYH, such as an Beta vulgaris TYH, for example BvCYP76AD^{W13L} as set forth in SEQ ID NO: 27; a Basella TYH, such as an Basella alba TYH, for example BaTYH as set forth in SEQ ID NO: 29; a Cleretum TYH, such as a Cleretum bellidiforme TYH, for example CbTYH as set forth in SEQ ID NO: 31; a Phytolacca TYH, such as a Phytolacca americana TYH, for example PaTYH as set forth in SEQ ID NO: 33, or a Phytolacca dioica TYH, for example PdTYH as set forth in SEQ ID NO: 45; an Optunia TYH, such as an Optunia ficus-indicaTYH, for example OfTYH as set forth in SEQ ID NO: 35; an Abronia TYH, such as an Abronia nealleyi TYH, for example AnTYH as set forth in SEQ ID NO: 37; a Acleisanthes TYH, such as a Acleisanthes obtusa TYH, for example AoTYH as set forth in SEQ ID NO: 39; a Mirabilis TYH, such as a Mirabilis multiflora TYH, for example MmTYH1 as set forth in SEQ ID NO: 41 or MmTYH2 as set forth in SEQ ID NO: 47; or an Ercilla TYH, such as an Ercilla volubilis TYH, for example EvTYH as set forth in SEQ ID NO: 43; and
a DOD selected from: an Mirabilis DOD, such as an Mirabilis jalapa DOD, for example MjDOD as set forth in SEQ ID NO: 1; a Beta DOD, such as a Beta vulgaris DOD, for example BvDOD1 as set forth in SEQ ID NO: 3, BvDOD2 as set forth in SEQ ID NO: 23, or BvDOD3 as set forth in SEQ ID NO 25; a Bougainvillea DOD, such as a Bougainvillea glabra DOD, for example BgDOD1 as set forth in SEQ ID NO: 5 or BgDOD2 as set forth in SEQ ID NO: 21; a Portulaca DOD, such as a Portulaca grandiflora DOD, for example PgDOD as set forth in SEQ ID NO: 7; a truncated DOD (DOD*), such as for example PgDOD* as set forth in SEQ ID NO: 9; a Spinacia DOD, such as a Spinacia oleracea DOD, for example SoDOD as set forth in SEQ ID NO: 11; an Amaranthus DOD, such as an Amaranthus tricolour DOD, for example AtDOD as set forth in SEQ ID NO: 13, or such as an Amaranthus hypochondriacus DOD, for example AhDOD as set forth in SEQ ID NO: 15; a Phytolacca DOD, such as a Phytolacca americana DOD, for example PaDOD as set forth in SEQ ID NO: 17; or a Suaeda DOD, such as a Suaeda salsa DOD, for example SsDOD as set forth in SEQ ID NO: 19;
   or functional variants thereof having at least 60% identity thereto.

In some embodiments, the cell expresses a Bougainvillea glycosyltransferase, such as a Bougainvillea glabra glycosyltransferase, for example BgGT2 (SEQ ID NO: 67); and one or both of:
a TYH selected from: a Beta TYH, such as an Beta vulgaris TYH, for example BvCYP76AD^{W13L} as set forth in SEQ ID NO: 27; a Basella TYH, such as an Basella alba TYH, for example BaTYH as set forth in SEQ ID NO: 29; a Cleretum TYH, such as a Cleretum bellidiforme TYH, for example CbTYH as set forth in SEQ ID NO: 31; a Phytolacca TYH, such as a Phytolacca americana TYH, for example PaTYH as set forth in SEQ ID NO: 33, or a Phytolacca dioica TYH, for example PdTYH as set forth in SEQ ID NO: 45; an Optunia TYH, such as an Optunia ficus-indicaTYH, for example OfTYH as set forth in SEQ ID NO: 35; an Abronia TYH, such as an Abronia nealleyi TYH, for example AnTYH as set forth in SEQ ID NO: 37; a Acleisanthes TYH, such as a Acleisanthes obtusa TYH, for example AoTYH as set forth in SEQ ID NO: 39; a Mirabilis TYH, such as a Mirabilis multiflora TYH, for example MmTYH1 as set forth in SEQ ID NO: 41 or MmTYH2 as set forth in SEQ ID NO: 47; or an Ercilla TYH, such as an Ercilla volubilis TYH, for example EvTYH as set forth in SEQ ID NO: 43; and
a DOD selected from: an Mirabilis DOD, such as an Mirabilis jalapa DOD, for example MjDOD as set forth in SEQ ID NO: 1; a Beta DOD, such as a Beta vulgaris DOD, for example BvDOD1 as set forth in SEQ ID NO: 3, BvDOD2 as set forth in SEQ ID NO: 23, or BvDOD3 as set forth in SEQ ID NO 25; a Bougainvillea DOD, such as a Bougainvillea glabra DOD, for example BgDOD1 as set forth in SEQ ID NO: 5 or BgDOD2 as set forth in SEQ ID NO: 21; a Portulaca DOD, such as a Portulaca grandiflora DOD, for example PgDOD as set forth in SEQ ID NO: 7; a truncated DOD (DOD*), such as for example PgDOD* as set forth in SEQ ID NO: 9; a Spinacia DOD, such as a Spinacia oleracea DOD, for example SoDOD as set forth in SEQ ID NO: 11; an Amaranthus DOD, such as an Amaranthus tricolour DOD, for example AtDOD as set forth in SEQ ID NO: 13, or such as an Amaranthus hypochondriacus DOD, for example AhDOD as set forth in SEQ ID NO: 15; a Phytolacca DOD, such as a Phytolacca americana DOD, for example PaDOD as set forth in SEQ ID NO: 17; or a Suaeda DOD, such as a Suaeda salsa DOD, for example SsDOD as set forth in SEQ ID NO: 19;
   or functional variants thereof having at least 60% identity thereto.

The term “functional variant having at least 60% identity” in relation to a given enzyme shall be understood to refer to functional variants having 60% identity or more to said enzyme, such as at least 61% identity, such as at least 62% identity, such as at least 63% identity, such as at least 64% identity, such as at least 65% identity, such as at least 66% identity, such as at least 67% identity, such as at least 68% identity, such as at least 69% identity, such as at least 70% identity, such as at least 71% identity, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% identity to the enzyme, or more.

A functional variant of a glycosyltransferase refers to a variant of glycosyltransferase which retains at least some of the activity of the parent enzyme. Thus, a functional variant of a glycosyltransferase can catalyze the same conversion as the glycosyltransferase from which it is derived, although the efficiency of the reaction may be different, e.g. the efficiency may be decreased or increased compared to the parent enzyme. Testing whether or not an enzyme is a functional variant of a glycosyltransferase can be tested using methods known in the art. For example, the glycosyltransferase variant can be expressed in a cell, wherein the cell medium contains the substrate of glycosyltransferase, i.e. L-tyrosine, or said substrate is produced in said cell. After incubating the cell for 24 hours, the amount of product, i.e. the amount of L-DOPA and/or L-dopaquinone, generated by the cell, i.e. by the glycosyltransferase variant, can be measured. If the glycosyltransferase variant generates the same product, i.e. L-DOPA and/or L-dopaquinone, as the glycosyltransferase does, if said glycosyltransferase is tested under the same conditions, i.e. expressed in a cell and incubated for 24 h, the glycosyltransferase variant is a functional variant of said glycosyltransferase.

### Method of Production

Provided herein is a method for production of one or more betalains in a yeast cell such as a Yarrowia lipolytica cell, said method comprising the steps of incubating a yeast cell in a medium, wherein said yeast cell expresses:
a. a first heterologous enzyme (TYH) capable of hydroxylating L-tyrosine and oxidizing L-DOPA, such as CYP76ADα;
b. a second heterologous enzyme which is a 4,5-DOPA extradiol dioxygenase (DOD); and
c. a third heterologous enzyme, having glycosyltransferase activity, such as an activity selected from a betanidin-5-O-glucosyltransferase (B50G) activity and a cyclo-DOPA-5-O-glucosyltransferase (cDOPA5OGT) activity, such as a scopoletin glucosyltransferase (SGT),
   whereby said cell is capable of producing one or more betalains, wherein said one or more betalains comprise one or more glycosylated betalains such as betanin and/or isobetanin,
   wherein the yeast cell further comprises a mutation resulting in reduced activity of 4-hydroxyphenylpyruvate dioxygenase (4-HPPD).

Also provided herein is a method for production of one or more betalains in a yeast cell, said method comprising the steps of incubating a yeast cell in a medium, wherein said yeast cell expresses:
a. a first heterologous enzyme (TYH) capable of hydroxylating L-tyrosine and oxidizing L-DOPA, such as CYP76ADα;
b. a second heterologous enzyme which is a 4,5-DOPA extradiol dioxygenase (DOD); and
c. a third heterologous enzyme, having glycosyltransferase activity, wherein said enzyme is selected from:
   i. Chenopodium quinoa glycosyltransferase CqSGT2 set forth in SEQ ID NO: 65, or a functional variant thereof having at least 70% sequence identity thereto;
   ii. Beta vulgaris glycosyltransferase BvSGT2 set forth in SEQ ID NO: 53, or a functional variant thereof having at least 70% identity thereto;
   iii. Bougainvillea glabra glycosyltransferase BgGT2 set forth in SEQ ID NO: 67, or a functional variant thereof having at least 70% identity thereto; and
   iv. Beta vulgaris glycosyltransferase BvSGT4 set forth in SEQ ID NO: 57, or a functional variant thereof having at least 70% identity thereto;
   whereby said cell is capable of producing one or more betalains, wherein said one or more betalains comprise one or more glycosylated betalains such as betanin and/or isobetanin.

Also provided herein is a method for production of one or more betalains in a yeast cell, said method comprising the steps of incubating a yeast cell in a medium, wherein said yeast cell expresses:
a first heterologous enzyme (TYH) capable of hydroxylating L-tyrosine and oxidizing L-DOPA, such as CYP76ADα; a second heterologous enzyme which is a 4,5-DOPA extradiol dioxygenase (DOD); and a third heterologous enzyme, having glycosyltransferase activity, such as an activity selected from a betanidin-5-O-glucosyltransferase (B50G) activity and a cyclo-DOPA-5-O-glucosyltransferase (cDOPA50GT) activity, such as a glycosyltransferase, such as a scopoletin glucosyltransferase (SGT), whereby said cell is capable of producing one or more betalains, wherein said one or more betalains comprise one or more glycosylated betalains such as betanin and/or isobetanin; and/or
a first heterologous enzyme (TYH) capable of hydroxylating L-tyrosine and oxidizing L-DOPA, such as CYP76ADα; and a second heterologous enzyme, which is a DOD having a truncation in its C-terminal end (DOD*), whereby said cell is capable of producing one or more betalains, wherein said one or more betalains comprise one or more betaxanthins.

In one embodiment, the method further comprises a step of recovering the one or more glycosylated betalains, such as the betanin and/or the isobetanin.

In one embodiment, the method yields one or more betalains, wherein said one or more betalains comprise one or more glycosylated betalains such as betanin and/or isobetanin, wherein the titer of the one or more betalains such as betanin and/or isobetanin is at least 0.5 mg/L, such as at least 1 mg/L, such as at least 1.5 mg/L, such as at least 5 mg/L, such as at least 10 mg/L, such as at least 25 mg/L, such as at least 50 mg/L, such as at least 100 mg/L, such as at least 250 mg/L, such as at least 500 mg/L, such as at least 750 mg/L, such as at least 1 g/L, such as at least 2 g/L, such as at least 3 g/L, such as at least 4 g/L, such as at least 5 g/L, such as at least 6 g/L, such as at least 7 g/L, such as at least 8 g/L, such as at least 9 g/L, such as at least 10 g/L, such as at least 11 g/L, such as at least 12 g/L, such as at least 13 g/L, such as at least 14 g/L, such as at least 15 g/L, such as at least 16 g/L, such as at least 17 g/L, such as at least 18 g/L, such as at least 19 g/L, such as at least 20 g/L, such as at least 25 g/L, such as at least 30 g/L, such as at least 35 g/L, such as at least 40 g/L, such as at least 45 g/L, such as at least 50 g/L, or more.

In another embodiment, the method increases the yield of the one or more betalains by at least 1.2-fold, such as at least 1.3-fold, such as at least 1.4-fold, such as at least 1.5-fold, such as at least 1.6-fold, such as at least 1.7-fold, such as at least 1.8-fold, such as at least 1.9-fold, such as at least 2-fold, such as at least 2.5-fold, such as at least 3-fold, such as at least 3.5-fold, such as at least 4-fold, such as at least 4.5-fold, such as at least 5-fold, such as at least 6-fold, such as at least 7-fold, such as at least 8-fold, such as at least 9-fold, such as at least 10-fold, such as at least 20-fold, such as at least 30-fold, such as at least 40-fold, such as at least 50-fold, wherein said one or more betalains comprise one or more betaxanthins., The increase may be determined by methods known in the art. For example, the increase can be determined by measuring the fluorescence per OD and comparing it to the fluorescence per OD obtained in a reference yeast cell expressing MjDOD+BvCYP76AD^{W13L} cultivated in similar or identical conditions.

Further provided herein is a method for producing at least 0.5 mg/L of one or more betalains, wherein said one or more betalains comprise a glycosylated betalain such as betanin and/or isobetanin, such as at least 1 mg/L, such as at least 1.5 mg/L, such as at least 5 mg/L, such as at least 10 mg/L, such as at least 25 mg/L, such as at least 50 mg/L, such as at least 100 mg/L, such as at least 250 mg/L, such as at least 500 mg/L, such as at least 750 mg/L, such as at least 1 g/L, such as at least 2 g/L, such as at least 3 g/L, such as at least 4 g/L, such as at least 5 g/L, such as at least 6 g/L, such as at least 7 g/L, such as at least 8 g/L, such as at least 9 g/L, such as at least 10 g/L, such as at least 11 g/L, such as at least 12 g/L, such as at least 13 g/L, such as at least 14 g/L, such as at least 15 g/L, such as at least 16 g/L, such as at least 17 g/L, such as at least 18 g/L, such as at least 19 g/L, such as at least 20 g/L, such as at least 25 g/L, such as at least 30 g/L, such as at least 35 g/L, such as at least 40 g/L, such as at least 45 g/L, such as at least 50 g/L, or more.

Also provided herein is a method is for producing at least 0.5 mg/L of betanin and/or isobetanin, such as at least 1 mg/L, such as at least 1.5 mg/L, such as at least 5 mg/L, such as at least 10 mg/L, such as at least 25 mg/L, such as at least 50 mg/L, such as at least 100 mg/L, such as at least 250 mg/L, such as at least 500 mg/L, such as at least 750 mg/L, such as at least 1 g/L, such as at least 2 g/L, such as at least 3 g/L, such as at least 4 g/L, such as at least 5 g/L, such as at least 6 g/L, such as at least 7 g/L, such as at least 8 g/L, such as at least 9 g/L, such as at least 10 g/L, such as at least 11 g/L, such as at least 12 g/L, such as at least 13 g/L, such as at least 14 g/L, such as at least 15 g/L, such as at least 16 g/L, such as at least 17 g/L, such as at least 18 g/L, such as at least 19 g/L, such as at least 20 g/L, such as at least 25 g/L, such as at least 30 g/L, such as at least 35 g/L, such as at least 40 g/L, such as at least 45 g/L, such as at least 50 g/L, or more.

Also provided herein is a method is for producing at least 0.5 mg/L of a betaxanthin, such as at least 1 mg/L, such as at least 1.5 mg/L, such as at least 5 mg/L, such as at least 10 mg/L, such as at least 25 mg/L, such as at least 50 mg/L, such as at least 100 mg/L, such as at least 250 mg/L, such as at least 500 mg/L, such as at least 750 mg/L, such as at least 1 g/L, such as at least 2 g/L, such as at least 3 g/L, such as at least 4 g/L, such as at least 5 g/L, such as at least 6 g/L, such as at least 7 g/L, such as at least 8 g/L, such as at least 9 g/L, such as at least 10 g/L, such as at least 11 g/L, such as at least 12 g/L, such as at least 13 g/L, such as at least 14 g/L, such as at least 15 g/L, such as at least 16 g/L, such as at least 17 g/L, such as at least 18 g/L, such as at least 19 g/L, such as at least 20 g/L, such as at least 25 g/L, such as at least 30 g/L, such as at least 35 g/L, such as at least 40 g/L, such as at least 45 g/L, such as at least 50 g/L, or more.

In some embodiments, L-tyrosine is supplied in the growth medium. In some embodiments, the growth medium is supplemented with at least 100 mg/L L-tyrosine, such as at least 200 mg/L L-tyrosine, such as at least 400 mg/L L-tyrosine, such as at least 600 mg/L L-tyrosine, such as at least 800 mg/L L-tyrosine, such as at least 1.2 g/L L-tyrosine, such as at least 1.4 L-tyrosine, such as at least 1.6 g/L L-tyrosine, such as at least 1.8 g/L L-tyrosine, such as at least 2 g/L L-tyrosine, such as at least 3 g/L L-tyrosine, such as at least 4 g/L L-tyrosine, such as at least 6 g/L L-tyrosine, such as at least 8 g/L L-tyrosine.

In one embodiment, the yeast cell is as described in the section “Yeast cell”, e.g. an S. cerevisiae cell or a Y. lipolytica cell.

In one embodiment, the enzyme having glycosyltransferase activity, such as the glycosyltransferase is as described in the section “Enzyme having glycosyltransferase activity”. In particular, the enzyme having glycosyltransferase activity may be selected from the glycosyltransferase as set forth in SEQ ID NO: 51 (BvSGT1), the glycosyltransferase as set forth in SEQ ID NO: 53 (BvSGT2), the glycosyltransferase as set forth in SEQ ID NO: 55 (BvSGT3), the glycosyltransferase as set forth in SEQ ID NO: 57 (BvSGT4), the glycosyltransferase from Chenopodium quinoa as set forth in SEQ ID NO: 65 (CqSGT2) and the glycosyltransferase from Bougainvillea glabra set forth in SEQ ID NO: 67 (BgGT2), or functional variants thereof as described herein above.

In one embodiment, the TYH is as described in the section “CYP76AD (TYH)”. In particular, the TYH may be selected from the TYHs as set forth in SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, and SEQ ID NO: 47, or functional variants thereof, as described herein above.

In one embodiment, the DOD and/or the DOD* is as described in the section “2,5-DOPA extradiol dioxygenase”. In particular, the DOD may be selected from the DODs as set forth in SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, and SEQ ID NO: 25, or functional variants thereof, as described herein above.

In one embodiment, the method comprises expressing in a yeast cell:
a. a TYH from Abronia nealleyi (AnTYH) as set forth in SEQ ID NO: 37; a DOD from Bougainvillea glabra (BgDOD2) as set forth in SEQ ID NO: 21; and a glycosyltransferase from Chenopodium quinoa as set forth in SEQ ID NO: 65, (CqSGT2); or
b. a TYH from Abronia nealleyi (AnTYH) as set forth in SEQ ID NO: 37; a DOD from Bougainvillea glabra (BgDOD2) as set forth in SEQ ID NO: 21; and a glycosyltransferase from Beta vulgaris (BvSGT2) as set forth in SEQ ID NO: 53; or
c. a TYH from Abronia nealleyi (AnTYH) as set forth in SEQ ID NO: 37; a DOD from Bougainvillea glabra (BgDOD2) as set forth in SEQ ID NO: 21; and a glycosyltransferase from Bougainvillea glabra as set forth in SEQ ID NO: 67, (BgGT2); or
d. a TYH from Abronia nealleyi (AnTYH) as set forth in SEQ ID NO: 37; a DOD from Bougainvillea glabra (BgDOD2) as set forth in SEQ ID NO: 21; and a glycosyltransferase from Beta vulgaris (BvSGT4) as set forth in SEQ ID NO: 57; or
e. a TYH from Abronia nealleyi (AnTYH) as set forth in SEQ ID NO: 37; a DOD from Mirabilis jalapa (MjDOD) as set forth in SEQ ID NO: 1; and a glycosyltransferase from Chenopodium quinoa as set forth in SEQ ID NO: 65, (CqSGT2); or
f. a TYH from Abronia nealleyi (AnTYH) as set forth in SEQ ID NO: 37; a DOD from Mirabilis jalapa (MjDOD) as set forth in SEQ ID NO: 1; and a glycosyltransferase from Beta vulgaris (BvSGT2) as set forth in SEQ ID NO: 53; or
g. a TYH from Abronia nealleyi (AnTYH) as set forth in SEQ ID NO: 37; a DOD from Mirabilis jalapa (MjDOD) as set forth in SEQ ID NO: 1; and a glycosyltransferase from Bougainvillea glabra as set forth in SEQ ID NO: 67, (BgGT2); or
h. a TYH from Abronia nealleyi (AnTYH) as set forth in SEQ ID NO: 37; a DOD from Mirabilis jalapa (MjDOD) as set forth in SEQ ID NO: 1; and a glycosyltransferase from Beta vulgaris (BvSGT4) as set forth in SEQ ID NO: 57; or
i. a TYH from Abronia nealleyi (AnTYH) as set forth in SEQ ID NO: 37; a DOD from Portulaca grandiflora (PgDOD) as set forth in SEQ ID NO: 7; and a glycosyltransferase from Chenopodium quinoa as set forth in SEQ ID NO: 65, (CqSGT2); or
j. a TYH from Abronia nealleyi (AnTYH) as set forth in SEQ ID NO: 37; a DOD from Portulaca grandiflora (PgDOD) as set forth in SEQ ID NO: 7; and a glycosyltransferase from Beta vulgaris (BvSGT2) as set forth in SEQ ID NO: 53; or
k. a TYH from Abronia nealleyi (AnTYH) as set forth in SEQ ID NO: 37; a DOD from Portulaca grandiflora (PgDOD) as set forth in SEQ ID NO: 7; and a glycosyltransferase from Bougainvillea glabra as set forth in SEQ ID NO: 67, (BgGT2); or
l. a TYH from Abronia nealleyi (AnTYH) as set forth in SEQ ID NO: 37; a DOD from Portulaca grandiflora (PgDOD) as set forth in SEQ ID NO: 7; and a glycosyltransferase from Beta vulgaris (BvSGT4) as set forth in SEQ ID NO: 57; or
m. a TYH from Ercilla volubisi (EvTYH) as set forth in SEQ ID NO: 43; a DOD from Bougainvillea glabra (BgDOD2) as set forth in SEQ ID NO: 21; and a glycosyltransferase from Chenopodium quinoa as set forth in SEQ ID NO: 65, (CqSGT2); or
n. a TYH from Ercilla volubisi (EvTYH) as set forth in SEQ ID NO: 43; a DOD from Bougainvillea glabra (BgDOD2) as set forth in SEQ ID NO: 21; and a glycosyltransferase from Beta vulgaris (BvSGT2) as set forth in SEQ ID NO: 53; or
o. a TYH from Ercilla volubisi (EvTYH) as set forth in SEQ ID NO: 43; a DOD from Bougainvillea glabra (BgDOD2) as set forth in SEQ ID NO: 21; and a glycosyltransferase from Bougainvillea glabra as set forth in SEQ ID NO: 67, (BgGT2); or
p. a TYH from Ercilla volubisi (EvTYH) as set forth in SEQ ID NO: 43; a DOD from Bougainvillea glabra (BgDOD2) as set forth in SEQ ID NO: 21; and a glycosyltransferase from Beta vulgaris (BvSGT4) as set forth in SEQ ID NO: 57; or
q. a TYH from Ercilla volubisi (EvTYH) as set forth in SEQ ID NO: 43; a DOD from Mirabilis jalapa (MjDOD) as set forth in SEQ ID NO: 1; and a glycosyltransferase from Chenopodium quinoa as set forth in SEQ ID NO: 65, (CqSGT2); or
r. a TYH from Ercilla volubisi (EvTYH) as set forth in SEQ ID NO: 43; a DOD from Mirabilis jalapa (MjDOD) as set forth in SEQ ID NO: 1; and a glycosyltransferase from Beta vulgaris (BvSGT2) as set forth in SEQ ID NO: 53; or
s. a TYH from Ercilla volubisi (EvTYH) as set forth in SEQ ID NO: 43; a DOD from Mirabilis jalapa (MjDOD) as set forth in SEQ ID NO: 1; and a glycosyltransferase from Bougainvillea glabra as set forth in SEQ ID NO: 67, (BgGT2); or
t. a TYH from Ercilla volubisi (EvTYH) as set forth in SEQ ID NO: 43; a DOD from Mirabilis jalapa (MjDOD) as set forth in SEQ ID NO: 1; and a glycosyltransferase from Beta vulgaris (BvSGT4) as set forth in SEQ ID NO: 57; or
u. a TYH from Ercilla volubisi (EvTYH) as set forth in SEQ ID NO: 43; a DOD from Portulaca grandiflora (PgDOD) as set forth in SEQ ID NO: 7; and a glycosyltransferase from Chenopodium quinoa as set forth in SEQ ID NO: 65, (CqSGT2); or
v. a TYH from Ercilla volubisi (EvTYH) as set forth in SEQ ID NO: 43; a DOD from Portulaca grandiflora (PgDOD) as set forth in SEQ ID NO: 7; and a glycosyltransferase from Beta vulgaris (BvSGT2) as set forth in SEQ ID NO: 53; or
w. a TYH from Ercilla volubisi (EvTYH) as set forth in SEQ ID NO: 43; a DOD from Portulaca grandiflora (PgDOD) as set forth in SEQ ID NO: 7; and a glycosyltransferase from Bougainvillea glabra as set forth in SEQ ID NO: 67, (BgGT2); or
x. a TYH from Ercilla volubisi (EvTYH) as set forth in SEQ ID NO: 43; a DOD from Portulaca grandiflora (PgDOD) as set forth in SEQ ID NO: 7; and a glycosyltransferase from Beta vulgaris (BvSGT4) as set forth in SEQ ID NO: 57; or functional variants thereof having at least 80% identity thereto, whereby said yeast cell is capable of producing one or more betalains, wherein said one or more betalains comprise a glycosylated betalain, such as betanin and/or isobetanin.

In one embodiment, the method comprises expressing in a yeast cell:
a. a first heterologous enzyme selected from the group consisting of SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, and SEQ ID NO: 47, or a functional variant thereof having at least 70% identity thereto, preferably wherein the first heterologous enzyme is an Abronia nealleyi TYH such as AnTYH as set forth in SEQ ID NO: 37; or an Ercilla volubis TYH such as EvTYH as set forth in SEQ ID NO: 43; or a functional variant thereof having at least 80% identity thereto; and
b. a second heterologous enzyme selected from the group consisting of SEQ ID NO: 1, SEQ ID NO: 3, SEQ ID NO: 5, SEQ ID NO: 7, SEQ ID NO: 11, SEQ ID NO: 13, SEQ ID NO: 15, SEQ ID NO: 17, SEQ ID NO: 19, SEQ ID NO: 21, SEQ ID NO: 23, and SEQ ID NO: 25, or a functional variant thereof having at least 70% identity thereto, preferably wherein the second heterologous enzyme is a Mirabilis jalapa DOD such as MjDOD as set forth in SEQ ID NO: 1; a Bougainvillea glabra DOD such as BgDOD2 as set forth in SEQ ID NO: 21; or a Portulaca grandiflora DOD such as PgDOD as set forth in SEQ ID NO: 7; or a functional variant thereof having at least 80% identity thereto
c. a third heterologous enzyme selected from:
   i. Chenopodium quinoa glycosyltransferase CqSGT2 set forth in SEQ ID NO: 65, or a functional variant thereof having at least 70% sequence identity thereto;
   ii. Beta vulgaris glycosyltransferase BvSGT2 set forth in SEQ ID NO: 53, or a functional variant thereof having at least 70% identity thereto;
   iii. Bougainvillea glabra glycosyltransferase BgGT2 set forth in SEQ ID NO: 67, or a functional variant thereof having at least 70% identity thereto; and
   iv. Beta vulgaris glycosyltransferase BvSGT4 set forth in SEQ ID NO: 57, or a functional variant thereof having at least 70% identity thereto;
      whereby said yeast cell is capable of producing one or more betalains, wherein said one or more betalains comprise a glycosylated betalain, such as betanin and/or isobetanin, wherein the titer of the one or more betalains is at least 4 mg/L, such as at least 5 mg/L, such as at least 6 mg/L, such as at least 7 mg/L, such as at least 8 mg/L, such as at least 9 mg/L, such as at least 10 mg/L, such as at least 15 mg/L, such as at least 20 mg/L, such as at least 25 mg/L, such as at least 50 mg/L, such as at least 100 mg/L, such as at least 500 mg/L, such as at least 1 g/L, such as at least 2 g/L, such as at least 5 g/L, such as at least 10 g/L, such as at least 15 g/L, such as at least 20 g/L, such as at least 25 g/L.

In one embodiment, the method comprises expressing in the yeast cell:
a. a TYH from Abronia nealleyi (AnTYH) as set forth in SEQ ID NO: 37; and a truncated DOD* as set forth in SEQ ID NO: 9 (PgDOD*); or
b. a TYH from Ercilla volubis (EvTYH) as set forth in SEQ ID NO 43; and a truncated DOD* as set forth in SEQ ID NO: 9 (PgDOD*); or
   or functional variants thereof having at least 80% identity thereto, whereby said yeast cell is capable of producing one or more betalains, wherein said one or more betalains comprise one or more betaxanthins.

In one embodiment, the method comprises expressing in a yeast cell a TYH from Abronia nealleyi (AnTYH) as set forth in SEQ ID NO: 37; and a truncated DOD* as set forth in SEQ ID NO: 9 (PgDOD*), or functional variants thereof having at least 80% identity thereto, whereby said yeast cell is capable of producing one or more betalains, wherein said one or more betalains comprise one or more betaxanthins, whereby said method increases the yield of the one or more the one or more betaxanthins by at least 2-fold, such as at least 2.5-fold, such as at least 3-fold, such as at least 3.5-fold, such as at least 4-fold, such as at least 4.5-fold, such as at least 5-fold, such as at least 6-fold, such as at least 7-fold, such as at least 8-fold, such as at least 9-fold, such as at least 10-fold, such as at least 20-fold, such as at least 30-fold, such as at least 40-fold, such as at least 50-fold. The increase may be determined by methods known in the art. In some embodiments, the increase is determined by measuring the fluorescence per OD and comparing it to the fluorescence per OD obtained in a reference yeast cell expressing MjDOD+BvCYP76AD^{W13L} cultivated in similar or identical conditions.

### Nucleic Acid

Provided herein is a system comprising nucleic acids encoding:
a. a TYH, preferably as described herein above, such as CYP76ADα capable of:
   i. hydroxylating L-tyrosine; and/or
   ii. oxidizing L-DOPA; and
b. a DOD, preferably as described herein above, capable of oxygenating L-DOPA; and
c. an enzyme having glycosyltransferase activity, such as a glycosyltransferase, preferably as described herein above, capable of:
   i. glycosylating cyclo-DOPA; and/or
   ii. glycosylating betanidin;
wherein said enzyme having glycosyltransferase activity is selected from:
   i) Chenopodium quinoa glycosyltransferase CqSGT2 set forth in SEQ ID NO: 66, or a functional variant thereof having at least 70% sequence identity thereto;
   ii) Beta vulgaris glycosyltransferase BvSGT2 set forth in SEQ ID NO: 54, or a functional variant thereof having at least 70% identity thereto;
   iii) Bougainvillea glabra glycosyltransferase BgGT2 set forth in SEQ ID NO: 68, or a functional variant thereof having at least 70% identity thereto; and
   iv) Beta vulgaris glycosyltransferase BvSGT4 set forth in SEQ ID NO: 58, or a functional variant thereof having at least 70% identity thereto.

In some embodiments, the system is comprised in a vector, such as a plasmid, or in the genome of the yeast cell.

In one embodiment, the enzyme having glycosyltransferase activity is as described in the section “Enzyme having glycosyltransferase activity”. In particular, the enzyme having glycosyltransferase activity may be selected from the glycosyltransferases as set forth in SEQ ID NO: 52 (BvSGT1), SEQ ID NO: 54 (BvSGT2), SEQ ID NO: 56 (BvSGT3), SEQ ID NO: 58 (BvSGT4), SEQ ID NO: 66 (CqSGT2) and SEQ ID NO: 68 (BgGT2), or functional variants thereof as described herein above.

In one embodiment, the TYH is as described in the section “CYP76AD (TYH)”. In particular, the TYH may be selected from the TYHs as set forth in SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 42, SEQ ID NO: 44, SEQ ID NO: 46, SEQ ID NO: 48, SEQ ID NO: 62, SEQ ID NO: 63, and SEQ ID NO: 64, or functional variants thereof, as described herein above.

In one embodiment, the DOD and/or the DOD* is as described in the section “2,5-DOPA extradiol dioxygenase”. In particular, the DOD may be selected from the DODs as set forth in SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 59, SEQ ID NO: 60 and SEQ ID NO: 61, or functional variants thereof, as described herein above.

In one embodiment, the system comprises:
a. a TYH from Abronia nealleyi (AnTYH) as set forth in SEQ ID NO: 38; a DOD from Bougainvillea glabra (BgDOD2) as set forth in SEQ ID NO: 22; and a glycosyltransferase from Chenopodium quinoa as set forth in SEQ ID NO: 66, (CqSGT2); or
b. a TYH from Abronia nealleyi (AnTYH) as set forth in SEQ ID NO: 38; a DOD from Bougainvillea glabra (BgDOD2) as set forth in SEQ ID NO: 22; and a glycosyltransferase from Beta vulgaris (BvSGT2) as set forth in SEQ ID NO: 54; or
c. a TYH from Abronia nealleyi (AnTYH) as set forth in SEQ ID NO: 38; a DOD from Bougainvillea glabra (BgDOD2) as set forth in SEQ ID NO: 22; and a glycosyltransferase from Bougainvillea glabra as set forth in SEQ ID NO: 68, (BgGT2); or
d. a TYH from Abronia nealleyi (AnTYH) as set forth in SEQ ID NO: 38; a DOD from Bougainvillea glabra (BgDOD2) as set forth in SEQ ID NO: 22; and a glycosyltransferase from Beta vulgaris (BvSGT4) as set forth in SEQ ID NO: 58; or
e. a TYH from Abronia nealleyi (AnTYH) as set forth in SEQ ID NO: 38; a DOD from Mirabilis jalapa (MjDOD) as set forth in SEQ ID NO: 2; and a glycosyltransferase from Chenopodium quinoa as set forth in SEQ ID NO: 66, (CqSGT2); or
f. a TYH from Abronia nealleyi (AnTYH) as set forth in SEQ ID NO: 38; a DOD from Mirabilis jalapa (MjDOD) as set forth in SEQ ID NO: 2; and a glycosyltransferase from Beta vulgaris (BvSGT2) as set forth in SEQ ID NO: 54; or
g. a TYH from Abronia nealleyi (AnTYH) as set forth in SEQ ID NO: 38; a DOD from Mirabilis jalapa (MjDOD) as set forth in SEQ ID NO: 2; and a glycosyltransferase from Bougainvillea glabra as set forth in SEQ ID NO: 68, (BgGT2); or
h. a TYH from Abronia nealleyi (AnTYH) as set forth in SEQ ID NO: 38; a DOD from Mirabilis jalapa (MjDOD) as set forth in SEQ ID NO: 2; and a glycosyltransferase from Beta vulgaris (BvSGT4) as set forth in SEQ ID NO: 58; or
i. a TYH from Abronia nealleyi (AnTYH) as set forth in SEQ ID NO: 38; a DOD from Portulaca grandiflora (PgDOD) as set forth in SEQ ID NO: 8; and a glycosyltransferase from Chenopodium quinoa as set forth in SEQ ID NO: 66, (CaSGT2); or
j. a TYH from Abronia nealleyi (AnTYH) as set forth in SEQ ID NO: 38; a DOD from Portulaca grandiflora (PgDOD) as set forth in SEQ ID NO: 8; and a glycosyltransferase from Beta vulgaris (BvSGT2) as set forth in SEQ ID NO: 54; or
k. a TYH from Abronia nealleyi (AnTYH) as set forth in SEQ ID NO: 38; a DOD from Portulaca grandiflora (PgDOD) as set forth in SEQ ID NO: 8; and a glycosyltransferase from Bougainvillea glabra as set forth in SEQ ID NO: 68, (BgGT2); or
l. a TYH from Abronia nealleyi (AnTYH) as set forth in SEQ ID NO: 38; a DOD from Portulaca grandiflora (PgDOD) as set forth in SEQ ID NO: 8; and a glycosyltransferase from Beta vulgaris (BvSGT4) as set forth in SEQ ID NO: 58; or
m. a TYH from Ercilla volubisi (EvTYH) as set forth in SEQ ID NO: 44; a DOD from Bougainvillea glabra (BgDOD2) as set forth in SEQ ID NO: 22; and a glycosyltransferase from Chenopodium quinoa as set forth in SEQ ID NO: 66, (CqSGT2); or
n. a TYH from Ercilla volubisi (EvTYH) as set forth in SEQ ID NO: 44; a DOD from Bougainvillea glabra (BgDOD2) as set forth in SEQ ID NO: 22; and a glycosyltransferase from Beta vulgaris (BvSGT2) as set forth in SEQ ID NO: 54; or
o. a TYH from Ercilla volubisi (EvTYH) as set forth in SEQ ID NO: 44; a DOD from Bougainvillea glabra (BgDOD2) as set forth in SEQ ID NO: 22; and a glycosyltransferase from Bougainvillea glabra as set forth in SEQ ID NO: 68, (BgGT2); or
p. a TYH from Ercilla volubisi (EvTYH) as set forth in SEQ ID NO: 44; a DOD from Bougainvillea glabra (BgDOD2) as set forth in SEQ ID NO: 22; and a glycosyltransferase from Beta vulgaris (BvSGT4) as set forth in SEQ ID NO: 58; or
q. a TYH from Ercilla volubisi (EvTYH) as set forth in SEQ ID NO: 44; a DOD from Mirabilis jalapa (MjDOD) as set forth in SEQ ID NO: 2; and a glycosyltransferase from Chenopodium quinoa as set forth in SEQ ID NO: 66, (CqSGT2); or
r. a TYH from Ercilla volubisi (EvTYH) as set forth in SEQ ID NO: 44; a DOD from Mirabilis jalapa (MjDOD) as set forth in SEQ ID NO: 2; and a glycosyltransferase from Beta vulgaris (BvSGT2) as set forth in SEQ ID NO: 54; or
s. a TYH from Ercilla volubisi (EvTYH) as set forth in SEQ ID NO: 44; a DOD from Mirabilis jalapa (MjDOD) as set forth in SEQ ID NO: 2; and a glycosyltransferase from Bougainvillea glabra as set forth in SEQ ID NO: 68, (BgGT2); or
t. a TYH from Ercilla volubisi (EvTYH) as set forth in SEQ ID NO: 44; a DOD from Mirabilis jalapa (MjDOD) as set forth in SEQ ID NO: 2; and a glycosyltransferase from Beta vulgaris (BvSGT4) as set forth in SEQ ID NO: 58; or
u. a TYH from Ercilla volubisi (EvTYH) as set forth in SEQ ID NO: 44; a DOD from Portulaca grandiflora (PgDOD) as set forth in SEQ ID NO: 8; and a glycosyltransferase from Chenopodium quinoa as set forth in SEQ ID NO: 66, (CqSGT2); or
v. a TYH from Ercilla volubisi (EvTYH) as set forth in SEQ ID NO: 44; a DOD from Portulaca grandiflora (PgDOD) as set forth in SEQ ID NO: 8; and a glycosyltransferase from Beta vulgaris (BvSGT2) as set forth in SEQ ID NO: 54; or
w. a TYH from Ercilla volubisi (EvTYH) as set forth in SEQ ID NO: 44; a DOD from Portulaca grandiflora (PgDOD) as set forth in SEQ ID NO: 8; and a glycosyltransferase from Bougainvillea glabra as set forth in SEQ ID NO: 68, (BgGT2); or
x. a TYH from Ercilla volubisi (EvTYH) as set forth in SEQ ID NO: 44; a DOD from Portulaca grandiflora (PgDOD) as set forth in SEQ ID NO: 8; and a glycosyltransferase from Beta vulgaris (BvSGT4) as set forth in SEQ ID NO: 58; or functional variants thereof having at least 80% identity thereto.

In one embodiment, the system comprises:
a. a TYH from Abronia nealleyi (AnTYH) as set forth in SEQ ID NO: 38; a truncated DOD* as set forth in SEQ ID NO: 10 or SEQ ID NO: 59 (PgDOD*); and an SGT from Beta vulgaris (BvSGT2) as set forth in SEQ ID NO: 54; or
b. a TYH from Ercilla volubis (EvTYH) as set forth in SEQ ID NO 44; a DOD from Bougainvillea glabra (BgDOD2) as set forth in SEQ ID NO: 22; and an SGT from Beta vulgaris (BvSGT2) as set forth in SEQ ID NO: 54;
   or functional variants thereof having at least 80% identity thereto.

In one embodiment, the system comprises:
a. a TYH from Abronia nealleyi (AnTYH) as set forth in SEQ ID NO: 38; and a truncated DOD* as set forth in SEQ ID NO: 10 (PgDOD*); or
b. a TYH from Ercilla volubis (EvTYH) as set forth in SEQ ID NO 44; and a truncated DOD* as set forth in SEQ ID NO: 10 (PgDOD*); or
   or functional variants thereof having at least 80% identity thereto.

In one embodiment, the system comprises a TYH from Abronia nealleyi (AnTYH) as set forth in SEQ ID NO: 38; and a truncated DOD* as set forth in SEQ ID NO: 10 (PgDOD*), or functional variants thereof having at least 80% identity thereto.

### CYP76AD (TYH)

In some embodiments, the heterologous TYH is encoded by a polynucleotide having at least 60% identity to a polynucleotide selected from the group of polynucleotides set forth in SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 42, SEQ ID NO: 44, SEQ ID NO: 46, SEQ ID NO: 48, SEQ ID NO: 62, SEQ ID NO: 63, and SEQ ID NO: 64, such as at least 61%, such as at least 62%, such as at least 63%, such as at least 64%, such as at least 65%, such as at least 66%, such as at least 67%, such as at least 68%, such as at least 69%, such as at least 70%, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% identity thereto.

In one embodiment, the heterologous TYH is encoded by a nucleic acid having at least 60% identity to the nucleic acid encoding a TYH from Abronia nealleyi, as set forth in SEQ ID NO: 38 and SEQ ID NO: 64.

In one embodiment, the heterologous TYH is encoded by a nucleic acid having at least 60% identity to the nucleic acid encoding a TYH from Acleisanthes obtusa, as set forth in SEQ ID NO: 40.

In one embodiment, the heterologous TYH is encoded by a nucleic acid having at least 60% identity to the nucleic acid encoding a TYH from Basella alba, as set forth in SEQ ID NO: 30.

In one embodiment, the heterologous TYH is encoded by a nucleic acid having at least 60% identity to the nucleic acid encoding a TYH from Beta vulgaris, as set forth in SEQ ID NO: 28 and SEQ ID NO: 62.

In one embodiment, the heterologous TYH is encoded by a nucleic acid having at least 60% identity to the nucleic acid encoding a TYH from Cleretum bellidiforme, as set forth in SEQ ID NO: 32.

In one embodiment, the heterologous TYH is encoded by a nucleic acid having at least 60% identity to the nucleic acid encoding a TYH from Ercilla volubilis, as set forth in SEQ ID NO: 44 and SEQ ID NO: 63.

In one embodiment, the heterologous TYH is encoded by a nucleic acid having at least 60% identity to the nucleic acid encoding a TYH from Mirabilis multiflora, as set forth in SEQ ID NO: 42.

In one embodiment, the heterologous TYH is encoded by a nucleic acid having at least 60% identity to the nucleic acid encoding a TYH from Mirabilis multiflora, as set forth in SEQ ID NO: 48.

In one embodiment, the heterologous TYH is encoded by a nucleic acid having at least 60% identity to the nucleic acid encoding a TYH from Optunia ficus-indica, as set forth in SEQ ID NO: 36.

In one embodiment, the heterologous TYH is encoded by a nucleic acid having at least 60% identity to the nucleic acid encoding a TYH from Phytolacca americana, as set forth in SEQ ID NO: 34.

In one embodiment, the heterologous TYH is encoded by a nucleic acid having at least 60% identity to the nucleic acid encoding a TYH from Phytolacca dioica, as set forth in SEQ ID NO: 46.

Herein, a nucleic acid having at least 60% identity to a given nucleic acid may have at least 61% identity, such as at least 62% identity, such as at least 63% identity, such as at least 64% identity, such as at least 65% identity, such as at least 66% identity, such as at least 67% identity, such as at least 68% identity, such as at least 69% identity, such as at least 70% identity, such as at least 71% identity, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% identity to the given nucleic acid, or more.

### 4,5-DOPA Extradiol Dioxygenase (DOD)

In some embodiments, the heterologous DOD is encoded by a polynucleotide having at least 60% identity to a polynucleotide selected from the group of polynucleotides set forth in SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 59, SEQ ID NO: 60 and SEQ ID NO: 61, such as at least 61%, such as at least 62%, such as at least 63%, such as at least 64%, such as at least 65%, such as at least 66%, such as at least 67%, such as at least 68%, such as at least 69%, such as at least 70%, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% identity thereto.

In one embodiment, the heterologous DOD is encoded by a nucleic acid having at least 60% identity to the nucleic acid encoding a DOD from Amaranthus hypochondriacus, as set forth in SEQ ID NO: 16.

In one embodiment, the heterologous DOD is encoded by a nucleic acid having at least 60% identity to the nucleic acid encoding a DOD from Amaranthus tricolour, as set forth in SEQ ID NO: 14.

In one embodiment, the heterologous DOD is encoded by a nucleic acid having at least 60% identity to the nucleic acid encoding a DOD from Beta vulgaris, as set forth in SEQ ID NO: 4.

In one embodiment, the heterologous DOD is encoded by a nucleic acid having at least 60% identity to the nucleic acid encoding a DOD from Beta vulgaris, as set forth in SEQ ID NO: 24.

In one embodiment, the heterologous DOD is encoded by a nucleic acid having at least 60% identity to the nucleic acid encoding a DOD from Beta vulgaris, as set forth in SEQ ID NO: 26.

In one embodiment, the heterologous DOD is encoded by a nucleic acid having at least 60% identity to the nucleic acid encoding a DOD from Bougainvillea glabra, as set forth in SEQ ID NO: 6 and SEQ ID NO: 60.

In one embodiment, the heterologous DOD is encoded by a nucleic acid having at least 60% identity to the nucleic acid encoding a DOD from Bougainvillea glabra, as set forth in SEQ ID NO: 22.

In one embodiment, the heterologous DOD is encoded by a nucleic acid having at least 60% identity to the nucleic acid encoding a DOD from Mirabilis jalapa, as set forth in SEQ ID NO: 2 and SEQ ID NO: 61.

In one embodiment, the heterologous DOD is encoded by a nucleic acid having at least 60% identity to the nucleic acid encoding a DOD from Phytolacca americana, as set forth in SEQ ID NO: 18.

In one embodiment, the heterologous DOD is encoded by a nucleic acid having at least 60% identity to the nucleic acid encoding a DOD from Portulaca grandiflora, as set forth in SEQ ID NO: 8.

In one embodiment, the heterologous truncated DOD (DOD*) is encoded by a nucleic acid having at least 60% identity to the nucleic acid as set forth in SEQ ID NO: 10 (PgDOD*) and SEQ ID NO: 59 (PgDOD*).

In one embodiment, the heterologous DOD is encoded by a nucleic acid having at least 60% identity to the nucleic acid encoding a DOD from Spinacia oleracea, as set forth in SEQ ID NO: 12.

In one embodiment, the heterologous DOD is encoded by a nucleic acid having at least 60% identity to the nucleic acid encoding a DOD from Suaeda salsa, as set forth in SEQ ID NO: 20.

Herein, a nucleic acid having at least 60% identity to a given nucleic acid may have at least 61% identity, such as at least 62% identity, such as at least 63% identity, such as at least 64% identity, such as at least 65% identity, such as at least 66% identity, such as at least 67% identity, such as at least 68% identity, such as at least 69% identity, such as at least 70% identity, such as at least 71% identity, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% identity to the given nucleic acid, or more.

### Enzyme Having Glycosyltransferase Activity

In some embodiments, the heterologous enzyme having glycosyltransferase activity, such as the glycosyltransferase, is encoded by a polynucleotide having at least 60% identity to a polynucleotide selected from the group of polynucleotides set forth in SEQ ID NO: 54, SEQ ID NO: 58, SEQ ID NO: 66 and SEQ ID NO: 68, such as at least 61%, such as at least 62%, such as at least 63%, such as at least 64%, such as at least 65%, such as at least 66%, such as at least 67%, such as at least 68%, such as at least 69%, such as at least 70%, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% identity thereto.

In one embodiment, the heterologous enzyme having glycosyltransferase activity is encoded by a nucleic acid having at least 60% identity to the nucleic acid encoding a glycosyltransferase from Beta vulgaris, as set forth in SEQ ID NO: 52.

In one embodiment, the heterologous enzyme having glycosyltransferase activity is encoded by a nucleic acid having at least 60% identity to the nucleic acid encoding a glycosyltransferase from Beta vulgaris, as set forth in SEQ ID NO: 54.

In one embodiment, the heterologous enzyme having glycosyltransferase activity is encoded by a nucleic acid having at least 60% identity to the nucleic acid encoding a glycosyltransferase from Beta vulgaris, as set forth in SEQ ID NO: 56.

In one embodiment, the heterologous enzyme having glycosyltransferase activity is encoded by a nucleic acid having at least 60% identity to the nucleic acid encoding a glycosyltransferase from Beta vulgaris, as set forth in SEQ ID NO: 58.

In one embodiment, the heterologous enzyme having glycosyltransferase activity is encoded by a nucleic acid having at least 60% identity to the nucleic acid encoding a glycosyltransferase from Chenopodium quinoa, as set forth in SEQ ID NO: 66.

In one embodiment, the heterologous enzyme having glycosyltransferase activity is encoded by a nucleic acid having at least 60% identity to the nucleic acid encoding a glycosyltransferase from Bougainvillea glabra, as set forth in SEQ ID NO: 68.

Herein, a nucleic acid having at least 60% identity to a given nucleic acid may have at least 61% identity, such as at least 62% identity, such as at least 63% identity, such as at least 64% identity, such as at least 65% identity, such as at least 66% identity, such as at least 67% identity, such as at least 68% identity, such as at least 69% identity, such as at least 70% identity, such as at least 71% identity, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% to the given nucleic acid, or more.

Further provided herein is the use of a polynucleotide as set forth in SEQ ID NO: 54, SEQ ID NO: 66; SEQ ID NO: 68 or SEQ ID NO: 58 for obtaining a protein capable of glycosylating a betalain and/or a betalain precursor, such as a protein capable of glycosylating betanidin and/or cyclo-DOPA, such as a protein with betanidin-5-O-glucosyltransferase activity and/or a protein with cyclo-DOPA 5-O-glucosyltransferase activity.

### Uses of Betalains, Betanin and Betaxanthins

The compounds produced by the present yeast cells or by the present methods have a wide range of applications. For instance, the betalains, such as betanin, isobetanin or one or more betaxanthins, can be used as food colorants, i.e. as natural food dyes. The betalains betanin and isobetanin are for example a permitted natural red food colorant, which is also used as a colorant in the cosmetics and pharmaceutical industry. Betalains have several advantages compared anthocyanins, another commonly used group of natural food dyes, including higher water solubility, higher tinctorial strength, and stability at a pH between 3 and 7.

Currently, the only existing commercial technology for production of betalains is via extraction from beetroot. This process is inefficient and provides an extract with undesirable flavour and aroma due to the presence of extract byproducts. Production of betalains in the cells and/or according to the methods disclosed herein provides higher product yield and titer, without undesired byproducts.

### EXAMPLES

The biosynthetic pathway of betalains (FIG. 3) starts with 3-hydroxylation of L-tyrosine to L-DOPA (L-3,4-dehydroxyphenylalanine), which is catalysed by the cytochrome P450 (CYP) enzyme family CYP76AD. L-DOPA is the main precursor of betalamic acid, the basic chromophore in the betalain pathway. L-DOPA is also the precursor to cyclo-DOPA that is catalysed by a similar enzyme family, CYP76AD. Sunnadeniya et al. (2016) made a structural study on the subfamily proteins of CYP76AD. They identified two major clades of α and β for this enzyme family, which exhibit different catalysing activities. CYP76AD clade α catalyses hydroxylation and oxidation of L-tyrosine to L-dopaquinone, while CYP76AD clade β only catalyses the hydroxylation of L-tyrosine to L-DOPA. This means that CYP76AD clade α, such as BvCYP76AD1 from B. vulgaris and MjCYP76AD3 from M. jalapa can form both betacyanins and betaxanthins (Sunnadeniya et al. 2016). Variants in clade β, such as BvCYP76AD5 and BvCYP76AD6 from B. vulgaris and MjCYP76AD15 from M. jalapa, however, can only form betaxanthins (Brockington et al. 2015; Timoneda et al. 2019). The expression and transcriptional regulation of α and β forms of CYP76AD proteins in the plants would allow them to produce different ratios and patterns of yellow or red pigments. Throughout this document, the genes encoding CYP76AD clade α and clade β as well as the corresponding CYP76AD enzymes are termed as TYH.

Betalamic acid synthesis from L-DOPA requires the action of the 4,5-DOPA extradiol dioxygenase enzyme (DOD) on L-DOPA. This enzyme catalyses the activation of 02 followed by incorporation of both atoms of oxygen into catechol derivatives. The result is the ring opening of L-DOPA to 4,5-seco-DOPA, followed by spontaneous tautomerism, nucleophilic addition, proton transfer and water elimination to form betalamic acid (Gandia-Herrero and Garcia-Carmona 2020).

### Example 1—Materials and Methods

### Strains and Media

To clone and store plasmids, E. coli strain DH5a was used. The cultivations were carried out at 37° C. in Lysogeny Broth (LB) broth or on agar-plates supplemented with 100 mg/L ampicillin as selection marker. The yeast strain CEN.PK113-5D (MATa ura3-52 HIS3 LEU2 TRP1 MAL2-8c SUC2) harboring episomal vector for Cas9 protein expression (Ptef1-Cas9-Tcyc1_kanMX) was used as the parent strain (ST8251) in this study (Milne et al. 2020). To keep the selection for Cas9, the cultivations for all the yeast strains were supplemented with 200 mg/L G418 (Sigma-Aldrich). The construction of yeast strains was carried out by EasyClone MarkerFree method (Jessop-Fabre et al. 2016). The yeast strains used in the study be seen in Table 1.

Starter cultures of constructed yeast strains were grown in yeast synthetic drop-out medium without uracil (Sigma-Aldrich). For colour analysis experiments, the MM (pABA⁻) medium, i.e. modified mineral medium without p-aminobenzoic acid, was used to culture the cells. This medium consisted of 20 g/L glucose, 7.5 g/L (NH₄)₂SO₄, 14.4 g/L KH₂PO₄, 0.5 g/L MgSO₄·7H₂O, 2 mL/L trace metal solution (3.0 g/L FeSO₄·7H₂O, 4.5 g/L ZnSO₄·7H₂O, 4.5 g/L CaCl₂·2H₂O, 0.84 g/L MnCl₂·2H₂O, 0.3 g/L CoCl₂·6H₂O, 0.3 g/L CuSO₄·5H₂O, 0.4 g/L Na₂MoO₄·2H₂O, 1.0 g/L H₃BO₃, 0.1 g/L KI, and 19.0 g/L Na₂EDTA·2H₂O), and 1 mL/L vitamin solution (0.05 g/L D-biotin, 1.0 g/L D-pantothenic acid hemicalcium salt, 1.0 g/L thiamin-HCl, 1.0 g/L pyridoxin-HCl, 1.0 g/L nicotinic acid, and 25.0 g/L myo-inositol). Para-aminobenzoic acid was excluded from the medium to avoid interaction with betaxanthin production.

### Synthetic Genes and DNA Materials

The heterologous genes (SEQ ID NOs: 1-58) were all synthesized by GeneArt (Life Technologies) in codon-optimized versions for S. cerevisiae. All DNA parts were PCR amplified using Phusion U DNA polymerase (ThermoFisher) according to the manufacturer's instructions. The DNA fragments (BioBricks) are listed in Table 2. The DNA fragments obtained by PCR were separated in 1%-agarose containing RedSafe™ (iNtRON Biotechnology), and purified using the Nucleospin Gel and PCR Clean-up kit (Macherey-Nagel). Intergrative vectors were constructed as described in EasyClone MarkerFree method (Jessop-Fabre et al. 2016). Vectors used in the study can be seen in Table 3.

TABLE 1
Yeast strains. The ↑ denotes overexpression.

| Strain | | Parent | Integrated DNA |
|---|---|---|---|
| name | Characteristics | strain | element |
| ST6512 | MATa ku70Δ::PrTEF1-cas9- | Y. lipolytica | ku70Δ::SpCas9- |
| | TTef12::PrGPD-dsdAMX-TLip2 | W29, Y- | EcDsdAMX4 |
| | | 63746, ATCC | |
| | | 20460 | |
| ST9632 | CEN.PK113-7D leu2Δ, Tadh1- | CEN.PK113- | pCfB2312 (TEF1p- |
| | DOD-Ptef1-Ppgk1-TYH-Tcyc1 | 7D | Cas9- |
| | | | CYC1t_kanMX), |
| | | | pCfB9195 (pX-2<- |
| | | | MjDOD-BvCYP76- |
| | | | AD^{W13L,F309L}->) |
| ST10319 | CEN.PK113-5D, ↑Cas9, | ST8251 | BB0629, BB4773, |
| | CAN1::(Ptef1-MjDOD-Tcyc1- | | BB4774, BB4732, |
| | Ptdh3-BvCYP76AD^{W13L}-Tadh1- | | BB0630 |
| | KIURA3) | | |
| ST10528 | CEN.PK113-5D, ↑Cas9, | iso2 | pCfB2312 (TEF1p- |
| | CAN1::(Ptef1-PgDOD*-Tcyc1- | | Cas9- |
| | Ptef1-BgDOD1(2x)-Tcyc1- | | CYC1t_kanMX) |
| | Ptdh3-AnTYH-Tadh1-Ptdh3- | | |
| | BaTYH-Tadh1-Ptdh3-CbTYH- | | |
| | Tadh1-KIURA3) | | |
| ST10529 | CEN.PK113-5D, ↑Cas9, | iso21 | pCfB2312 (TEF1p- |
| | CAN1::(Ptef1-PgDOD*-Tcyc1- | | Cas9- |
| | Ptef1-BgDOD1(2x)-Tcyc1- | | CYC1t_kanMX) |
| | Ptdh3-AnTYH(2x)-Tadh1-Ptdh3- | | |
| | CbTyH-Tadh1-KIURA3) | | |
| ST10613 | CEN.PK113-5D, ↑Cas9, | ST10528 | pCfB10368 (X- |
| | CAN1::(Ptef1-PgDOD*-Tcyc1- | | 3_BvSGT1) |
| | Ptef1-BgDOD1(2x)-Tcyc1- | | |
| | Ptdh3-AnTYH-Tadh1-Ptdh3- | | |
| | BaTYH-Tadh1-Ptdh3-CbTYH- | | |
| | Tadh1-KIURA3), | | |
| | Ptef1-BvSGT1-Tadh1 | | |
| ST10614 | CEN.PK113-5D, ↑Cas9, | ST10528 | pCfB10369 (X- |
| | CAN1::(Ptef1-PgDOD*-Tcyc1- | | 3_BvSGT2) |
| | Ptef1-BgDOD1(2x)-Tcyc1- | | |
| | Ptdh3-AnTYH-Tadh1-Ptdh3- | | |
| | BaTYH-Tadh1-Ptdh3-CbTYH- | | |
| | Tadh1-KIURA3), | | |
| | Ptef1-BvSGT2-Tadh1 | | |
| ST10615 | CEN.PK113-5D, ↑Cas9, | ST10528 | pCfB10370 (X- |
| | CAN1::(Ptef1-PgDOD*-Tcyc1- | | 3_BvSGT3) |
| | Ptef1-BgDOD1(2x)-Tcyc1- | | |
| | Ptdh3-AnTYH-Tadh1-Ptdh3- | | |
| | BaTYH-Tadh1-Ptdh3-CbTYH- | | |
| | Tadh1-KIURA3), | | |
| | Ptef1-BvSGT3-Tadh1 | | |
| ST10616 | CEN.PK113-5D, ↑Cas9, | ST10528 | pCfB10371 (X- |
| | CAN1::(Ptef1-PgDOD*-Tcyc1- | | 3_BvSGT4) |
| | Ptef1-BgDOD1(2x)-Tcyc1- | | |
| | Ptdh3-AnTYH-Tadh1-Ptdh3- | | |
| | BaTYH-Tadh1-Ptdh3-CbTYH- | | |
| | Tadh1-KIURA3), | | |
| | Ptef1-BvSGT4-Tadh1 | | |
| ST10617 | CEN.PK113-5D, ↑Cas9, | ST10528 | pCfB10106 (X- |
| | CAN1::(Ptef1-PgDOD*-Tcyc1- | | 3_Ptef1-DbB5GT) |
| | Ptef1-BgDOD1(2x)-Tcyc1- | | |
| | Ptdh3-AnTYH-Tadh1-Ptdh3- | | |
| | BaTYH-Tadh1-Ptdh3-CbTYH- | | |
| | Tadh1-KIURA3), | | |
| | Ptef1-DbB5GT-Tadh1 | | |
| ST10618 | CEN.PK113-5D, ↑Cas9, | ST10529 | pCfB10368 (X- |
| | CAN1::(Ptef1-PgDOD*-Tcyc1- | | 3_BvSGT1) |
| | Ptef1-BgDOD1(2x)-Tcyc1- | | |
| | Ptdh3-AnTYH(2x)-Tadh1-Ptdh3- | | |
| | CbTyH-Tadh1-KIURA3), Ptef1- | | |
| | BvSGT1-Tadh1 | | |
| ST10619 | CEN.PK113-5D, ↑Cas9, | ST10529 | pCfB10369 (X- |
| | CAN1::(Ptef1-PgDOD*-Tcyc1- | | 3_BvSGT2) |
| | Ptef1-BgDOD1(2x)-Tcyc1- | | |
| | Ptdh3-AnTYH(2x)-Tadh1-Ptdh3- | | |
| | CbTyH-Tadh1-KIURA3), Ptef1- | | |
| | BvSGT2-Tadh1 | | |
| ST10620 | CEN.PK113-5D, ↑Cas9, | ST10529 | pCfB10370 (X- |
| | CAN1::(Ptef1-PgDOD*-Tcyc1- | | 3_BvSGT3) |
| | Ptef1-BgDOD1(2x)-Tcyc1- | | |
| | Ptdh3-AnTYH(2x)-Tadh1-Ptdh3- | | |
| | CbTyH-Tadh1-KIURA3), Ptef1- | | |
| | BvSGT3-Tadh1 | | |
| ST10621 | CEN.PK113-5D, ↑Cas9, | ST10529 | pCfB10371 (X- |
| | CAN1::(Ptef1-PgDOD*-Tcyc1- | | 3_BvSGT4) |
| | Ptef1-BgDOD1(2x)-Tcyc1- | | |
| | Ptdh3-AnTYH(2x)-Tadh1-Ptdh3- | | |
| | CbTyH-Tadh1-KIURA3), Ptef1- | | |
| | BvSGT4-Tadh1 | | |
| ST10622 | CEN.PK113-5D, ↑Cas9, | ST10529 | pCfB10106 (X- |
| | CAN1::(Ptef1-PgDOD*-Tcyc1- | | 3_Ptef1-DbB5GT) |
| | Ptef1-BgDOD1(2x)-Tcyc1- | | |
| | Ptdh3-AnTYH(2x)-Tadh1-Ptdh3- | | |
| | CbTyH-Tadh1-KIURA3), Ptef1- | | |
| | DbB5GT-Tadh1 | | |
| ST10780 | CEN.PK113-5D, ↑Cas9, | ST8251 | BB0629, BB4735, |
| | CAN1::(Ptef1-PgDOD-Tcyc1- | | BB4774, BB4732, |
| | Ptdh3-AnTYH-Tadh1-KIURA3) | | BB0630 |
| ST11015 | MATA ku70Δ::PrTEF1-Cas9- | ST6512 | pCfB10619(IntC2_P |
| | TTef12::PrGPD-DsdA-TLip2 ↑ | | gDOD*_ |
| | PgDOD* ↑ BvCYP76AD^{W13L} | | BvCYP76AD^{W13L}) |
| ST11016 | MATA ku70Δ::PrTEF1-Cas9- | ST6512 | pCfB10620(IntC2_P |
| | TTef12::PrGPD-DsdA-TLip2 ↑ | | gDOD*_ EvTYH) |
| | PgDOD* ↑EvTYH | | |
| ST11018 | MATA ku70Δ::PrTEF1-Cas9- | ST6512 | pCfB10622(IntC2_B |
| | TTef12::PrGPD-DsdA-TLip2 ↑ | | gDOD2_ |
| | BgDOD2 ↑ BvCYP76AD^{W13L} | | BvCYP76AD^{W13L}) |
| ST11019 | MATA ku70Δ::PrTEF1-Cas9- | ST6512 | pCfB10623(IntC2_B |
| | TTef12::PrGPD-DsdA-TLip2 ↑ | | gDOD2_EvTYH) |
| | BgDOD2 ↑EvTYH | | |
| ST11020 | MATA ku70Δ::PrTEF1-Cas9- | ST6512 | pCfB10621(IntC2_B |
| | TTef12::PrGPD-DsdA-TLip2 ↑ | | gDOD2_AnTYH) |
| | BgDOD2 ↑AnTYH | | |
| ST11021 | MATA ku70Δ::PrTEF1-Cas9- | ST6512 | pCfB10625(IntC2_M |
| | TTef12::PrGPD-DsdA-TLip2 ↑ | | jDOD_ |
| | MjDOD ↑ BvCYP76AD^{W13L} | | BvCYP76AD^{W13L}) |
| ST11022 | MATA ku70Δ::PrTEF1-Cas9- | ST6512 | pCfB10626(IntC2_M |
| | TTef12::PrGPD-DsdA-TLip2 ↑ | | jDOD_EvTYH) |
| | MjDOD ↑EvTYH | | |
| ST11023 | MATA ku70Δ::PrTEF1-Cas9- | ST6512 | pCfB10624(IntC2_M |
| | TTef12::PrGPD-DsdA-TLip2 ↑ | | jDOD_AnTYH) |
| | MjDOD ↑AnTYH | | |
| ST11193 | MATA ku70Δ::PrTEF1-Cas9- | ST11022 | pCfB10607(IntD_Bv |
| | TTef12::PrGPD-DsdA-TLip2 | | SGT2) |
| | ↑MjDOD ↑EvTYH | | |
| | ↑BvSGT2 | | |
| ST11663 | MATA ku70Δ::PrTEF1-Cas9- | ST11022 | pCfB8977 (Int E_1- |
| | TTef12::PrGPD-DsdA-TLip2 | | tPex20<- |
| | ↑MjDOD ↑EvTYH | | YIARO7_G139S<- |
| | ↑YIARO7_G139S | | PrGPD::PrTEFin-> |
| | ↑YIARO4_K221L | | YIARO4_K221L-> |
| | | | tLip2) |
| ST11664 | MATA ku70Δ::PrTEF1-Cas9- | ST11193 | pCfB8977 (Int E_1- |
| | TTef12::PrGPD-DsdA-TLip2 | | tPex20<- |
| | ↑MjDOD ↑EvTYH | | YIARO7_G139S<- |
| | ↑BvSGT2 ↑YIARO7_G139S | | PrGPD::PrTEFin-> |
| | ↑YIARO4_K221L | | YIARO4_K221L-> |
| | | | tLip2) |
| ST11939 | MATA ku70Δ::PrTEF1-Cas9- | ST11022 | pCfB11114 (IntF_3- |
| | TTef12::PrGPD-DsdA-TLip2 | | TPex20<- |
| | ↑MjDOD ↑EvTYH | | MjDOD_YI<- |
| | ↑MjDOD ↑EvTYH | | PrGPD::PrTEFin-> |
| | | | EvTYH_YI->TLip2) |
| ST11940 | MATA ku70Δ::PrTEF1-Cas9- | ST11193 | pCfB11113 (IntF_3- |
| | TTef12::PrGPD-DsdA-TLip2 | | TPex20<- |
| | ↑MjDOD ↑EvTYH | | BvSGT2_YI<- |
| | ↑BvSGT2 ↑MjDOD ↑EvTYH | | PrTEFin,<-TPex20<- |
| | ↑BvSGT2 | | MjDOD_YI- |
| | | | PrGPD::PrTEFin-> |
| | | | EvTYH_YI->TLip2) |
| ST11941 | MATA ku70Δ::PrTEF1-Cas9- | ST11663 | pCfB11114 (IntF_3- |
| | TTef12::PrGPD-DsdA-TLip2 | | TPex20<- |
| | ↑MjDOD ↑EvTYH | | MjDOD_YI<- |
| | ↑YIARO7_G139S | | PrGPD::PrTEFin-> |
| | ↑YIARO4_K221L | | EvTYH_YI->TLip2) |
| | ↑MjDOD ↑EvTYH | | |
| ST11942 | MATA ku70Δ::PrTEF1-Cas9- | ST11664 | pCfB11113 (IntF_3- |
| | TTef12::PrGPD-DsdA-TLip2 | | TPex20<- |
| | ↑MjDOD ↑EvTYH | | BvSGT2_YI<- |
| | ↑BvSGT2 ↑YIARO7_G139S | | PrTEFin,<-TPex20<- |
| | ↑YIARO4_K221L | | MjDOD_YI- |
| | ↑MjDOD ↑EvTYH ↑BvSGT2 | | PrGPD::PrTEFin-> |
| | | | EvTYH_YI->TLip2) |
| ST12153 | CEN.PK113-5D, ↑Cas9, | ST10529 | pCfB11102 (X- |
| | CAN1::(Ptef1-PgDOD*-Tcyc1- | | 3_BvSGT5) |
| | Ptef1-BgDOD1(2x)-Tcyc1- | | |
| | Ptdh3-AnTYH(2x)-Tadh1-Ptdh3- | | |
| | CbTyH-Tadh1-KIURA3), Ptef1- | | |
| | BvSGT5-Tadh1 | | |
| ST12154 | CEN.PK113-5D, ↑Cas9, | ST10529 | pCfB11103 (X- |
| | CAN1::(Ptef1-PgDOD*-Tcyc1- | | 3_CqSGT1)) |
| | Ptef1-BgDOD1(2x)-Tcyc1- | | |
| | Ptdh3-AnTYH(2x)-Tadh1-Ptdh3- | | |
| | CbTyH-Tadh1-KIURA3), Ptef1- | | |
| | CqSGT1-Tadh1 | | |
| ST12155 | CEN.PK113-5D, ↑Cas9, | ST10529 | pCfB11104 (X- |
| | CAN1::(Ptef1-PgDOD*-Tcyc1- | | 3_PaSGT1) |
| | Ptef1-BgDOD1(2x)-Tcyc1- | | |
| | Ptdh3-AnTYH(2x)-Tadh1-Ptdh3- | | |
| | CbTyH-Tadh1-KIURA3), Ptef1- | | |
| | PaSGT1-Tadh1 | | |
| ST12156 | CEN.PK113-5D, ↑Cas9, | ST10529 | pCfB11105 (X- |
| | CAN1:(Ptef1-PgDOD*-Tcyc1- | | 3_SoSGT1) |
| | Ptef1-BgDOD1(2x)-Tcyc1- | | |
| | Ptdh3-AnTYH(2x)-Tadh1-Ptdh3- | | |
| | CbTyH-Tadh1-KIURA3), Ptef1- | | |
| | SoSGT1-Tadh1 | | |
| ST12157 | CEN.PK113-5D, ↑Cas9, | ST10529 | pCfB11106 (X- |
| | CAN1::(Ptef1-PgDOD*-Tcyc1- | | 3_VrSGT1) |
| | Ptef1-BgDOD1(2x)-Tcyc1- | | |
| | Ptdh3-AnTYH(2x)-Tadh1-Ptdh3- | | |
| | CbTyH-Tadh1-KIURA3), Ptef1- | | |
| | VrSGT1-Tadh1 | | |
| ST12260 | CEN.PK113-5D, ↑Cas9, | ST10529 | pCfB11617 (X-3_ |
| | CAN1::(Ptef1-PgDOD*-Tcyc1- | | CqSGT2) |
| | Ptef1-BgDOD1(2x)-Tcyc1- | | |
| | Ptdh3-AnTYH(2x)-Tadh1-Ptdh3- | | |
| | CbTyH-Tadh1-KIURA3), Ptef1- | | |
| | CqSGT2-Tadh1 | | |
| ST12291 | CEN.PK113-5D, ↑Cas9, | ST10529 | pCfB11618 (X-3_ |
| | CAN1::(Ptef1-PgDOD*-Tcyc1- | | CqSGT3) |
| | Ptef1-BgDOD1(2x)-Tcyc1- | | |
| | Ptdh3-AnTYH(2x)-Tadh1-Ptdh3- | | |
| | CbTyH-Tadh1-KIURA3), Ptef1- | | |
| | CqSGT3-Tadh1 | | |
| ST12292 | CEN.PK113-5D, ↑Cas9, | ST10529 | pCfB11619 (X-3_ |
| | CAN1::(Ptef1-PgDOD*-Tcyc1- | | CqSGT4) |
| | Ptef1-BgDOD1(2x)-Tcyc1- | | |
| | Ptdh3-AnTYH(2x)-Tadh1-Ptdh3- | | |
| | CbTyH-Tadh1-KIURA3), Ptef1- | | |
| | CqSGT4-Tadh1 | | |
| ST12293 | CEN.PK113-5D, ↑Cas9, | ST10529 | pCfB11620 (X-3_ |
| | CAN1::(Ptef1-PgDOD*-Tcyc1- | | CqSGT5) |
| | Ptef1-BgDOD1(2x)-Tcyc1- | | |
| | Ptdh3-AnTYH(2x)-Tadh1-Ptdh3- | | |
| | CbTyH-Tadh1-KIURA3), Ptef1- | | |
| | CqSGT5-Tadh1 | | |
| ST12294 | CEN.PK113-5D, ↑Cas9, | ST10529 | pCfB11621 (X-3_ |
| | CAN1::(Ptef1-PgDOD*-Tcyc1- | | CqSGT6) |
| | Ptef1-BgDOD1(2x)-Tcyc1- | | |
| | Ptdh3-AnTYH(2x)-Tadh1-Ptdh3- | | |
| | CbTyH-Tadh1-KIURA3), Ptef1- | | |
| | CqSGT6-Tadh1 | | |
| ST12295 | CEN.PK113-5D, ↑Cas9, | ST10529 | pCfB11622 (X-3_ |
| | CAN1::(Ptef1-PgDOD*-Tcyc1- | | SoSGT2) |
| | Ptef1-BgDOD1(2x)-Tcyc1- | | |
| | Ptdh3-AnTYH(2x)-Tadh1-Ptdh3- | | |
| | CbTyH-Tadh1-KIURA3), Ptef1- | | |
| | SoSGT2-Tadh1 | | |
| ST12296 | CEN.PK113-5D, ↑Cas9, | ST10529 | pCfB11623 (X- |
| | CAN1::(Ptef1-PgDOD*-Tcyc1- | | 3_SoSGT3) |
| | Ptef1-BgDOD1(2x)-Tcyc1- | | |
| | Ptdh3-AnTYH(2x)-Tadh1-Ptdh3- | | |
| | CbTyH-Tadh1-KIURA3), Ptef1- | | |
| | SoSGT3-Tadh1 | | |
| ST12297 | CEN.PK113-5D, ↑Cas9, | ST10529 | pCfB11624 (X-3_ |
| | CAN1::(Ptef1-PgDOD*-Tcyc1- | | SoSGT4) |
| | Ptef1-BgDOD1(2x)-Tcyc1- | | |
| | Ptdh3-AnTYH(2x)-Tadh1-Ptdh3- | | |
| | CbTyH-Tadh1-KIURA3), Ptef1- | | |
| | SoSGT4-Tadh1 | | |
| ST12298 | CEN.PK113-5D, ↑Cas9, | ST10529 | pCfB11625 (X-3_ |
| | CAN1::(Ptef1-PgDOD*-Tcyc1- | | SoSGT5) |
| | Ptef1-BgDOD1(2x)-Tcyc1- | | |
| | Ptdh3-AnTYH(2x)-Tadh1-Ptdh3- | | |
| | CbTyH-Tadh1-KIURA3), Ptef1- | | |
| | SoSGT5-Tadh1 | | |
| ST12299 | CEN.PK113-5D, ↑Cas9, | ST10529 | pCfB11626 (X-3_ |
| | CAN1::(Ptef1-PgDOD*-Tcyc1- | | SoSGT6) |
| | Ptef1-BgDOD1(2x)-Tcyc1- | | |
| | Ptdh3-AnTYH(2x)-Tadh1-Ptdh3- | | |
| | CbTyH-Tadh1-KIURA3), Ptef1- | | |
| | SoSGT6-Tadh1 | | |
| ST12300 | CEN.PK113-5D, ↑Cas9, | ST10529 | pCfB11627 (X-3_ |
| | CAN1::(Ptef1-PgDOD*-Tcyc1- | | SoSGT7) |
| | Ptef1-BgDOD1(2x)-Tcyc1- | | |
| | Ptdh3-AnTYH(2x)-Tadh1-Ptdh3- | | |
| | CbTyH-Tadh1-KIURA3), Ptef1- | | |
| | SoSGT7-Tadh1 | | |
| ST12301 | CEN.PK113-5D, ↑Cas9, | ST10529 | pCfB11628 (X-3 |
| | CAN1::(Ptef1-PgDOD*-Tcyc1- | | CsSGT1) |
| | Ptef1-BgDOD1(2x)-Tcyc1- | | |
| | Ptdh3-AnTYH(2x)-Tadh1-Ptdh3- | | |
| | CbTyH-Tadh1-KIURA3), Ptef1- | | |
| | CsSGT1-Tadh1 | | |
| ST12302 | CEN.PK113-5D, ↑Cas9, | ST10529 | pCfB11629 (X-3_ |
| | CAN1::(Ptef1-PgDOD*-Tcyc1- | | CiSiSGT1) |
| | Ptef1-BgDOD1(2x)-Tcyc1- | | |
| | Ptdh3-AnTYH(2x)-Tadh1-Ptdh3- | | |
| | CbTyH-Tadh1-KIURA3), Ptef1- | | |
| | CiSiSGT1-Tadh1 | | |
| ST12303 | CEN.PK113-5D, ↑Cas9, | ST10529 | pCfB11630 (X-3_ |
| | CAN1::(Ptef1-PgDOD*-Tcyc1- | | CiCISGT1) |
| | Ptef1-BgDOD1(2x)-Tcyc1- | | |
| | Ptdh3-AnTYH(2x)-Tadh1-Ptdh3- | | |
| | CbTyH-Tadh1-KIURA3), Ptef1- | | |
| | CiCISGT1-Tadh1 | | |
| ST12304 | CEN.PK113-5D, ↑Cas9, | ST10529 | pCfB11631 (X- |
| | CAN1::(Ptef1-PgDOD*-Tcyc1- 3_ | | EgSGT1) |
| | Ptef1-BgDOD1(2x)-Tcyc1- | | |
| | Ptdh3-AnTYH(2x)-Tadh1-Ptdh3- | | |
| | CbTyH-Tadh1-KIURA3), Ptef1- | | |
| | EgSGT1-Tadh1 | | |
| ST12305 | CEN.PK113-5D, ↑Cas9, | ST10529 | pCfB11632 (X-3_ |
| | CAN1::(Ptef1-PgDOD*-Tcyc1- | | CpSGT1) |
| | Ptef1-BgDOD1(2x)-Tcyc1- | | |
| | Ptdh3-AnTYH(2x)-Tadh1-Ptdh3- | | |
| | CbTyH-Tadh1-KIURA3), Ptef1- | | |
| | CpSGT1-Tadh1 | | |
| ST12306 | CEN.PK113-5D, ↑Cas9, | ST10529 | pCfB11633 (X-3_ |
| | CAN1::(Ptef1-PgDOD*-Tcyc1- | | MeSGT1) |
| | Ptef1-BgDOD1(2x)-Tcyc1- | | |
| | Ptdh3-AnTYH(2x)-Tadh1-Ptdh3- | | |
| | CbTyH-Tadh1-KIURA3), Ptef1- | | |
| | MeSGT1-Tadh1 | | |
| ST12307 | CEN.PK113-5D, ↑Cas9, | ST10529 | pCfB11634 (X-3_ |
| | CAN1::(Ptef1-PgDOD*-Tcyc1- | | RaSGT1) |
| | Ptef1-BgDOD1(2x)-Tcyc1- | | |
| | Ptdh3-AnTYH(2x)-Tadh1-Ptdh3- | | |
| | CbTyH-Tadh1-KIURA3), Ptef1- | | |
| | RaSGT1-Tadh1 | | |
| ST12308 | CEN.PK113-5D, ↑Cas9, | ST10529 | pCfB11635 (X- |
| | CAN1::(Ptef1-PgDOD*-Tcyc1- | | 3_TCSGT1) |
| | Ptef1-BgDOD1(2x)-Tcyc1- | | |
| | Ptdh3-AnTYH(2x)-Tadh1-Ptdh3- | | |
| | CbTyH-Tadh1-KIURA3), Ptef1- | | |
| | TcSGT1-Tadh1 | | |
| ST12173 | CEN.PK113-5D, ↑Cas9, | ST10529 | pCfB11393 (X- |
| | CAN1::(Ptef1-PgDOD*-Tcyc1- | | 3_BgGT1) |
| | Ptef1-BgDOD1(2x)-Tcyc1- | | |
| | Ptdh3-AnTYH(2x)-Tadh1-Ptdh3- | | |
| | CbTyH-Tadh1-KIURA3), Ptef1- | | |
| | BgGT1-Tadh1 | | |
| ST12174 | CEN.PK113-5D, ↑Cas9, | ST10529 | pCfB11394 (X- |
| | CAN1::(Ptef1-PgDOD*-Tcyc1- | | 3_BgGT2) |
| | Ptef1-BgDOD1(2x)-Tcyc1- | | |
| | Ptdh3-AnTYH(2x)-Tadh1-Ptdh3- | | |
| | CbTyH-Tadh1-KIURA3), Ptef1- | | |
| | BgGT2-Tadh1 | | |
| ST12175 | CEN.PK113-5D, ↑Cas9, | ST10529 | pCfB11395 (X- |
| | CAN1::(Ptef1-PgDOD*-Tcyc1- | | 3_BgGT3) |
| | Ptef1-BgDOD1(2x)-Tcyc1- | | |
| | Ptdh3-AnTYH(2x)-Tadh1-Ptdh3- | | |
| | CbTyH-Tadh1-KIURA3), Ptef1- | | |
| | BgGT3-Tadh1 | | |
| ST12309 | MATA ku70Δ::PrTEF1-Cas9- | ST11942 | BB5999 (4-HPPD- |
| | TTef12::PrGPD-DsdA-TLip2 | | HphSyn-LoxP) |
| | ↑MjDOD ↑EvTYH | | |
| | ↑BvSGT2 ↑YIARO7_G139S | | |
| | ↑YIARO4_K221L | | |
| | ↑MjDOD ↑EvTYH ↑BvSGT2 Δ4- | | |
| | HPPD | | |

TABLE 2
BioBricks.

| | Fw- | Rv- | |
|---|---|---|---|
| BioBrick name | primer | primer | Template DNA |
| BB8 (Ptef1<-) | PR-5 | PR-6 | CEN.PK113-5D |
| | | | genome |
| BB4273 (DbB5GT_U1) | PR-25382 | PR-25383 | Synthetic gene |
| BB4686 (BvDOD1_gene1) | PR-27250 | PR-27251 | Synthetic gene |
| BB4687 (BgDOD1_gene1) | PR-27252 | PR-27253 | Synthetic gene |
| BB4688 (PgDOD_gene1) | PR-27254 | PR-27255 | Synthetic gene |
| BB4689 (SoDOD_gene1) | PR-27256 | PR-27257 | Synthetic gene |
| BB4690 (AtDOD_gene1) | PR-27258 | PR-27259 | Synthetic gene |
| BB4691 (AhDOD_gene1) | PR-27260 | PR-27261 | Synthetic gene |
| BB4692 (PaDOD_gene1) | PR-27262 | PR-27263 | Synthetic gene |
| BB4693 (SsDOD_gene1) | PR-27264 | PR-27265 | Synthetic gene |
| BB4694 (BgDOD2_gene1) | PR-27266 | PR-27267 | Synthetic gene |
| BB4695 (BvDOD2_gene1) | PR-27268 | PR-27269 | Synthetic gene |
| BB4696 (BvDOD3_gene1) | PR-27270 | PR-27271 | Synthetic gene |
| BB4730 (MjDOD_gene1) | PR-25066 | PR-27394 | Synthetic gene |
| BB4731 (TyrH(W13L)_gene2) | PR-27395 | PR-27396 | Synthetic gene |
| BB4697 (AnTyH_gene2) | PR-27272 | PR-27273 | Synthetic gene |
| BB4698 (AoTyH_gene2) | PR-27274 | PR-27275 | Synthetic gene |
| BB4699 (BaTyH_gene2) | PR-27276 | PR-27277 | Synthetic gene |
| BB4700 (CbTyH_gene2) | PR-27278 | PR-27279 | Synthetic gene |
| BB4701 (EvTyH_gene2) | PR-27280 | PR-27281 | Synthetic gene |
| BB4702 (MmTyH1_gene2) | PR-27282 | PR-27283 | Synthetic gene |
| BB4703 (MmTyH2_gene2) | PR-27284 | PR-27285 | Synthetic gene |
| BB4704 (OfTyH_gene2) | PR-27286 | PR-27287 | Synthetic gene |
| BB4705 (PaTyH_gene2) | PR-27288 | PR-27289 | Synthetic gene |
| BB4706 (PdTyH_gene2) | PR-27290 | PR-27291 | Synthetic gene |
| BB0622 (Ptef1) | PR-7578 | PR-7579 | CEN.PK113-5D |
| | | | genome |
| BB0623 (Tadh1) | PR-7580 | PR-7581 | CEN.PK113-5D |
| | | | genome |
| BB0618 (Ptdh3) | PR-7570 | PR-7571 | CEN.PK113-5D |
| | | | genome |
| BB0624 (Tcyc1) | PR-7582 | PR-7583 | CEN.PK113-5D |
| | | | genome |
| BB0629 (CAN1-UP-A) | PR-7540 | PR-7596 | CEN.PK113-5D |
| | | | genome |
| BB0630 (CAN1-DW-E) | PR-7597 | PR-7545 | CEN.PK113-5D |
| | | | genome |
| BB3907 (<-PrGPD::PrTEFin->) | PR-23965 | PR-23967 | pCfB8977 |
| BB3908 (PrTEFin->single) | PR-23995 | PR-23967 | pCfB8977 |
| BB4732 (C-KIURA3-E) | PR-27409 | PR-27410 | p0018 |
| BB4733 (A-Ptef1-BvDOD1- | PR-7587 | PR-7588 | USER and Ligation |
| Tcyc1-B) | | | of BB0622, BB0624, |
| | | | BB4686 |
| BB4734 (A-Ptef1-BgDOD1- | PR-7587 | PR-7588 | USER and Ligation |
| Tcyc1-B) | | | of BB0622, BB0624, |
| | | | BB4687 |
| BB4735 (A-Ptef1-PgDOD- | PR-7587 | PR-7588 | USER and Ligation |
| Tcyc1-B) | | | of BB0622, BB0624, |
| | | | BB4688 |
| BB4736 (A-Ptef1-SoDOD- | PR-7587 | PR-7588 | USER and Ligation |
| Tcyc1-B) | | | of BB0622, BB0624, |
| | | | BB4689 |
| BB4737 (A-Ptef1-AtDOD-Tcyc1- | PR-7587 | PR-7588 | USER and Ligation |
| B) | | | of BB0622, BB0624, |
| | | | BB4690 |
| BB4738 (A-Ptef1-AhDOD- | PR-7587 | PR-7588 | USER and Ligation |
| Tcyc1-B) | | | of BB0622, BB0624, |
| | | | BB4691 |
| BB4739 (A-Ptef1-PaDOD- | PR-7587 | PR-7588 | USER and Ligation |
| Tcyc1-B) | | | of BB0622, BB0624, |
| | | | BB4692 |
| BB4740 (A-Ptef1-SsDOD- | PR-7587 | PR-7588 | USER and Ligation |
| Tcyc1-B) | | | of BB0622, BB0624, |
| | | | BB4693 |
| BB4741 (A-Ptef1-BgDOD2- | PR-7587 | PR-7588 | USER and Ligation |
| Tcyc1-B) | | | of BB0622, BB0624, |
| | | | BB4694 |
| BB4742 (A-Ptef1-BvDOD2- | PR-7587 | PR-7588 | USER and Ligation |
| Tcyc1-B) | | | of BB0622, BB0624, |
| | | | BB4695 |
| BB4743 (A-Ptef1-BvDOD3- | PR-7587 | PR-7588 | USER and Ligation |
| Tcyc1-B) | | | of BB0622, BB0624, |
| | | | BB4696 |
| BB4733 (A-Ptef1-BvDOD1- | PR-7587 | PR-7588 | USER and Ligation |
| Tcyc1-B) | | | of BB0622, BB0624, |
| | | | BB4686 |
| BB4734 (A-Ptef1-BgDOD1- | PR-7587 | PR-7588 | USER and Ligation |
| Tcyc1-B) | | | of BB0622, BB0624, |
| | | | BB4687 |
| BB4735 (A-Ptef1-PgDOD- | PR-7587 | PR-7588 | USER and Ligation |
| Tcyc1-B) | | | of BB0622, BB0624, |
| | | | BB4688 |
| BB4736 (A-Ptef1-SoDOD- | PR-7587 | PR-7588 | USER and Ligation |
| Tcyc1-B) | | | of BB0622, BB0624, |
| | | | BB4689 |
| BB4741 (A-Ptef1-BgDOD2- | PR-7587 | PR-7588 | USER and Ligation |
| Tcyc1-B) | | | of BB0622, BB0624, |
| | | | BB4694 |
| BB4742 (A-Ptef1-BvDOD2- | PR-7587 | PR-7588 | USER and Ligation |
| Tcyc1-B) | | | of BB0622, BB0624, |
| | | | BB4695 |
| BB4743 (A-Ptef1-BvDOD3- | PR-7587 | PR-7588 | USER and Ligation |
| Tcyc1-B) | | | of BB0622, BB0624, |
| | | | BB4696 |
| BB4744 (B-Ptdh3-AnTyH- | PR-7589 | PR-7590 | USER and Ligation |
| Tadh1-C) | | | of BB0618, BB0623, |
| | | | BB4697 |
| BB4745 (B-Ptdh3-AoTyH- | PR-7589 | PR-7590 | USER and Ligation |
| Tadh1-C) | | | of BB0618, BB0623, |
| | | | BB4698 |
| BB4746 (B-Ptdh3-BaTyH- | PR-7589 | PR-7590 | USER and Ligation |
| Tadh1-C) | | | of BB0618, BB0623, |
| | | | BB4699 |
| BB4747 (B-Ptdh3-CbTyH- | PR-7589 | PR-7590 | USER and Ligation |
| Tadh1-C) | | | of BB0618, BB0623, |
| | | | BB4700 |
| BB4748 (B-Ptdh3-EvTyH- | PR-7589 | PR-7590 | USER and Ligation |
| Tadh1-C) | | | of BB0618, BB0623, |
| | | | BB4701 |
| BB4749 (B-Ptdh3-MmTyH1- | PR-7589 | PR-7590 | USER and Ligation |
| Tadh1-C) | | | of BB0618, BB0623, |
| | | | BB4702 |
| BB4750 (B-Ptdh3-MmTyH2- | PR-7589 | PR-7590 | USER and Ligation |
| Tadh1-C) | | | of BB0618, BB0623, |
| | | | BB4703 |
| BB4751 (B-Ptdh3-OfTyH- | PR-7589 | PR-7590 | USER and Ligation |
| Tadh1-C) | | | of BB0618, BB0623, |
| | | | BB4704 |
| BB4752 (B-Ptdh3-PaTyH- | PR-7589 | PR-7590 | USER and Ligation |
| Tadh1-C) | | | of BB0618, BB0623, |
| | | | BB4705 |
| BB4753 (B-Ptdh3-PdTyH- | PR-7589 | PR-7590 | USER and Ligation |
| Tadh1-C) | | | of BB0618, BB0623, |
| | | | BB4706 |
| BB4773 (A-Ptef1-MjDOD-Tcyc1- | PR-7587 | PR-7588 | USER and Ligation |
| B) | | | of BB0622, BB0624, |
| | | | BB4730 |
| BB4774 (B-Ptdh3-TyH(W13L)- | PR-7589 | PR-7590 | USER and Ligation |
| Tadh1-C) | | | of BB0618, BB0623, |
| | | | BB4731 |
| BB4999 (YI_AnTYH_G2) | PR-27826 | PR-27827 | Synthetic gene |
| BB5000 (YI_BgDOD2_G1) | PR-27828 | PR-27829 | Synthetic gene |
| BB5001 (YI_EvTYH_G2) | PR-27830 | PR-27831 | Synthetic gene |
| BB5049 (YI_MjDOD_G1) | PR-27980 | PR-27981 | Synthetic gene |
| BB5050 (YI_BvTYH_G2) | PR-27982 | PR-27983 | Synthetic gene |
| BB5217 (YI_PgDOD_mut_G1) | PR-28832 | PR-28833 | Synthetic gene |
| BB5051 (BvSGT1_Gene1) | PR-27946 | PR-27947 | Synthetic gene |
| BB5052 (BvSGT2_Gene1) | PR-27948 | PR-27949 | Synthetic gene |
| BB5053 (BvSGT3_Gene1) | PR-27950 | PR-27951 | Synthetic gene |
| BB5054 (BvSGT4_Gene1) | PR-27952 | PR-27953 | Synthetic gene |
| BB5746 (BvSGT5_Gene1) | PR-29836 | PR-29837 | Synthetic gene |
| BB5747 (CqSGT1_Gene1) | PR-29838 | PR-2983 | Synthetic gene |
| BB5748 (PaSGT1_Gene1) | PR-29840 | PR-29841 | Synthetic gene |
| BB5749 (SoSGT1_Gene1) | PR-29842 | PR-29843 | Synthetic gene |
| BB5750 (VrSGT1_Gene1) | PR-29844 | PR-29845 | Synthetic gene |
| BB5973 (CqSGT2_Gene1) | PR-31226 | PR-31227 | Synthetic gene |
| BB5974 (CqSGT3_Gene1) | PR-31228 | PR-31229 | Synthetic gene |
| BB5975 (CqSGT4_Gene1) | PR-31230 | PR-31231 | Synthetic gene |
| BB5976 (CqSGT5_Gene1) | PR-31232 | PR-31233 | Synthetic gene |
| BB5977 (CqSGT6_Gene1) | PR-31234 | PR-31235 | Synthetic gene |
| BB5978 (SoSGT2_Gene1) | PR-31236 | PR-31237 | Synthetic gene |
| BB5979 (SoSGT3_Gene1) | PR-31238 | PR-31239 | Synthetic gene |
| BB5980 (SoSGT4_Gene1) | PR-31240 | PR-31241 | Synthetic gene |
| BB5981 (SoSGT5_Gene1) | PR-31242 | PR-31243 | Synthetic gene |
| BB5982 (SoSGT6_Gene1) | PR-31244 | PR-31245 | Synthetic gene |
| BB5983 (SoSGT7_Gene1) | PR-31246 | PR-31247 | Synthetic gene |
| BB5984 (CsSGT1_Gene1) | PR-31248 | PR-31249 | Synthetic gene |
| BB5985 (CiSiSGT1_Gene1) | PR-31250 | PR-31251 | Synthetic gene |
| BB5986 (CiCISGT1_Gene1) | PR-31252 | PR-31253 | Synthetic gene |
| BB5987 (EgGT1_Gene1) | PR-31254 | PR-31255 | Synthetic gene |
| BB5988 (CpSGT1_Gene1) | PR-31256 | PR-31257 | Synthetic gene |
| BB5989 (MeSGT1_Gene1) | PR-31258 | PR-31259 | Synthetic gene |
| BB5990 (RaSGT1_Gene1) | PR-31260 | PR-31261 | Synthetic gene |
| BB5991 (TcSGT1_Gene1) | PR-31262 | PR-31263 | Synthetic gene |
| BB5709 (BgGT1_Gene1) | PR-30008 | PR-30009 | Synthetic gene |
| BB5710 (BgGT2_Gene1) | PR-30010 | PR-30011 | Synthetic gene |
| BB5711 (BgGT3_Gene1) | PR-30012 | PR-30013 | Synthetic gene |
| BB5893 (TPex20<-MjDOD_YI<- | PR-25056 | PR-27831 | pCfB11114 |
| PrGDP::PrTEFin->EvTYH_YI) | | | |
| BB5894 (BvSGT2_YI (W/O | PR-29863 | PR-29970 | Synthetic gene |
| ATG) | | | |
| BB5898 (PrTEFin_single-U1) | PR-23967 | PR-25055 | pCfB8977 |
| BB5996 (4-HPPD-UP) | PR-31410 | PR-31419 | W29 genome |
| BB5997 (4-HPPD-DW) | PR-31420 | PR-31413 | W29 genome |
| BB5998 (HphSyn-Loxp- | PR-31421 | PR-31422 | pCfB5935 (pIntA-1- |
| cassette) | | | HphMx-TPex20- |
| | | | TLip2) |
| BB5999 (4-HPPD-HphSyn- | PR-31410 | PR-31413 | USER and Ligation |
| LoxP) | | | of BB5996, BB5997, |
| | | | BB5998 |

TABLE 3
Integrative, episomal and helper (gRNA) vectors.

| | | | DNA | |
|---|---|---|---|---|
| | | | fragments | |
| | Type of | | cloned into | |
| Name | vector | Parent vector | parent vector | Source |
| pCfB2310 | Helper (gRNA) | — | — | Stovicek et |
| (SNR52p- | vector | | | al. 2015 |
| gRNA.CAN1- | | | | |
| SUP4t_natMX) | | | | |
| pCfB2312 | episomal | — | — | Stovicek et |
| (TEF1p-Cas9- | vector for | | | al. 2015 |
| CYC1t_kanMX) | Cas9p | | | |
| | expression | | | |
| pCfB3041 (p- | Helper (gRNA) | — | — | Jessop- |
| gRNA X-3) | vector | | | Fabre et al. |
| | | | | 2016 |
| pCfB6627 | Helper (gRNA) | — | — | Holkenbrink- |
| (pNat- | vector | | | et al. 2018 |
| YLgRNA2_Int | | | | |
| C_2) | | | | |
| pCfB6633 | Helper (gRNA) | — | — | Holkenbrink |
| (pNat- | vector | | | et al. 2018 |
| YLgRNA2_Int | | | | |
| E_1) | | | | |
| pBP8003 | Helper (gRNA) | — | — | This study |
| (pNat- | vector | | | |
| YLgRNA4- | | | | |
| IntF_3) | | | | |
| pCfB8977 (Int | Integrative | pCfB6677 | — | Saez-Saez et |
| E_1-tPex20<- | vector | (pIntE_1- | | al. 2020 |
| YIARO7_G139 | | TPex20-TLip2) | | |
| S<- | | | | |
| PrGPD :: PrTEF | | | | |
| in-> | | | | |
| YIARO4_K22 | | | | |
| 1L->tLip2) | | | | |
| pCfB10368 (X- | Integrative | pCfB3035 (X- | BB8, BB5051 | This study |
| 3_BvSGT1) | vector | 34- | | |
| | | MarkerFree) | | |
| pCfB10369 (X- | Integrative | pCfB3035 (X- | BB8, BB5052 | This study |
| 3_BvSGT2) | vector | 34- | | |
| | | MarkerFree) | | |
| pCfB10370 (X- | Integrative | pCfB3035 (X- | BB8, BB5053 | This study |
| 3_BvSGT3) | vector | 34- | | |
| | | MarkerFree) | | |
| pCfB10371 (X- | Integrative | pCfB3035 (X- | BB8, BB5054 | This study |
| 3_BvSGT4) | vector | 34- | | |
| | | MarkerFree) | | |
| pCfB10106 (X- | Integrative | pCfB3035 (X- | BB8, BB4273 | This study |
| 3_Ptef1- | vector | 34- | | |
| DbB5GT) | | MarkerFree) | | |
| pCfB10607(Int | Integrative | pCfB6684 | BB3908, | This study |
| D_BvSGT2) | vector | (pIntD_1- | BB5219 | |
| | | TPex20-TLip2) | | |
| pCfB10619(Int | Integrative | pCfB6682 | BB3907, | This study |
| C2_PgDOD* | vector | (pIntC_2- | BB5217, | |
| BvCYP76ADW1 | | TPex20-TLip2) | BB5050 | |
| 3L) | | | | |
| pCfB10620(Int | Integrative | pCfB6682 | BB3907, | This study |
| C2_PgDOD *_ | vector | (pIntC_2- | BB5217, | |
| EvTYH) | | TPex20-TLip2) | BB5001 | |
| pCfB10622(Int | Integrative | pCfB6682 | BB3907, | This study |
| C2_BgDOD2 | vector | (pIntC_2- | BB5000, | |
| BvCYP76ADW1 | | TPex20-TLip2) | BB4999 | |
| 3L) | | | | |
| pCfB10623(Int | Integrative | pCfB6682 | BB3907, | This study |
| C2_BgDOD2_ | vector | (pIntC_2- | BB5000, | |
| EvTYH) | | TPex20-TLip2) | BB5050 | |
| pCfB10621(Int | Integrative | pCfB6682 | BB3907, | This study |
| C2_BgDOD2_ | vector | (pIntC_2- | BB5000, | |
| AnTYH) | | TPex20-TLip2) | BB5001 | |
| pCfB10625(Int | Integrative | pCfB6682 | BB3907, | This study |
| C2_MjDOD_ | vector | (pIntC_2- | BB5049, | |
| BvCYP76ADW1 | | TPex20-TLip2) | BB4999 | |
| 3L) | | | | |
| pCfB10626(Int | Integrative | pCfB6682 | BB3907, | This study |
| C2_MjDOD_E | vector | (pIntC_2- | BB5049, | |
| vTYH) | | TPex20-TLip2) | BB5050 | |
| pCfB10624(Int | Integrative | pCfB6682 | BB3907, | This study |
| C2_MjDOD_A | vector | (pIntC_2- | BB5049, | |
| nTYH) | | TPex20-TLip2) | BB5001 | |
| pCfB11102 (X- | Integrative | pCfB3035 (X- | BB8, BB5746 | This study |
| 3_BvSGT5) | vector | 3-Marker Free) | | |
| pCfB11103 (X- | Integrative | pCfB3035 (X- | BB8, BB5747 | This study |
| 3_CqSGT1)) | vector | 3-Marker Free) | | |
| pCfB11104 (X- | Integrative | pCfB3035 (X- | BB8, BB5748 | This study |
| 3_PaSGT1) | vector | 3-Marker Free) | | |
| pCfB11105 (X- | Integrative | pCfB3035 (X- | BB8, BB5749 | This study |
| 3_SoSGT1) | vector | 3-Marker Free) | | |
| pCfB11106 (X- | Integrative | pCfB3035 (X- | BB8, BB5750 | This study |
| 3_VrSGT1) | vector | 3-Marker Free) | | |
| pCfB11113 | Integrative | pBP8009 | BB4323, | This study |
| (IntF_3- | vector | (pIntF_3- | BB5898, | |
| TPex20<- | | TPex20-TLip2) | BB5893, | |
| BvSGT2_YI<- | | | BB5894 | |
| PrTEFin, <- | | | | |
| TPex20<- | | | | |
| MjDOD_YI- | | | | |
| PrGPD::PrTEF | | | | |
| in->EvTYH_YI-> | | | | |
| TLip2) | | | | |
| pCfB11114 | Integrative | pBP8009 | BB4323, | This study |
| (IntF_3- | vector | (pIntF_3- | BB3907, | |
| TPex20<- | | TPex20-TLip2) | BB5607, | |
| MjDOD_YK<- | | | BB5611 | |
| PrGPD: PrTEF | | | | |
| in->EvTYH_YI-> | | | | |
| TLip2) | | | | |
| pCfB11617 (X- | Integrative | pCfB3035 (X- | BB8, BB5973 | This study |
| 3_ CqSGT2) | vector | 3-Marker Free) | | |
| pCfB11618 (X- | Integrative | pCfB3035 (X- | BB8, BB5974 | This study |
| 3_ CqSGT3) | vector | 3-Marker Free) | | |
| pCfB11619 (X- | Integrative | pCfB3035 (X- | BB8, BB5975 | This study |
| 3_ CqSGT4) | vector | 3-Marker Free) | | |
| pCfB11620 (X- | Integrative | pCfB3035 (X- | BB8, BB5976 | This study |
| 3_ CqSGT5) | vector | 3-Marker Free) | | |
| pCfB11621 (X- | Integrative | pCfB3035 (X- | BB8, BB5977 | This study |
| 3_ CqSGT6) | vector | 3-Marker Free) | | |
| pCfB11622 (X- | Integrative | pCfB3035 (X- | BB8, BB5978 | This study |
| 3_ SoSGT2) | vector | 3-Marker Free) | | |
| pCfB11623 (X- | Integrative | pCfB3035 (X- | BB8, BB5979 | This study |
| 3_SoSGT3) | vector | 3-Marker Free) | | |
| pCfB11624 (X- | Integrative | pCfB3035 (X- | BB8, BB5980 | This study |
| 3_ SoSGT4) | vector | 3-Marker Free) | | |
| pCfB11625 (X- | Integrative | pCfB3035 (X- | BB8, BB5981 | This study |
| 3_SoSGT5) | vector | 3-Marker Free) | | |
| pCfB11626 (X- | Integrative | pCfB3035 (X- | BB8, BB5982 | This study |
| 3_ SoSGT6) | vector | 3-Marker Free) | | |
| pCfB11627 (X- | Integrative | pCfB3035 (X- | BB8, BB5983 | This study |
| 3_SoSGT7) | vector | 3-Marker Free) | | |
| pCfB11628 (X- | Integrative | pCfB3035 (X- | BB8, BB5984 | This study |
| 3_ CsSGT1) | vector | 3-Marker Free) | | |
| pCfB11629 (X- | Integrative | pCfB3035 (X- | BB8, BB5985 | This study |
| 3_ CiSiSGT1) | vector | 3-Marker Free) | | |
| pCfB11630 (X- | Integrative | pCfB3035 (X- | BB8, BB5986 | This study |
| 3_ CiCISGT1) | vector | 3-Marker Free) | | |
| pCfB11631 (X- | Integrative | pCfB3035 (X- | BB8, BB5987 | This study |
| 3_EgSGT1) | vector | 3-Marker Free) | | |
| pCfB11632 (X- | Integrative | pCfB3035 (X- | BB8, BB5988 | This study |
| 3_ CpSGT1) | vector | 3-Marker Free) | | |
| pCfB11633 (X- | Integrative | pCfB3035 (X- | BB8, BB5989 | This study |
| 3_ MeSGT1) | vector | 3-Marker Free) | | |
| pCfB11634 (X- | Integrative | pCfB3035 (X- | BB8, BB5990 | This study |
| 3_RaSGT1) | vector | 3-Marker Free) | | |
| pCfB11635 (X- | Integrative | pCfB3035 (X- | BB8, BB5991 | This study |
| 3_TcSGT1) | vector | 3-Marker Free) | | |
| pCfB11393 (X- | Integrative | pCfB3035 (X- | BB8, BB5709 | This study |
| 3_BgGT1) | vector | 3-Marker Free) | | |
| pCfB11394 (X- | Integrative | pCfB3035 (X- | BB8, BB5710 | This study |
| 3_BgGT2) | vector | 3-Marker Free) | | |
| pCfB11395 (X- | Integrative | pCfB3035 (X- | BB8, BB5711 | This study |
| 3_BgGT3) | vector | 3-Marker Free) | | |

### Screening Enzyme Variants for CYP76ADα (TYH) and 4,5-Dopa-Extradiol-Oxygenase (DOD)

To find enzymes for 4,5-dopa-extradiol-oxygenase (DOD), 229 plant protein sequences were screened. Final screened variants of DOD proteins and the corresponding codon-optimized sequences for S. cerevisiae are listed as SEQ ID NO: 3-26.

In a similar way, for CYP76ADα (termed as TYH), 35 sequences of plant CYP76AD1 were screened. Final screened variants of TYH proteins and the corresponding codon-optimized sequences for S. cerevisiae are listed as SEQ ID NO: 29-48.

### Screening Enzyme Variants for Glucosyltransferases

To find novel glucosyltransferases for production of betacyanins in yeast in Example 3, we used the protein sequence of betanidin/cyclo-DOPA glucosyltransferase from Beta vulgaris (GenBank: AA088911.1). This gene was BLASTed into the Beta vulgaris genome, and for each of the blast hits, 5 coding DNA sequences located on up- and downstream hit were inspected. Of these genes, four CDSs with glucosyltransferase in the annotation were identified, all of them being “scopoletin glucosyl transferase”. The sequences of these proteins and the corresponding codon-optimized sequences for S. cerevisiae are named BvSGT1:BvSGT4 (SEQ ID NO: 51-58).

### Screening Enzyme Variants for Scopoletin Glucosyltransferases

To find more scopoletin glucosyltransferases that might be able to produce betacyanins in yeast, BvSGT2 (SEQ ID NO: 54) was BLASTed for RefSeq in plants (taxid 3193). From the top 100 hits, all hits from the Chenopodiaceae family (excluding Beta vulgaris sequences BvSGT1-4 (SEQ ID No: 52-58) were taken. For the remaining organisms, the hit with the highest sequence identity to BvSGT2 was taken. This resulted in a list of 24 genes, coding for proteins annotated as “scopoletin glucosyltransferases” or as “scopoletin glucosyltransferase-like”. The sequences of these proteins and the corresponding codon-optimized sequences for S. cerevisiae are named BvSGT5, CqSGT1, CqSGT2, CqSGT3, CqSGT4, CqSGT5, CqSGT6, PaSGT1, VrSGT1, SoSGT1, SoSGT2, SoSGT3, SoSGT4, SoSGT5, SoSGT6, SoSGT7, CsSGT1, CiSiSGT1, CiCISGT1, EgSGT1, CpSGT1, MeSGT1, RaSGT1, and TcSGT1.

Screening Enzyme Variants for Glucosyltransferases in Red Bougainvillea glabra

To find glucosyltransferases responsible for betacyanin production in the red flowering plant Bougainvillea glabra, its transcriptome was BLASTed for the UGTs BvSGT1-4 (SEQ ID NO: 52-58), DbB5GT (SEQ ID NO: 49, 50) and the literature UGTs described by the GenInfo identifiers: gi:18033791, gi:46430997, gi: 46430995, gi: 62086401, gi:62086403. All belong to the UDP-glucoronosyl and UDP-glucosyl transferase family (PF00201). The resulting sequence hits were filtered for a length of at least 400 amino acids, those starting with a start codon were extracted and identical sequences removed. Of the remaining sequences, 3 were selected for their expression level and potentially interesting characteristics and named BgGT1, BgGT2 and BgGT3.

### Constructing the Yeast Libraries

To screen for the enzyme variants for tyrosine hydroxylase (TYH) and 4,5-dopa-extradiol-oxygenase (DOD) in Example 2, the genes encoding the two proteins were integrated into CAN1 site of ST8251 genome using the combinatorial method described by Kildegaard et al. (2019). The method comprised transforming the strain ST8251 with gRNA plasmid for targeting CAN1 locus (pCfB2310(SNR52p-gRNA.CAN1-SUP4t_natMX)), and five overexpression cassettes for in vivo assembly.

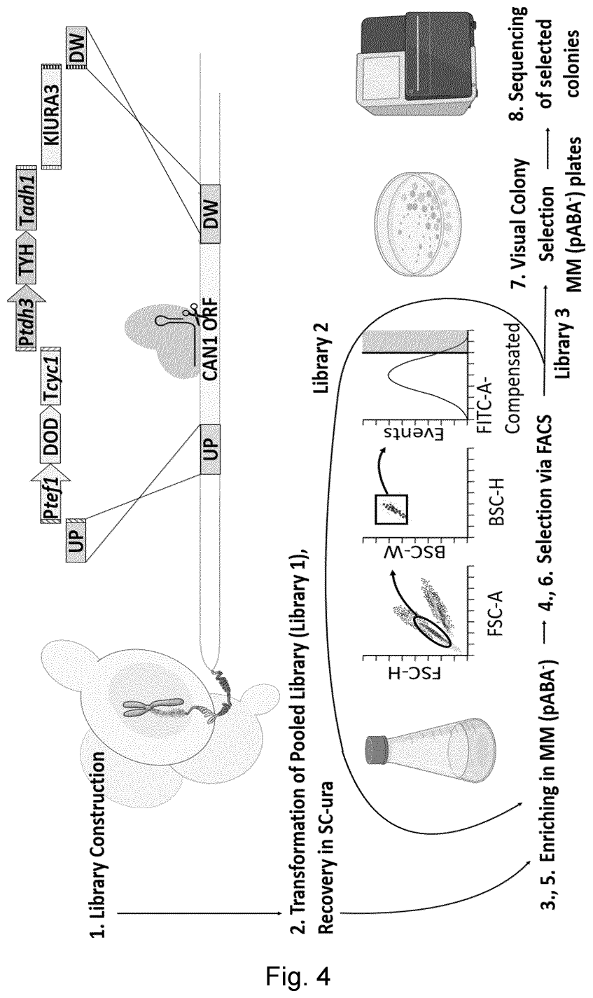

The five parts of the overexpression cassettes consisted of (FIG. 4):
i) upstream homology arm (BB0629, Table 2)
ii) DOD variants under the control of TEF1 promoter (Ptef1) and CYC1 terminator (Tcyc1), (BB4733:BB4743, Table 2)
iii) TYH variants under the control of TDH3 promoter (Ptdh3) and ADH1 terminator (Tadh1), (BB4744:BB4753, Table 2)
iv) auxotrophic marker Klura3 from Kluyveromyces lactis (BB4732, Table 2)
v) downstream homology arm (BB0630, Table 2)

The specific overhangs flanking each part are designed to be introduced at 5′ end of the forward and reverse primers as described by Kildegaard et al. 2019 (Kildegaard et al. 2019). To transform the yeast library, parent strain ST8251 was grown in 25 mL YPD medium supplemented with G418 for 4 hours at 30° C. and 250 rpm, to get an optical density of 1.0-1.5. The cells were then harvested and transformed with pooled DNA library using the standard lithium acetate method described in EasyClone MarkerFree method (Jessop-Fabre et al. 2016). The pooled DNA library consisted of 10 μg of gRNA vector pCfB2310, and ca. 5 picomoles of Ptef1-DOD-Tcyc1 and Ptdh3-TYH-Tadh1, and 15 picomoles of the fragments for upstream- and downstream homology arms and Klura3 marker. This mixture was purified by ethanol precipitation and resuspended in 148 μL of water. The transformed cells were cultivated overnight in 25 mL of synthetic dropout medium without uracil (SC-ura) at 30° C. and 250 rpm. The culture was then diluted 1:10 into 5 mL of MM medium, with the rest of the culture mixed 1:1 with 50% glycerol and stored at −80° C. The next day, the culture was again diluted 1:50 into 5 mL MM medium and grown for 16 h. This culture was used for fluorescence-assisted cell sorting (FACS).

### Flow Cytometry and Library Sorting

To remove secreted betaxanthins the cells were washed two times with PBS buffer (pH 7.5) by centrifuging at 3,000×g for 5 minutes. The cells were then resuspended PBS buffer for analysis or sorting. Library cells were then sorted using Sony SH800 Cell Sorter (Sony, Tokyo, Japan) to identify the most promising enzyme variants in regards to desired phenotype. The measurements were performed using a 488 nm laser and a 525/50 band-pass filter. As cell doublets might significantly mislead the sorting experiments, events were first gated for live cells by linear alignment of FSC-height vs. FSC-area, and then gated for singlets by discriminating single and double cells in SSC-width vs. SSC-height. The gate sizes were set to capture approximately 40% and 80% of the population, respectively. Cells passing the live and singlet selection gates were then sorted for the top 1-7% of the fluorescence distribution. 10,000 events were sorted and collected to culture tube containing 2 mL SC-ura medium, and were grown overnight. The sorting process was repeated once more on these cultures, to ensure for enrichment of high-fluorescent single cells. The second-round sorted cells were plated on Nunc™ OmniTray™ single-well plates (Thermo scientific) containing MM (pABA⁻) agar medium, with a density of 3,000-5,000 events per plate. The plated cells were incubated at 30° C. for 4-5 days, until single colonies were obtained. Based on visual selection, the most intense yellow coloured single isolates were selected and proceeded with fluorescence measurement.

### Cultivation and Fluorescence Measurement on Plate Reader

To measure fluorescence (betaxanthin) in strains in Example 2 the selected isolates were cultivated overnight in 96-deep-well plate containing 400 μL MM (pABA⁻) with air-penetrable metal lid (EnzyScreen, The Netherlands) at 30° C. and 250 rpm. Next day, the cultures were diluted to fresh MM (pABA⁻) to get an optical density (OD₆₀₀) of 0.1, and were incubated at 30° C. and 250 rpm for 48 hours. The optical density (OD₆₀₀) and fluorescence (485-515 nm) were measured in a plate reader (BioTek ELx 808) at 24 and 48 hours, and data are reported as fluorescence/OD₆₀₀. For the Examples 3, 4 and 5, the cultivation was done in the same manner, but in 24-deep-well plates containing 2 mL MM (pABA⁻) with starting OD₆₀₀ value of 0.4, and fermentation terminated at 48 hours.

### Identifying the DOD and TYH Variants in Selected Strains

The selected isolates with desired phenotype (highest production of betaxanthins) were cultivated in 5 mL of MM (pABA⁻) for 48 hours at 30° C. and 250 rpm. The genomic DNA of the cells were then extracted by Quick-DNA™ fungal/Bacterial Miniprep Kit (ZYMO Research). The 5.17 kb fragment containing the DOD and TYH genes was amplified using the primers PR-7540 and PR-24714 and genomic DNA of each isolate as the PCR template. The fragments were then sequenced by Sanger method using the primers PR-225, PR-339, PR-28955, PR-28956 (Eurofins Genomics, Ebersberg, Germany).

### HPLC Analysis of Betanin Titer

As pure betanin standard is not commercially available, we used the red beet extract diluted with dextrin (Sigma-Aldrich, product ID:901266-5G). To quantify the betanin content in this mixture, we used the Beer-Lambert equation by assuming the molar extinction coefficient E=6.5×10⁴ M⁻¹ cm⁻¹ for betanin (Gonçalves et al. 2012). The stock solution of red beet extract in MM (pABA⁻) medium was prepared in 10 g/L concentration, from which samples of 2 g/L, 1 g/L and 0.5 g/L were made. By reading the absorbance of these samples at λ=536 nm in cuvettes with 1 cm path length, the betanin content in 1 g/L of red beet extract was calculated to be 1.674 mg/L (see below).

| Red beet | | Calculated betanin content |
|---|---|---|
| samples | Absorbance₅₃₆ₙₘ | (mg/L) |
| 2 g/L | 0.394 ± 0.001 | 3.337 ± 0.008 |
| 1 g/L | 0.198 ± 0.000 | 1.674 ± 0.005 |
| 0.5 g/L | 0.1 ± 0.001 | 0.847 ± 0.008 |

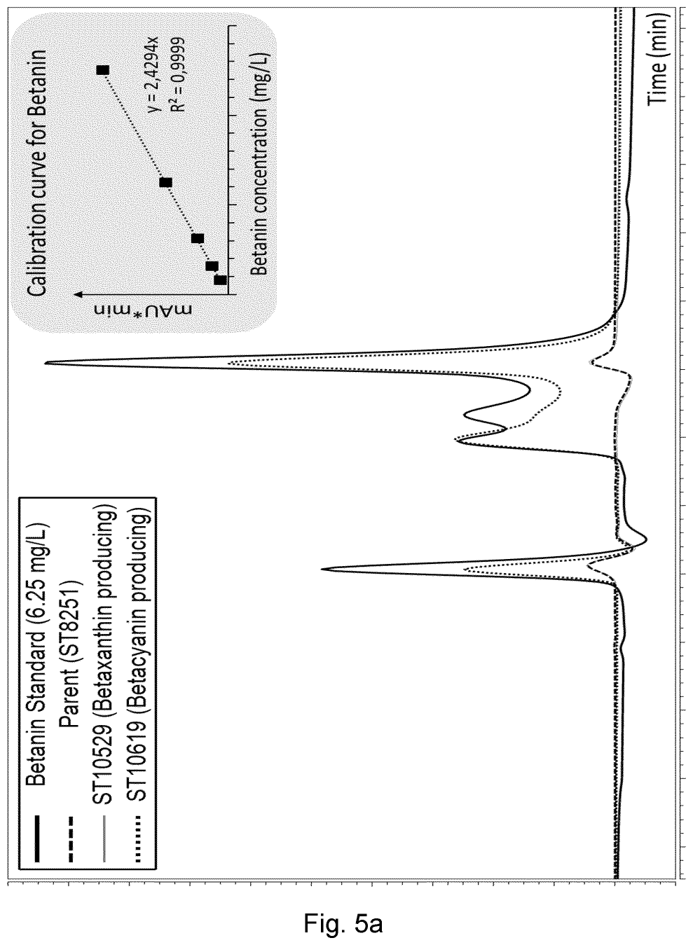

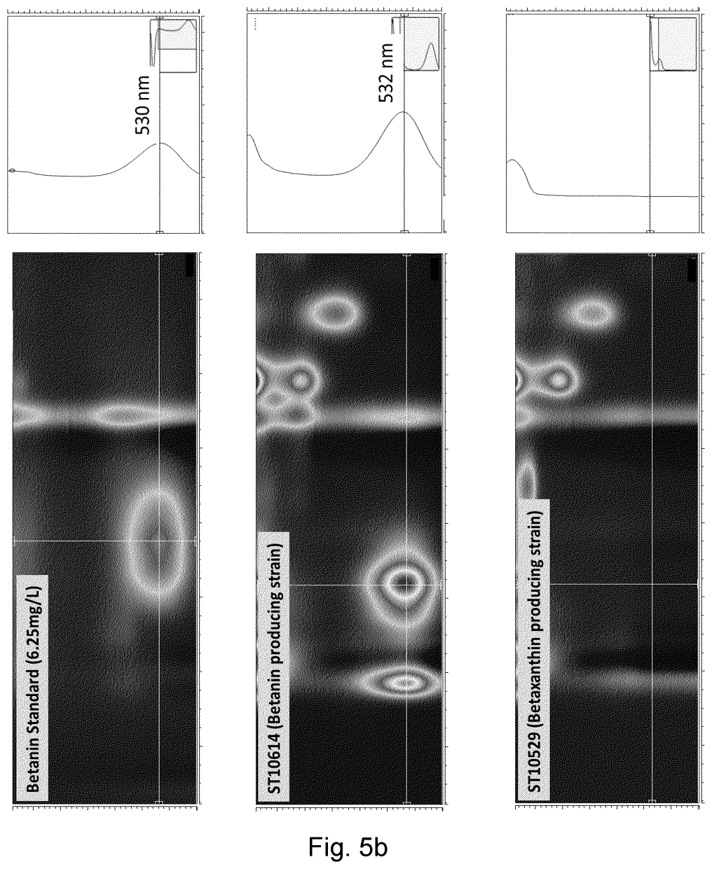

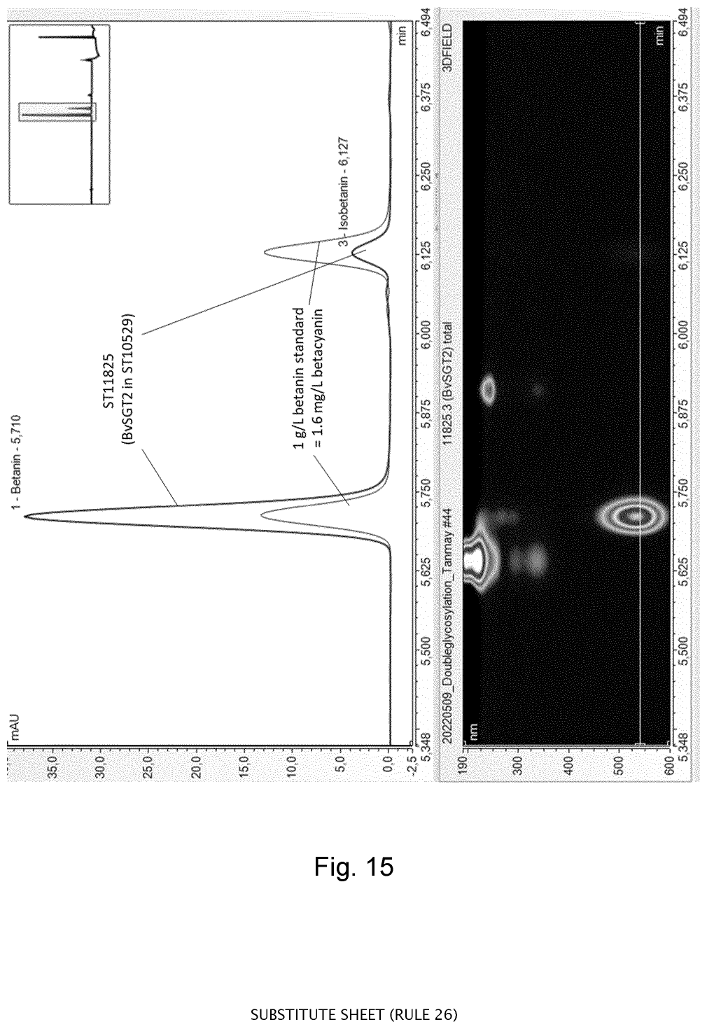

To quantify the betanin content in yeast cultures (FIG. 5a-b), the broth was centrifuged at 10,000 g for 10 minutes to precipitate all the cells and debris, and the supernatant was moved to amber-coloured vials for HPLC analysis. Quantification was done in Dionex Ultimate 3000 HPLC (Thermo Fisher Scientific), equipped with a Zorbax® column with particle size 5 μm. For Example 3, the following HPLC method was used: the column oven temperature was set at 30° C. and the flow rate to 1 mL/min, with 10 μL of sample injection. Solvent A was 0.1% formic acid, and solvent B was acetonitrile. Solvent composition was initially A=80.0%, and B=20.0%, which was kept until 1 min. Then, solvent composition was changed following a linear gradient until A=60.0%, and B=40.0% at 2.0 min. These conditions were kept constant until 5.5 min, at which point the solvent composition was again increased linearly until A=10.0%, and B=90.0% at 7.5 min. The condition was kept constant for 1.5 min (7.5-9 min), after which the composition was retrieved to the initial conditions (A=80.0%, B=20.0%) at 9.2 min, and remained unchanged until the end of the run (9.2-12 min). The retention time and absorbance for detection of betanin was 1.26 min, 540 nm. For Example 10, a modified HPLC method was used: the column oven temperature was set at 30° C. and the flow rate to 1 mL/min, with 10 μL of sample injection. The absorbance at 390 nm, 410 nm, 480 nm and 540 nm was measured with a UV-Vis detector. Solvent A was 0.1% formic acid, and solvent B was acetonitrile. Solvent composition was initially A=98.0%, and B=2.0%, which was kept for 2 min. Then, solvent composition was changed following a linear gradient until A=90.0%, and B=10.0% at 5.0 min. In a second linear gradient, solvent composition changed until A=85.0% and B=15.0% at 8.0 min. At 8.2 min, solvent composition was increased to A=2.0%, and B=98.0%. The condition was kept constant until 9.5 min after which the initial composition was retrieved (A=98.0%, B=2.0%) and remained unchanged until the end of the run (11.5 min). With this HPLC method, the betanin standard showed two large peaks with almost the same peak area. The first peak had a retention time of 5.7 min and likely corresponds to betanin while the second peak has a retention time of 6.1 min and likely corresponds to isobetanin, as shown for production of betanin in ST11825 (FIG. 15).

For both HPLC methods, peak areas were used for compound quantification using an external standard calibration method. Analysis of HPLC results was performed using the software Chromeleon 7 (ThermoFisher Scientific).

### Example 2—Selection of DOD-TYH Combinations for Betaxanthin Production

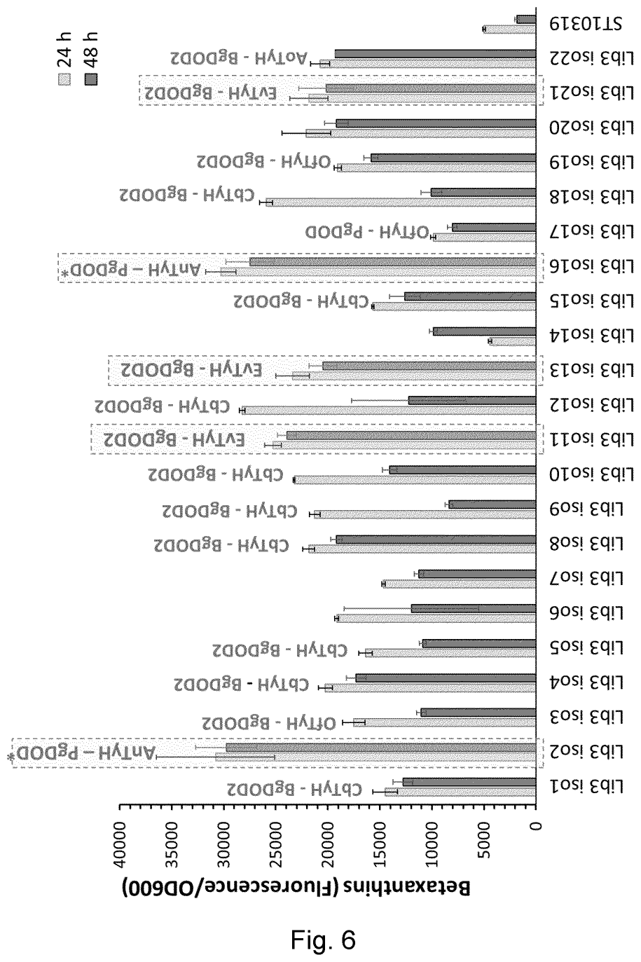

As the reference strain for the highest betalain production reported in literatures so far (DeLoache et al. 2015), we used the strain ST10319, expressing BvCYP76AD^{W13L}(SEQ ID NO: 27,28) and MjDOD (SEQ ID NO: 1,2) in the same strain background (ST8251) as the library. After the yeast library sorting, the results showed that in overall the fluorescence values have increased for sorted cells (Library 3: Mean=5660, Median=5097) compared to the reference strain (ST10319: Mean=455, Median=405). Next, we selected 22 single isolates from library 3, and looked for the fluorescence (betaxanthin titer) enhancement. The results show that for the majority of isolates there was higher fluorescence compared to the reference strain ST10319 (FIG. 6). Next, we selected the highest fluorescent isolates and identified the origin of integrated DOD and TYH orthologs. The results also show an overlap on the origin of DOD and TYH enzymes that lead to improvement in betaxanthin titer. Of the five highest betaxanthin-producing isolates (iso2, 11, 13, 16, 21), we selected iso2 and iso21. The selected combination of enzymes in these two isolates were identified as:
iso2. PgDOD*-AnTYH: a mutant version of DOD from Portulaca grandiflora (SEQ ID NO: 9,10) co-expressed with TYH from Abronia nealleyi (SEQ ID NO: 37,38). This yeast isolate was named ST10528 (Table 1).
iso21. BgDOD2-EvTYH: DOD2 from Bougainvillea glabra (SEQ ID NO: 21,22) co-expressed with TYH from Ercilla volubilis (SEQ ID NO: 43,44). This yeast isolate was named ST10529 (Table 1).

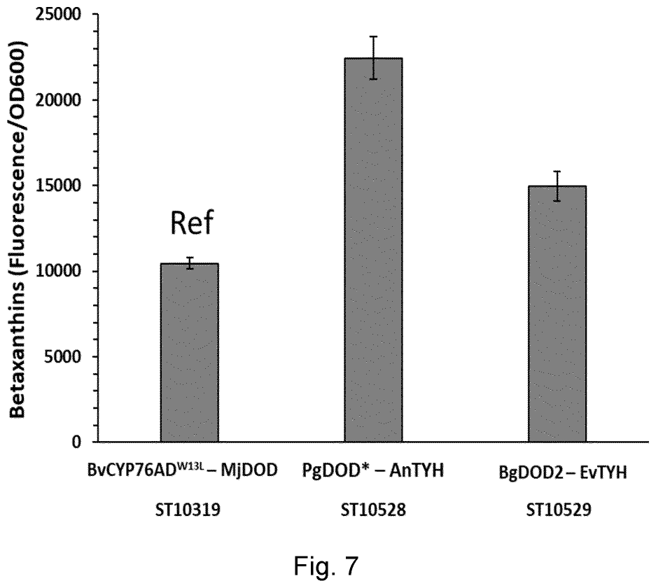

The two isolates iso2 and iso21 were then transformed with pCfB2312 for Cas9 protein expression, as the isolates lost the plasmid due to the lack of G418 antibiotic selection throughout the consecutive cultures for library propagation and sorting The production of betaxanthins in strains ST10528 and ST10529 was higher than in strain ST10319 expressing BvCYP76AD^{W13L} (SEQ ID NO: 28) and MjDOD (SEQ ID NO: 2) (FIG. 7).

### Example 3—Novel Glucosyltransferases for Production of Betacyanins

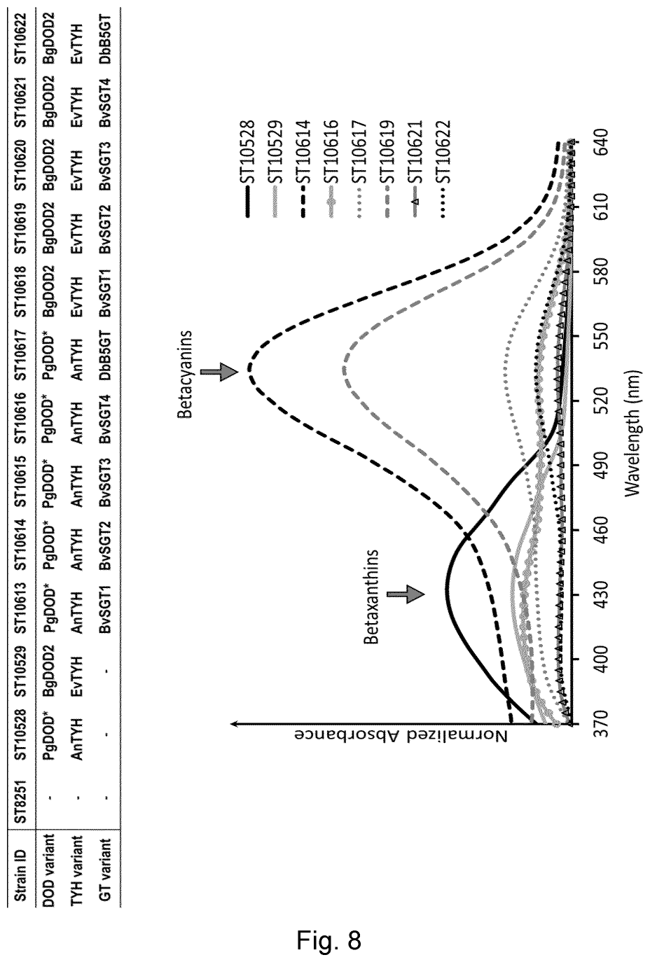

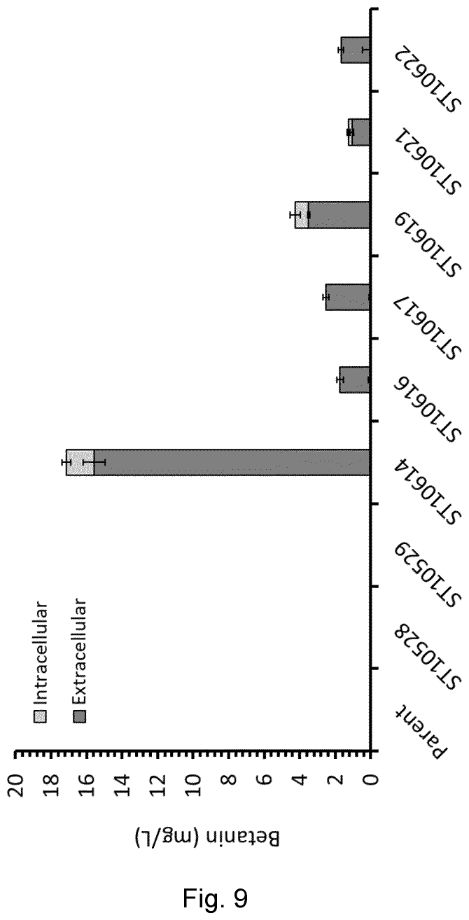

By searching for novel glucosyltransferase (GT) in genome of Beta vulgaris by the method described in Example 1, we could identify four scopoletin glucosyltransferases (BvSGT1:BvSGT4, SEQ ID NO: 51-58). To assess the activity of these GTs, we used the betanin-5-glucosyltransferase from Cleretum bellidiforme as a reference (DbB5GT, SEQ ID NO: 49, 50), since this enzyme was reported to have a high efficiency for production of betacyanins (Grewal et al. 2018). The BvSGTs together with DbB5GT genes were transformed in both betaxanthin-producing strains ST10528 and ST10529. The results show that the fermentation broth of the strains with integrated BvSGT2 and BvSGT4 appeared red, which was also confirmed by a bathochromic shift from 430 nm (betaxanthins) to 535 nm (for betacyanins, FIG. 8). The order of red colour intensity was: BvSGT2>BvSGT4>DbB5GT. Betanin concentration in the broth was measured by HPLC after 48 hours of cultivation in MM (pABA⁻) medium supplemented with 20 g/L glucose (FIG. 9). The highest betanin titer was obtained for strain ST10614 with BvSGT2 (total of 17.14±0.85 mg/L), which is several times higher than the corresponding strain expressing DbB5GT (total of 2.42±0.38 mg/L). The analysis of intracellular content of betanin shows that about 10-15% of total betanin was trapped intracellularly, with the rest being secreted to the fermentation broth.

### Example 4—Improved Yeast Strains

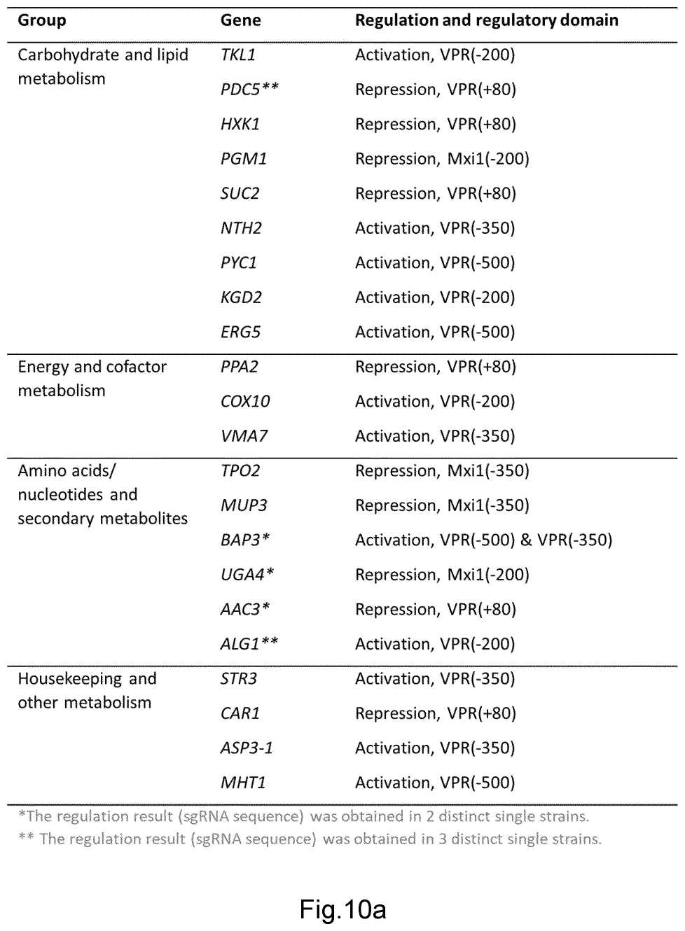

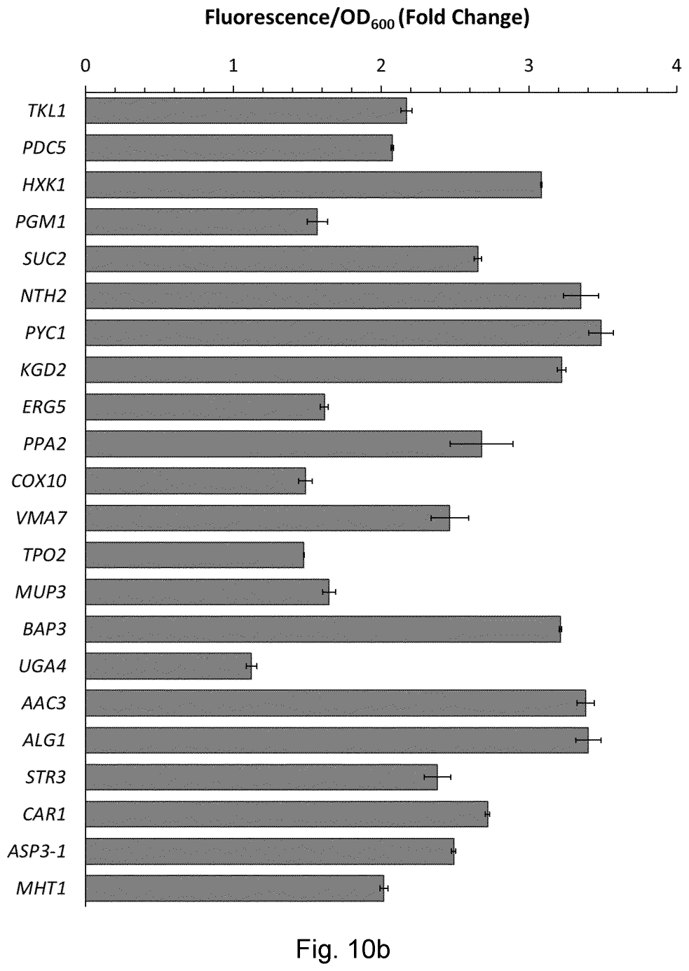

To identify targets for improving betalain production, we used the genome-wide enzyme perturbation method by dCas9-VPR or dCas9-Mxi1 vector libraries reported by Bowman et al., 2020 (Bowman et al. 2020). For this method, the strain ST9632 with integrated biosensor for betaxanthin production (DeLoache et al. 2015) was used and the yeast library was constructed and sorted with similar method described in Example 1, except for that the of synthetic dropout medium without leucine was used for library enrichment phases. Throughout the sorting process, we observed that in overall, the fluorescence fold change over the control strain for single cells selected from dCas9-VPR library (Mean=2.61, Median=2.53, SD=0.86) was slightly higher than dCas9-Mxi1 library (Mean=1.64, Median=1.52, SD=0.62). Next, we selected single strains showing the highest fluorescence enhancement, and continued with plasmid isolation and sequencing of sgRNA. The results (FIG. 10a-b) are categorized into four functional groups: i) Carbohydrate and lipid metabolism, ii) Energy and cofactor metabolism, iii) Amino acid/nucleotide and secondary metabolites, and iv) Housekeeping and other metabolism. Twenty-two regulatory changes were identified that improved the production of betaxanthins. These changes can be implemented alone or in combinations into the yeast strains to improve the production of betaxanthins and betacyanins. The changes can be implemented on the genome-level to create stable strains, which can then be screened again by the same method to identify further beneficial changes.

### Example 5—Producing Betalains in Non-Conventional Yeast

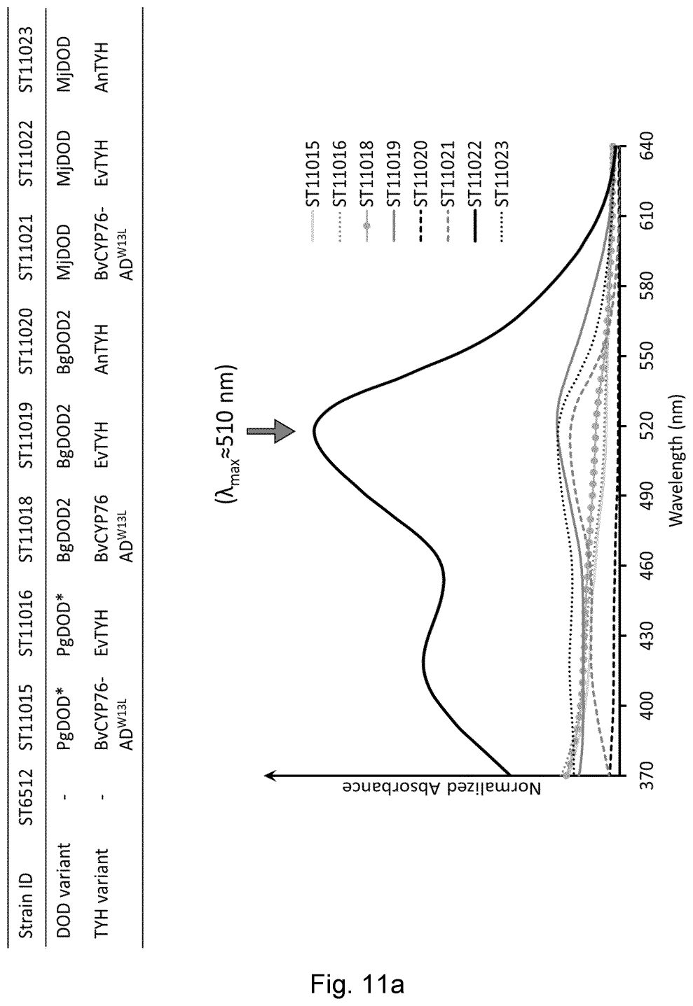

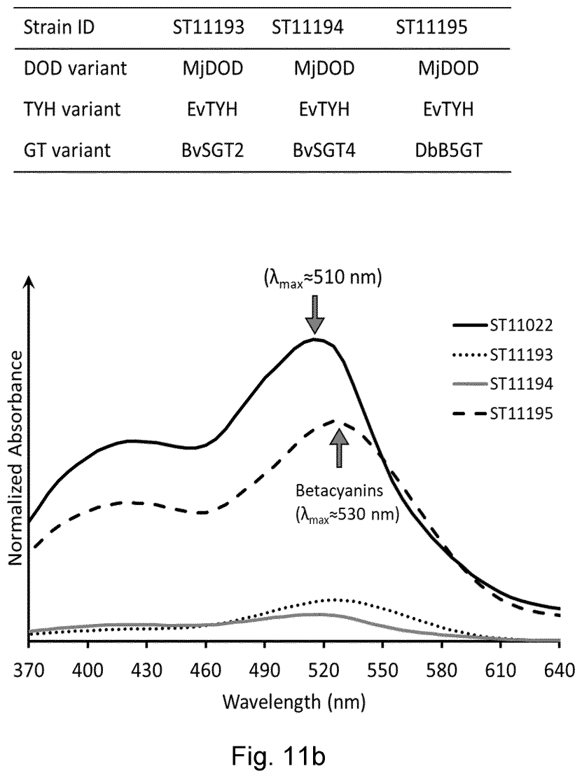

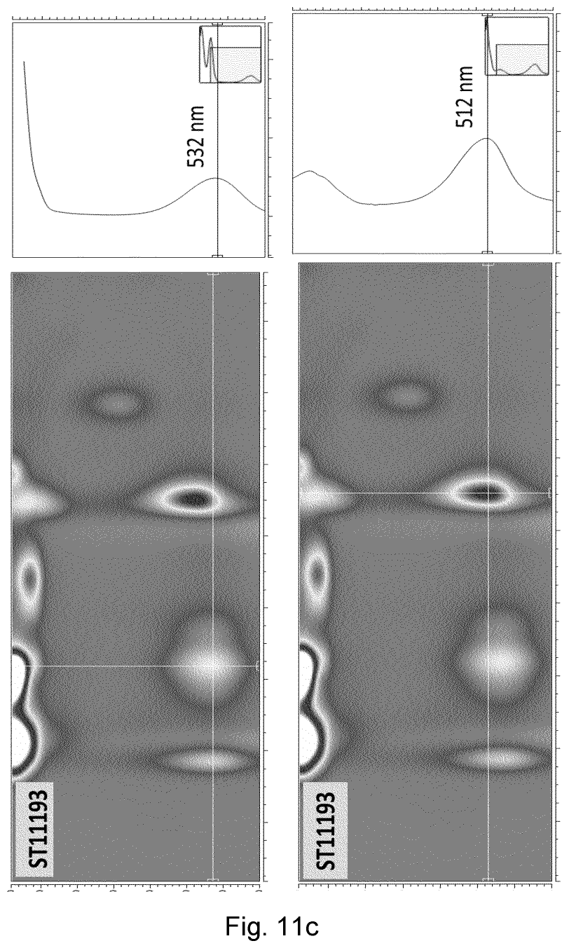

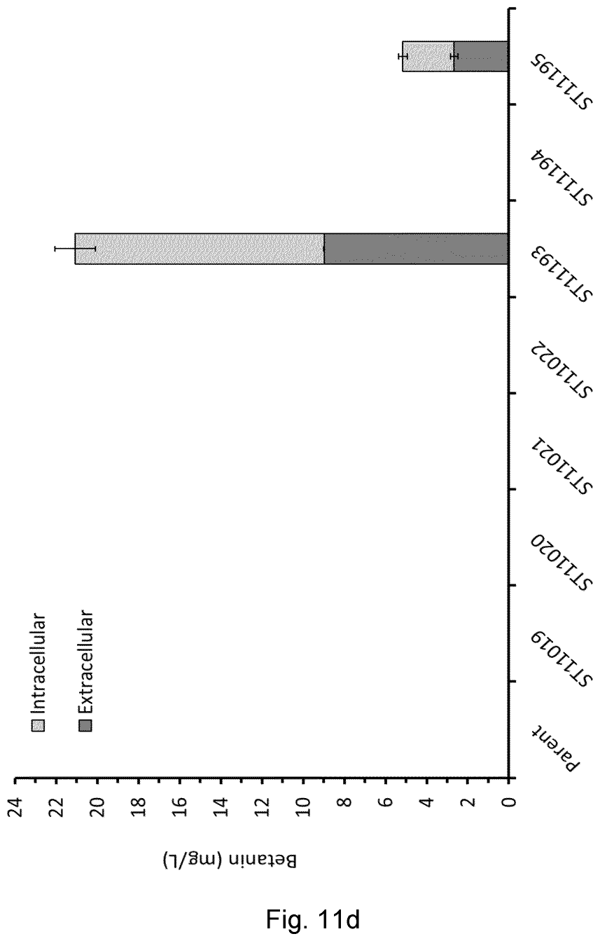

To test the production of betalains in other yeasts than S. cerevisiae, we engineered the non-conventional yeast Yarrowia lipolytica for betalain production. The selected gene orthologs of 4,5-DOPA extradiol dioxygenase: PgDOD* (SEQ ID NO: 59) and BgDOD2 (SEQ ID NO: 60), CYP76ADα: EvTYH (SEQ ID NO: 63) and AnTYH (SEQ ID NO: 64) together with the reference variants: MjDOD (SEQ ID NO: 61) and BvCYP76AD^{W13L} (SEQ ID NO: 62) were integrated into genome of the Y. lipolytica W29 strain expressing Cas9 (ST6512, Table 1) to get strains ST11015-11023 (Table 1). The genes were codon-optimized for Y. lipolytica using the online tool of GeneArt from ThermoFisher Scientific. The constructed strains were then cultivated for betaxanthin production (FIG. 11a). The integration of PgDOD* in Y. lipolytica did not result in a colour change in the culture broth. This might be due to the failure in optimizing the codon usage of this heterologous gene for expression in Y. lipolytica, which has been shown to have significant influence on protein expression in this host (De Pourcq et al. 2012). Moreover, to our surprise, the strains with BgDOD2-EvTYH (ST11019), MjDOD-BvCYP76AD^{W13L} (ST11021), MjDOD-EvTYH (ST11022) and MjDOD-AnTYH (ST11023) did show a pink/red-coloured broth (FIG. 11b). These strains (particularly ST11022), showed a maximum absorbance at λ=510 nm, which is different from λₘₐₓ=530 nm for betacyanins. After expression of glycosyltransferases BvSGT2 and DbB5GT in strain ST11022, we obtained a broth with a UV-Vis peak at λₘₐₓ=530 nm (betacyanins) (FIG. 11c). The HPLC analysis confirmed betanin production of 21.07±1.00 mg/L for ST11193, and 5.15±0.39 mg/L for ST11195 (FIG. 11d), after 48 hours of cultivation in MM (pABA⁻) medium supplemented with 20 g/L glucose showed. Despite the higher total titer of betanin produced by Y. lipolytica strains, more than 50% of this content was intracellular, which necessitates the extraction process for all fermentation processes. It should also be noted that in strains ST11019, ST11020, ST11021, ST11022, ST11193, ST11194 and ST11195, the red betalain with λₘₐₓ=510 nm was present in the fermentation broth.

### Example 6—Resequencing of High-Producing Phenotypes

To verify the genes and their copy number we re-sequenced four high-producing isolates; ST8251, ST10528-lib3-iso2, ST10528-lib3-iso16 and ST10529-lib3-iso21; using NGS sequencing (Illumina, MiSeq V2, paired end). We used the CEN.PK113-7D genome from NCBI (BioProject PRJNA393501) and added an artificial chromosome consisting of each of the codon-optimized genes AhDOD (SEQ ID NO: 16), AnTyh (SEQ ID NO: 38), AoTyH (SEQ ID NO: 40), AtDOD (SEQ ID NO: 14), BaTyH (SEQ ID NO: 30), BgDOD1 (SEQ ID NO: 6), BgDOD2 (SEQ ID NO: 22), BvCYP76AD1^{W13L}(SEQ ID NO: 62), BvDOD1 (SEQ ID NO: 4), BvDOD2 (SEQ ID NO: 24), BvDOD3 (SEQ ID NO: 26), CbTyH (SEQ ID NO: 32), EvTyH (SEQ ID NO: 44), MjDOD (SEQ ID NO: 61), MmTyH1 (SEQ ID NO: 42), MmTyH2 (SEQ ID NO: 48), OfTyH (SEQ ID NO: 36), PaDOD (SEQ ID NO: 18), PaTyH (SEQ ID NO: 34), PdTyH (SEQ ID NO: 46), PgDOD (SEQ ID NO: 8), PgDOD* (SEQ ID NO: 10), SoDOD (SEQ ID NO: 12), and SsDOD (SEQ ID NO: 20).

The reads were mapped to the CEN.PK113-7D genome with the artificial chromosomes using Bowtie2 (Langmead, B et al., 2012). To experimentally verify the presence of more than one variant of TYHs and DODs in the isolates, PCR amplification of all the genes used for library construction was carried out. After amplification, biobricks were sent for sequencing. The sequencing results indicated the presence of the following variants in each isolate:

| Strain | Isolate | Variants |
|---|---|---|
| ST8251 | — | — |
| ST10528 | Lib3-iso2 | BgDOD1(2x), PgDOD* (1x), AnTyH (1x), BaTYH(1x), |
| | | CbTyH(1x) |
| ST10528 | Lib3-iso16 | BgDOD1(2x), PgDOD*(1x), AnTyH (1x), AoTyH(1x), |
| | | BaTYH(1x), CbTyH(1x) |
| ST10529 | Lib3-iso21 | BgDOD1(2x), PgDOD*(1x), AnTyH (2x), CbTyH(1x) |

This showed that not only the nature of the genes but also their copy number is important for high production.

### Example 7—Plant Glucosyltransferases from Different Species for Production of Betacyanins

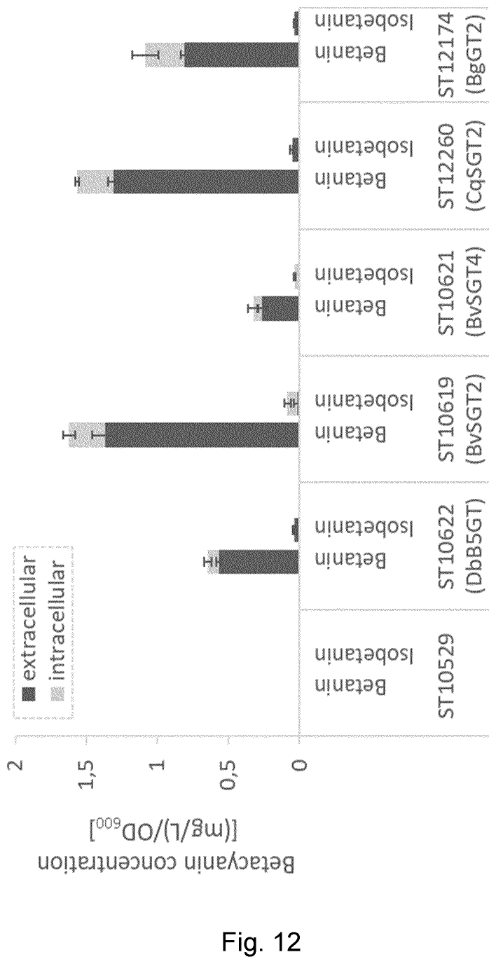

After the scopoletin glycosyltransferases BvSGT2 and BvSGT4 had been found to be able to glycosylate betanidin and to produce betanin when expressed in yeast strains that have a DOD and a TYH, we searched for more scopoletin glycosyltransferases as described in Example 1. Twenty-four scopoletin glycosyltransferases or scopoletin glycosyltransferase-like enzymes from different plant species were identified. These genes were codon optimized for yeast and transformed into the betaxanthin-producing strain ST10529. Furthermore, 3 GTs from Bougainvillea glabra, BgGT1, BgGT2 and BgGT3, were transformed into ST10529 together with DbB5GT and BvSGT2 and BvSGT4 as references. Of the 27 tested GTs, only strains with integrated BgGT2 or CqSGT2 appeared red on the transformation plates. This was also confirmed by cultivation of ST12260 (CqSGT2) and ST12174 (BgGT2) in comparison with BvSGT2, BvSGT4 and DbB5GT. Betanin concentration in the fermentation broth was measured by HPLC after 72 hours of cultivation in MM (pABA⁻) medium supplemented with 20 g/L glucose (FIG. 12). After normalizing the betanin formation to an OD₆₀₀ of 1, strains with CqSGT2 and BvSGT2 had the highest titer with a total betanin concentration of 1.58±0.05 mg/L and 1.62±0.13 mg/L respectively. The strain with BgGT2 (total of 1.08±0.11 mg/L), also resulted in a higher titer than the reference GT DbB5GT.

| Name | Species | Accession number |
|---|---|---|
| DbB5GT | Dorotheanthus/Cleretum bellidiformis | CAB56231.1 |
| BvSGT1 | Beta vulgaris | XP 010681315.1 |
| BvSGT2 | B. vulgaris | XP_010691725.1 |
| BvSGT3 | B. vulgaris | XP_010691729.1 |
| BvSGT4 | B. vulgaris | XP_010691730.1 |
| BvSGT5 | B. vulgaris | XP 010691724.1 |
| CqSGT1 | C. quinoa | XP_021718283.1 |
| PaSGT1 | Prunus avium | XP_021823916.1 |
| SoSGT1 | Spinacia oleracea | XP_021851291.1 |
| VrSGT1 | Vitis riparia | XP_034682237.1 |
| CqSGT2 | Chenopodium quinoa | XP_021753346.1 |
| CqSGT3 | C. quinoa | XP_021753343.1 |
| CqSGT4 | C. quinoa | XP_021753345.1 |
| CqSGT5 | C. quinoa | XP_021753348.1 |
| CqSGT6 | C. quinoa | XP_021714530.1 |
| SoSGT2 | S. oleracea | XP_021851289.1 |
| SoSGT3 | S. oleracea | XP_021851290.1 |
| SoSGT4 | S. oleracea | XP_021851293.1 |
| SoSGT5 | S. oleracea | XP_021839484.1 |
| SoSGT6 | S. oleracea | XP_021863511.1 |
| SoSGT7 | S. oleracea | XP_021851288.1 |
| CsSGT1 | Camellia sinensis | XP_028053269.1 |
| CISISGT1 | Citrus sinensis | XP_006487031.1 |
| CiclSGT1 | Citrus clementina | XP_006422969.1 |
| EgSGT1 | Eucalyptus grandis | XP_039172473.1 |
| CpSGT1 | Carica papaya | XP_021887294.1 |
| MeSGT1 | Manihot esculenta | XP_021624601.1 |
| RaSGT1 | Rhodamnia argentea | XP_030525034.1 |
| TcSGT1 | Theobroma cacao | XP_007042481.2 |
| BgGT1 | Bougainvillea glabra (red) | — |
| BgGT2 | B. glabra (red) | — |
| BgGT3 | B. glabra (red) | — |

### Example 8—Engineering the Oleaginous Yeast Yarrowia lipolytica for Improved Betalain Production

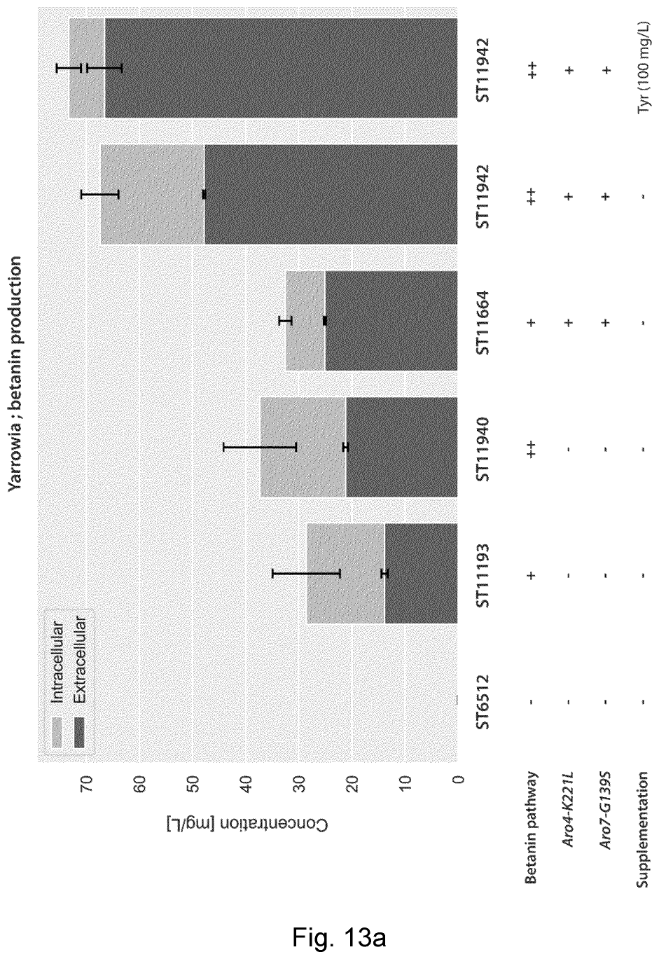

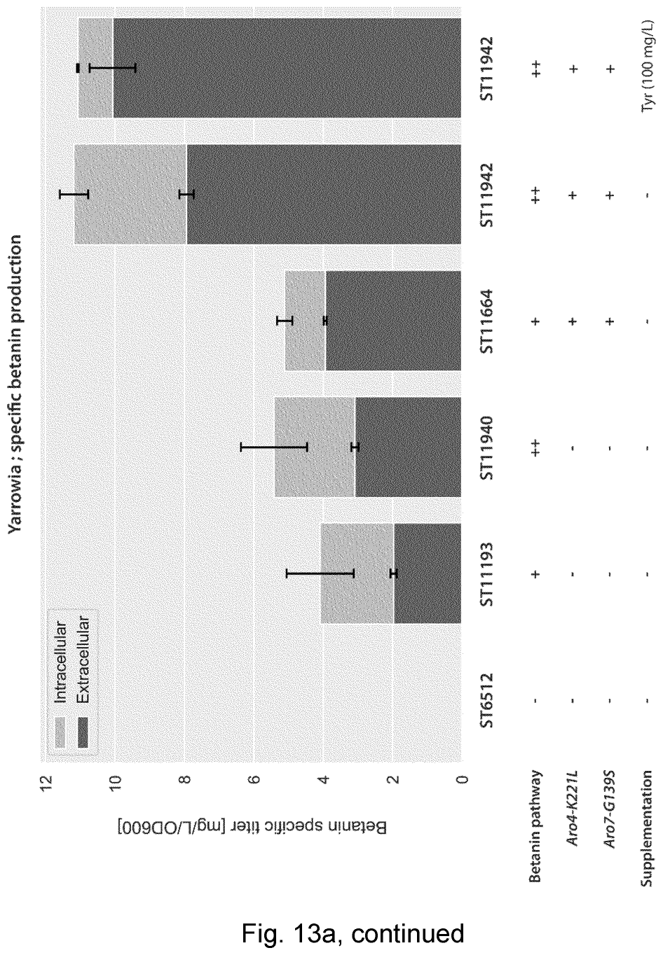

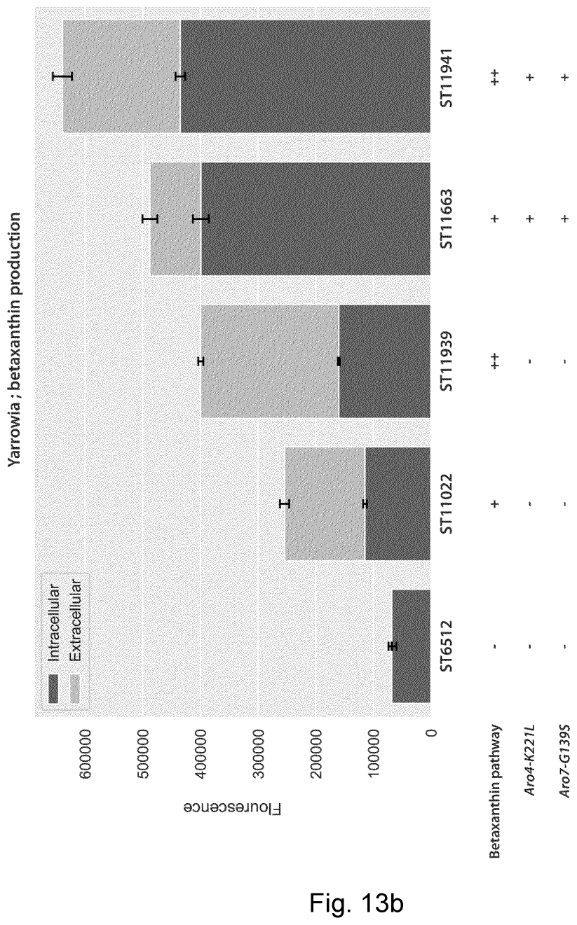

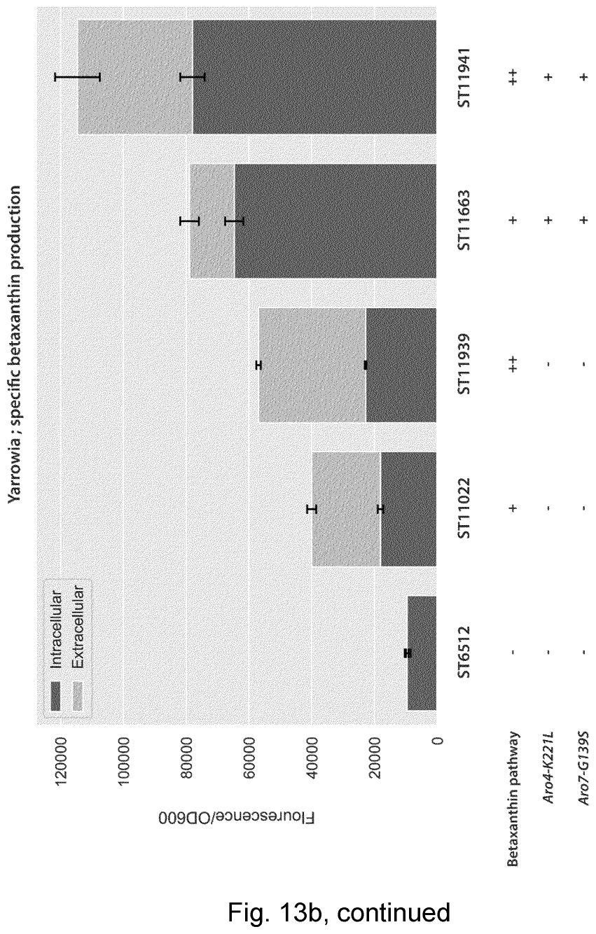

As an oleaginous yeast, Yarrowia lipolytica is naturally endowed with high metabolic flux towards malonyl-CoA and the pentose phosphate pathway (PPP). These traits have proven particularly relevant to the production of shikimate-derived compounds, and it is becoming increasingly evident that Y. lipolytica is an attractive host for high-level production of aromatic compounds (Sáez-Sáez et al. 2020; Gu et al. 2020). Encouraged by the initial betalain production in Y. lipolytica, the best producing betaxanthin (MjDOD-EvTYH; ST11022) and betanin strain (MjDOD-EvTYH-BvSGT2; ST11193) were selected for further engineering. To start, the L-tyrosine precursor supply was enhanced via the simultaneous implementation of feed-back resistant Aro4 (YIARO4^{K221L}) and Aro7 (YIARO7^{G139S}) alleles in respectively ST11022 and ST11193—resulting in the strains ST11663 and ST11664. Hereafter, an additional copy of the heterologous betaxanthin (MjDOD-EvTYH) pathway was implemented in ST11022 and ST11663—resulting in strains ST11939 and ST11941—and an additional copy of the heterologous betanin (MjDOD-EvTYH-BvSGT2) pathway was implemented in ST11193 and ST1166—resulting in the strains ST11940 and ST11942. The engineered Y. lipolytica strains were noticeably more yellow or red, depending on the implemented betalain pathway. For micro-titer plate cultivation of betalain-producing Y. lipolytica strains, cells were inoculated from a 2 mL MM pre-culture into 2 mL MM (-pABA) containing 20 g/L glucose to a starting OD600 of 0.1 in a 24-deep well microtiter plate with an air-penetrable lid and incubated for 48 h with shaking at 250 rpm. For cultivation of the most heavily engineered betanin-producing Y. lipolytica strain, ST11942, the cultivation media was additionally supplemented with 100 mg/L L-tyrosine to probe potential metabolic bottlenecks. The cultivation broth was processed as previously described. Betaxanthin production was quantified based on fluorescence (excitation=463 nm; emission=512 nm) and betanin production quantified based on absorbance at 535 nm, relative to a dilution row of red beet extract diluted with dextrin. The betanin production was estimated at 67.41±3.51 mg/L for ST11942, the most heavily engineered Y. lipolytica strain, without L-tyrosine supplementation (FIG. 13a). With 100 mg/L L-tyrosine supplemented the media, ST11942 produced 73.28±2.29 mg/L of betanin. The improvement pattern observed in the betanin-producing strains was mostly consistent with the improvement pattern observed in the betaxanthin-producing strains (FIG. 13b).

### Example 9—Tyrosine Supplementation Further Improves Betalain Production in Yarrowia lipolytica

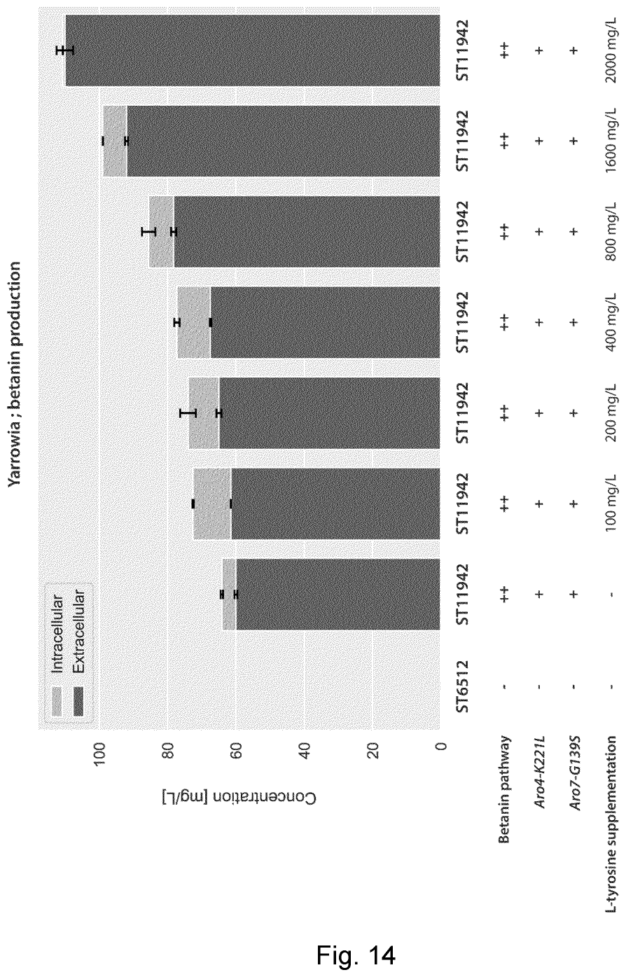

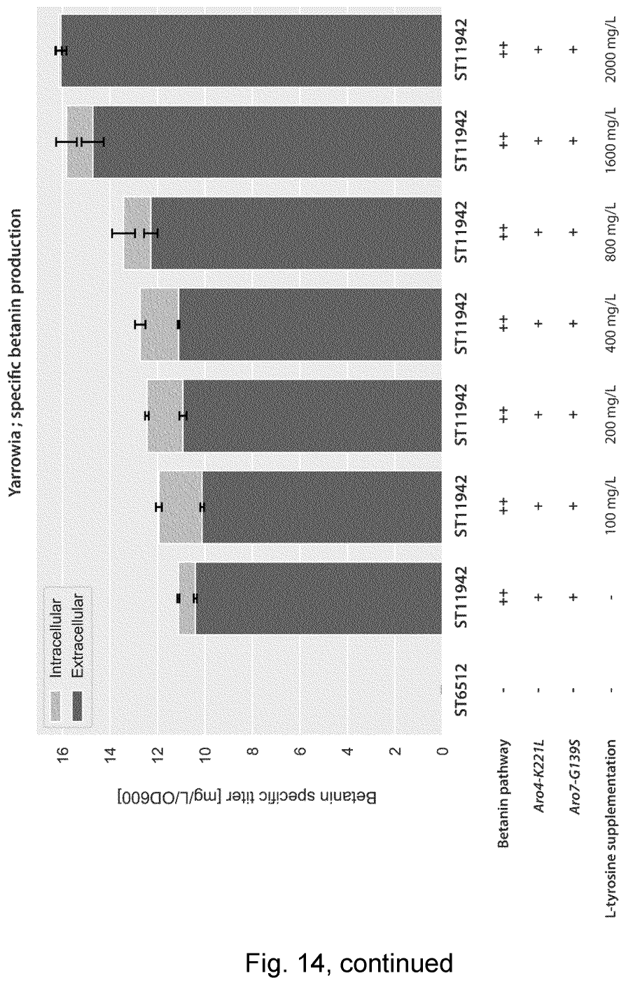

To further evaluate the potential of engineering Y. lipolytica for improved betalain production by enhancing the L-tyrosine precursor supply, ST11942 was cultivated in MM media with increasing amounts of L-tyrosine supplementation. Here, MM media was prepared containing 100 mg/L, 200 mg/L, 400 mg/L, 800 mg/L, 1600 mg/L, and 2000 mg/L of L-tyrosine, and ST11942 cultivated as described in Example 8. Due to the poor solubility of L-tyrosine in water, a stock solution of 50 g/L L-tyrosine was prepared in 1M HCL. Following L-tyrosine supplementation, the pH of the MM was returned to pH 6 via the addition of 1M NaOH. L-tyrosine that precipitated after pH-readjustment was slowly resolubilized during cultivation, as the media reacidified due to the organic acids produced by Y. lipolytica during fermentation. Betanin production was quantified based on absorbance at 535 nm, relative to a dilution row of red beet extract diluted with dextrin, and estimated at 110.65±2.40 mg/L when 2000 mg/L of L-tyrosine was supplemented (FIG. 14). No discernable Y. lipolytica growth impairment was observed as measured by the final OD₆₀₀, even when the media was supplemented with 2000 mg/L L-tyrosine—indicating an inherent tolerance to this amino acid.

### Example 10—Disruption of 4-Hydroxyphenylpyruvate Acid Dioxygenase (4-HPPD) Improves Betalain Production

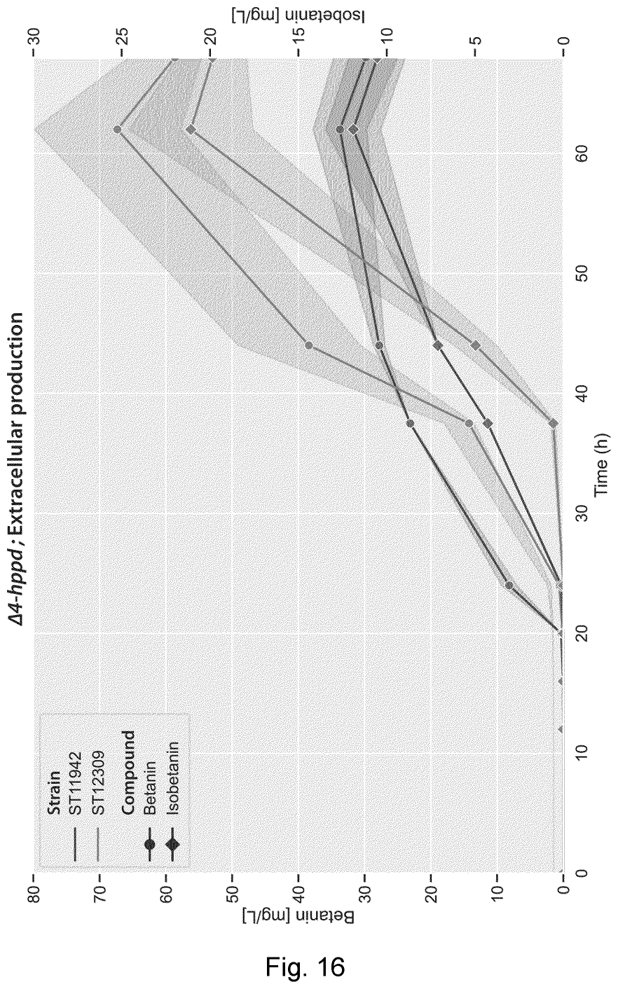

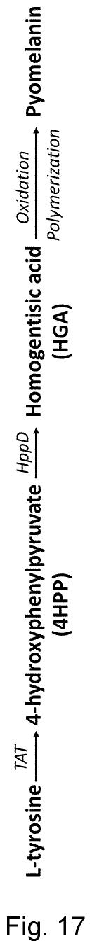

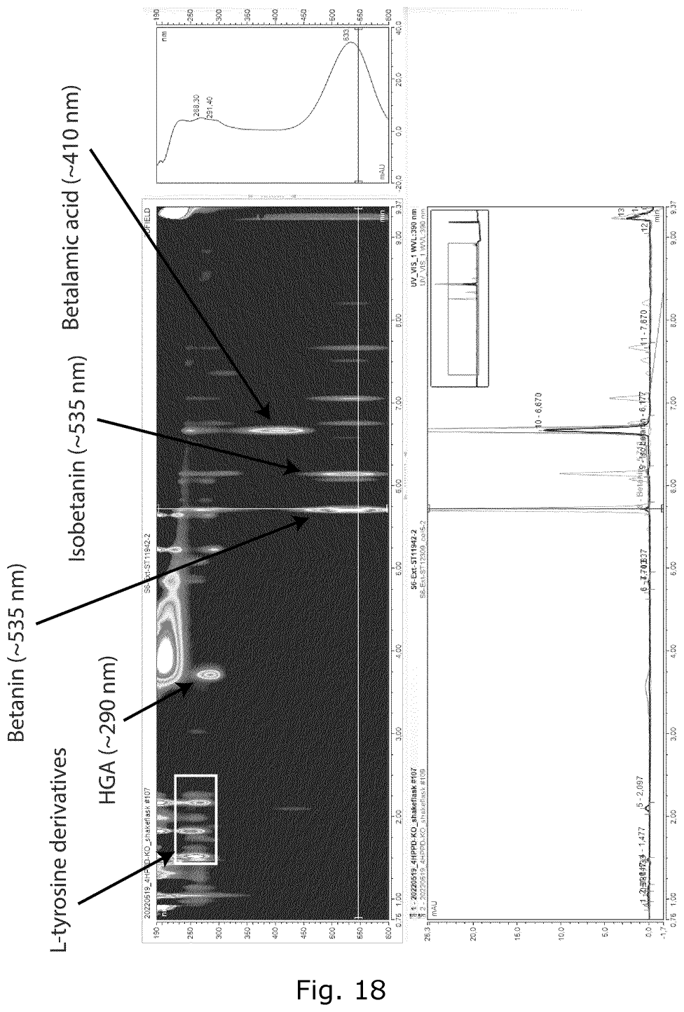

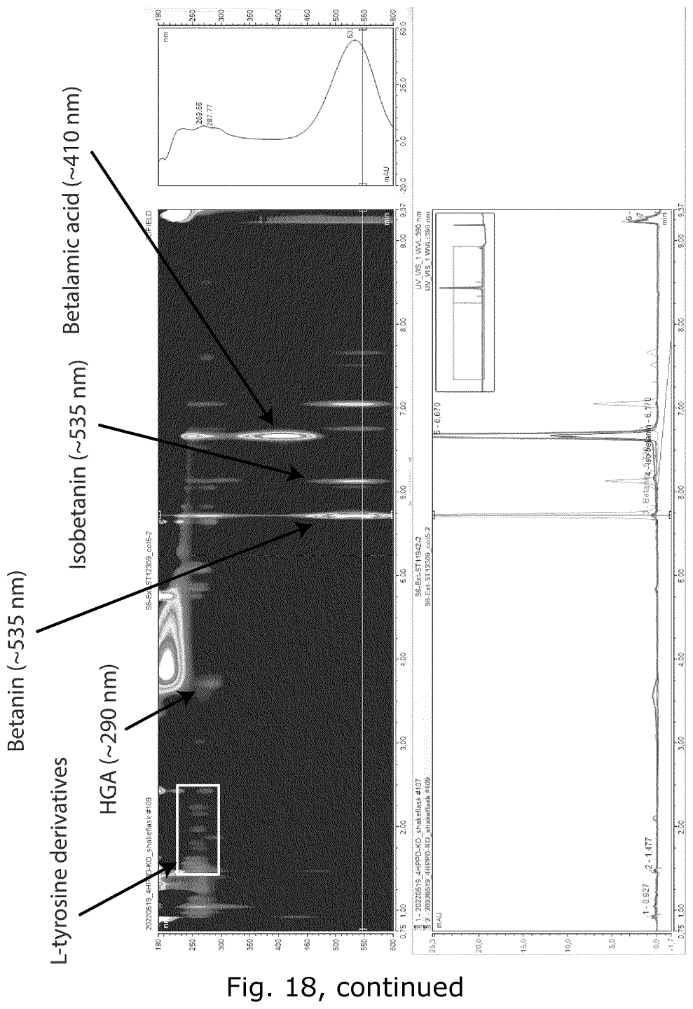

While Y. lipolytica surprisingly turned out to be an excellent host for the production of betalains (Example 9), under cultivation in bioreactors, it simultaneously produced brown pigments as by-products. These brown pigments are likely melanins. The formation of pyomelanin under certain cultivation conditions by wild-type Y. lipolytica has been described previously (Tahar et al. 2020). Here, a tyrosine aminotransferase first converts L-tyrosine into 4-hydroxyphenylpyruvic acid, which is subsequently oxygenated by a 4-hydroxyphenylpyruvic acid dioxygenase (4-HPPD), yielding homogentisic acid (HGA) (Larroude et al. 2021). Once homogentisic acid accumulates sufficiently in the extracellular environment it is autoxidized and polymerized into various melanins—hereamong allomelanins, and the sub-class pyomelanin. To retain and improve L-tyrosine precursor supply for betalain production and to prevent the formation of melanins, we hypothesized that 4-HPPD can be disrupted by replacing parts or the entirety of the coding sequence. To this end, 5-600 bp upstream and downstream of 4-HPPD was PCR amplified, generating USER overhangs compatible with a hygromycin expression cassette flanked by LoxP sites. The 4-HPPD upstream region, hygromycin cassette, and 4-HPPD downstream region were digested with USER enzyme, ligated together with T4 DNA ligase, and the resulting ligation mixture used as template to amplify the 4-HPPD repair template. The repair template was transformed into ST11942 and transformants with 4-HPPD disruption identified via hygromycin selection and colony PCR—resulting in ST12309. To assess the effect of 4-HPPD disruption on betanin production and degradation, ST11942 and ST12309 were cultivated comparatively. Here, ST11942 and four separate, correct transformants of ST12309 were cultured over-night in liquid YPD media, plated on YPD-agar plates, and then a single colony was inoculated into 50 mL MM (-pABA) containing 20 g/L glucose to a starting OD660 of 0.1. Cultivations were carried out in duplicate in 500 mL shakeflasks, which were incubated at 30° C. with shaking at 200 rpm. Samples were taken throughout the cultivation, and cultivation broth processed as previously described. Betanin and isobetanin production was assessed by HPLC (FIG. 16), and at its highest (62 h) ST11942 yielded 33.7 mg/L and 11.9 mg/L of betanin and isobetanin, respectively. In comparison, ST12309 yielded, at its highest (62 h), 67.3 mg/L and 21.1 mg/L of betanin and isobetanin, respectively. In conclusion, the deletion of 4-HPPD doubled the betanin titer. An almost identical HPLC method used to quantify betalains in this work, has previously been used to characterize HGA production in Y. lipolytica (Larroude et al. 2021). As seen on the HPLC chromatogram comparing the extracellular metabolites in ST11942 and ST12309 at 44 h (FIG. 18), L-tyrosine-related peaks (Larroude et al. 2021; Taher et al. 2020) were in general significantly reduced in ST12309 compared to in ST11942, and notably a peak potentially fitting with HGA (290 nm; Taher et al. 2020) was likewise significantly reduced in ST12309. Meanwhile, peaks corresponding to betalamic acid, betanin, and isobetanin were significantly increased in ST12309 compared to ST11942.

### REFERENCES

Ben Tahar, I., Kus-Liśkiewicz, M., Lara, Y., Javaux, E., & Fickers, P. (2020). Characterization of a nontoxic pyomelanin pigment produced by the yeast Yarrowia lipolytica. Biotechnology Progress, 36(2). https://doi.org/10.1002/btpr.2912
Bowman, Emily K., Matthew Deaner, Jan-Fang Cheng, Robert Evans, Ernst Oberortner, Yasuo Yoshikuni, and Hal S. Alper. 2020. “Bidirectional Titration of Yeast Gene Expression Using a Pooled CRISPR Guide RNA Approach.” Proceedings of the National Academy of Sciences 117 (31): 18424-30. https://doi.org/10.1073/pnas.2007413117.
Brockington, Samuel F., Ya Yang, Fernando Gandia-Herrero, Sarah Covshoff, Julian M. Hibberd, Rowan F. Sage, Gane K. S. Wong, Michael J. Moore, and Stephen A. Smith. 2015. “Lineage-Specific Gene Radiations Underlie the Evolution of Novel Betalain Pigmentation in Caryophyllales.” The New Phytologist 207 (4): 1170-80. https://doi.org/10.1111/nph.13441.
DeLoache, William C., Zachary N. Russ, Lauren Narcross, Andrew M. Gonzales, Vincent J. J. Martin, and John E. Dueber. 2015. “An Enzyme-Coupled Biosensor Enables (S)-Reticuline Production in Yeast from Glucose.” Nature Chemical Biology 11 (7): 465-71. https://doi.org/10.1038/nchembio.1816.
De Pourcq, Karen, Wouter Vervecken, Isabelle Dewerte, Albena Valevska, Annelies Van Hecke, and Nico Callewaert. 2012. “Engineering the Yeast Yarrowia Lipolytica for the Production of Therapeutic Proteins Homogeneously Glycosylated with Man8GlcNAc2 and Man5GlcNAc2.” Microbial Cell Factories 11 (1): 53. https://doi.org/10.1186/1475-2859-11-53.
Dos Santos, Cleudia Destro, Marliya Ismail, Aline Schilling Cassini, Ligia Damasceno Ferreira Marczak, Isabel Cristina Tessaro, and Mohammed Farid. 2018. “Effect of Thermal and High Pressure Processing on Stability of Betalain Extracted from Red Beet Stalks.” Journal of Food Science and Technology 55 (2): 568-77. https://doi.org/10.1007/s13197-017-2966-0.
Gandia-Herrero, Fernando, and Francisco Garcia-Carmona. 2020. “The Dawn of Betalains.” New Phytologist 227 (3): 664-66. https://doi.org/10.1111/nph.16295.
Gonçalves, Leticia Christina Pires, Marco Aurélio de Souza Trassi, Nathana Barbosa Lopes, Felipe Augusto Dörr, Monica Teixeira dos Santos, Wilhelm Josef Baader, Vani Xavier Oliveira, and Erick Leite Bastos. 2012. “A Comparative Study of the Purification of Betanin.” Food Chemistry 131 (1): 231-38. https://doi.org/10.1016/j.foodchem.2011.08.067.
Grewal, Parbir S., Cyrus Modavi, Zachary N. Russ, Nicholas C. Harris, and John E. Dueber. 2018. “Bioproduction of a Betalain Colour Palette in Saccharomyces Cerevisiae.” Metabolic Engineering 45: 180-88. https://doi.org/10.1016/j.ymben.2017.12.008.
Gu, Y., Ma, J., Zhu, Y., Ding, X., & Xu, P. (2020). Engineering Yarrowia lipolytica as a Chassis for de Novo Synthesis of Five Aromatic-Derived Natural Products and Chemicals. ACS Synthetic Biology, 9(8), 2096-2106. https://doi.org/10.1021/acssynbio.Oc00185
Jessop-Fabre, Mathew M., Tadas Jakočiūnas, Vratislav Stovicek, Zongjie Dai, Michael K. Jensen, Jay D. Keasling, and Irina Borodina. 2016. “EasyClone-MarkerFree: A Vector Toolkit for Marker-Less Integration of Genes into Saccharomyces Cerevisiae via CRISPR-Cas9.” Biotechnology Journal. https://doi.org/10.1002/biot.201600147.
Khan, Mohammad Imtiyaj, and P. Giridhar. 2015. “Plant Betalains: Chemistry and Biochemistry.” Phytochemistry 117 (September): 267-95. https://doi.org/10.1016/j.phytochem.2015.06.008.
Khan, Mohammad Imtiyaj, P. S. C. Sri Harsha, P. Giridhar, and G. A. Ravishankar. 2012. “Pigment Identification, Nutritional Composition, Bioactivity, and in Vitro Cancer Cell Cytotoxicity of Rivina humilis L. Berries, Potential Source of Betalains.” LWT 47 (2): 315-23. https://doi.org/10.1016/j.lwt.2012.01.025.
Kildegaard, Kanchana Rueksomtawin, Larissa Ribeiro Ramos Tramontin, Ksenia Chekina, Mingji Li, Tobias Justus Goedecke, Mette Kristensen, and Irina Borodina. 2019. “CRISPR/Cas9-RNA Interference System for Combinatorial Metabolic Engineering of Saccharomyces Cerevisiae.” Yeast (Chichester, England) 36 (5): 237-47. https://doi.org/10.1002/yea.3390.
Langmead, B., Salzberg, S. Fast gapped-read alignment with Bowtie2. Nat Methods 9, 357-359 (2012). https://doi.org/10.1038/nmeth.1923
Larroude, M., Onésime, D., Rué, O., Nicaud, J. M., & Rossignol, T. (2021). A Yarrowia lipolytica strain engineered for pyomelanin production. Microorganisms, 9(4). https://doi.org/10.3390/microorganisms9040838
Milne, N., P. Thomsen, N. Mølgaard Knudsen, P. Rubaszka, M. Kristensen, and I. Borodina. 2020. “Metabolic Engineering of Saccharomyces Cerevisiae for the de Novo Production of Psilocybin and Related Tryptamine Derivatives.” Metabolic Engineering 60 (July): 25-36. https://doi.org/10.1016/j.ymben.2019.12.007.
Sáez-Sáez, J., Wang, G., Marella, E. R., Sudarsan, S., Cernuda Pastor, M., & Borodina, I. (2020). Engineering the oleaginous yeast Yarrowia lipolytica for high-level resveratrol production. Metabolic Engineering, 62 (August), 51-61. https://doi.org/10.1016/j.ymben.2020.08.009
Sunnadeniya, Rasika, Alexander Bean, Matthew Brown, Neda Akhavan, Gregory Hatlestad, Antonio Gonzalez, V. Vaughan Symonds, and Alan Lloyd. 2016. “Tyrosine Hydroxylation in Betalain Pigment Biosynthesis Is Performed by Cytochrome P450 Enzymes in Beets (Beta Vulgaris).” PLoS ONE 11 (2). https://doi.org/10.1371/journal.pone.0149417.
Timoneda, Alfonso, Tao Feng, Hester Sheehan, Nathanael Walker-Hale, Boas Pucker, Samuel Lopez-Nieves, Rui Guo, and Samuel Brockington. 2019. “The Evolution of Betalain Biosynthesis in Caryophyllales.” The New Phytologist 224 (1): 71-85. https://doi.org/10.1111/nph.15980.

Sequence list

| SEQ ID NO. | Description |
|---|---|
| 1 | MjDOD protein from Mirabilis jalapa (garden four-o'clock), NCBI |
| | reference sequence: B6FOW8.1 |
| 2 | Gene for MjDOD from Mirabilis jalapa (garden four-o'clock), |
| | codon-optimized for S. cerevisiae |
| 3 | BvDOD1 protein from Beta vulgaris, NCBI reference sequence: |
| | Q70FG7.1 |
| 4 | Gene for BvDOD1 from Beta vulgaris, codon-optimized for S. |
| | cerevisiae |
| 5 | BgDOD1 protein from Bougainvillea glabra, |
| | GenBank: ASW22755.1 |
| 6 | Gene for BgDOD1 from Bougainvillea glabra, codon-optimized for |
| | S. cerevisiae |
| 7 | PgDOD protein from Portulaca grandiflora, |
| | NCBI reference sequence: CAE45178.1 |
| 8 | Gene for PgDOD from Portulaca grandiflora, codon-optimized for |
| | S. cerevisiae |
| 9 | PgDOD* protein (mutated version) from Portulaca grandiflora |
| 10 | Gene for PgDOD* (mutated version: T732A) from Portulaca |
| | grandiflora, codon-optimized for S. cerevisiae |
| 11 | SoDOD protein from Spinacia oleracea (spinach), NCBI reference |
| | sequence: XP_021836119.1 |
| 12 | Gene for SoDOD from Spinacia oleracea (spinach), codon- |
| | optimized for S. cerevisiae |
| 13 | AtDOD protein from Amaranthus tricolour, GenBank: AJW81119.1 |
| 14 | Gene for AtDOD from Amaranthus tricolour, codon-optimized for |
| | S. cerevisiae |
| 15 | AhDOD protein from Amaranthus hypochondriacus (grain |
| | amaranth), |
| | GenBank: ADZ48644.1 |
| 16 | Gene for AhDOD from Amaranthus hypochondriacus (grain |
| | amaranth), codon-optimized for S. cerevisiae |
| 17 | PaDOD protein from Phytolacca americana (American |
| | pokeweed), NCBI reference sequence: BAH66635.1 |
| 18 | Gene for PaDOD from Phytolacca americana (American |
| | pokeweed), codon-optimized for S. cerevisiae |
| 19 | SsDOD protein from Suaeda salsa, |
| | GenBank: ACO59903.1 |
| 20 | Gene for SsDOD from Suaeda salsa, codon-optimized for S. |
| | cerevisiae |
| 21 | BgDOD2 protein from Bougainvillea glabra, GenBank: |
| | BAG80687.1 |
| 22 | Gene for BgDOD2 from Bougainvillea glabra, codon-optimized for |
| | S. cerevisiae |
| 23 | BvDOD2 protein from Beta vulgaris, GenBank: AET43293.1 |
| 24 | Gene for BvDOD2 protein from Beta vulgaris, codon-optimized for |
| | S. cerevisiae |
| 25 | BvDOD3 protein from Beta vulgaris, |
| | GenBank: AET43287.1 |
| 26 | Gene for BvDOD3 from Beta vulgaris, codon-optimized for S. |
| | cerevisiae |
| 27 | BvCYP76ADW13L protein from Beta vulgaris, |
| | GenBank: AET43289.1 |
| 28 | Gene for BvCYP76ADW13L from Beta vulgaris, codon-optimized for |
| | S. cerevisiae |
| 29 | BaTYH protein from Basella alba, GenBank: AJD87470.1 |
| 30 | Gene for BaTYH from Basella alba, codon-optimized for S. |
| | cerevisiae |
| 31 | CbTYH protein from Cleretum bellidiforme, |
| | GenBank: AJD87468.1 |
| 32 | Gene for CbTYH from Cleretum bellidiforme, codon-optimized for |
| | S. cerevisiae |
| 33 | PaTYH protein from Phytolacca americana, |
| | GenBank: AJD87467.1 |
| 34 | Gene for PaTYH from Phytolacca americana, codon-optimized for |
| | S. cerevisiae |
| 35 | OfTYH protein from Opuntia ficus-indica, |
| | GenBank: AJD87464.1 |
| 36 | Gene for OfTYH from Opuntia ficus-indica, codon-optimized for S. |
| | cerevisiae |
| 37 | AnTYH protein from Abronia nealleyi, |
| | GenBank: AKI33952.1 |
| 38 | Gene for AnTYH from Abronia nealleyi, codon-optimized for S. |
| | cerevisiae |
| 39 | AoTYH protein from Acleisanthes obtusa, |
| | GenBank: AKI33950.1 |
| 40 | Gene for AoTYH from Acleisanthes obtusa, codon-optimized for |
| | S. cerevisiae |
| 41 | MmTYH1 protein from Mirabilis multiflora, |
| | GenBank: AKI33948.1 |
| 42 | Gene for MmTYH1 from Mirabilis multiflora, codon-optimized for |
| | S. cerevisiae |
| 43 | EvTYH protein from Ercilla volubilis, GenBank: AKI33945.1 |
| 44 | Gene for EvTYH protein from Ercilla volubilis, codon-optimized for |
| | S. cerevisiae |
| 45 | PdTYH protein from Phytolacca dioica, |
| | GenBank: AKI33942.1 |
| 46 | Gene for PdTYH from Phytolacca dioica, codon-optimized for S. |
| | cerevisiae |
| 47 | MmTYH2 protein from Mirabilis multiflora, |
| | GenBank: AKI33937.1 |
| 48 | Gene for MmTYH2 from Mirabilis multiflora, codon-optimized for |
| | S. cerevisiae |
| 49 | DbB5GT protein from Cleretum bellidiforme, |
| | GenBank: CAB56231.1 |
| 50 | Gene for DbB5GT from Cleretum bellidiforme, codon-optimized |
| | for S. cerevisiae |
| 51 | BvSGT1 protein from Beta vulgaris, |
| | NCBI reference sequence: XP_010681315.1 |
| 52 | Gene for BvSGT1 from Beta vulgaris, codon-optimized for S. |
| | cerevisiae |
| 53 | BvSGT2 protein from Beta vulgaris, |
| | NCBI reference sequence: XP_010691725.1 |
| 54 | Gene for BvSGT2 from Beta vulgaris, codon-optimized for S. |
| | cerevisiae |
| 55 | BvSGT3 protein from Beta vulgaris, |
| | NCBI reference sequence: XP_010691729.1 |
| 56 | Gene for BvSGT3 from Beta vulgaris, codon-optimized for S. |
| | cerevisiae |
| 57 | BvSGT4 protein from Beta vulgaris, NCBI reference sequence: |
| | XP_010691730.1 |
| 58 | Gene for BvSGT4 from Beta vulgaris, codon-optimized for S. |
| | cerevisiae |
| 59 | Gene for PgDOD* (mutated version: T732A) from Portulaca |
| | grandiflora, codon-optimized for Y. lipolytcia |
| 60 | Gene for BgDOD2 from Bougainvillea glabra, codon-optimized for |
| | Y. lipolytica |
| 61 | Gene for MjDOD from Mirabilis jalapa (garden four-o'clock), |
| | codon-optimized for Y. lipolytica |
| 62 | Gene for BvCYP76ADW13L from Beta vulgaris, codon-optimized for |
| | Y. lipolytica |
| 63 | Gene for EvTyH protein from Ercilla volubilis, codon-optimized for |
| | Y. lipolytica |
| 64 | Gene for AnTyH from Abronia nealleyi, codon-optimized for Y. |
| | lipolytica |
| 65 | CqSGT2 protein sequence from Chenopodium quinoa, NCBI |
| | reference sequence: XP_021753346.1 |
| 66 | Gene for CqSGT2 from Chenopodium quinoa, codon-optimized |
| | for S. cerevisiae |
| 67 | BgGT2 protein from Bougainvillea glabra |
| 68 | Gene for BgGT2 from Bougainvillea glabra, codon-optimized for |
| | S. cerevisiae |
| 69 | 4-hydroxyphenylpyruvate dioxygenase (4-HPPD) protein |
| | sequence from Yarrowia lipolytica, UniPROT reference number: |
| | A0A 1H6PW31 |
| 70 | Gene for 4-hydroxyphenylpyruvate dioxygenase from Yarrowia |
| | lipolytica, UniPROT reference number: YALI1_B28454g |
| 71 | 3-deoxy-7-phosphoheptulonate synthase (Aro4) protein sequence |
| | from Yarrowia lipolytica, UniPROT/NCBI reference |
| | number: A0A1D8N9Z6; AOW02456.1 |
| 72 | Gene for Aro4 from Yarrowia lipolytica, UniPROT reference |
| | number: YALI1_C09308g |
| 73 | Chorismate mutase (Aro7) protein sequence from Yarrowia |
| | lipolytica, UniPROT/NCBI reference |
| | number: A0A1D8NIT5; AOW05549.1 |
| 74 | Gene for Aro7 from Yarrowia lipolytica, UniPROT reference |
| | number: YALI1_E20751g |
| 75 | 3-deoxy-7-phosphoheptulonate synthase (Aro4) protein sequence |
| | from Saccharomyces cerevisiae, UniPROT reference |
| | number: QHB07012.1 |
| 76 | Gene for Aro4 from Saccharomyces cerevisiae, UniPROT/NCBI |
| | reference number: SCENOB03630; YBR249C |
| 77 | Chorismate mutase (Aro7) protein sequence |
| | from Saccharomyces cerevisiae, UniPROT reference |
| | number: QHB12310.1 |
| 78 | Gene for Aro7 from Saccharomyces cerevisiae, UniPROT/NCBI |
| | reference number: SCEN0P03550; YPR060C |
| 79 | BgGT1 protein from Bougainvillea glabra |
| 80 | BgGT3 protein from Bougainvillea glabra |

### Items

1. A yeast cell capable of producing one or more betalains, said yeast cell expressing:
a. a first heterologous enzyme (TYH) capable of hydroxylating L-tyrosine and oxidizing L-DOPA, such as CYP76ADα;
b. a second heterologous enzyme which is a 4,5-DOPA extradiol dioxygenase (DOD); and
c. a third heterologous enzyme having glycosyltransferase activity, wherein said enzyme is selected from:
   i. Chenopodium quinoa glycosyltransferase CqSGT2 set forth in SEQ ID NO: 65, or a functional variant thereof having at least 70% sequence identity thereto;
   ii. Beta vulgaris glycosyltransferase BvSGT2 set forth in SEQ ID NO: 53, or a functional variant thereof having at least 70% identity thereto;
   iii. Bougainvillea glabra glycosyltransferase BgGT2 set forth in SEQ ID NO: 67, or a functional variant thereof having at least 70% identity thereto; and
   iv. Beta vulgaris glycosyltransferase BvSGT4 set forth in SEQ ID NO: 57, or a functional variant thereof having at least 70% identity thereto;
whereby said cell is capable of producing one or more betalains, wherein said one or more betalains comprise one or more glycosylated betalains such as betanin and/or isobetanin.

2. A yeast cell capable of producing one or more betalains, said yeast cell expressing:
a. a first heterologous enzyme (TYH) capable of hydroxylating L-tyrosine and oxidizing L-DOPA, such as CYP76ADα; a second heterologous enzyme which is a 4,5-DOPA extradiol dioxygenase (DOD); and a third heterologous enzyme, having glycosyltransferase activity, such as an activity selected from a betanidin-5-O-glucosyltransferase (B50G) activity and a cyclo-DOPA-5-O-glucosyltransferase (cDOPA50GT) activity, such as a glycosyltransferase, such as a scopoletin glucosyltransferase (SGT), whereby said cell is capable of producing one or more betalains, wherein said one or more betalains comprise one or more glycosylated betalains such as betanin and/or isobetanin; and/or
b. a first heterologous enzyme (TYH) capable of hydroxylating L-tyrosine and oxidizing L-DOPA, such as CYP76ADα; and a second heterologous enzyme which is a DOD having a truncation in its C-terminal end (DOD*), whereby said cell is capable of producing one or more betalains, wherein said one or more betalains comprise one or more betaxanthins.

3. The yeast cell according to any one of the preceding items, wherein:
the TYH is capable of converting L-tyrosine to L-3,4-dihydroxyphenylalanine (L-DOPA) and/or converting L-DOPA to L-Dopaquinone;
the DOD and/or the DOD* is capable of converting L-DOPA to 4,5-seco-DOPA;
the enzyme having glycosyltransferase activity, such as the glycosyltransferase is capable of converting cyclo-DOPA to cyclo-DOPA-5-O-glucoside and/or glycosylating betanidin, thereby converting betanidin to a glycosylated betalains, such as betanin and/or isobetanin;
and wherein one or more of the following reactions are spontaneous reactions:
conversion of 4,5-seco-DOPA to betalamic acid;
conversion of betalamic acid to one or more of a betaxanthin, betanidin or betanin;
conversion of L-Dopaquinone to cyclo-DOPA;
conversion of cyclo-DOPA to betanidin.

4. The yeast cell according to any one of the preceding items, wherein the genus of said yeast cell is selected from the group consisting of Saccharomyces, Pichia, Yarrowia, Kluyveromyces, Candida, Rhodotorula, Rhodosporidium, Cryptococcus, Trichosporon and Lipomyces.

5. The yeast cell according to any one of the preceding items, wherein the yeast is selected from the group consisting of Saccharomyces cerevisiae, Saccharomyces boulardi, Pichia pastoris, Kluyveromyces marxianus, Cryptococcus albidus, Candida tropicalis, Lipomyces lipofera, Lipomyces starkeyi, Rhodosporidium toruloides, Rhodotorula glutinis, Trichosporon pullulan and Yarrowia lipolytica.

6. The yeast cell according to any one of the preceding items, wherein the enzyme having glycosyltransferase activity is native to a plant, such as of the genus Abronia, Beta, Bougainvillea, Ercilla, or Portacula, such as Abronia nealleyi, Beta vulgaris, Bougainvillea glabra, Ercilla volubilis, or Portacula grandiflora, or a functional variant thereof having at least 80% identity thereto.

7. The yeast cell according to any one of the preceding items, wherein the enzyme having glycosyltransferase activity is selected from:
a. a Beta vulgaris glycosyltransferase such as BvSGT2 as set forth in SEQ ID NO: 53 or a functional variant thereof having at least 80% identity thereto;
b. a Beta vulgaris glycosyltransferase such as BvSGT4 as set forth in SEQ ID NO: 57 or a functional variant thereof having at least 80% identity thereto.

8. The yeast cell according to any one of the preceding items, wherein the TYH is native to a plant, such as of the genus Abronia, Acleisanthes, Basella, Beta, Cleretum, Ercilla, Mirabilis, Optunia, or Phytolacca, such as Abronia nealleyi, Acleisanthes obtusa, Basella alba, Beta vulgaris, Cleretum bellidiforme, Ercilla volubis, Mirabilis multiflora, Optunia ficus-indica, or Phytolacca dioica, or a functional variant thereof having at least 80% identity thereto.

9. The yeast cell according to any one of the preceding items, wherein the TYH is selected from:
a. an Abronia nealleyi TYH such as AnTYH as set forth in SEQ ID NO: 37, or a functional variant thereof having at least 80% identity thereto;
b. an Ercilla volubis TYH such as EvTYH as set forth in SEQ ID NO: 43, or a functional variant thereof having at least 80% identity thereto.

10. The yeast cell according to any one of the preceding items, wherein the DOD is native to a plant, such as of the genus Amaranthus, Beta, Bougainvillea, Mirabilis Phytolacca, Portulaca, Spinacia, or Suaeda, such as Amaranthus hypochondriacus, Amaranthus tricolour, Beta vulgaris, Bougainvillea glabra, Mirabilis jalapa, Phytolacca americana, Portulaca grandiflora, Spinacia oleracea, or Suaeda salsa, or a functional variant thereof having at least 80% identity thereto.

11. The yeast cell according to any one of the preceding items, wherein the DOD* has a mutation resulting in an early stop codon.

12. The yeast cell according any one of the preceding items, wherein the DOD* has a truncation of at least 5 amino acids at the C-terminal end, such as at least 6, such as at least 7, such as at least 8, such as at least 9, such as at least 10, such as at least 12, such as at least 14, such as at least 16, such as at least 18, such as at least 20, such as at least 25, such as at least 30, such as at least 35, such as at least 40, such as at least 45, such as at least 50 amino acids.

13. The yeast cell according to any one of the preceding items, wherein the DOD* is:
a. a truncation of a Mirabilis jalapa DOD such as MjDOD as set forth in SEQ ID NO: 1, or a functional variant thereof having at least 80% identity thereto; or
b. a truncation of a Portulaca grandiflora DOD such as PgDOD as set forth in SEQ ID NO: 7, or a functional variant thereof having at least 80% identity thereto;
c. a Bougainvillea glabra DOD such as BgDOD2 as set forth in SEQ ID NO: 21, or a functional variant thereof having at least 80% identity thereto.

14. The yeast cell according to any one of the preceding items, wherein the DOD is selected from:
a. a Mirabilis jalapa DOD such as MjDOD as set forth in SEQ ID NO: 1 or a functional variant thereof having at least 80% identity thereto; and
b. a Portulaca grandiflora DOD such as PgDOD as set forth in SEQ ID NO: 7 or a functional variant thereof having at least 80% identity thereto; and
c. a DOD having a truncation in its C-terminal end (DOD*) such as PgDOD* as set forth in SEQ ID NO: 9 or a functional variant thereof having at least 80% identity thereto; and
d. a Bougainvillea glabra DOD such as BgDOD2 as set forth in SEQ ID NO: 21 or a functional variant thereof having at least 80% identity thereto.

15. The yeast cell according to any one of the preceding items, wherein the first heterologous enzyme is selected form the group consisting of SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, and SEQ ID NO: 47, or a functional variant thereof having at least 70% identity thereto.

16. The yeast cell according to any one of the preceding items, wherein the first heterologous enzyme is an Abronia nealleyi TYH such as AnTYH as set forth in SEQ ID NO: 37; or an Ercilla volubis TYH such as EvTYH as set forth in SEQ ID NO: 43; or a functional variant thereof having at least 80% identity thereto.

17. The yeast cell according to any one of the preceding items, wherein the second heterologous enzyme is selected form the group consisting of SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, and SEQ ID NO: 25, or a functional variant thereof having at least 70% identity thereto.

18. The yeast cell according to any one of the preceding items, wherein the second heterologous enzyme is a Mirabilis jalapa DOD such as MjDOD as set forth in SEQ ID NO: 1; a Bougainvillea glabra DOD such as BgDOD2 as set forth in SEQ ID NO: 21; a Portulaca grandiflora DOD such as PgDOD as set forth in SEQ ID NO: 7; or a DOD having a truncation in its C-terminal end (DOD*) such as PgDOD* set forth in SEQ ID NO: 9; or a functional variant thereof having at least 80% identity thereto.

19. The yeast cell according to any one of the preceding items, wherein the third heterologous enzyme is a Beta vulgaris SGT such as BvSGT2 as set forth in SEQ ID NO: 53; or BvSGT4 as set forth in SEQ ID NO: 57; or a functional variant thereof having at least 80% identity thereto.

20. The yeast cell according to any one of the preceding items, wherein the third heterologous enzyme is selected form the group consisting of SEQ ID NO: 53, SEQ ID NO: 65, SEQ ID NO: 67 and SEQ ID NO: 57, or a functional variant thereof having at least 70% identity thereto.

21. The yeast cell according to any one of the preceding items, wherein said yeast cell expresses:
a. a TYH from Abronia nealleyi (AnTYH) as set forth in SEQ ID NO: 37; a truncated DOD* as set forth in SEQ ID NO: 9 (PgDOD*); and an SGT from Beta vulgaris (BvSGT2) as set forth in SEQ ID NO: 53; or
b. a TYH from Abronia nealleyi (AnTYH) as set forth in SEQ ID NO: 37; a DOD from Bougainvillea glabra (BgDOD2) as set forth in SEQ ID NO: 21; and an SGT from Beta vulgaris (BvSGT2) as set forth in SEQ ID NO: 53; or
c. a TYH from Abronia nealleyi (AnTYH) as set forth in SEQ ID NO: 37; a truncated DOD* as set forth in SEQ ID NO: 9 (PgDOD*); and an SGT from Beta vulgaris (BvSGT4) as set forth in SEQ ID NO: 57; or
d. a TYH from Abronia nealleyi (AnTYH) as set forth in SEQ ID NO: 37; a DOD from Bougainvillea glabra (BgDOD2) as set forth in SEQ ID NO: 21; and an SGT from Beta vulgaris (BvSGT4) as set forth in SEQ ID NO: 57; or
e. a TYH from Ercilla volubis (EvTYH) as set forth in SEQ ID NO 43; a truncated DOD* as set forth in SEQ ID NO: 9 (PgDOD*); and an SGT from Beta vulgaris (BvSGT2) as set forth in SEQ ID NO: 53; or
f. a TYH from Ercilla volubis (EvTYH) as set forth in SEQ ID NO 43; a DOD from Bougainvillea glabra (BgDOD2) as set forth in SEQ ID NO: 21; and an SGT from Beta vulgaris (BvSGT2) as set forth in SEQ ID NO: 53; or
g. a TYH from Ercilla volubis (EvTYH) as set forth in SEQ ID NO 43; a truncated DOD* as set forth in SEQ ID NO: 9 (PgDOD*); and an SGT from Beta vulgaris (BvSGT4) as set forth in SEQ ID NO: 57; or
h. a TYH from Ercilla volubis (EvTYH) as set forth in SEQ ID NO 43; a DOD from Bougainvillea glabra (BgDOD2) as set forth in SEQ ID NO: 21; and an SGT from Beta vulgaris (BvSGT4) as set forth in SEQ ID NO: 57;
   or functional variants thereof having at least 80% identity thereto, whereby said yeast cell is capable of producing one or more betalains, wherein said one or more betalains comprise a glycosylated betalain, such as betanin.

22. The yeast cell according to any one of the preceding items, wherein said yeast cell expresses:
a. a TYH from Abronia nealleyi (AnTYH) as set forth in SEQ ID NO: 37; and a truncated DOD* as set forth in SEQ ID NO: 9 (PgDOD*); or
b. a TYH from Ercilla volubis (EvTYH) as set forth in SEQ ID NO 43; and a truncated DOD* as set forth in SEQ ID NO: 9 (PgDOD*); or
or functional variants thereof having at least 80% identity thereto, whereby said yeast cell is capable of producing one or more betalains, wherein said one or more betalains comprise one or more betaxanthins.

23. The yeast cell according to any one of the preceding items, wherein the first heterologous enzyme is encoded by SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 42, SEQ ID NO: 44, SEQ ID NO: 46, or SEQ ID NO: 48, preferably wherein the first heterologous enzyme is encoded by SEQ ID NO: 38 or SEQ ID NO: 44; the second heterologous enzyme is encoded by SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, or SEQ ID NO: 26, preferably wherein the second heterologous enzyme is encoded by SEQ ID NO: 10 or SEQ ID NO: 22; and/or the third heterologous enzyme is encoded by SEQ ID NO: 54 or SEQ ID NO: 58; or homologues thereof having at least 80% identity thereto.

24. The yeast cell according to any one of the preceding items, wherein at least one of the genes encoding the TYH, the DOD, or the glycosyltransferase is present in high copy number.

25. The yeast cell according to any one of the preceding items, wherein at least one of the genes encoding the TYH, the DOD, or the glycosyltransferase is under the control of an inducible promoter.

26. The yeast cell according to any one of the preceding items, wherein at least one of the genes encoding the TYH, the DOD, or the glycosyltransferase is codon-optimized for said yeast cell.

27. The yeast cell according to any one of the preceding items, wherein the genes encoding the TYH, the DOD, or the glycosyltransferase are each independently comprised within the genome of the yeast cell or within a vector comprised within the yeast cell.

28. A method for production of one or more betalains in a yeast cell, said method comprising the steps of incubating a yeast cell in a medium, wherein said yeast cell expresses:
a. a first heterologous enzyme (TYH) capable of hydroxylating L-tyrosine and oxidizing L-DOPA, such as CYP76ADα; a second heterologous enzyme which is a 4,5-DOPA extradiol dioxygenase (DOD); and a third heterologous enzyme, having glycosyltransferase activity, such as an activity selected from a betanidin-5-O-glucosyltransferase (B50G) activity and a cyclo-DOPA-5-O-glucosyltransferase (cDOPA50GT) activity, such as a glycosyltransferase, such as a scopoletin glucosyltransferase (SGT), whereby said cell is capable of producing one or more betalains, wherein said one or more betalains comprise one or more glycosylated betalains such as betanin and/or isobetanin; and/or
b. a first heterologous enzyme capable of hydroxylating L-tyrosine and oxidizing L-DOPA, such as CYP76ADα; a second heterologous enzyme which is a DOD having a truncation in its C-terminal end (DOD*); whereby said cell is capable of producing one or more betalains, wherein said one or more betalains comprise one or more betaxanthins.

29. The method according to item 28, further comprising a step of recovering the one or more glycosylated betalains, such as the betanin and/or the isobetanin, and/or the one or more betaxanthins.

30. The method according to any one of items 28 to 29, wherein the yeast cell is a yeast cell as defined in any one of items 1 to 27.

31. The method according to any one of items 28 to 30, wherein the enzyme having glycosyltransferase activity is an enzyme having glycosyltransferase activity, such as a glycosyltransferase, such as an SGT, as defined in any one of the preceding items.

32. The method according to any one of items 28 to 31, wherein the TYH is a TYH as defined in any one of the preceding items.

33. The method according to any one of items 28 to 32, wherein the DOD is a DOD as defined in any one of the preceding items.

34. The method according to any one of items 28 to 33, wherein the method yields one or more betalains, wherein said one or more betalains comprise one or more glycosylated betalains such as betanin and/or isobetanin, wherein the titer of the one or more betalains such as betanin and/or isobetanin is at least 0.5 mg/L, such as at least 1 mg/L, such as at least 1.5 mg/L, such as at least 5 mg/L, such as at least 10 mg/L, such as at least 25 mg/L, such as at least 50 mg/L, such as at least 100 mg/L, such as at least 250 mg/L, such as at least 500 mg/L, such as at least 750 mg/L, such as at least 1 g/L, such as at least 2 g/L, such as at least 3 g/L, such as at least 4 g/L, such as at least 5 g/L, such as at least 6 g/L, such as at least 7 g/L, such as at least 8 g/L, such as at least 9 g/L, such as at least 10 g/L, such as at least 11 g/L, such as at least 12 g/L, such as at least 13 g/L, such as at least 14 g/L, such as at least 15 g/L, such as at least 16 g/L, such as at least 17 g/L, such as at least 18 g/L, such as at least 19 g/L, such as at least 20 g/L, such as at least 25 g/L, such as at least 30 g/L, such as at least 35 g/L, such as at least 40 g/L, such as at least 45 g/L, such as at least 50 g/L, or more.

35. The method according to any one of items 28 to 33, wherein the method increases the yield of the one or more betalains such as betanin and/or isobetanin by at least 1.2-fold, such as at least 1.3-fold, such as at least 1.4-fold, such as at least 1.5-fold, such as at least 1.6-fold, such as at least 1.7-fold, such as at least 1.8-fold, such as at least 1.9-fold, such as at least 2-fold, such as at least 2.5-fold, such as at least 3-fold, such as at least 3.5-fold, such as at least 4-fold, such as at least 4.5-fold, such as at least 5-fold, such as at least 6-fold, such as at least 7-fold, such as at least 8-fold, such as at least 9-fold, such as at least 10-fold, such as at least 20-fold, such as at least 30-fold, such as at least 40-fold, such as at least 50-fold, wherein said one or more betalains comprise one or more betaxanthins, preferably wherein the increase is determined by measuring the fluorescence per OD and comparing it to the fluorescence per OD obtained in a reference yeast cell expressing MjDOD+BvCYP76AD^{W13L} cultivated in similar or identical conditions.

36. A system comprising nucleic acids encoding:
a. a TYH, such as CYP76ADα capable of:
   i. hydroxylating L-tyrosine; and/or
   ii. oxidizing L-DOPA; and
b. a DOD capable of oxygenating L-DOPA; and
c. an glycosyltransferase capable of:
   i. glycosylating cyclo-DOPA; and/or
   ii. glycosylating betanidin.

37. The system according to item 36, wherein the TYH is encoded by a polynucleotide selected from SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 42, SEQ ID NO: 44, SEQ ID NO: 46, SEQ ID NO: 48, SEQ ID NO: 62, SEQ ID NO: 63, and SEQ ID NO: 64, or homologues thereof having at least 80% identity thereto, such as at least 85% identity, such as at least 90% identity, such as at least 91% identity, such as at least 92% identity, such as at least 93% identity, such as at least 94% identity, such as at least 95% identity, such as at least 96% identity, such as at least 97% identity, such as at least 98% identity, such as at least 99% identity thereto.

38. The system according to any one of items 36 to 37, wherein the DOD is encoded by a polynucleotide selected from SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 59, SEQ ID NO: 60, and SEQ ID NO: 61, or homologues thereof having at least 80% identity thereto, such as at least 85% identity, such as at least 90% identity, such as at least 91% identity, such as at least 92% identity, such as at least 93% identity, such as at least 94% identity, such as at least 95% identity, such as at least 96% identity, such as at least 97% identity, such as at least 98% identity, such as at least 99% identity thereto.

39. The system according to any one of items 36 to 38, wherein the enzyme having glycosyltransferase activity is encoded by a polynucleotide selected from SEQ ID NO: 54, SEQ ID NO: 66, SEQ ID NO: 68 and SEQ ID NO: 58, or homologues thereof having at least 80% identity thereto, such as at least 85% identity, such as at least 90% identity, such as at least 91% identity, such as at least 92% identity, such as at least 93% identity, such as at least 94% identity, such as at least 95% identity, such as at least 96% identity, such as at least 97% identity, such as at least 98% identity, such as at least 99% identity thereto.

40. The system according to any one of items 36 to 39, wherein said system is comprised in a vector, such as a plasmid, or in the genome of the yeast cell.

41. Use of a polynucleotide as set forth in SEQ ID NO: 54, SEQ ID NO: 66, SEQ ID NO: 68 or SEQ ID NO: 58 for obtaining a protein capable of glycosylating a betalain and/or a betalain precursor, such as a protein capable of glycosylating betanidin and/or cyclo-DOPA, such as a protein with betanidin-5-O-glucosyltransferase activity and/or a protein with cyclo-DOPA 5-O-glucosyltransferase activity.

42. Use of an enzyme having glycosyltransferase activity as a betanidin-5-O-glucosyltransferase (B50G) and/or a cyclo-DOPA 5-O-glucosyltransferase (cDOPA5OGT), preferably wherein said enzyme having glycosyltransferase activity is selected from the glycosyltransferases from Beta vulgaris set forth in SEQ ID NO: 53 (BvSGT2) SEQ ID NO: 57 (BvSGT4), the glycosyltransferase from Chenopodium quinoa set forth in SEQ ID NO: 65 and the glycosyltransferase from Bougainvillea glabra set forth in SEQ ID NO: 67, or functional variants having at least 80% identity thereto.

43. Use of an enzyme having glycosyltransferase activity to catalyse the conversion of cyclo-DOPA to cyclo-DOPA-5-O-glucoside and/or glycosylating betanidin and/or to catalyse the glycosylation of betanidin, wherein said enzyme is selected from:
i. Chenopodium quinoa glycosyltransferase CqSGT2 set forth in SEQ ID NO: 65, or a functional variant thereof having at least 70% sequence identity thereto;
ii. Beta vulgaris glycosyltransferase BvSGT2 set forth in SEQ ID NO: 53, or a functional variant thereof having at least 70% identity thereto;
iii. Bougainvillea glabra glycosyltransferase BgGT2 set forth in SEQ ID NO: 67, or a functional variant thereof having at least 70% identity thereto; and
iv. Beta vulgaris glycosyltransferase BvSGT4 set forth in SEQ ID NO: 57, or a functional variant thereof having at least 70% identity thereto.

44. Use of a DOD variant (DOD*) to catalyse the conversion of L-DOPA to 4,5-seco-DOPA, which is a DOD truncation mutant of having a truncation of at least 5 amino acids at the C-terminal end, such as at least 6, such as at least 7, such as at least 8, such as at least 9, such as at least 10, such as at least 12, such as at least 14, such as at least 16, such as at least 18, such as at least 20, such as at least 25, such as at least 30, such as at least 35, such as at least 40, such as at least 45, such as at least 50 amino acids at the C-terminal end.

45. The use according to item 44 wherein the DOD is native to a plant, such as of the genus Amaranthus, Beta, Bougainvillea, Mirabilis Phytolacca, Portulaca, Spinacia, or Suaeda, such as Amaranthus hypochondriacus, Amaranthus tricolour, Beta vulgaris, Bougainvillea glabra, Mirabilis jalapa, Phytolacca americana, Portulaca grandiflora, Spinacia oleracea, or Suaeda salsa, or a functional variant thereof having at least 80% identity thereto.

46. The use according to any one of items 44 to 45, wherein said DOD* is as set forth in SEQ ID NO: 9 (PgDOD*).

47. A betalain, such as a betacyanin such as betanidin, betanin or isobetanin, or a betaxanthin obtainable by the method according to any one of items 28 to 35.

48. Use of a betalain, such as a betacyanin such as betanidin or betanin or isobetanin, or a betaxanthin obtainable by the method according to any one of items 28 to 35.

49. Use of a heterologous TYH, DOD, DOD*, and/or glycosyltransferase as defined in any one of items 1 to 27 in a method of production of one or more betalains.

50. The use according to item 49, wherein the heterologous TYH, DOD, DOD*, and/or glycosyltransferase are as defined in any one of items 1 to 27 in a method of production of one or more betalains, wherein said one or more betalains comprise one or more glycosylated betalains, such as betanin and/or isobetanin.

51. The use according to any one of item 49 to 50, wherein the heterologous TYH, DOD, DOD*, and/or glycosyltransferase are as defined in any one of items 1 to 27 in a method of production of one or more betalains, wherein said one or more betalains comprise one or more betaxanthins.

52. The use according to any one of items 49 to 51, wherein the glycosyltransferase, such as the SGT is:
a. a Beta vulgaris SGT such as BvSGT2 as set forth in SEQ ID NO: 53, or a functional variant thereof; or
b. a Beta vulgaris SGT such as BvSGT4 as set forth in SEQ ID NO: 57, or a functional variant thereof.

53. The use according to any one of items 49 to 52, wherein the DOD* is a DOD truncation mutant having a truncation of at least 5 amino acids at the C-terminal end, such as at least 6, such as at least 7, such as at least 8, such as at least 9, such as at least 10, such as at least 12, such as at least 14, such as at least 16, such as at least 18, such as at least 20, such as at least 25, such as at least 30, such as at least 35, such as at least 40, such as at least 45, such as at least 50 amino acids at the C-terminal end.

54. The use according to any one of items 49 to 53, wherein the DOD is native to a plant, such as of the genus Amaranthus, Beta, Bougainvillea, Mirabilis Phytolacca, Portulaca, Spinacia, or Suaeda, such as Amaranthus hypochondriacus, Amaranthus tricolour, Beta vulgaris, Bougainvillea glabra, Mirabilis jalapa, Phytolacca americana, Portulaca grandiflora, Spinacia oleracea, or Suaeda salsa, or a functional variant thereof having at least 80% identity thereto.

55. The use according to any one of items 49 to 54, wherein the DOD* is:
a. a truncation of a Mirabilis jalapa DOD such as MjDOD as set forth in SEQ ID NO: 43, or a functional variant thereof having at least 80% identity thereto; or
b. a truncation of a Portulaca grandiflora DOD such as PgDOD as set forth in SEQ ID NO: 7, or a functional variant thereof having at least 80% identity thereto;
c. a Bougainvillea glabra DOD such as BgDOD2 as set forth in SEQ ID NO: 21, or a functional variant thereof having at least 80% identity thereto.

56. The use according to any one of items 49 to 51 and 53 to 55 wherein the DOD* is PgDOD* as set forth in SEQ ID NO: 9 (PgDOD*).

57. The use according to any one of items 49 to 56, wherein said method is performed in vitro or in a cell, such as a prokaryotic or a eukaryotic cell.

58. A kit of parts comprising:
a. the yeast cell according to any one of items 1 to 26; and/or
b. the nucleic acid system according to any one of items 36 to 40, wherein said system is for modifying a yeast cell; and
c. instructions for use; and
d. optionally, the yeast cell to be modified.

59. A method for producing at least 0.5 mg/L of one or more betalains, wherein said one or more betalains comprise a glycosylated betalain such as betanin and/or isobetanin, such as at least 1 mg/L, such as at least 1.5 mg/L, such as at least 5 mg/L, such as at least 10 mg/L, such as at least 25 mg/L, such as at least 50 mg/L, such as at least 100 mg/L, such as at least 250 mg/L, such as at least 500 mg/L, such as at least 750 mg/L, such as at least 1 g/L, such as at least 2 g/L, such as at least 3 g/L, such as at least 4 g/L, such as at least 5 g/L, such as at least 6 g/L, such as at least 7 g/L, such as at least 8 g/L, such as at least 9 g/L, such as at least 10 g/L, such as at least 11 g/L, such as at least 12 g/L, such as at least 13 g/L, such as at least 14 g/L, such as at least 15 g/L, such as at least 16 g/L, such as at least 17 g/L, such as at least 18 g/L, such as at least 19 g/L, such as at least 20 g/L, such as at least 25 g/L, such as at least 30 g/L, such as at least 35 g/L, such as at least 40 g/L, such as at least 45 g/L, such as at least 50 g/L, or more, wherein the method is according to any one of 28 to 35.

60. The method according to item 59, wherein the method is for producing at least 0.5 mg/L of betanin and/or isobetanin, such as at least 1 mg/L, such as at least 1.5 mg/L, such as at least 5 mg/L, such as at least 10 mg/L, such as at least 25 mg/L, such as at least 50 mg/L, such as at least 100 mg/L, such as at least 250 mg/L, such as at least 500 mg/L, such as at least 750 mg/L, such as at least 1 g/L, such as at least 2 g/L, such as at least 3 g/L, such as at least 4 g/L, such as at least 5 g/L, such as at least 6 g/L, such as at least 7 g/L, such as at least 8 g/L, such as at least 9 g/L, such as at least 10 g/L, such as at least 11 g/L, such as at least 12 g/L, such as at least 13 g/L, such as at least 14 g/L, such as at least 15 g/L, such as at least 16 g/L, such as at least 17 g/L, such as at least 18 g/L, such as at least 19 g/L, such as at least 20 g/L, such as at least 25 g/L, such as at least 30 g/L, such as at least 35 g/L, such as at least 40 g/L, such as at least 45 g/L, such as at least 50 g/L, or more.

61. The method according to any one of items 59 to 60, wherein the method is for producing at least 0.5 mg/L of a betaxanthin, such as at least 1 mg/L, such as at least 1.5 mg/L, such as at least 5 mg/L, such as at least 10 mg/L, such as at least 25 mg/L, such as at least 50 mg/L, such as at least 100 mg/L, such as at least 250 mg/L, such as at least 500 mg/L, such as at least 750 mg/L, such as at least 1 g/L, such as at least 2 g/L, such as at least 3 g/L, such as at least 4 g/L, such as at least 5 g/L, such as at least 6 g/L, such as at least 7 g/L, such as at least 8 g/L, such as at least 9 g/L, such as at least 10 g/L, such as at least 11 g/L, such as at least 12 g/L, such as at least 13 g/L, such as at least 14 g/L, such as at least 15 g/L, such as at least 16 g/L, such as at least 17 g/L, such as at least 18 g/L, such as at least 19 g/L, such as at least 20 g/L, such as at least 25 g/L, such as at least 30 g/L, such as at least 35 g/L, such as at least 40 g/L, such as at least 45 g/L, such as at least 50 g/L, or more.

1-21. (canceled)

22. A yeast cell capable of producing one or more betalains, said yeast cell expressing:
a. a first heterologous enzyme (TYH) capable of hydroxylating L-tyrosine and oxidizing L-DOPA;
b. a second heterologous enzyme which is a 4,5-DOPA extradiol dioxygenase (DOD); and
c. a third heterologous enzyme having glycosyltransferase activity, wherein said enzyme is selected from:
i. Chenopodium quinoa glycosyltransferase CqSGT2 set forth in SEQ ID NO: 65, or a functional variant thereof having at least 80% sequence identity thereto;
ii. Beta vulgaris glycosyltransferase BvSGT2 set forth in SEQ ID NO: 53, or a functional variant thereof having at least 80% identity thereto;
iii. Bougainvillea glabra glycosyltransferase BgGT2 set forth in SEQ ID NO: 67, or a functional variant thereof having at least 80% identity thereto; and
iv. Beta vulgaris glycosyltransferase BvSGT4 set forth in SEQ ID NO: 57, or a functional variant thereof having at least 80% identity thereto;
whereby said cell is capable of producing one or more betalains, wherein said one or more betalains comprise one or more glycosylated betalains.

23. The yeast cell according to claim 22, wherein the one or more glycosylated betalains is betanin or isobetanin.

24. The yeast cell according to claim 22, wherein the genus of said yeast cell is selected from Saccharomyces, Pichia, Yarrowia, Kluyveromyces, Candida, Rhodotorula, Rhodosporidium, Cryptococcus, Trichosporon and Lipomyces.

25. The yeast cell according to claim 22, wherein the species of said yeast cell is selected from Saccharomyces cerevisiae, Saccharomyces boulardi, Candida tropicalis, Pichia pastoris, Kluyveromyces marxianus, Cryptococcus albidus, Lipomyces lipofera, Lipomyces starkeyi, Rhodosporidium toruloides, Rhodotorula glutinis, Trichosporon pullulan and Yarrowia lipolytica.

26. The yeast cell according to claim 22, wherein the TYH or the DOD is native to a plant.

27. The yeast cell according to claim 22, wherein:
a. the TYH is native to a plant of the genus Abronia, Acleisanthes, Basella, Beta, Cleretum, Ercilla, Mirabilis, Optunia, or Phytolacca; or
b. the DOD is native to a plant of the genus Amaranthus, Beta, Bougainvillea, Mirabilis Phytolacca, Portulaca, Spinacia, or Suaeda.

28. The yeast cell according to claim 22, wherein:
a. the TYH is native to a plant of the species Abronia nealleyi, Acleisanthes obtusa, Basella alba, Beta vulgaris, Cleretum bellidiforme, Ercilla volubis, Mirabilis multiflora, Optunia ficus-indica, or Phytolacca dioica; or
b. the DOD is native to a plant of the species Amaranthus hypochondriacus, Amaranthus tricolour, Beta vulgaris, Bougainvillea glabra, Mirabilis jalapa, Phytolacca americana, Portulaca grandiflora, Spinacia oleracea, or Suaeda salsa.

29. The yeast cell according to claim 22, wherein:
a. the TYH is selected from:
i. AnTYH as set forth in SEQ ID NO: 37, or a functional variant thereof having at least 80% identity thereto;
ii. EvTYH as set forth in SEQ ID NO: 43, or a functional variant thereof having at least 80% identity thereto;
or
b. the DOD is selected from:
i. MjDOD as set forth in SEQ ID NO: 1, or a functional variant thereof having at least 80% identity thereto; and
ii. PgDOD as set forth in SEQ ID NO: 7, or a functional variant thereof having at least 80% identity thereto; and
iii. BgDOD2 as set forth in SEQ ID NO: 21, or a functional variant thereof having at least 80% identity thereto.

30. The yeast cell according to claim 22, wherein:
a. the TYH is selected from SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, or SEQ ID NO: 47, or a functional variant thereof having at least 70% identity thereto; and/or
b. the DOD is selected from SEQ ID NO: 1, SEQ ID NO: 3, SEQ ID NO: 5, SEQ ID NO: 7, SEQ ID NO: 11, SEQ ID NO: 13, SEQ ID NO: 15, SEQ ID NO: 17, SEQ ID NO: 19, SEQ ID NO: 21, SEQ ID NO: 23, or SEQ ID NO: 25, or a functional variant thereof having at least 70% identity thereto.

31. The yeast cell according to claim 22, wherein said yeast cell expresses:
a. a TYH from Abronia nealleyi (AnTYH) as set forth in SEQ ID NO: 37; a DOD from Bougainvillea glabra (BgDOD2) as set forth in SEQ ID NO: 21; and a glycosyltransferase from Chenopodium quinoa as set forth in SEQ ID NO: 65, (CqSGT2); or
b. a TYH from Abronia nealleyi (AnTYH) as set forth in SEQ ID NO: 37; a DOD from Bougainvillea glabra (BgDOD2) as set forth in SEQ ID NO: 21; and a glycosyltransferase from Beta vulgaris (BvSGT2) as set forth in SEQ ID NO: 53; or
c. a TYH from Abronia nealleyi (AnTYH) as set forth in SEQ ID NO: 37; a DOD from Bougainvillea glabra (BgDOD2) as set forth in SEQ ID NO: 21; and a glycosyltransferase from Bougainvillea glabra as set forth in SEQ ID NO: 67, (BgGT2); or
d. a TYH from Abronia nealleyi (AnTYH) as set forth in SEQ ID NO: 37; a DOD from Bougainvillea glabra (BgDOD2) as set forth in SEQ ID NO: 21; and a glycosyltransferase from Beta vulgaris (BvSGT4) as set forth in SEQ ID NO: 57; or
e. a TYH from Abronia nealleyi (AnTYH) as set forth in SEQ ID NO: 37; a DOD from Mirabilis jalapa (MjDOD) as set forth in SEQ ID NO: 1; and a glycosyltransferase from Chenopodium quinoa as set forth in SEQ ID NO: 65, (CqSGT2); or
f. a TYH from Abronia nealleyi (AnTYH) as set forth in SEQ ID NO: 37; a DOD from Mirabilis jalapa (MjDOD) as set forth in SEQ ID NO: 1; and a glycosyltransferase from Beta vulgaris (BvSGT2) as set forth in SEQ ID NO: 53; or
g. a TYH from Abronia nealleyi (AnTYH) as set forth in SEQ ID NO: 37; a DOD from Mirabilis jalapa (MjDOD) as set forth in SEQ ID NO: 1; and a glycosyltransferase from Bougainvillea glabra as set forth in SEQ ID NO: 67, (BgGT2); or
h. a TYH from Abronia nealleyi (AnTYH) as set forth in SEQ ID NO: 37; a DOD from Mirabilis jalapa (MjDOD) as set forth in SEQ ID NO: 1; and a glycosyltransferase from Beta vulgaris (BvSGT4) as set forth in SEQ ID NO: 57; or
i. a TYH from Abronia nealleyi (AnTYH) as set forth in SEQ ID NO: 37; a DOD from Portulaca grandiflora (PgDOD) as set forth in SEQ ID NO: 7; and a glycosyltransferase from Chenopodium quinoa as set forth in SEQ ID NO: 65, (CqSGT2); or
j. a TYH from Abronia nealleyi (AnTYH) as set forth in SEQ ID NO: 37; a DOD from Portulaca grandiflora (PgDOD) as set forth in SEQ ID NO: 7; and a glycosyltransferase from Beta vulgaris (BvSGT2) as set forth in SEQ ID NO: 53; or
k. a TYH from Abronia nealleyi (AnTYH) as set forth in SEQ ID NO: 37; a DOD from Portulaca grandiflora (PgDOD) as set forth in SEQ ID NO: 7; and a glycosyltransferase from Bougainvillea glabra as set forth in SEQ ID NO: 67, (BgGT2); or
l. a TYH from Abronia nealleyi (AnTYH) as set forth in SEQ ID NO: 37; a DOD from Portulaca grandiflora (PgDOD) as set forth in SEQ ID NO: 7; and a glycosyltransferase from Beta vulgaris (BvSGT4) as set forth in SEQ ID NO: 57; or
m. a TYH from Ercilla volubisi (EvTYH) as set forth in SEQ ID NO: 43; a DOD from Bougainvillea glabra (BgDOD2) as set forth in SEQ ID NO: 21; and a glycosyltransferase from Chenopodium quinoa as set forth in SEQ ID NO: 65, (CqSGT2); or
n. a TYH from Ercilla volubisi (EvTYH) as set forth in SEQ ID NO: 43; a DOD from Bougainvillea glabra (BgDOD2) as set forth in SEQ ID NO: 21; and a glycosyltransferase from Beta vulgaris (BvSGT2) as set forth in SEQ ID NO: 53; or
o. a TYH from Ercilla volubisi (EvTYH) as set forth in SEQ ID NO: 43; a DOD from Bougainvillea glabra (BgDOD2) as set forth in SEQ ID NO: 21; and a glycosyltransferase from Bougainvillea glabra as set forth in SEQ ID NO: 67, (BgGT2); or
p. a TYH from Ercilla volubisi (EvTYH) as set forth in SEQ ID NO: 43; a DOD from Bougainvillea glabra (BgDOD2) as set forth in SEQ ID NO: 21; and a glycosyltransferase from Beta vulgaris (BvSGT4) as set forth in SEQ ID NO: 57; or
q. a TYH from Ercilla volubisi (EvTYH) as set forth in SEQ ID NO: 43; a DOD from Mirabilis jalapa (MjDOD) as set forth in SEQ ID NO: 1; and a glycosyltransferase from Chenopodium quinoa as set forth in SEQ ID NO: 65, (CqSGT2); or
r. a TYH from Ercilla volubisi (EvTYH) as set forth in SEQ ID NO: 43; a DOD from Mirabilis jalapa (MjDOD) as set forth in SEQ ID NO: 1; and a glycosyltransferase from Beta vulgaris (BvSGT2) as set forth in SEQ ID NO: 53; or
s. a TYH from Ercilla volubisi (EvTYH) as set forth in SEQ ID NO: 43; a DOD from Mirabilis jalapa (MjDOD) as set forth in SEQ ID NO: 1; and a glycosyltransferase from Bougainvillea glabra as set forth in SEQ ID NO: 67, (BgGT2); or
t. a TYH from Ercilla volubisi (EvTYH) as set forth in SEQ ID NO: 43; a DOD from Mirabilis jalapa (MjDOD) as set forth in SEQ ID NO: 1; and a glycosyltransferase from Beta vulgaris (BvSGT4) as set forth in SEQ ID NO: 57; or
u. a TYH from Ercilla volubisi (EvTYH) as set forth in SEQ ID NO: 43; a DOD from Portulaca grandiflora (PgDOD) as set forth in SEQ ID NO: 7; and a glycosyltransferase from Chenopodium quinoa as set forth in SEQ ID NO: 65, (CqSGT2); or
v. a TYH from Ercilla volubisi (EvTYH) as set forth in SEQ ID NO: 43; a DOD from Portulaca grandiflora (PgDOD) as set forth in SEQ ID NO: 7; and a glycosyltransferase from Beta vulgaris (BvSGT2) as set forth in SEQ ID NO: 53; or
w. a TYH from Ercilla volubisi (EvTYH) as set forth in SEQ ID NO: 43; a DOD from Portulaca grandiflora (PgDOD) as set forth in SEQ ID NO: 7; and a glycosyltransferase from Bougainvillea glabra as set forth in SEQ ID NO: 67, (BgGT2); or
x. a TYH from Ercilla volubisi (EvTYH) as set forth in SEQ ID NO: 43; a DOD from Portulaca grandiflora (PgDOD) as set forth in SEQ ID NO: 7; and a glycosyltransferase from Beta vulgaris (BvSGT4) as set forth in SEQ ID NO: 57;
or functional variants thereof having at least 80% identity thereto, whereby said yeast cell is capable of producing one or more betalains, wherein said one or more betalains comprise one or more glycosylated betalains.

32. The yeast cell according to claim 22, wherein said yeast cell expresses at least two different enzymes having glycosyltransferase activity.

33. The yeast cell according to claim 22, wherein said yeast cell has:
a. a mutation resulting in reduced activity of 4-hydroxyphenylpyruvate dioxygenase (4-HPPD); or
b. a mutation resulting in increased production of L-tyrosine.

34. A yeast cell capable of producing one or more betalains, said yeast cell expressing:
a. a first heterologous enzyme (TYH) capable of hydroxylating L-tyrosine and oxidizing L-DOPA;
b. a second heterologous enzyme which is a 4,5-DOPA extradiol dioxygenase (DOD); and
c. a third heterologous enzyme having glycosyltransferase activity;
whereby said cell is capable of producing one or more betalains, wherein said one or more betalains comprise one or more glycosylated betalains, and
wherein said yeast cell comprises a mutation resulting in reduced activity of 4-hydroxyphenylpyruvate dioxygenase (4-HPPD).

35. A method for production of one or more betalains in a yeast cell according to claim 34, said method comprising the steps of incubating said yeast cell in a medium, whereby said yeast cell expresses:
a. a first heterologous enzyme (TYH) capable of hydroxylating L-tyrosine and oxidizing L-DOPA;
b. a second heterologous enzyme which is a 4,5-DOPA extradiol dioxygenase (DOD); and
c. a third heterologous enzyme, having glycosyltransferase activity,
whereby said cell is capable of producing one or more betalains, wherein said one or more betalains comprise one or more glycosylated betalains.

36. A method for production of one or more betalains in a yeast cell, said method comprising the steps of incubating a yeast cell in a medium, wherein said yeast cell expresses:
a. a first heterologous enzyme (TYH) capable of hydroxylating L-tyrosine and oxidizing L-DOPA;
b. a second heterologous enzyme which is a 4,5-DOPA extradiol dioxygenase (DOD); and
c. a third heterologous enzyme, having glycosyltransferase activity, wherein said enzyme is selected from:
i. Chenopodium quinoa glycosyltransferase CqSGT2 set forth in SEQ ID NO: 65, or a functional variant thereof having at least 80% sequence identity thereto;
ii. Beta vulgaris glycosyltransferase BvSGT2 set forth in SEQ ID NO: 53, or a functional variant thereof having at least 80% identity thereto;
iii. Bougainvillea glabra glycosyltransferase BgGT2 set forth in SEQ ID NO:
67, or a functional variant thereof having at least 80% identity thereto; and
iv. Beta vulgaris glycosyltransferase BvSGT4 set forth in SEQ ID NO: 57, or a functional variant thereof having at least 80% identity thereto;
whereby said cell is capable of producing one or more betalains, wherein said one or more betalains comprise one or more glycosylated betalains.

37. The method according to claim 36, wherein:
a. the yeast cell is selected from Saccharomyces cerevisiae, Saccharomyces boulardi, Candida tropicalis, Pichia pastoris, Kluyveromyces marxianus, Cryptococcus albidus, Lipomyces lipofera, Lipomyces starkeyi, Rhodosporidium toruloides, Rhodotorula glutinis, Trichosporon pullulan and Yarrowia lipolytica;
b. the yeast cell comprises a mutation resulting in reduced activity of 4-hydroxyphenylpyruvate dioxygenase (4-HPPD);
c. the one or more glycosylated betalains is betanin or isobetanin;
d. the TYH is selected from SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, and SEQ ID NO: 47, or a functional variant thereof having at least 70% identity thereto;
or
e. the DOD is selected from SEQ ID NO: 1, SEQ ID NO: 3, SEQ ID NO: 5, SEQ ID NO: 7, SEQ ID NO: 11, SEQ ID NO: 13, SEQ ID NO: 15, SEQ ID NO: 17, SEQ ID NO: 19, SEQ ID NO: 21, SEQ ID NO: 23, and SEQ ID NO: 25, or a functional variant thereof having at least 70% identity thereto.

38. The method according to claim 36, wherein:
a. the TYH is selected from:
i. AnTYH as set forth in SEQ ID NO: 37, or a functional variant thereof having at least 80% identity thereto;
ii. EvTYH as set forth in SEQ ID NO: 43, or a functional variant thereof having at least 80% identity thereto;
or
b. the DOD is selected from:
i. MjDOD as set forth in SEQ ID NO: 1, or a functional variant thereof having at least 80% identity thereto; and
ii. PgDOD as set forth in SEQ ID NO: 7, or a functional variant thereof having at least 80% identity thereto; and
iii. BgDOD2 as set forth in SEQ ID NO: 21, or a functional variant thereof having at least 80% identity thereto.

39. The method according to claim 36, wherein the yeast cell is as defined in claim 31.

40. The method according to claim 36, wherein the method yields one or more betalains, wherein said one or more betalains comprise one or more glycosylated betalains, wherein the titer of the one or more betalains is at least 0.5 mg/L.

41. The method according to claim 36, wherein the medium is supplemented with L-tyrosine.


SEQUENCE LISTING (80 sequences)

SEQ ID NO: 1 - protein, 267 aa, Mirabilis jalapa
MKGTYYINHGDPLMYLKKHIKLRQFLEGWQENVVIEKPKSILIISAHWDTNVPTVNFVEHCDTIHDFDDYPDPLYQIQYRAPGAPNLAKKVEELLKESGMECEIDTKRGLDHAAWFPLMFMYPEANIPICELSVQPSKDGIHHYNVGKALSPLLQQGVLIIGSGGTVHPSDDTPHCPNGVAPWAIEFDNWLEDALLSGRYEDVNNFKKLAPNWEISHPGQEHLYPLHVALGAAGKNPKTQLIHRSWAANGVFGYSTYNFTPTTQKTD

SEQ ID NO: 2 - DNA, 805 bp, Artificial
  Codon-optimized sequence
atgaagggaacctactacatcaaccacggtgacccattgatgtacttgaagaagcacatcaagttgagacaattcttggaaggttggcaagaaaacgttgttatcgaaaagccaaagtctatcttgatcatcagtgcacactgggacaccaacgttccaaccgttaacttcgttgaacactgtgacaccatccacgacttcgacgactacccagacccattgtaccaaatccaataccgcgctccaggtgctccaaacttggctaagaaggttgaagaattgcttaaggaatctggtatggaatgtgaaatcgacaccaagagaggtttggaccacgctgcttggttcccattgatgttcatgtacccagaagctaacatcccaatctgtgaattgtctgttcaaccatctaaggacggtatccaccactacaacgttggtaaagctttgtctccattgttgcaacaaggtgttttgatcatcggttctggtggaaccgttcacccatctgacgacaccccacactgtccaaacggtgttgctccatgggctatcgagttcgacaactggttggaagacgctttgctctcgggtagatacgaagacgttaacaacttcaagaagttggctccaaactgggaaatctctcacccaggtcaagaacacttgtacccattgcacgttgctttgggtgctgctggtaagaacccaaagacccaattgatccaccggtcttgggctgctaacggtgttttcggttactctacctacaacttcaccccaaccacccaaaagaccgacgtaa

SEQ ID NO: 3 - protein, 268 aa, Beta vulgaris
MGSEDNIKETFFISHGTPMMAIDDSKPSKKFLESWREKIFSKKPKAILVISAHWETDQPSVNVVDINDTIYDFRGFPARLYQFKYSAPGSPELANRIQDLLAGSGFKSVNTDKKRGLDHGAWVPLMLMYPEADIPVCQLSVQSHLDGTHHYKLGQALAPLKDEGVLIIGSGSATHPSNGTPPCSDGVAPWAAAFDSWLETALTNGSYEEVNKYETKAPNWKLAHPWPEHFYPLHVAMGAAGENSKAELIHNSWDGGIMSYGSYKFTST

SEQ ID NO: 4 - DNA, 807 bp, Artificial
  Codon-optimized sequence
atgggttctgaagataatattaaagaaacatttttcatctctcatggcactccaatgatggctattgatgattccaagccatctaagaagtttttggaatcttggagagaaaagattttctctaagaaaccaaaagctatcttggttatctctgcccattgggaaactgatcagccgtccgttaatgttgttgatattaatgatactatttacgatttcaggggttttccagctagattgtaccaatttaagtactcagctccaggttctcccgaattagctaacagaattcaagatttgttagccggctctggttttaaatctgttaatactgataagaaaaggggtctcgatcatggtgcttgggttccattgatgttaatgtatccagaagctgatattccagtttgtcaattgtccgttcaatcacatttggatggtactcatcattacaaattgggtcaggctttagctccattgaaagatgaaggcgtccttattattggttctggttctgctacccatccatccaatggtactccaccatgttctgatggtgttgccccatgggctgctgcttttgattcttggttagagactgccttgactaatggttcttacgaagaagttaacaagtatgagactaaagctccaaactggaagttggcccatccatggccagaacatttttacccattacatgtcgccatgggtgctgctggcgaaaattctaaagctgaattgattcataattcttgggacggtggtattatgtcttatggttcttataaatttacttctacttaa

SEQ ID NO: 5 - protein, 268 aa, Bougainvillea glabra
MGGEKKMKGTYYLAHGDPIMYINKSIKLRHFLEGWKENVLTEKPKCILVISAHWDTDVPTVNLVEQCDTIHDFDDYPDPLYQIKYPAPGAPKLAMKVQELLKGGGFKCEVDTKRGLDHAVWFPLMFMYPEADIPICELSIQTSKDGTHHYNVGKALSPLLNDDVLIIASGGAVHPSDDTPHFPNGVAPWALEFDNWLEGALLSGRYEDVKEFKKLAPNWEISHPGQEHLYPLHVALGAAGNNVKTELIHQTWAANGVFGYSSYKFTST

SEQ ID NO: 6 - DNA, 807 bp, Artificial
  Codon-optimized sequence
atgggtggtgaaaagaaaatgaaaggtacttattatttggcccacggtgatccaattatgtatattaataaatctattaaattgagacacttcttggagggttggaaagaaaatgtcttgactgaaaagccaaaatgtattttggtcatttctgcacactgggatactgatgttccaaccgttaacttagttgaacaatgtgatacgattcatgattttgatgattatccagatccattgtatcagattaaatatccagctccaggtgctccaaagttggctatgaaagttcaagaattgttaaaaggtggcggctttaagtgtgaagttgatactaaaagaggcttggatcacgctgtttggtttccattaatgtttatgtacccagaagccgatattccaatttgtgaattgtccattcaaacttctaaagatggtactcatcactacaacgtcggtaaggctttgtctccattgttgaatgatgacgtcttgattattgcttctggtggcgctgttcatccatctgatgatactccacattttccaaatggtgttgccccctgggctttggaatttgataattggttggaaggtgctttgttgtctggtagatatgaagatgttaaggagtttaagaaattggctccaaattgggaaatcagccatccaggtcaagaacatttgtatccattacacgtcgctctgggtgctgctggtaataatgttaagactgaattgatccatcaaacttgggctgctaatggtgtttttggttattcttcttataaatttacttctacttaa

SEQ ID NO: 7 - protein, 271 aa, Portulaca grandiflora
MGVGKEVSFKESFFLSHGNPAMLADESFIARNFLLGWKKNVFPVKPKSILVVSAHWETDVPCVSAGQYPNVIYDFTEVPASMFQMKYPAPGCPKLAKRVQELLIAGGFKSAKLDEERGFDHSSWVPLSMMCPEADIPVCQLSVQPGLDATHHFNVGRALAPLKGEGVLFIGSGGAVHPSDDTPHWFDGVAPWAAEFDQWLEDALLEGRYEDVNNYQTKAPEGWKLAHPIPEHFLPLHVAMGAGGEKSKAELIYRTWDHGTLGYASYKFTSI

SEQ ID NO: 8 - DNA, 816 bp, Artificial
  Codon-optimized sequence
atgggtgtaggtaaagaagtttcttttaaagaatccttcttcttgtctcacggtaatccagctatgttggccgacgaatcctttattgctagaaatttcttactgggctggaaaaagaatgtttttccagttaaacccaaatctattctggttgtttctgctcactgggaaactgatgttccatgtgtttctgctggtcaataccccaacgtgatttatgattttactgaagttccagcttccatgttccaaatgaaatatccagctccaggttgtccaaaattggctaaaagagttcaagaattgttgattgcaggtggctttaaatctgctaagctggatgaagaaaggggttttgatcattcttcttgggttcccttatctatgatgtgtccagaagctgatattccagtttgtcagttatctgttcaaccaggtttggatgctactcatcattttaatgttggtagagctttagccccattgaaaggtgaaggtgttttatttattggctccggtggcgctgttcatccatctgatgatactccacattggtttgatggcgttgctccatgggctgctgaattcgaccaatggttggaagatgctttgttagaaggtagatatgaagacgtcaataattatcaaactaaagctccagaaggttggaaattggctcatccaattccagagcacttcttgccattgcatgttgctatgggtgctggcggcgagaaatctaaagctgaattgatttatagaacttgggatcatggtaccttaggttatgcttcttataaatttacttctatttaa

SEQ ID NO: 9 - protein, 250 aa, Portulaca grandiflora
MGVGKEVSFKESFFLSHGNPAMLADESFIARNFLLGWKKNVFPVKPKSILVVSAHWETDVPCVSAGQYPNVIYDFTEVPASMFQMKYPAPGCPKLAKRVQELLIAGGFKSAKLDEERGFDHSSWVPLSMMCPEADIPVCQLSVQPGLDATHHFNVGRALAPLKGEGVLFIGSGGAVHPSDDTPHWFDGVAPWAAEFDQWLEDALLEGRYEDVNNYQTKAPEGWKLAHPIPEHFLPLHVAMGLAARNLKLN

SEQ ID NO: 10 - DNA, 815 bp, Artificial
  Codon-optimized sequence
atgggtgtaggtaaagaagtttcttttaaagaatccttcttcttgtctcacggtaatccagctatgttggccgacgaatcctttattgctagaaatttcttactgggctggaaaaagaatgtttttccagttaaacccaaatctattctggttgtttctgctcactgggaaactgatgttccatgtgtttctgctggtcaataccccaacgtgatttatgattttactgaagttccagcttccatgttccaaatgaaatatccagctccaggttgtccaaaattggctaaaagagttcaagaattgttgattgcaggtggctttaaatctgctaagctggatgaagaaaggggttttgatcattcttcttgggttcccttatctatgatgtgtccagaagctgatattccagtttgtcagttatctgttcaaccaggtttggatgctactcatcattttaatgttggtagagctttagccccattgaaaggtgaaggtgttttatttattggctccggtggcgctgttcatccatctgatgatactccacattggtttgatggcgttgctccatgggctgctgaattcgaccaatggttggaagatgctttgttagaaggtagatatgaagacgtcaataattatcaaactaaagctccagaaggttggaaattggctcatccaattccagagcacttcttgccattgcatgttgctatggggctggcggcgagaaatctaaagctgaattgatttatagaacttgggatcatggtaccttaggttatgcttcttataaatttacttctatttaa

SEQ ID NO: 11 - protein, 310 aa, Spinacia oleracea
MILHNFQQPSTLLLQIPSFSATPEIKRTFLKSKFPKNSNLKIMAGQESIKETFFISHGTPMMAIDESKPSRKFLESWREKIYSKKPKAILVISAHWETDSPSVNSVDVNDTVYDFGGFPARLYQFKYPAPGFPDLAKRVQELLTASGFQSVHTDKKRGLDHGAWVPLMLMYPEADIPVCQLSVQSHLDGKYHFNLGRALAPLKDEGVLIIGSGSATHPSNGTPHCNDGVAPWAADFDLWLETALTSGRYEEVNKCERKAPNWKLAHPWPEHFYPLHVAMGAAGENSKAELIHNSWDHGTMSYGSYKFSPN

SEQ ID NO: 12 - DNA, 933 bp, Artificial
  Codon-optimized sequence
atgattttgcataattttcaacaaccatctactttgttgctgcaaattccatctttttctgctactccagaaatcaaaaggactttcttgaagtccaagttccctaaaaattctaatttgaaaattatggccggtcaagagtccattaaagaaacatttttcatttctcatggtactccaatgatggctattgatgaatctaaaccttcccgcaaatttttagaatcttggagagaaaagatttattcaaagaagccaaaagctattttggttatctctgcccattgggaaactgattctccctctgttaattctgttgatgttaatgatactgtttatgactttggcggctttccagctagattgtatcaattcaagtaccccgccccaggttttccagatttagctaagagagttcaagagttattgactgcttctggttttcagtctgttcacactgataagaaaaggggtttggaccacggtgcctgggttccattgatgttgatgtatccagaagctgacatcccagtttgtcaattgtctgttcaatctcacttggatggtaaatatcattttaatttaggtagggccttagccccattaaaagatgaaggtgttttgattattggctctggttcagctactcatccatctaacggtactccacactgtaacgatggtgttgctccatgggctgccgatttcgacttgtggctagaaactgctttgacttctggccgttacgaagaagttaataaatgtgaaagaaaggctccaaattggaagcttgctcatccatggccagaacatttctatccattgcatgttgctatgggtgctgccggtgagaattccaaagctgaattaattcataattcttgggatcacggtactatgtcttatggttcttataaattttctccaaattaa

SEQ ID NO: 13 - protein, 268 aa, Amaranthus tricolour
MGSQEIIKETFFISHGTPRMTIEASKPARKFLESWRDKIYFKKPKAILVISAHWETDFPSVNAVDINDTIYDFYGFPAPMYQFKYPAPGSPDLAQRVQELLTASGFKSVNVDKKRGLDHGAWVPLMLMYPNADIPVCQLSVQSHLDGMYHYKLGRALAPLKEEGVLIIGSGSATHPSNNTPHYYDGVAPWAADFDHWLETALTNGSYEEVNKCESKAPNWKLAHPWPEHFYPLHVAMGAAGENWKAELIHNSWDHGTMSYGSYKFVSS

SEQ ID NO: 14 - DNA, 807 bp, Artificial
  Codon-optimized sequence
atgggttctcaagaaattattaaagaaactttctttatttctcacggcactccaagaatgactattgaagcttctaaacccgctagaaaatttctagaatcttggagagataagatctactttaagaaaccaaaagctatcctggttatctctgcccattgggaaactgatttcccatctgttaatgcagtggatattaatgatactatctacgatttctatggctttccagctccaatgtaccaattcaaatatcctgctccaggttctccagatttggcccagagagttcaagaattgttgactgcttctggttttaagtccgtcaatgttgataagaaaagaggtttagatcatggcgcttgggttccattgatgttaatgtatccaaacgctgatattccagtttgtcaattatctgttcaatctcacttggatggtatgtatcattacaaattgggtagagctttagctcccctgaaagaagaaggtgttttaattattggttctggttctgccactcacccatctaataatactccacattattacgacggcgttgctccatgggctgctgattttgatcattggttggaaaccgctttgacaaatggttcttacgaagaggttaataaatgtgaatctaaagctccaaattggaagttggctcatccatggccagaacatttctacccactacatgttgctatgggtgctgctggtgagaattggaaagctgaattgattcataattcttgggatcatgggactatgtcttatggttcttataaatttgtttcttcttaa

SEQ ID NO: 15 - protein, 268 aa, Amaranthus hypochondriacus
MGSQEIIKETFFISHGTPRMTIEESKPARKFLESWRDKIYCKKPKAILVISAHWETDFPSVNAVDINDTIYDFYGFPAPMYQFKYPAPGSPDLAQRVQELLTASGFKSVNVDKKRGLDHGAWVPLMLMYPNADIPVCQLSVQSHLDGMYHYKLGRALAPLKEEGVLIIGSGSATHPSNSTPHYYDGVAPWAADFDQWLETALTNGSYEEVNKCERKAPNWKLAHPWPEHFYPLHVAMGAAGENWKAELIHNSWDHGTMSYGSYKFVSS

SEQ ID NO: 16 - DNA, 807 bp, Artificial
  Codon-optimized sequence
atgggttctcaagaaattattaaagaaactttctttatttctcacggcactccaagaatgactattgaagaatctaagccagctagaaagttcttggaatcttggagagataagatttattgtaagaaaccaaaagctattttggtcatttccgctcattgggaaactgatttcccatccgttaatgctgttgatattaatgatactatttacgatttctatggttttccagcaccaatgtatcaattcaaatatccagctccaggttctcccgatttggctcaaagggttcaagaattgttgactgcttctggtttcaaatctgttaatgttgataaaaagagaggtttggatcatggcgcttgggttccattgatgttgatgtacccaaatgctgatattccagtttgtcaattatctgttcaatcccatttggatggtatgtatcattacaagttgggtagagctttagctccattgaaagaggaaggcgtcttgattattggttctggttccgctactcatccctctaattctactccacattattatgatggcgttgctccatgggctgctgattttgatcaatggttggaaactgctttaactaatggttcttatgaagaagtcaataaatgcgagagaaaagctccaaattggaaattggctcatccatggccagaacatttttaccccttacatgttgctatgggtgctgctggtgagaattggaaagctgaattgattcataattcatgggatcatggtactatgtcttatggttcttataaatttgtttcttcataa

SEQ ID NO: 17 - protein, 266 aa, Phytolacca americana
MDVKDMIRETFYISHGTPMMAINKSVPARSFLKGWREEVYSKKPKSILVISAHWETDLPTISAVNHSDLIYDFYGFPAPMYQLKYPAPGAPDLATRVQELLTVSGFKCALDKKRGLDHGSWVPLMFMYPDANIPVCQLSLQSHLDGTHHYKLGRALAPLKEEGVLVIGSGSSVHPSNDTPHAVGVAPWAAEFDNWLEEALTSGRYEDVNNYQTKAPNWKIAHPWPEHFYPLHVAMGAAGENAKAELIHRSWEHGTLGYACYKFTSS

SEQ ID NO: 18 - DNA, 801 bp, Artificial
  Codon-optimized sequence
atggatgttaaagacatgattagagaaactttttatatttcccacggtactccaatgatggctattaataagtctgttccagctagatcatttttgaaaggttggagagaagaagtctattctaaaaagccaaaatctatcttggtgatttccgctcattgggaaactgatttaccaacaatttctgcagtgaatcattctgacttgatttacgatttttatggttttccagccccaatgtatcagttgaaatatccagctccaggtgctccagacttggctaccagagttcaagaattattaactgtttctggtttcaagtgcgctttggataagaaaagaggtttggatcacggttcttgggttccattgatgtttatgtatccagatgccaatattccagtttgtcaattgtctctgcagtctcacttggatggtactcatcattataagttaggtagggctttggcaccattgaaagaagaaggtgttttggtcattgggtctggttcttctgttcatccatctaatgacaccccacatgctgttggtgttgctccatgggctgccgagttcgataattggttggaagaagctttaacctctggcagatacgaagatgttaataattatcaaaccaaagctccaaactggaaaattgctcatccatggcccgaacacttttatccattgcatgttgctatgggtgctgccggtgaaaatgccaaagctgaattgattcatagatcttgggagcatggtaccttgggttatgcttgttataaatttacttcttcataa

SEQ ID NO: 19 - protein, 271 aa, Suaeda salsa
MGSNNNNEKIKETFFLSHGTPMMIIEESQPARKFLESWTEKIYSKKPKAILVISAHWETDHPAVTSVEVNDTIHDFYGFPAPMYQFKYPAPGSPDLAKRVQELLTASGFKSVQTDKKRGLDHGAWVPLMFMYPEPEIPVCQLSVQSHLDGKFHYNLGRALAPLKDEGVLIIGSGSATHPSNATPHSYDGVAPWAADFDCWLETSLTNGRYEEVNKCETKAPNWKLAHPWPEHFYPLHVAMGAAGEDWKAELIHNSWDHGTMSYGSYKFTSS

SEQ ID NO: 20 - DNA, 816 bp, Artificial
  Codon-optimized sequence
atgggttctaataataataatgaaaagattaaagagactttctttttgtctcatggtactccaatgatgattatcgaggaatctcaaccagctagaaaatttttggagtcttggaccgagaaaatttattccaagaaaccaaaagctattttggttatctctgcccattgggaaactgatcatccagctgtcacctctgttgaggttaatgatactattcatgatttttatgggtttcccgctccaatgtatcaatttaaatatccagctcccggttccccagatttggctaaaagagttcaagaattattgaccgcatccggttttaaatctgttcaaactgataagaagcgcggcttagatcatggtgcttgggttcccttgatgtttatgtacccagaaccagaaattccagtctgtcaattgtccgttcaatctcatttagatggcaagtttcattataatttaggtagagccttagctcccttgaaggatgaaggtgttttgattattggctctggctctgctactcatccatctaatgctactccccattcctatgacggtgttgctccatgggctgctgacttcgattgttggttagaaacttctttgactaacggtagatatgaagaggttaataaatgtgaaaccaaagctccaaattggaaattggctcatccatggccagaacacttttatccattgcatgttgctatgggggccgctggcgaagattggaaagctgaattgattcataattcctgggatcacggtactatgtcttacggttcctataaattcacttcttcataa

SEQ ID NO: 21 - protein, 268 aa, Bougainvillea glabra
MGGEKKMKGTYYIAHGDPIMYINKSIKLRHFLEEWKENVVMEKPICILVISAHWDTDVPTVNLVEHCDTIHDFDDYPDPLYQIKYPAPGAPKLAMKVQELLKGGGFKCEVDTKRGLDHAAWFPLMLMYPEADIPICELSVQTNKDGTHHYNLGKALSPLLNDDVLIIGSGGAVHPSDDTPHCPNGVAPWALQFDNWLEDALLSGRYEDVKEFKKMAPNWEISHPGQEHLYPLHVALGAAGNNVKTELIHQTWAANGVFGYSSYKFTST

SEQ ID NO: 22 - DNA, 807 bp, Artificial
  Codon-optimized sequence
atgggtggtgaaaagaaaatgaaaggtacttattatattgctcatggtgacccaattatgtatattaataagtctattaaattgaggcattttctagaagaatggaaagaaaatgttgtcatggaaaaaccaatttgtattttagttatttctgctcattgggatactgatgtcccaacagtcaatttggttgaacattgcgacactattcacgactttgatgattatcccgatccattgtatcaaattaaatatccagctccaggtgctcccaaattggccatgaaagttcaagaattgttgaagggtggtggtttcaaatgtgaagttgatactaaaagaggcttagaccatgctgcttggtttccattaatgctgatgtatccagaagctgatattccaatttgtgaattgtctgttcaaaccaacaaagatggtactcatcattataacttgggtaaagctttgtctccattactgaatgatgacgttctgattattggttctggtggtgccgttcatccttctgatgatactccacattgtccaaatggtgtcgctccctgggctttgcaatttgataattggttggaagacgccttgctatctggtaggtatgaagacgttaaagaatttaaaaagatggcccctaattgggaaatctctcatccaggtcaagaacatctctatcccttgcacgttgctttgggtgctgctggtaacaacgttaaaactgaattaattcatcaaacatgggctgctaacggtgtttttggttattcttcttataaatttacttctacttaa

SEQ ID NO: 23 - protein, 275 aa, Beta vulgaris
MKMMNGEDANDQMIKESFFITHGNPILTVEDTHPLRPFFETWREKIFSKKPKAILIISGHWETVKPTVNAVHINDTIHDFDDYPAAMYQFKYPAPGEPELARKVEEILKKSGFETAETDQKRGLDHGAWVPLMLMYPEADIPVCQLSVQPHLDGTYHYNLGRALAPLKNDGVLIIGSGSATHPLDETPHYFDGVAPWAAAFDSWLRKALINGRFEEVNIYESKAPNWKLAHPFPEHFYPLHVVLGAAGEKWKAELIHSSWDHGTLCHGSYKFTSA

SEQ ID NO: 24 - DNA, 828 bp, Artificial
  Codon-optimized sequence
atgaaaatgatgaatggtgaagatgctaatgatcaaatgattaaagaatctttcttcattactcatggtaacccaattttaactgttgaagatactcatccattgagaccattctttgaaacttggagagaaaagatttttagtaagaagccaaaagctattttgattatttccggccactgggaaactgttaaaccaactgttaatgccgtccatatcaatgatactattcatgattttgatgattatccagctgccatgtatcaatttaaatatccagctccaggcgagccagaattagctagaaaagttgaagaaattttgaaaaagtccggtttcgaaactgctgaaactgatcaaaagagaggtttggatcatggtgcctgggttccattgatgttgatgtatccagaagctgatatcccagtctgtcaattatctgttcaaccacatttagatggtacttaccattataatttgggtagagccctggctcccttgaaaaatgatggtgtattgattattggttccggttctgccactcacccattggatgaaaccccacattattttgatggtgttgctccatgggctgctgcttttgattcttggttgagaaaagccttgattaacggtagattcgaagaagttaatatttatgaatctaaagctcccaattggaaattggctcatccattcccagaacacttttatccattgcatgttgttttaggtgctgctggtgaaaaatggaaagctgaattaatccattcctcctgggatcatggtactttgtgtcatggttcttataaatttacttctgcttaa

SEQ ID NO: 25 - protein, 275 aa, Beta vulgaris
MKMMNGEDATDQMIKESFFITHGNPILTVEDTHPLRPFFETWREKIFSKKPKAILIISGHWETVKPTVNAVHINDTIHDFDDYPAAMYLFKYPAPGAPELARKVEEILKKSGFETAETDEKRGLDHGAWVPLMLMYPEADIPVCQLSVQPHLDGTYHYNLGRALAPLKNDGVLIIGSGSATHPLDETPHYFDGVAPWAAAFDSWLRKALINGRFEEVNIYETKAPNWKLAHPFPEHFYPLHVVLGAAGEKWKAELIHSSWDHGTLCHGSYKFTSA

SEQ ID NO: 26 - DNA, 828 bp, Artificial
  Codon-optimized sequence
atgaaaatgatgaatggtgaagatgctactgaccaaatgattaaggaatcatttttcattactcacggcaacccaattttgactgttgaagatactcatcccttgagacccttctttgaaacttggagagagaaaatttttagtaaaaagccaaaagctattttgatcatttccggtcattgggaaactgttaagccaaccgttaatgccgttcatattaatgataccattcatgattttgatgattatccagctgctatgtatttgtttaaatacccagctccaggcgccccagaattagctagaaaagttgaagaaattttgaaaaagtctggttttgaaactgctgaaactgatgagaagagaggtttggatcatggtgcttgggttccattgatgttgatgtatccagaagctgatattcccgtttgtcagttatctgttcaaccacacctggatggtacttatcattataatttgggtagagctttggccccgttgaagaatgatggtgttttgattattggttccggtagcgctactcatccattagatgaaactccacattattttgatggcgttgctccatgggctgctgctttcgattcttggctgagaaaagctttgattaatggtaggtttgaagaagttaacatttatgagactaaagcccccaattggaaattggctcatccattcccagaacatttctacccattgcatgttgttttaggtgctgctggtgaaaaatggaaagctgaattaattcattcttcttgggatcacggtactttgtgtcatggctcttataaattcacctctgcttaa

SEQ ID NO: 27 - protein, 497 aa, Beta vulgaris
MDHATLAMILAILFISFHFIKLLFSQQTTKLLPPGPKPLPIIGNILEVGKKPHRSFANLAKIHGPLISLRLGSVTTIVVSSADVAKEMFLKKDHPLSNRTIPNSVTAGDHHKLTMSWLPVSPKWRNFRKITAVHLLSPQRLDACQTFRHAKVQQLYEYVQECAQKGQAVDIGKAAFTTSLNLLSKLFFSVELAHHKSHTSQEFKELIWNIMEDIGKPNYADYFPILGCVDPSGIRRRLACSFDKLIAVFQGIICERLAPDSSTTTTTTTDDVLDVLLQLFKQNELTMGEINHLLVDIFDAGTDTTSSTFEWVMTELIRNPEMMEKAQEEIKQVLGKDKQIQESDIINLPYLQAIIKETLRLHPPTVFLLPRKADTDVELYGYIVPKDAQILVNLWAIGRDPNAWQNADIFSPERFIGCEIDVKGRDFGLLPFGAGRRICPGMNLAIRMLTLMLATLLQFFNWKLEGDISPKDLDMDEKFGIALQKTKPLKLIPIPRY

SEQ ID NO: 28 - DNA, 1494 bp, Artificial
  Codon-optimized sequence
atggatcacgcaacattagctatgatattagcaattttgtttatttcatttcattttattaaattattattctcacaacaaacaacaaaattgttacctccaggtcctaaacctttaccaattattggtaatatcttggaagtaggaaagaagcctcatagatcatttgcaaatttggctaagatccacggtcctttaatctctttgaggttaggatcagttaccaccattgttgtatcatctgctgatgtcgccaaagagatgttcttgaagaaggatcaccctttatcaaacaggacaattccaaattcagtaactgctggtgaccaccacaaattgactatgtcttggttgcctgtttcacctaaatggagaaacttcagaaagataactgctgttcatttattgtctccacaaagattagatgcatgccagacctttagacatgcaaaggttcagcaattatatgaatacgttcaagagtgcgctcagaagggtcaggcagttgacattggtaaagctgccttcacaacttctttgaatttattgtctaaattattcttttctgttgaattggcccatcataaatcacatacctctcaagagtttaaggaattaatttggaatattatggaagacatcggtaaaccaaactacgccgattacttcccaattttaggttgcgtcgatccatcaggaattagaagaaggttggcatgttcttttgataagttaattgcagtatttcaaggaataatatgtgagaggttagctccagattcatcaactacaactactacaactacagatgatgtcttggacgtattattgcaattgtttaagcagaatgaattaactatgggagagattaaccacttgttagtcgatattttcgatgccggtactgatactacatcatctacctttgagtgggttatgactgagttaataagaaaccctgaaatgatggaaaaggcccaagaggaaataaaacaggtattgggtaaggataagcaaatccaagagtcagacataataaatttgccatacttgcaagcaatcattaaggaaactttgaggttgcacccacctacagttttcttgttgcctagaaaagccgacacagacgtagaattgtacggatacattgttccaaaggatgctcaaattttggtaaacttgtgggccataggtagagatccaaatgcttggcaaaatgccgatattttctcacctgaaagatttataggttgtgagattgatgtaaagggaagagattttggtttattgccttttggtgctggtagaagaatttgccctggtatgaatttggccataaggatgttgacattgatgttggccacattattgcaattcttcaactggaaattagagggagacatttcacctaaagatttggacatggatgaaaagtttggtatagccttgcagaaaactaaacctttaaaattgatcccaattccaagatactaa

SEQ ID NO: 29 - protein, 506 aa, Basella alba
MDNTTLAILLSTSYFIIYLIITKLGFHYKVLPKNPKQRLRLPPGPKPLPIIGNVLELGSKPHRSFTNLAKVHGPLISLRLGSVTTIIVSSSHVAKEMFLKNDQSLSSNRTIPHSVTAGDHHKLTMSWLPVSPKWRSFRKITTFHLLSPQRLDACCSLRQAKVQQLFEYVLQCSRTGQPVDIGKAAFTTSLNLLSKLFFSLELAHHRSTKSQEFKDLIWNIMEDIGKPNIADHFPCLKYFDPSGIRRRLASSFERLIEVFQDIIRQRMSLSFGSSHNNDVLDVLLGLYNQKELTMDEINHLLVDIFDAGTDTTSSTFEWSMAELMKNRRIMEKAQAEILHVLGKNSYIQESDISKLPYLRAIIKETLRLHPPTVFLLPRKADADVELYGYVVPKDAQILVNLWALGRDPAVWENPDEFSPDRFMGSEIDVKGRDFGLLPFGAGRRICPGMNLAIRMLTLMLATLLRSFDWKLPEGEAPAQLDMDEKFGIALQKTTPLKIIPIFKNSI

SEQ ID NO: 30 - DNA, 1521 bp, Artificial
  Codon-optimized sequence
atggataatactactttggctatcttactatccactagctattttattatctacttgatcattactaaattagggttccattataaggtcttgccaaagaatccaaagcagaggcttagattaccaccaggtccaaagccactgccaatcataggtaatgttttggaattaggttctaagccccacagaagttttactaatttagctaaagttcatggacccttgatttctttaagattgggctctgtcactactattattgtttcatcttctcatgttgctaaagaaatgtttttgaaaaatgatcagtcattgtctagcaatagaactattccacattctgtcaccgctggtgatcatcataaattgacaatgtcttggttaccggtttctcccaaatggagatcattcagaaaaattactacttttcatttgctatctccacagaggttggatgcttgttgttctttgcgccaagctaaagtccaacaattgtttgaatacgttttacagtgtagcagaactggtcaaccagttgatattggaaaagcagcctttactacctctttaaatttgttgtctaagctcttcttttctttggaattagctcatcatcggtctacaaaatctcaagaatttaaagatttaatttggaatattatggaggatattggcaagccaaacattgctgaccattttccctgtttaaaatattttgatccatctggtattaggcgcaggttagcttcttcatttgaaagattgattgaggttttccaagatataattagacaacggatgagcttgtcttttggttcttctcacaataatgatgtcttggatgttttgttaggtttatataaccagaaggaacttacaatggacgaaattaaccatctcttggttgatatttttgacgctggtactgataccacttctagtacttttgaatggtcaatggctgaattaatgaaaaatagaagaattatggaaaaggctcaagcagaaattttacacgttttgggcaaaaattcatacatccaagaatctgatatttctaaattgccctatttgagagctattattaaagaaactttaaggctgcatccacctactgtctttttgctaccaagaaaagctgacgctgatgttgaattgtatggttacgttgtgccaaaggatgctcaaattttggttaacctatgggccttgggtagagatccagctgtttgggaaaatccagatgaattttctccagataggtttatgggttctgagattgatgtcaaaggtagagattttggtctattgccatttggtgctggtagaagaatctgccccggtatgaatttagctattagaatgctaacattaatgttagccacattgttaagatcctttgattggaagttgcctgaaggtgaagctccagcacagctggatatggatgaaaaatttggaattgcattacagaagactactccattgaaaattattccaatttttaaaaattcaatttaa

SEQ ID NO: 31 - protein, 495 aa, Phytolacca americana
MDYTTLVMILSIVFFCYNLFNLLFTRKNTKLPPGPKTIPIFGNIFELGKKPHQSFANLAKIHGPLMSLKLGSVTTIVVSSAEVAREMFLKNDQLLSNRTVPNSVTAGDHHKTTMSWLPVSQKWRNFRKITAVHLLSPQRLDSCQALRQAKVKQLFNYIHECAQKGEAVDIGKAAFTTSLNLLSNLFFSVELANHKSSSSQEFKQLIWNIMEDIGKPNYADYFPVLKYVDPSGIRRRLASNFNKLIDVFQGFIRLRMSTNSSCGATNPNDVLDVLLNLYKGDDLNMDEINHLLVDIFDAGTDTTSSTFEWAMAELVKNPKMMKKAQAEIQQVLGKDSIIRESDIPNMPYLQAIIKETLRLHPPTVFLLPRKADADVELYGYVVPKNAQILVNLWALGRDPLVWKSPNVFKPERFLGSEIDFKGRDFGLLPFGAGRRICPGMNLAYRMLTLMLATLLQSFDWKVADGTNPQDMDMDEKFGIALQKTTPLQIIPVYKY

SEQ ID NO: 32 - DNA, 1488 bp, Artificial
  Codon-optimized sequence
atggattatactactttagttatgattttgtctattgtgttcttttgttacaatctctttaatttgttgtttacaagaaagaatactaaattacctccaggtccaaagactattccaatttttggcaacatttttgaattaggtaagaaacctcatcagagctttgccaacttagctaaaattcacggcccacttatgtctttaaaattgggttctgttacgacaattgtcgtgtcctctgctgaagttgctagggagatgtttttgaaaaacgatcaattgttatctaacaggactgttccaaattctgttacagctggtgatcaccacaagaccaccatgtcttggttgccagtttctcaaaagtggaggaattttagaaaaattacagcagttcatttattatctccacaaagattagattcttgtcaagctttgagacaagccaaagttaaacaattattcaactacattcacgaatgtgctcaaaaaggcgaagctgtggatattggcaaagctgcttttactacatctttgaacttgttatcaaacctctttttcagcgttgaactggctaatcataaatcttctagctcacaggaatttaaacaactgatttggaacattatggaagatattggtaaacctaactacgctgactacttcccagttttaaaatacgttgatccatccggcatcagaagaagattagcttccaattttaacaaattgattgatgttttccagggtttcattagactaagaatgtctactaactcttcttgtggggcaactaatccaaatgacgtcttggatgttttgttgaatttgtataaaggtgatgatttgaatatggatgaaataaatcatctattggttgacatttttgacgcaggtactgatactacttcttccacttttgaatgggctatggccgagttggttaagaatccaaaaatgatgaagaaagcccaagctgaaattcaacaagtcttgggtaaggattctattattagagaatctgacattccaaacatgccatatttgcaagctattattaaagaaaccttgagattgcacccacctactgttttcttgttgccaagaaaggctgatgctgatgtagaattgtatggctacgttgttcccaaaaatgctcaaattttggttaacttgtgggctcttggtagagatccattagtgtggaagtctcctaatgtttttaaaccagaaagatttttaggttctgagatcgactttaaaggtagagattttggcttattgccatttggtgctggtaggagaatttgcccaggtatgaatttagcttatcgcatgctgactttaatgctagctactctattacaatcctttgattggaaggttgctgatggtactaacccacaagacatggacatggatgaaaaatttggcattgccttacaaaagactactccattgcaaattattccagtttataaatattaa

SEQ ID NO: 33 - protein, 496 aa, Phytolacca americana
MDHTTLAMILSVIFLLYNLVKAIFSQSNTKLPPGPKPVPIFGNIFELGDKPHRSFANLAKIHGPLITLKLGSVTTIVVSSAEVAKEMFLTNDQLLANRNVPNSVTAGDHHKLTMSWLPVSPKWKTFRKITAVHLLSPQRLDACQALRHTKVKQLYEYVQECAKRGEAVDIGKAAFTTSLNLLSNLFFSVELANHTSSSSQEFKELIWDIMEDIGKPNYADYFPVLKCVDPWGIRRRLESNFDKLIEVFQSFIRKRLSTEPFSASAKTPNDVLDVLLNLLKEEELNMGEINHLLVDIFDAGTDTTSSTFEWAMAELVRNPEMMKKAQDEIEQVLGKDAIIQESDIPKMPYLQAIIKETLRLHPPTVFLLPRKASSNVELYGYVVPKNAQILVNLWAIGRDPTVWDNPNMFSPERFLNSDIDVKGRDFGLLPFGAGRRICPGMNLAYRMLTLMLATLLQSFDWKLGDGVNPKDLDMDEKFGIALQKTKPLQVIPVLKY

SEQ ID NO: 34 - DNA, 1491 bp, Artificial
  Codon-optimized sequence
atggatcacactacattggcaatgattttgtccgttatcttcttgttgtacaatttagttaaagctatcttttctcaatctaatactaagttgccaccaggtccaaagccagttccaatttttggtaatatttttgaattgggtgataagccacatagatcttttgccaatctggccaagatccatggtccgttgattactcttaagttgggttcagttacaactattgttgtttcttctgctgaagttgctaaagaaatgttcttaactaatgatcaactattagctaatagaaatgttcccaactctgttacagctggtgatcatcataagttaactatgtcctggttaccagtttctccgaagtggaaaacttttagaaaaattactgcagtacatttgttgtctccacaaagattggatgcttgtcaggctttgaggcataccaaagttaaacaattgtacgaatatgttcaagaatgcgcaaaaagaggtgaagcagtggacattggtaaagctgcttttacaacttctttgaatttgttgtccaatttattcttctctgttgaattagctaaccatacttcttcttcttcacaagagtttaaggaattaatttgggatattatggaagatattggtaagccaaattatgccgattattttccagtcttgaagtgtgttgatccatggggtattagaagacgtttggaatctaactttgataagttgattgaggtgtttcaatcttttattagaaagaggttgtctacagaaccattttctgcatcagctaaaactccaaatgatgtgttagatgtgttgttaaatttgttgaaggaagaagaattaaatatgggtgaaattaaccacttattggttgatattttcgacgccggcactgatacaacttcttcaacctttgagtgggctatggcagaattggttagaaatccggagatgatgaaaaaggctcaagatgagattgaacaagtcttaggtaaagatgcaattattcaagaatctgatatcccaaagatgccatacttacaagctatcattaaggaaaccttgagattgcacccaccaacagttttccttttgccaagaaaagcatcttctaacgttgaactatatggttatgtcgttcccaaaaacgctcaaattttagttaatttatgggcaattggcagggatcctacagtttgggataatcctaacatgttctccccagagagatttttaaattctgatattgatgttaaagggagggattttggattgttgccatttggtgctgggagacgtatctgtccaggtatgaatttggcatacagaatgttgactttaatgttagcaaccttgttgcaatcctttgattggaagttgggtgatggtgttaacccgaaggatttggatatggatgagaaatttggtattgctctgcagaaaactaaacctcttcaagtcatccccgttttgaaatattaa

SEQ ID NO: 35 - protein, 508 aa, Opuntia ficus-indica
MDTPTLSYFISAITFYYIAFQIVKLGFNVIMTSKKTKRRRLPLPPGPKPLPIIGNVFELGPKPHRSFASLAKVYGPLMSLRLGSVTTIIVSSSDVAKEMFLKNDQPLSSTRTIPNSVTAGDHHKLTMSWLPVSPKWRSFRKITTFHLLSPQRLDACSGLRQAKVQQLYEYVLECSRTGQAVDIGKAAFTTSLNLLSKLFFSLELANHTSDKSQEFKELIWNIMEDIGKPNYADYFPCLKYFDPSGIRRRLACSFEKLIEVFQVIIRQRLSLSSSGTNDHNNDVLDVLLDLYQQKELSMEEINHLLVDIFDAGTDTTSSTFEWAMAELIKNPRMMETAQAEIKLILGKDLHIQESDIPKLPYLRAIIKETLRLHPPTVFLLPRKADADVELYGYTVPKNAQILVNLWALGRDPKVWENPDVFSPERFLGCDIDVKGRNFGLLPFGAGRRICPGMNLAYRMLTLMLATLLQSFDWKLPNEMNPQNLDMDEKFGIALQKTKPLQIIPLSKD

SEQ ID NO: 36 - DNA, 1527 bp, Artificial
  Codon-optimized sequence
atggatactccaactttgtcttattttatttctgccatcacattctattacattgcatttcaaattgttaaattgggttttaacgttattatgacctcgaagaagactaaaagaagaagattaccattgccacctggcccaaaacccttgccaattattggtaatgttttcgaattaggtccaaagccacatagatcttttgcatcattggccaaagtctacggtccattgatgtctttgagattaggttcagtcactaccattattgtctcttcttcagatgtggctaaggaaatgtttttgaaaaatgatcaacccttgtcttccacaagaactattccaaattccgttactgctggtgatcatcataaattgactatgagctggttacccgtctctccaaaatggagatctttcaggaaaattacaactttccatttattatccccacaaaggttggatgcttgttctggtttaagacaagcgaaagtgcaacaattatatgaatatgttttagagtgctcaagaacaggtcaagctgttgatattggtaaagctgctttcactacttcattgaatttattgtctaagttgtttttctcattggaattggctaaccatacttctgataagtcacaagaattcaaggaattgatttggaatattatggaagacatcggtaagccaaactacgctgattactttccatgtttgaagtatttcgatccatctggtattaggcgccgtttagcttgttcttttgaaaaattgattgaagtttttcaggtcattattagacaaaggttatccctctcttcttctggtactaacgatcataataatgatgttttagatgttttgttagacttatatcaacagaaagaattgtctatggaggaaatcaatcatttgttagttgatatttttgacgctggtactgatactacttctagtacctttgaatgggcgatggccgaattaattaaaaacccaagaatgatggaaactgctcaggccgaaattaaattaattttgggtaaagatttacatattcaagaatctgacatcccgaaactgccatacttgagagctattattaaagaaactttgcgcttgcatccaccaactgtctttttgttgccaaggaaagctgacgctgatgttgaattgtatggctatactgtcccaaagaacgctcaaattttggttaatttatgggccttgggtagggatccaaaagtttgggaaaatccagatgttttctcaccagagagatttttgggttgtgacattgatgttaagggtaggaattttggtttattaccatttggtgctggtcgaagaatttgtccaggtatgaatttggcttataggatgctaactttaatgttggcaacattgttgcaatcttttgattggaaattaccaaatgaaatgaacccacaaaatttagatatggacgaaaagttcggcattgctttacaaaagactaaaccgttgcagattattcctttgtctaaagattaa

SEQ ID NO: 37 - protein, 495 aa, Abronia nealleyi
MDQTTLAMLLSALYLLYNLYKVIFTQSNSKLPPGPKPLPIFGNISELGAKPHRSFANLAKIHGPLITLKLGSVTTIVVSSAKVAEEMFLKNDLPLANRNVPNSVTAGDHHKLTMSWLPVSPKWKTFRKITAVHLLSPQRLDACQALRHAKVKQLYEYVYDCAKKGEAVDIGKAAFTTSLNLLSNLFFSVELAQHTSTSSQHFKQLIWDIMEDIGKPNYADYFPALKCVDPWGIRRRLAANFERLIDVFQDFIRPRLSMNPSSVTSASDVLDVLLNLYKEKELNMGEVNHLLVDIFDAGTDTTSSTFEWAMAELVRHPETMKKAQDEIEQVLGKDATIQEADIPKMPYLQAIIKETLRLHPPTVFLLPRKAATNVELYGYVVPKDAQILVNLWAIGRDPLVWDQPNVFSPERFLNSDVDVKGRDFGLLPFGAGRRICPGMNLAYRMLTLMLATLLQSFEWKVENGEKAEDLDMDEKFGIALQKTKPLQIIPVLKYC

SEQ ID NO: 38 - DNA, 1488 bp, Artificial
  Codon-optimized sequence
atggatcaaactactttggctatgttgttgtctgccctctacttgttgtataacttgtataaagttattttcacccagagcaactccaaattgccaccaggtccaaaacctttaccaatttttggtaatatctctgaattaggtgctaagccccatagatcttttgctaatttagctaaaatccatggcccgttgattactttgaaattgggatctgtgaccacgatcgtggtttcttcagctaaagtggccgaagaaatgttcctgaagaatgatttaccattagctaataggaacgttccaaattctgttactgctggtgaccaccataaattaactatgtcttggttaccagtctctccaaagtggaaaacttttagaaaaattaccgccgtgcatttgttatcaccacaaagattagatgcttgtcaagctttgaggcacgcaaaagtaaaacaattgtatgaatatgtgtacgattgcgctaaaaagggtgaagctgttgatattggtaaagcagctttcactacatcgttgaatttattatctaatttgttcttctcagttgaattggctcaacatacttcgacttcttcccaacattttaaacaattgatttgggacattatggaagatattggtaagccaaactacgctgactactttccagctttgaaatgtgtcgatccatggggtattcgtagaagattggctgctaattttgaaaggttgattgacgtttttcaagattttattagaccgagattgtcaatgaatccatcctccgtcacttctgcttctgatgttctcgacgttttgttaaatttgtataaagaaaaagagcttaatatgggcgaagttaatcacttgttagtcgatatttttgatgctggtaccgatactacttcttctacatttgaatgggctatggctgagttggtccgtcatccagaaactatgaagaaagctcaggatgagatcgaacaagttttgggtaaagatgccactattcaagaagctgatattccaaaaatgccctatctgcaggctattattaaagaaacattgagattgcacccaccaactgttttcttgttgccaagaaaagcagctaccaacgtcgaactgtatggttatgttgttcctaaggatgctcagatcttggttaatttgtgggctattggtagagatccattggtttgggatcaaccaaacgtcttctctccagaaagatttttgaattcagatgtggatgttaaaggtagagattttggtttgttaccttttggcgctggtagaagaatttgtccaggtatgaacctagcttaccggatgttgactttgatgttagccactttattgcaatcttttgaatggaaagttgaaaatggtgaaaaagccgaagatttggatatggatgagaaattcggtatcgctttacagaaaacaaaaccattacaaattattccagttttgaaatattgttaa

SEQ ID NO: 39 - protein, 495 aa, Acleisanthes obtusa
MDQTTLAMLLSALYLLYNLYKVIFTQSNSKLPPGPKPLPILGNIFEVGNKPHRSFANLAKIHGPLITLKLGSVTTIVVSSAKVAEEMFLKNDLPLANRNVPNSVTAGDHHKLTMSWLPVSPKWKTFRKITAVHLLSPQRLDACQALRHAKVKQLHEYVQDCAKKGQAVDIGKAAFTTSLNLLSNLFFSVELAQHTSSSSQHFKELIWDIMEDIGKPNYADYFPALKCVDPWGIRRRLAANFERLIDVFQGFIRQRLSINSSTVTSASDVLDVLLNLYKEKELNMGEINHLLVDIFDAGTDTTSSTFEWAMAELVRHPEIMKKAQDEIEQVLGKDAIIQEADIPKMPYLQAIIKETLRLHPPTVFLLPRKATTNVELYGYVVPKNAQILVNLWAIGRDPLVWDNPNKFSPERFLNSDIDVKGRDFGLLPFGAGRRICPGMNLAYRILTLMLATLLQSFEWMVENGENPEDLDMDEKFGIALQKTKPLEIIPVIKHR

SEQ ID NO: 40 - DNA, 1488 bp, Artificial
  Codon-optimized sequence
atggatcaaactactttggccatgttgctttccgcgttgtatctattatacaacttgtacaaagtcattttcactcaaagcaattctaaattgccaccaggtccaaagccactacccatcttaggtaatatttttgaagttggcaataaaccccatcgctcctttgctaatttggctaagattcacggcccattgattacattgaaacttggttccgtcactacaatcgttgtttcttctgctaaagttgccgaagaaatgttcttaaagaatgatttaccattggctaatagaaacgttccaaattctgttacagccggtgatcatcataagttgactatgtcttggttaccagtttcccctaaatggaaaacatttagaaaaattactgccgttcacttgttgagcccacaaagattggatgcttgtcaagccttgagacatgctaaagttaaacaattgcatgaatatgtacaagattgtgccaagaaaggtcaggctgtagatattggtaaagctgcttttacaacctcattgaacttgctatctaatttatttttctctgttgaattagcccaacatacttcttcctcttctcagcattttaaagaattaatttgggatattatggaggatattggtaagccaaattacgctgattattttcccgctttaaaatgtgttgatccatggggtatcagaagaagacttgcagctaacttcgaaagactgattgacgtctttcaaggttttattcgccagcggttgtctattaatagctctactgttacctccgcttcagatgttttagatgtgttgctgaatttatataaggaaaaagaattgaatatgggtgaaataaatcacctattggttgatattttcgatgccggtactgatacgacatcttctacatttgaatgggctatggctgaattggttagacatccagaaattatgaagaaagcgcaagatgaaattgagcaagttttggggaaagacgctatcattcaagaagctgatattccgaagatgccctacttgcaagctattattaaagaaactttgcgattacatcctccaaccgtctttttgttgccaagaaaagctactaccaatgttgaattgtacggttatgttgttccaaagaatgctcagatcctggttaatttgtgggctattggtagagacccattggtatgggacaatcctaataaattttctccagagagattcttgaattctgatattgatgttaaaggtcgtgacttcggtttattaccatttggtgccggtagaagaatttgtccaggtatgaatttggcctacagaatcctgactttaatgttggcaactttactgcaatctttcgaatggatggttgaaaatggtgaaaacccagaagatttggatatggatgaaaagttcggcattgctttgcagaaaacaaaacctttggaaattatcccagttattaaacatagataa

SEQ ID NO: 41 - protein, 495 aa, Mirabilis multiflora
MDQTTLAMLLSALYLLYNLFKVIFTQSNSKLPPGPKPLPILGNIFELGDKPHRSFNNLAKIHGPLITLKLGSVTTIVVSSAKVAEEMFLKNDLPLANRNVPNSVTAGDHHKLTMSWLPVSPKWKTFRKITAVHLLSPQRLDACQALRHAKVKQLYQYVQDCAKKGEAVDIGKAAFTTSLNLLSNLFFSVELAQHTSSSSQHFKELIWDIMEDIGKPNYADYFPALKCVDPWGIRRRLAANFERLIQVFQNFIRQRLSTDPSSVTNASDVLDVLLNLYKEKELNMGEINHLLVDIFDAGTDTTSSTFEWAMAELVRHPEIMKKAQDEIEQVLGKDATIQEADIPKMPYLQAIIKETLRLHPPTVFLLPRKATTNVELYGYVVPKNAQILVNLWAIGRDPLVWDQPNKFSPERFLNSDIDVKGRDFGLLPFGAGRRICPGMNLAYRMLTLMLATLLQSFEWKVQNGEKPEDLDMDEKFGIALQKTKPLEIIPVLKYR

SEQ ID NO: 42 - DNA, 1488 bp, Artificial
  Codon-optimized sequence
atggatcaaactactttggctatgttattatccgccctataccttttgtataatttgttcaaggtcatttttactcaatctaattctaaattgccgccaggtccaaaaccattgccaattttaggcaatatctttgaattgggtgacaaaccacatagatcttttaataatttggccaaaattcatggtccattaatcacattaaaattaggttctgttacaactattgttgtttcttctgctaaagttgcagaagaaatgtttttgaagaatgacttgcccttggctaatagaaatgtaccaaattctgttactgctggtgatcaccataaattgactatgtcttggttgcccgtttctccaaaatggaaaacttttagaaagattaccgctgtgcacttgttgtctccacaaagacttgatgcctgccaggccttgagacatgctaaggttaaacaactgtaccagtacgtgcaagattgtgctaagaaaggtgaagccgttgatattggtaaagctgcgttcaccacttccctcaatttattgtctaacttgttcttttctgtcgaactagctcaacatacttcttctagctcccaacattttaaagaattaatttgggatattatggaagatataggtaaaccaaattatgctgattattttcctgccttgaaatgtgttgatccatggggtattagaagaagattagctgctaactttgaaagattgatccaagtttttcaaaacttcattagacaaagattatctactgatccatcttccgtcacaaatgcctctgatgttctagacgttttattgaacttgtataaagaaaaagagttaaacatgggtgagattaatcatttgttagttgatattttcgatgccggtactgatactacatcttcaacttttgaatgggctatggctgaattagtgcgtcacccagaaattatgaagaaagctcaagatgaaattgaacaagttctcggtaaagatgccacaattcaagaagctgatattcccaaaatgccatatttgcaagctatcattaaagaaactttgagattgcatcccccaactgtcttcttgctaccaagaaaagccaccaccaatgttgaattgtatggttatgttgttcctaaaaatgcccagattttggttaatttatgggctattgggagggatccattagtttgggatcaaccaaataagttttctccagagcgctttttaaattctgacatcgatgtcaaaggccgtgattttggtttattgccatttggggctggtagaagaatttgtccaggtatgaacctagcttatagaatgctaaccttaatgttagctactttgttgcaatcatttgaatggaaggtgcaaaatggcgaaaaacctgaagatttggatatggacgaaaagtttggtattgctttgcagaaaacaaaacctttagaaattattccagttttgaaatatagataa

SEQ ID NO: 43 - protein, 494 aa, Ercilla volubilis
MDHTTLAMILSAIFLLYNLAKAIFSHSNTKLPPGPKPVPIFGNIFELGEKPHRSFANLAKIHGPLITLKLGSVTTIVVSSAEVAKEMFLKNDLPLANRNVPNSVTAGDHHKLTMSWLPVSPKWKTFRKITAVHLLSPQRLDSCQALRHTKVKQLHQYVQECAKRGEPVDIGKAAFTTSLNLLSNLFFSVELANHTSSSSQEFKELIWEIMEDIGKPNYADYFPILKCVDPWGIRRRLASNFDKLIEVFQGFIRKRLSTGSFSAITPNDVLDVLLNLLKEKELNMGEINHLLVDIFDAGTDTTSSTFEWAMAELVRNQEMMKKAQDEIEQVLGKDAIIQESDIPKMPYLQAIIKETLRLHPPTVFLLPRKATSNVELYGYVVPKNAQILVNLWAIGRDPKVWDNPNMFSPERFLNSEIDVKGRDFGLLPFGAGRRICPGMNLAYRMLTLMLATLLQSFDWKLGDGVNPKDLDMEEKFGIALQKTKPLQVIPVLKY

SEQ ID NO: 44 - DNA, 1485 bp, Artificial
  Codon-optimized sequence
atggatcatactactttagctatgattttgtctgctattttcctgttatataatttagctaaagccatttttagtcattctaacactaaacttccaccaggtccaaagcccgtccccatttttggtaatatttttgaattgggtgaaaaaccccatcgatcattcgctaatttggctaagatccacggtccactaattactttgaagttgggttctgtcaccactattgttgtatcttctgcagaagtcgcaaaggaaatgtttttgaaaaatgatttgccattggccaacaggaatgttccaaatagtgttactgccggtgatcaccacaaactaactatgtcatggttaccagtttctcccaaatggaaaacatttagaaaaataaccgctgtccatttattgtctccacaaagattggattcttgccaagctttaaggcatacaaaagttaaacaattacaccagtacgttcaagaatgtgctaaaagaggtgaaccagttgacattggtaaagctgcttttacaacctccttgaatttattatccaatttgttcttttccgttgaattagccaatcatactagttcatcttctcaggaatttaaagaattgatttgggaaattatggaagacattggaaaacccaattatgctgattattttccaatcctgaagtgcgttgatccatggggcattaggagaagattggcttcaaactttgataagttaattgaagtctttcaaggttttattcgcaagcgtttgtctactggttctttttctgccattactccaaacgatgttttagatgtgttattgaatttgttgaaagaaaaggaattaaatatgggtgaaattaaccatttgttagttgatatttttgatgctggtactgatactacttcatctactttcgaatgggccatggctgaattagttaggaaccaagaaatgatgaaaaaggctcaagatgagattgagcaggttttaggtaaggacgctatcatccaagaatctgatattccaaaaatgccatatttgcaagcaatcattaaagaaaccttgagattacacccgccaacagttttcttattaccaagaaaagctacctctaacgttgaattatatggttatgttgttccaaagaacgctcaaattttagttaatttgtgggctatcggtagagatccaaaagtttgggataatccaaatatgttctctccagaaaggtttttgaattctgaaatcgacgttaagggtagagattttggtttgttaccatttggtgcaggtagaagaatttgtccaggtatgaatttggcttataggatgttgacacttatgctagctacattgttacagtcttttgattggaagttaggcgatggtgtcaatccaaaagatctggatatggaagaaaagtttggtattgctttacaaaagactaaacctctgcaagttattccagtgttaaaatattaa

SEQ ID NO: 45 - protein, 497 aa, Phytolacca dioica
MDHTTLAMILSAIFLLYNLVKLAIFSQSNTKLPPGPKPLPIFGNIFELGDKPHRSFANLAKIHGPLITLKLGSITTIVVSSAEVAKEMFLKNDQPLANRNVPNSVTAGDHHKLTMSWLPVSPKWKTFRKITAVHLLSPQRLDACQALRHTNVKQLHEYVQECAQRGQPVDIGKAAFTTSLNLLSNLFFSVELANHTSSNSQEFKELIWDIMEDIGKPNYADYFPVLKCVDPWGIRRRLESNFDKLIEVFQGFIRKRLSTGSFSASETTPNDVLDVLLNLFKEKELNMGEINHLLVDIFDAGTDTTSSTFEWAMAELVRNPDMMKKAQDEIEQVLGRDAIIQESDIPKMPYLQAIIKETLRLHPPTVFLLPRKATTNVDLYGYVVPKNAQILVNLWAIGRDPTVWDNPNMFSPERFLNSDIDVKGRDFGLLPFGAGRRICPGMNLAYRMLTLMLATLLQSFNWKLGDGVNPKDLDMDEKFGIALQKTKPLQVIPVFKY

SEQ ID NO: 46 - DNA, 1494 bp, Artificial
  Codon-optimized sequence
atggatcatacaacattggctatgatcttatccgctatatttttgttgtacaatttggttaaattagctatttttagccaatcaaatactaaattgccaccaggtccaaagcctttgccaatttttggtaatatttttgaattgggcgataaaccacacagatcttttgctaatctagcaaaaattcatggtccattgattacattgaagttgggttctattacaactattgttgtgtcttccgctgaagttgctaaggagatgtttttgaaaaatgatcaaccattagctaacaggaatgtgccaaattctgttactgcgggtgatcaccacaaattgactatgtcctggttgccagtttctcccaaatggaaaacatttagaaaaattaccgccgttcatttgttaagtccacagagactagatgcctgtcaagctttgagacacaccaatgtcaagcaactccatgaatatgttcaagaatgtgctcaaagaggtcaaccagttgatattggtaaagctgcctttaccaccagcttaaatttgttgtcaaatttatttttctccgtcgagttggctaatcatacttcctctaactcccaagaatttaaagaattaatttgggatattatggaagatattggcaaaccaaattatgctgattacttcccagtgttaaaatgtgttgatccatggggtattcgtagacggttagaatccaacttcgataagttaattgaagtttttcaaggctttatccgtaagcgtttgtctactggttctttttcagcttctgaaaccacccctaatgatgttttggatgttttactgaatttgttcaaagaaaaggaattgaacatgggagaaattaaccacttgttagttgatatttttgatgcaggtaccgatactacatcttccacttttgaatgggctatggctgagttggtccgtaatccagatatgatgaagaaagctcaggatgaaattgaacaagttttgggtagagatgccattatccaagaatcagatattccaaagatgccatatctgcaagctatcattaaggaaactttgaggttacatccaccaactgttttcttactgcctagaaaagctactactaatgttgacctttatggctacgttgttcctaaaaatgctcaaattttggttaatttgtgggctatcggcagggacccaacagtttgggataatccaaacatgttttctccagagagatttttaaattctgatatagatgtcaagggccgggattttggtttattaccatttggtgctggtcggcgcatttgtccaggtatgaatttggcttacaggatgttgactctaatgttagctactttattgcaatcttttaattggaaattgggtgatggtgttaacccaaaggatttggatatggatgaaaaattcggtatcgctttgcaaaagacaaaacctttacaagtgattcccgtctttaagtactaa

SEQ ID NO: 47 - protein, 496 aa, Mirabilis multiflora
MDFLTLVMILSMIFFFYNLLKMIFTTHSDAQLPPGPKPMPIFGNIFELGEKPHRSFANLAKTHGPLMSLRLGRVTTIVVSSAEVAKEMFLKNDQSLADRSVPNSVTAGDHHKLTMSWLPVSPKWKNFRKITAVHLLSPQRLDACNALRHAKVKQLYEYVQECALKGEAVDIGKAAFTTSLNLLSNLFFSVELANHTSNTSQEFKQLIWDIMEDIGKPNYADYFPLLKYVDPSGIRRRLAANFDKLIDVFQSFICKRLSSAYSSATSLDDVLDVLLKLYKEKELNMGEINHLLVDIFDAGTDTTSNTFEWAMSELIRNPTMMKRAQNEIALVLGKDNGTIQESDIANMPYLQAIIKETLRLHPPTVFLLPRKAITYVKLYGYIVPKNAQILVNLWAIGRDPKVWKNPNEFLPDRFLNSDIDVKGRDFGLLPFGAGRRICPGMNLAYRMLTLMLATLLQSFDWKLPHGNSPMDLDMDEKFGIALQKTKPLEIIPVIKY

SEQ ID NO: 48 - DNA, 1491 bp, Artificial
  Codon-optimized sequence
atggactttttgactttagttatgattttatctatgattttcttcttttataacttgttaaaaatgatttttactacccattctgatgctcaattgccaccagggccaaaaccaatgccaatttttggtaatatttttgagttgggcgagaaaccacatagatcttttgccaatctagctaagacccatggtccattgatgtctttgaggttaggtcgtgttactactattgttgtttctagcgctgaagttgctaaagaaatgtttttgaaaaatgatcaatctttggcggacaggtctgttccaaattcagtaactgccggggaccatcacaaattgacaatgtcttggttacccgtttcacccaaatggaagaactttagaaaaattaccgctgttcatttgttgtctccacaaagattggacgcttgtaacgctttgagacatgccaaagttaaacaattgtatgaatacgttcaagaatgtgccttaaaaggtgaagctgttgatattggcaaagccgcattcaccacttcgttgaatttattgtctaatttgttcttttctgttgaacttgctaaccacacttcgaatacgtcgcaagaatttaaacaattgatttgggatattatggaggatattggtaaaccaaactacgccgattattttcctttgttgaaatacgttgatccaagcggtattagaagaagattagccgccaattttgataagttaattgatgtttttcagtcttttatctgcaaacgtttgtcttctgcttattcctccgctacttccttggatgacgttttggatgttttgttaaaattgtataaagaaaaggaactcaatatgggtgaaattaatcatttattggtcgatatttttgacgctggtaccgatacaacttctaatacttttgaatgggcaatgtctgaattgattagaaatcccaccatgatgaagagagcacagaatgaaatcgctttagttctaggtaaagataacggtaccattcaagaatctgatatcgccaacatgccatatcttcaagctatcattaaagaaacactgagattacatccacctactgtatttttattgccaaggaaagctattacctatgttaaattatatggctatattgttccaaagaatgcccaaatcttggttaatttatgggctattggtagggatcccaaagtatggaaaaatccaaatgaatttctgcctgatagatttttgaattctgacattgacgttaaaggtagagattttggtttgttaccctttggtgctggtagaagaatttgtccaggcatgaatttagcttatagaatgttaactttaatgttagctaccttgttgcaatcttttgattggaaattgccccatggcaattcaccaatggatttagatatggacgaaaaattcggtattgctttacagaaaacgaagccattggaaatcattccagttattaaatattaa

SEQ ID NO: 49 - protein, 489 aa, Cleretum bellidiforme
MGTHSTAPDLHVVFFPFLAHGHMIPSLDIAKLFAARGVKTTIITTPLNASMFTKAIEKTRKNTETQMEIEVFSFPSEEAGLPLGCENLEQAMAIGANNEFFNAANLLKEQLENFLVKTRPNCLVADMFFTWAADSTAKFNIPTLVFHGFSFFAQCAKEVMWRYKPYKAVSSDTEVFSLPFLPHEVKMTRLQVPESMRKGEETHFTKRTERIRELERKSYGVIVNSFYELEPDYADFLRKELGRRAWHIGPVSLCNRSIEDKAQRGRQTSIDEDECLKWLNSKKPDSVIYICFGSTGHLIAPQLHEIATALEASGQDFIWAVRGDHGQGNSEEWLPPGYEHRLQGKGLIIRGWAPQVLILEHEATGGFLTHCGWNSALEGISAGVPMVTWPTFAEQFHNEQLLTQILKVGVAVGSKKWTLKPSIEDVIKAEDIEKAVREVMVGEEGEERRRRAKKLKEMAWRAIEEGGSSYSDLSALIEELKGYHTSEKE

SEQ ID NO: 50 - DNA, 1476 bp, Artificial
  Codon-optimized sequence
aaaacaatgggtactcattctactgctccagacttgcatgttgttttttttccatttttggcccacggtcatatgatcccatctttggatattgctaagttgtttgctgctagaggtgttaagactaccattattactactccattgaacgcctctatgttcaccaaagctattgaaaagactaggaagaacactgaaacccagatggaaatcgaagttttctcattcccatctgaagaagctggtttgccattgggttgtgaaaatttggaacaagctatggctattggtgccaacaatgaatttttcaacgctgccaacttgctgaaagaacagttggaaaatttcttggtcaagaccagaccaaattgcttggttgctgatatgttttttacttgggctgctgattctaccgccaagtttaacattccaactttggttttccacggcttttctttctttgctcaatgcgctaaagaagtcatgtggcgttacaaaccttacaaggctgtttcttctgataccgaggttttttctttgccattcttgccacatgaagtcaagatgactagattgcaagttccagaatctatgaggaagggtgaagaaactcatttcactaagagaaccgaaaggatcagagaattggagagaaaatcttacggtgtgatcgtcaactctttctacgaattggaaccagattacgccgacttcttgagaaaagaattgggtagacgtgcttggcatattggtccagtttctttgtgtaacagatccattgaagataaggcccaaagaggtagacaaacctctattgatgaagatgaatgcctgaagtggctgaattctaaaaagccagattccgttatctacatctgctttggttctaccggtcatttgattgctccacaattgcacgaaattgctactgctttggaagcttctggtcaagatttcatttgggctgttagaggtgatcatggtcaaggtaattctgaagaatggttgccaccaggttacgaacatagattacaaggtaagggtttgatcattagaggttgggctccacaagttttgattttggaacatgaagctactggtggtttcttgactcattgtggttggaattctgcattggaaggtatttctgctggtgttccaatggttacttggccaacttttgctgaacaattccataacgaacagttgttgacccaaatcttgaaagttggtgttgccgttggttctaagaaatggactttgaagccatccatcgaagatgttattaaggccgaagatatcgaaaaggccgttagagaagttatggttggagaagaaggtgaagaacgtagaagaagggctaaaaagttgaaagaaatggcttggagagctattgaggaaggtggttcttcttattctgatttgtccgccttgatcgaagaattgaaaggttaccatacctccgagaaagagtga

SEQ ID NO: 51 - protein, 494 aa, Beta vulgaris
MGAEPQRLHVVFFPLMAAGHLIPTLDIAKLFAAHHVKTTIITTPLNAPCFTKPLESYKNLGHRIDIEIIPFPSKEAGLPEGLENFDQFTSDQMAVKFLKATELLQESFEKFLEKHKPNCIVTDMLMPFTNNVAAKFNIPRIVFHGCSYFALCMMHTLLKYQPHKSLLSDDEEFLVPNLPHEINLTRSRLPDMMRGQGDKELNDAWMKIFIHAMEAEENSFGVIMNSFYELEPEYVEYYRNVMGRKAWHIGPVSLCNRENEAKFQRGKDSSINEHECLKWLDSKKPKSVVYICFGSLAEVPTLQLREIAMGLEASEQDFIWVVRRGKENVEEEKIEEWLPYDFEDRMEGKGLIIRGWAPQVLILDHEAIGAFVTHCGWNSTLEGISCGVPMVTWPVFAEQFYNEKLVTEVLKTGVAVGAKKWSRILEVNLKSEDIKNAIRRVMVGEEALVLRSKAKKLKELARKAVEIGGSSYSDMHSLIQDLSSYNANGYKQYL

SEQ ID NO: 52 - DNA, 1485 bp, Artificial
  Codon-optimized sequence
atgggtgctgaaccacaaagattgcacgttgttttttttccattgatggctgccggtcatttgattccaactttggatattgctaagttgttcgctgctcatcatgttaagactaccattattactaccccattgaacgctccatgtttcacaaaaccattggagtcttacaagaacttgggtcacagaattgacatcgagattatcccatttccatccaaagaagctggtttgccagaaggtttggaaaactttgatcaattcacctccgatcaaatggccgttaagtttttgaaggctactgagttgttgcaagagtcctttgaaaagttcttggaaaagcacaagccaaactgtatcgttaccgatatgttgatgccattcactaacaatgttgctgccaagttcaacatcccaagaatcgtttttcatggctgttcttacttcgctttgtgtatgatgcataccttgttgaaataccagccacacaagtctttgttgtccgatgatgaagaatttctggttccaaatttgccacacgaaatcaacttgaccagatctagattgccagatatgatgagaggtcaaggtgacaaagaattgaatgatgcttggatgaagattttcattcatgccatggaagccgaagaaaactctttcggtgttatcatgaacagcttctacgaattggaacctgaatacgttgagtactacagaaacgttatgggtagaaaggcttggcatattggtccagtttctttgtgtaacagagaaaacgaagccaagttccaaagaggtaaggattcctctattaacgaacacgaatgtttgaagtggctggattctaaaaagccaaagtccgttgtttacatctgctttggttctttggctgaagttccaacattgcagttgagagaaattgctatgggtttagaagcttccgaacaagatttcatctgggttgttagaaggggtaaagaaaacgtcgaagaagaaaagattgaagagtggttgccatacgacttcgaagatagaatggaaggtaagggtttgattattagaggttgggctccacaggttttgatcttggatcatgaagctattggtgctttcgttactcattgtggttggaactctactttggaaggtatttcttgtggtgttcctatggttacttggccagtttttgctgaacaattctacaacgaaaagttggtcaccgaagttttgaaaactggtgttgctgttggtgctaaaaagtggtctagaatcttggaagtcaacttgaagtccgaggatattaagaacgccattagaagagttatggttggtgaagaagctttggtcttgagatctaaagccaagaagttgaaagaattggctagaaaggcagttgaaatcggtggttcttcttactctgatatgcactccttgattcaggacttgtcatcttacaatgctaacggttacaagcagtacctgtga

SEQ ID NO: 53 - protein, 476 aa, Beta vulgaris
MDDKSQQLHIVLFPFMAHGHMIPTLDIARLFAARGVKTTLITTPRNAPTFLTAIEKGNKSGAPTINVEVFNFQAQSFGLPEGCENLEQALGPGIRDRFFKAAAMLRDQLEHFLEKTRPNCLVADMFFPWATDSAAKFNIPRLVFHGHCLFALCALEIIRLHEPYNNASSDEEPFLLPHLPHEIELTRLQFSEELWKNGGDSDYKERSKAIKESELKCYGVLVNSFYELEPDYAEYFRKDLGRRAWNIGPVSLYNRSNEEKAQRGKQASIDEHECLKWLNSKKPNSVIYICFGSTMHMIPSQLNEIAMGLEASGKDFIWVVRNEDDLGEFEHRMEGKGLIIRGWAPQVLILEHEVIGAFVTHCGWNSTIEGIAAGVPMVTWPVFAEQFLNEKLITRVLRIGIPVGAKKWDCKPCEEYVVKKNDIEKALREVMEGNEAEERRTRAKEYKEMAWKALQEGGSSYSDLSALIDELRGLST

SEQ ID NO: 54 - DNA, 1431 bp, Artificial
  Codon-optimized sequence
atggacgacaagtcccaacaattgcacatagttttgtttccattcatggcccatggtcatatgattccaactttggatattgctaggttgtttgctgctagaggtgttaagactactttgattactactccaagaaacgctccaactttcttgactgctattgaaaagggtaacaaatctggtgctccaaccattaacgttgaggtttttaacttccaagctcagtcttttggtttgcctgaaggttgtgaaaatttggaacaagctttaggtccaggtatcagagatagatttttcaaagctgctgccatgttgagagatcaattggaacatttcttggaaaagaccagaccaaattgcttggttgctgatatgttttttccatgggctactgattctgctgccaagttcaatattccaagattggtttttcatggccattgcttgttcgctttgtgtgctttggaaatcatcagattgcatgaaccttacaacaacgcctcttctgatgaagaaccatttttgttaccacacttgccacacgaaattgaattgaccagattgcagttctctgaagagttgtggaagaatggtggtgattccgattacaaagaaagatccaaggccatcaaagaatccgaattgaaatgttacggtgtcctggttaactccttctatgaattggaaccagattacgccgaatacttcagaaaagatttgggtagacgtgcatggaacattggtccagtttcattatacaacaggtccaacgaagaaaaggctcaaagaggtaaacaagcctccattgatgaacatgaatgtctgaagtggctgaactctaagaagccaaattccgttatctacatctgtttcggttctaccatgcatatgatcccatctcaattgaacgaaatcgctatgggtttagaagcttccggtaaagatttcatctgggttgttagaaacgaagatgacttgggtgaattcgaacatagaatggaaggtaagggtttgattattagaggttgggctccacaagtcttgatcttggaacatgaagttattggtgctttcgttactcattgtggttggaactctaccattgaaggtattgctgctggtgttccaatggttacttggccagtttttgctgaacaattcttgaacgagaagttgatcaccagagttttgagaattggtattccagttggtgctaaaaagtgggattgcaaaccatgtgaagaatacgttgttaagaagaacgatatcgaaaaggccttgagagaagttatggaaggcaatgaagctgaagaaagaagaactagagccaaagagtacaaagagatggcttggaaggctttacaagaaggtggttcatcttactctgatttgtccgctttgatcgatgaattgagaggtttgtctacctaa

SEQ ID NO: 55 - protein, 487 aa, Beta vulgaris
MSAEPQDFHVVLFPFMAHGHMIPMLDIARLFTARDNVKATIITTQLNIATITKDIESNKTTRTPIFNIELFKFPAQEVGLPEGFENLEQVLRPEFFDKFFEATELLNEQLEKYLEKVRPNCLVADMFFPWATDSAAKFNIPRLVFHGTSIIALCAEEIIRLYEPFNNVSSDEEPFVLPLLPHEIQLRRSQISEGLTENEETDYKRKIANIKESELKSYGVIINSFYEMEPDYAKFYSKELRRRSWNVGPVSLSNRSIEDKNQGDKQALIDEHECLQWLNSKKPDSVTYICFGSTTSFIAPQLHEIAMALEASGEDFIWVVKDDENVKGEEWLPTGFKQRMEGKGFIITGWAPQVLILEHEAIRAFMTHCGWNSILEGISAGVPMVTWPIFAEQFYNEKLVNQILKTSVPIGAKKWSRRHSLEDLIKCDAIEKALKEIMVGKEAEERRTRAKNLKKKAYMAVEEGGSSYSDLTALINELRGYRLQEQE

SEQ ID NO: 56 - DNA, 1464 bp, Artificial
  Codon-optimized sequence
atgtccgctgaaccacaagatttccacgttgttttgtttccattcatggctcatggtcatatgatcccaatgttggatattgctaggttgttcactgctagagataacgttaaggctaccattatcactacccaattgaacattgccaccatcaccaaggatatcgaatctaacaaaactactaggacgcccattttcaacatcgagttgtttaagtttccagctcaagaagttggtttgccagaaggttttgaaaacttggaacaagttttgaggccagagttcttcgacaagttttttgaagctactgagttgttgaacgagcagttggaaaagtacttggaaaaggttagaccaaactgcttggttgctgatatgttttttccatgggctactgattctgctgccaagtttaacattccaagattggtttttcatggcacctccattattgctttgtgtgccgaagaaattatcaggttgtatgaacccttcaacaacgtttcctctgatgaagaaccattcgttttgccattactgccacacgaaattcagttgagaagatcccaaatttctgaaggtttgaccgaaaacgaagaaaccgattacaagagaaagatcgccaacatcaaagagtccgaattgaaatcttacggcgtcatcatcaacagcttctacgaaatggaaccagattacgctaagttctactccaaagaattgcgtagaagatcttggaacgttggtccagtttctttgtccaacagatccatcgaagataagaatcaaggtgataagcaagccttgatcgatgaacatgaatgcttgcaatggctgaattctaagaagccagattccgttacctacatttgtttcggttctaccacttctttcattgctccacaattgcacgaaattgctatggctttggaagcttctggtgaagatttcatttgggttgttaaggatgacgaaaacgtcaaaggtgaagaatggttgccaactggttttaagcaaagaatggaaggtaagggtttcattattactggttgggctccacaagtcttgattttggaacatgaagctattagagccttcatgactcattgtggttggaactctattttggaaggtatttctgctggtgttccaatggttacttggccaatttttgctgaacagttctacaacgagaaactggtcaaccagatcttgaaaacctctgttccaattggtgctaaaaagtggtctagaaggcattccttggaagatttgattaagtgtgatgccattgagaaggccctgaaagaaatcatggttggtaaagaagctgaagaaagaagaaccagagctaagaacttgaagaaaaaggcttacatggctgttgaagaaggtggttcatcttactctgatttgaccgctttgatcaacgaattgagaggttacagattgcaagagcaagagtga

SEQ ID NO: 57 - protein, 485 aa, Beta vulgaris
MGAEEFHVVLFPFMAHGHMIPILDIARLFAVRDNVKATIITTPLNVTNFTKDNESNMESSAPIVNIEVFRFPAQEVGLPEGFENLEKVMKPELLNKFFKAAGMLNEQLEQYLEKIRPNCLVADMFFPWATESAAKFNIPRLVFHGTSNFALCAQEIVRLYKPFKNVTSDEEPFVLPSLPHDIQMRRLQIPEDLWKYDGTEFEKRMDSLKDSEIKSYGVLVNSFYELEPDYAEFFKKELGRKTWNIGPVSLSNRSVKDKSQREKEPLIDQHECLKWLNSKKPDSVVYICFGSTASFIAPQLHEMALALEVSGQEFIWIVKNDENGRSKEWLPPGFEQRTEGKGLIIRGWVPQVLILEHEAIGAFVTHCGWNSTLEGISAGVPMVTWPIFAEQFYNEKLVNQILNTGVPVGAKKWSRTPSIEDIIKMEAIEEALTDIMVGTEAEERRTRAKKLKILAYKAVEEDGSSYSDLTALINELSANRLQEQE

SEQ ID NO: 58 - DNA, 1458 bp, Artificial
  Codon-optimized sequence
atgggtgccgaagaattccacgttgttttgtttccattcatggctcatggtcatatgatcccaattttggatattgctaggttgttcgccgttagagataatgttaaggctaccattattacgaccccattgaatgttaccaacttcaccaaggataacgagtccaatatggaatcttctgctccaatcgttaacatcgaagtttttagattcccagctcaagaagttggtttgccagaaggttttgaaaacttggaaaaggttatgaagcccgagttgttgaacaagttttttaaggctgctggtatgttgaacgaacagttggaacaatacttggagaagattagaccaaactgcttggttgctgatatgttttttccatgggctactgaatctgctgccaagtttaacattccaagattggtttttcatggcacctctaactttgctttgtgtgctcaagaaatcgtcaggttgtacaagccttttaagaacgttacctctgacgaagaaccattcgttttgccatctttgccacatgatatccagatgagaagattgcaaatccctgaagatttgtggaagtacgatggtactgaattcgaaaagagaatggactccttgaaggactctgaaatcaaatcttacggtgtcttggtcaactccttctatgaattggaaccagattacgccgagttcttcaaaaaagaattgggtagaaagacctggaacatcggtccagtttctttgtctaatagatccgttaaggacaagtcccagagagaaaaagaacctttgattgatcaacacgagtgcttgaagtggttgaattctaaaaagccagattccgttgtctacatctgttttggttctaccgcttcttttattgctccacaattgcacgaaatggctttggctttggaagtttctggtcaagaattcatttggatcgtcaagaatgacgagaacggtagatctaaagaatggttgccacctggttttgaacaaagaactgaaggtaagggtttgatcattagaggttgggttccacaagtcttgatcttggaacatgaagctattggtgctttcgttactcattgtggttggaactctactttggaaggtatttctgctggtgttccaatggttacttggccaatttttgctgaacagttctacaacgagaaactggtcaatcagattttgaataccggtgttccagttggtgctaaaaaatggtctagaaccccatccattgaggacattattaagatggaagccattgaagaagccttgaccgatattatggttggtacagaagctgaagaaagaagaactagagccaagaagctaaagattttggcttacaaggctgtcgaagaagatggttcatcttactctgatttgaccgccttgattaacgaattgtctgctaacagattgcaagagcaagagtga

SEQ ID NO: 59 - DNA, 816 bp, Artificial
  Codon-optimized sequence
atgggcgtcggcaaggaagtgtctttcaaggaatctttcttcctgtctcacggcaaccccgccatgctggccgacgagtcttttatcgcccgaaacttcctgctcggctggaagaagaacgttttccccgtcaagcccaagtctatcctggtggtgtctgcccactgggagactgacgtgccctgcgtgtctgccggacagtaccccaacgtgatctacgacttcaccgaggtgcccgcctctatgttccagatgaagtaccccgctcctggctgccccaagctggccaagcgagtgcaagagctgctgattgccggcggattcaagtctgccaagctggacgaggaacgaggcttcgaccactcttcttgggtgcccctgtctatgatgtgccccgaggccgacattcccgtgtgccagctgtctgtgcagcccggcctggacgctacccaccacttcaacgtgggccgagcactggcccctctgaagggcgagggcgtgctgttcatcggctctggcggcgctgtgcacccctctgacgacacccctcactggttcgacggcgtggctccctgggccgctgagttcgaccagtggctcgaggacgccctgctcgagggccgatacgaggacgtgaacaactaccagaccaaggctcccgaaggctggaagctggctcaccctattccagagcacttcctgcctctgcacgtggccatgggcctcgccgctcgaaacctgaagctgaactaattcatcgagctgggcatcatggtgccctaagtgatgctgctgatcaacctgctgctgttctaa

SEQ ID NO: 60 - DNA, 807 bp, Artificial
  Codon-optimized sequence
atgggcggcgagaagaagatgaagggcacctactacattgcccacggcgaccccatcatgtacatcaacaagtctatcaagctgcgacacttcctggaagagtggaaggaaaacgtggtgatggaaaagcccatctgcatcctggtgatctctgcccactgggacaccgacgtgcccaccgtgaacctggtcgagcactgcgacaccatccacgacttcgacgactaccccgatcctctgtaccagatcaagtaccccgctcctggcgctcccaagctggccatgaaggtgcaagagctgctgaagggcggaggcttcaagtgcgaggtggacaccaagcgaggcctggaccacgccgcatggttccctctgatgctgatgtaccccgaggccgacattcccatctgcgagctgtctgtgcagaccaacaaggacggcacccaccactacaacctcggcaaggctctgtctcccctgctgaacgacgacgtgctgatcatcggctctggcggcgctgtgcacccctctgacgacacccctcactgccccaacggcgtggctccctgggctctgcagttcgacaactggctcgaggacgccctgctgtctggccgatacgaggacgtgaaggaattcaagaagatggctcccaactgggagatctctcaccccggccaagagcatctgtaccctctgcacgtggccctgggagccgccggaaacaacgtcaagaccgagctgatccaccagacctgggccgctaacggcgtgttcggctactcttcttacaagttcacctctacctaa

SEQ ID NO: 61 - DNA, 807 bp, Artificial
  Codon-optimized sequence
atgaagggcacctactacatcaaccacggcgaccctctgatgtacctgaagaagcacatcaagctgcgacagtttctggaaggctggcaagagaacgtggtgatcgagaagcccaagtctatcctgatcatctctgcccactgggacaccaacgtgcccaccgtgaacttcgtcgagcactgcgacaccatccacgacttcgacgactaccccgatcctctgtaccagatccagtacagagcccctggcgctcccaacctggccaagaaggtcgaggaactgctgaaggaatctggcatggaatgcgagatcgacaccaagcgaggcctggaccacgccgcatggttccctctcatgttcatgtaccccgaggccaacattcccatctgcgagctgtctgtgcagccctctaaggacggcatccaccactacaacgtcggcaaggctctgtctcccctgctccagcagggcgtgctgatcatcggctctggcggcaccgtgcatccctctgacgacacccctcactgccccaacggcgtggctccctgggccatcgagttcgacaactggctcgaggacgctctgctgtctggccgatacgaggacgtgaacaacttcaagaagctggctcccaactgggagatctctcaccccggccaagagcatctgtaccctctgcacgtggccctgggcgctgccggcaagaaccccaagactcagctgatccaccgatcttgggccgctaacggcgtgttcggctactctacctacaacttcacccctaccactcaaaagaccgacgtgtaa

SEQ ID NO: 62 - DNA, 1494 bp, Artificial
  Codon-optimized sequence
atggaccacgctaccctggccatgatcctggctatcctgttcatctcgttccacttcatcaagctgctgttctctcagcagaccactaagctgctgcctccaggacctaagcctctgcctatcatcggcaacatcctcgaggtgggcaagaagccccaccgatctttcgccaacctggccaagattcacggacccctgatctccctgcgactgggctctgtgactaccatcgtggtgtcctctgccgacgtggccaaggaaatgttcctgaagaaggaccatcctctgtctaaccgaaccattcctaactctgtgaccgctggcgaccaccacaagctgaccatgtcttggctgcccgtgtctcccaagtggcgaaacttccgaaagatcaccgccgtgcatctgctgtctccccagcgactggacgcctgccagaccttccgacacgccaaggtgcagcagctgtacgagtacgtgcaagagtgcgcccaaaagggccaagccgtggacatcggcaaggccgccttcaccacctcgctgaacctgctgtctaagctgttcttctcggtcgagctggctcaccacaagtctcacacctctcaagagttcaaggaactgatctggaacatcatggaagatatcggcaagcccaactacgccgactacttccccatcctgggctgcgtggacccctctggcatccgacgacgactggcctgctctttcgacaagctgatcgccgtgttccagggcatcatctgcgagcgactggctcccgactcttctaccaccactactaccactaccgacgacgtgctggacgtcctgctgcagctgttcaagcagaacgagctgactatgggcgagatcaaccacctcctggtggacatcttcgacgccggcaccgacaccacctcttccaccttcgagtgggtgatgaccgagctgattcgaaaccccgagatgatggaaaaggcccaagaggaaatcaagcaggtcctcggcaaggacaagcagatccaagagtctgacatcatcaacctgccttacctgcaggccatcatcaaggaaaccctccgactgcaccctcctaccgtgtttctgctgccccgaaaggccgacaccgacgtcgagctgtacggctacatcgtgcccaaggacgcccagatcctggtgaacctgtgggccatcggacgagatcccaacgcctggcagaacgccgacatcttctcgcccgagcgattcatcggctgcgagatcgacgtgaagggccgagacttcggactgctgcccttcggagccggccgacgaatctgccccggcatgaacctcgccatccgaatgctgaccctgatgctggccactctgctgcagttcttcaactggaagctcgagggcgacatctctcccaaggacctggacatggacgagaagttcggaatcgccctgcaaaagactaagcccctgaagctgatccccattcctcgatactga

SEQ ID NO: 63 - DNA, 1485 bp, Artificial
  Codon-optimized sequence
atggaccacaccactctggccatgatcctgtctgccatcttcctgctgtacaacctggccaaggccatcttctctcactctaacaccaagctgcctccaggacctaagcctgtgcctatcttcggcaacatcttcgagctgggcgagaagccccaccgatctttcgccaacctggctaagattcacggacccctgatcaccctgaagctgggctctgtgactaccatcgtggtgtcctctgccgaggtggccaaggaaatgttcctgaagaacgacctgcctctggccaaccgaaacgtgcccaactctgtgaccgctggcgaccaccacaagctgaccatgtcttggctgcccgtgtctcccaagtggaagaccttccgaaagatcaccgccgtgcatctgctgtctccccagcgactggactcttgtcaggccctgcgacacaccaaggtgaagcagctgcaccagtacgtgcaagagtgcgccaagcgaggcgagcccgtggacatcggcaaggccgccttcaccacctcgctgaacctgctgtctaacctgttcttctcggtcgagctggctaaccacacctcttcgtcctctcaagagttcaaggaactgatctgggagatcatggaagatatcggcaagcccaactacgccgactacttccccatcctgaagtgcgtggacccctggggcatccgacgacgactggcctctaacttcgacaagctgatcgaggtgttccagggcttcatccgaaagcgactgtctaccggctctttctctgccatcactcccaacgacgtgctggacgtgctcctgaacctcctgaaggaaaaggaactcaacatgggcgagatcaaccacctcctggtggacattttcgacgccggcaccgacaccacctcttctaccttcgagtgggccatggctgagctggtgcgaaaccaagagatgatgaagaaggcccaggacgagatcgagcaggtcctcggaaaggacgccatcatccaagagtctgacatccccaagatgccctacctgcaggccatcatcaaggaaaccctgcgactgcaccctcctaccgtgtttctgctgccccgaaaggccacctctaacgtcgagctgtacggctacgtggtgcccaagaacgcccagatcctggtgaacctgtgggccatcggtcgagatcccaaggtgtgggacaaccccaacatgttctcgcccgagcgattcctgaactccgagatcgacgtgaagggccgagacttcggactgctgcccttcggagccggccgacgaatctgccccggcatgaacctggcctaccgaatgctgaccctgatgctggctaccctgctgcagtctttcgactggaagctcggcgacggcgtgaaccccaaggacctggacatggaagagaagttcggcattgccctgcaaaagaccaagcctctgcaggtcatccccgtgctgaagtactaa

SEQ ID NO: 64 - DNA, 1488 bp, Artificial
  Codon-optimized sequence
atggaccagaccactctggccatgctgctgtctgccctgtacctgctgtacaacctgtacaaggtgatcttcacccagtctaactctaagctgcctcctggacctaagcctctgcctatcttcggcaacatctctgagctgggcgctaagccccaccgatctttcgccaacctggccaagattcacggacccctgatcaccctgaagctgggctctgtgactaccatcgtggtgtcctctgccaaggtggccgaggaaatgttcctgaagaacgacctgcctctggccaaccgaaacgtgcccaactctgtgaccgctggcgaccaccacaagctgaccatgtcttggctgcccgtgtctcccaagtggaagaccttccgaaagatcaccgccgtgcatctgctgtctccccagcgactggacgcctgtcaggccctgcgacacgccaaggtgaagcagctgtacgagtacgtgtacgactgcgccaagaagggcgaagccgtcgacatcggcaaggccgccttcaccacctcgctgaacctgctctcgaacctgttcttctcggtcgagctggcccagcacacctctacctcttcgcagcacttcaagcagctgatctgggacatcatggaagatatcggcaagcccaactacgccgactacttccccgctctgaagtgcgtggacccctggggcatccgacgacgactggccgccaacttcgagcgactgatcgacgtgttccaggacttcattcgaccccgactgtctatgaacccctcttctgtgacctctgcctctgacgtgctggacgtgctcctgaacctctacaaggaaaaggaactgaacatgggcgaagtgaaccacctcctggtggacatcttcgacgccggcaccgacaccacctcttctaccttcgagtgggccatggctgagctggtgcgacaccccgagactatgaagaaggcccaggacgagatcgagcaggtcctcggaaaggacgccaccatccaagaggccgacattcccaagatgccctacctgcaggccatcatcaaggaaaccctgcgactgcaccctcctaccgtgtttctgctgccccgaaaggccgctaccaacgtcgagctgtacggctacgtcgtgcccaaggacgcccagatcctggtgaacctgtgggctatcggtcgagatcccctggtgtgggaccagcctaacgtgttctcgcccgagcgattcctgaactccgacgtggacgtgaagggccgagacttcggactgctgcccttcggagccggccgacgaatctgccccggcatgaacctggcctaccgaatgctgaccctgatgctggctaccctgctgcagtctttcgagtggaaggtcgagaacggcgagaaggctgaggacctggacatggacgagaagttcggcattgccctgcaaaagactaagcccctgcagatcatccccgtgctgaagtactgctaa

SEQ ID NO: 65 - protein, 476 aa, Chenopodium quinoa
MGAEPQQLHAVFFPFLAHGHMIPTLDIARLFAARGVKTTIITTPLNAHTFTTAVEKGNKSGAPLIDIEVFRFPAQEAGLPEGCENLEQALGPGEMEKFFKAAAMLREQLEQFLEKIRPNCLVADMFFTWATDSAAKFNIPRLIFHGTSYFSLCALEVVRLYEPYTKVTSDDEPFLLPYLPHEIHMTRLQLSEDLWKNGGKTEYKKRNNAVKESELKSFGVIVNSFYELEPDYADYFRKDLGRRAWHIGPVSLYNRSIEQKSNRGKQASIGEHECLKWLDSRKPDSVIYICFGSTIHLIPPQLHEVAMALEASGQEFIWVVRNEDDLGGFEQKMSGKGLIIRGWAPQVLILEHEAIGAFVTHCGWNSTLEGISAGLPMVTWPMFAEQFYNEKLITRILKIGVPVGAKKWTTTPSIEDVIPQNAIAKALREIMVGDAAEERRCRAKEYKKMAWKALQEGGSSYSDLSALIDELRGLST

SEQ ID NO: 66 - DNA, 1431 bp, Artificial
  Codon-optimized sequence
atgggcgctgaaccccaacagttacacgccgtgttcttccctttcttagctcacggacacatgatacctacgttagatatcgctaggttgtttgctgccaggggtgtgaaaaccacaattattaccacgccacttaacgctcacactttcactacagccgtagagaagggtaataaatctggtgctccattgattgacattgaggtgttccgttttccagcgcaagaggcaggactacctgagggctgtgagaacctagagcaggctttaggccccggagagatggaaaagttcttcaaagcagcggccatgctgagggaacagctagagcaatttctagaaaagattagacccaactgtctagtcgcagacatgttcttcacatgggcaaccgattcagccgccaaattcaacatccctcgtctgatatttcatgggacctcatacttctccctatgcgctctagaggtcgttcgtttatacgagccatatactaaggttacaagcgatgacgaacctttcttacttccgtatttgccgcatgagattcacatgacgcgtttgcagctaagtgaggatttgtggaagaacggtggtaagacggaatacaagaagagaaataacgctgtgaaggagtcggagcttaaatcattcggtgtaatcgtgaacagtttctacgaacttgagccggactacgccgattacttcaggaaggatttgggtagaagggcgtggcatatcgggcccgtgtccttatacaacagaagcatcgagcaaaagtcaaacaggggcaagcaagcgtctattggtgagcatgaatgcctgaagtggctggacagtagaaagccggactccgtgatctatatctgtttcggctcaactattcacctgattcccccgcaattacacgaggtcgcaatggccctggaagcatcgggccaggagtttatttgggttgtgaggaatgaagatgaccttggcggattcgagcagaagatgtctggcaaaggtttaattataaggggatgggcgcctcaggtcttgattttggaacacgaagctatcggtgcatttgtcacgcattgtggctggaactccaccctggaaggaatttcagctggattacctatggtaacgtggcctatgttcgctgaacagttttacaacgaaaagttgataaccaggattttaaagatcggtgtgcctgtgggagcaaagaagtggacgacaaccccatccattgaagacgtgattccgcagaacgcaatagcaaaggctttacgtgagattatggtaggggatgcagcagaggagagaagatgccgtgcaaaggagtacaagaagatggcgtggaaggctctgcaggagggaggaagttcttatagtgacttgtctgcacttattgatgaattgcgtgggcttagtacctaa

SEQ ID NO: 67 - protein, 479 aa, Bougainvillea glabra
MESESKTEREHILMVPFMAQGHLRPFLELGLLLHSRTHFSITLLTTPLNAQYLRQQHHPNTGVRIVELPFNSADHGLPPGIENTSKLPLPLVVSLFHSSSSLEPHLRNFISLNFTPTKPPLCIIHDVFFGWVDSVAKSVGSTAIAFTTGGAYGTAAYASIWNELPHRNLSDDHDFPLPGFPDTHRFRRTQLHRFVRYADGDDDWSRFFQPQIRLSMGSFGWLCNSVQEIEPLGFQILRNYINLPIWGIGPLIATPVHIDNECIQWLSTKQPDSVLYISFGSQNTVSPTQMMELAAGLESSEKPFLWVIRAPFGFDINGEMRPEWLPEGFEERIKEKKQGILVHKWGPQLEILNHKSTGGFLTHCGWNSVLESLREGVPMLGWPLAAEQAYNLKYMEEEMGVAVEIARGLEGEISKEKAKRIVEMVLEREQGSKGWEMKNRAVEMGNKLNEAMKECKGSSIKATDDFLDFIMSKRSKPIG

SEQ ID NO: 68 - DNA, 1440 bp, Artificial
  Codon-optimized sequence
atggaatccgaatctaagaccgaaagggaacatattttgatggttccattcatggctcaaggtcatcttagaccatttttggaattgggtttgttgttgcattctaggacccatttctctattaccttgttgactactccattgaacgcccaatatttgagacaacaacatcatccaaacaccggcgttagaatagttgaattgccttttaactctgccgatcatggtttgccaccaggtattgaaaacacttctaaattgccattgcctctggtcgtttctttgttccattcttcttcatctttggaaccacacttgaggaacttcatctctttgaatttcactccaactaagccaccattgtgcattatccatgatgttttctttggttgggttgattccgttgctaaatctgttggttctactgctattgcttttactactggtggtgcttatggtactgctgcttatgcttctatttggaacgaattgccacacagaaacttgtccgatgatcatgattttccattgccaggttttccagatactcacagattcagaagaacccaattgcacagatttgttagatacgctgatggtgatgatgactggtctagatttttccaaccacagatcagattgtccatgggttcttttggttggttgtgtaactccgttcaagaaattgaaccattgggtttccagatcctgagaaactatattaacttgccaatttggggtatcggtccattgattgctactccagttcatattgataacgaatgcatccaatggttgtctaccaaacaaccagattccgtcttgtacatttcattcggttctcaaaacaccgtgtctccaactcaaatgatggaattggctgctggtttggaatcttctgaaaagccatttttgtgggttattagagccccatttggtttcgatattaacggtgaaatgaggccagaatggttgccagaaggttttgaagaaagaatcaaagaaaagaagcagggcatcttagttcataagtggggtccacaattggaaatcttgaaccataagtctacaggtggtttcttgactcattgcggttggaattctgttttggagtctttgagagaaggtgtcccaatgttaggttggccattagctgctgaacaagcttacaatttgaagtacatggaagaagaaatgggcgttgctgttgaaattgctagaggtttggaaggtgaaatctctaaagaaaaggccaagagaatcgtcgaaatggtcttggaaagagaacaaggttctaaaggttgggaaatgaagaacagagcagttgaaatgggtaacaagttgaacgaagctatgaaggaatgcaagggttcatctattaaggctaccgatgatttcttggacttcatcatgtctaagaggtctaagccaattggttaa

SEQ ID NO: 69 - protein, 394 aa, Yarrowia lipolytica
MSPSVEVTPAHTPTSYEVTNSLDSYRGYDHVHWYVGNAKQAASFYITRMGFSPIAYKGLETGSRDVTTHVVGNGQVRFAFSSALRTGEPQADEIHAHLVKHGDAVKDVAFEVDNVEQLFSAAVKKGVRVISEPKVLKDAHGSVTYAVISTYGDTTHTLIERGSYEGAFLPGFVDTSANKDPIAAFLPNIELMHIDHCVGNQDWNEMDNACKYYEETLGFHRFWSVDDKDICTEFSALKSVVMASPNEKIKMPVNEPAVGKKKSQIEEYIDFYDGPGIQHIALRTDCILDTVRDLRARGVEFISVPGSYYENMKERLAKSSLKLEEKFEDIQALNILIDFDEGGYLLQLFTKPLMDRPTVFIEIIQRRNFEGFGAGNFKSLFEAIEREQAKRGNL

SEQ ID NO: 70 - DNA, 1182 bp, Yarrowia lipolytica
atgtcaccttccgtcgaagtcacccctgcacacacacccacctcgtacgaggtgaccaactcgctagacagctatcggggctatgaccacgtccactggtacgttggtaatgccaagcaggccgcctccttctacatcacccgaatgggattctctcccatcgcctacaagggtcttgagactggctctcgagacgtgaccacccatgtcgtgggcaacggccaggtcagatttgcattctcgtctgccctgagaaccggagagccccaagccgacgagatccacgcccatctggtcaagcacggtgacgccgtcaaggatgtggcctttgaggtcgacaatgtggagcagcttttctctgctgctgtcaagaagggcgtccgagtgatttccgagcccaaggtgctcaaggacgcacatggctccgtcacctacgccgtcatctctacctacggagataccactcacactctgattgagcgaggcagctacgagggcgcctttcttcccggatttgtcgacacctccgccaacaaggaccccatcgcggccttcctgcccaacattgagctcatgcacatcgaccactgcgttggaaaccaggattggaacgagatggataacgcatgcaagtactacgaggagactctgggcttccaccggttctggtcggtcgacgataaggacatttgcaccgagttctctgctctcaagtccgtggtcatggcctcacccaacgagaagatcaagatgcccgtcaacgagccggccgtgggcaagaaaaagtcccagattgaggagtacattgacttttacgacggccccggtatccagcacattgctctgcggaccgactgtatcctggacaccgtccgagatctgcgggctcgtggcgtggagttcatttcggtccctggatcttactacgagaacatgaaggagcggctggcaaagtcatctctgaagctcgaggagaagtttgaggatatccaggcgctcaacattctgattgatttcgacgagggcggatatctgctgcagctgttcaccaagcctttgatggaccggcccaccgtgttcattgagatcatccagcgacgaaactttgagggttttggcgccggcaacttcaagtctctgtttgaggccattgagcgggagcaggccaagcgaggcaacctt

SEQ ID NO: 71 - protein, 357 aa, Yarrowia lipolytica
MSRSSSPNASSAEDVRILGYDPLLAPALLQTEVASTKNARETVSKGRKDSIDVITGKSDKLLCIVGPCSLHDPKAAMEYAQRLKELSDKLSGELVIVMRAYLEKPRTTVGWKGLINDPDMDESFNINKGLRLSRKVFCDLTDLGLPIASEMLDTISPQFLADLLSLGAIGARTTESQLHRELASGLSFPVGFKNGTDGTLGVAVDAVQAASHPHHFMGVTKQGVAAITTTKGNENCFIILRGGKKGTNYDAESVAECKKATESMLMVDCSHGNSNKDYRNQPKVSKAVAEQVAAGEKKIIGVMIESNIHEGNQKVPKEGPSALKYGVSITDACVSWETTVDMLTELANAVKERRNKN

SEQ ID NO: 72 - DNA, 1071 bp, Yarrowia lipolytica
atgtcccgttcctcctctcccaacgcctcctctgctgaggacgtgcgaattctgggctacgaccccctcctcgctcccgctcttctccagactgaggttgcctccaccaaaaacgcccgagagaccgtctccaagggccgaaaggactccattgatgtcatcaccggcaagtccgacaagttgctgtgcattgtcggtccctgctccctccacgaccccaaggccgccatggagtacgcccagcgactcaaggagctgtctgacaagctgtctggtgagctcgtcatcgttatgcgagcctacctcgagaagccccgaaccaccgttggctggaagggtctgatcaacgaccccgacatggacgagtctttcaacatcaacaagggtctgcgactctcccgaaaggtcttctgcgaccttaccgatctgggtctgcccattgcctccgagatgctcgataccatttctccccagttcctggccgacctgctctccctgggtgccattggtgctcgaaccaccgagtcccagctgcaccgagagctcgcctccggtctgtctttccccgttggtttcaagaacggaaccgacggtactctgggtgttgccgttgatgctgtccaggccgcctctcaccctcaccacttcatgggtgtcaccaagcagggtgttgccgccatcaccaccaccaagggtaacgagaactgcttcatcattctgcgaggaggtaagaagggcaccaactacgacgccgagtccgtcgccgagtgcaagaaggccaccgagtccatgctcatggttgactgctctcacggcaactccaacaaggactaccgaaaccagcccaaggtttccaaggccgttgctgagcaggttgctgctggcgagaagaagatcatcggtgtcatgatcgagagtaatatccacgagggcaaccagaaggtccccaaggagggcccctctgcccttaaatacggtgtctccatcaccgacgcctgtgtctcttgggagaccaccgtggacatgctcaccgagctggccaacgccgtcaaggagcgacgaaacaagaac

SEQ ID NO: 73 - protein, 256 aa, Yarrowia lipolytica
MDFTKADTVLDLANIRDSLVRMEDTIVFNLIERAQFCRSEFVYKAGNSDIPGFKGSYLDWFLQESEKVHAKLRRYAAPDEQAFFPDDLPEAILPPIDYAPILAPYSKEVSVNDEIKKIYTDDIVPLVCAGTGDQPENYGSVMVCDIETLQALSRRIHFGKFVAESKFLSETERFTELIKNKDIAGIEAAITNSKVEETILARLGEKALAYGTDPTLRWSQRTQGKVDSEVVKRIYKEWVIPLTKKVEVDYLLRRLE

SEQ ID NO: 74 - DNA, 768 bp, Yarrowia lipolytica
atggacttcactaaagccgacaccgttctggatctcgccaacatccgagactcgctggtccgaatggaggacactattgtcttcaatctgattgagcgggctcagttctgccgttccgagtttgtgtacaaggccggcaactcggacattcccggcttcaagggctcttacctcgactggtttctgcaggagtcggaaaaggtgcacgccaaactgcgtcggtacgctgccccggacgagcaggccttcttccccgacgatctacccgaggccattctgccccccatcgattatgcgccaattctggcaccctacagcaaggaggtgagcgtcaacgacgagattaaaaagatttacaccgacgacattgtgcccctggtgtgtgctggcactggagatcagcccgagaactatgggtcggtcatggtgtgcgacatcgagacgctgcaggcgctgtcgcgacgaatccactttggcaagtttgtggccgagtccaagtttctgagtgaaaccgagcgattcaccgagctcatcaagaacaaggacattgctggtattgaggcggccatcacaaactccaaggtggaagagacgattctggcccggctgggagaaaaggcactggcctacggcacagaccccactctccggtggtcgcagagaacccagggaaaggttgattccgaggttgtcaagcgaatctacaaggagtgggtgattccactcaccaagaaggtcgaggtggactacctgctccggcggttggag

SEQ ID NO: 75 - protein, 370 aa, Saccharomyces cerevisiae
MSESPMFAANGMPKVNQGAEEDVRILGYDPLASPALLQVQIPATPTSLETAKRGRREAIDIITGKDDRVLVIVGPCSIHDLEAAQEYALRLKKLSDELKGDLSIIMRAYLEKPRTTVGWKGLINDPDVNNTFNINKGLQSARQLFVNLTNIGLPIGSEMLDTISPQYLADLVSFGAIGARTTESQLHRELASGLSFPVGFKNGTDGTLNVAVDACQAAAHSHHFMGVTKHGVAAITTTKGNEHCFVILRGGKKGTNYDAKSVAEAKAQLPAGSNGLMIDYSHGNSNKDFRNQPKVNDVVCEQIANGENAITGVMIESNINEGNQGIPAEGKAGLKYGVSITDACIGWETTEDVLRKLAAAVRQRREVNKK

SEQ ID NO: 76 - DNA, 1110 bp, Saccharomyces cerevisiae
atgagtgaatctccaatgttcgctgccaacggcatgccaaaggtaaatcaaggtgctgaagaagatgtcagaattttaggttacgacccattagcttctccagctctccttcaagtgcaaatcccagccacaccaacttctttggaaactgccaagagaggtagaagagaagctatagatattattaccggtaaagacgacagagttcttgtcattgtcggtccttgttccatccatgatctagaagccgctcaagaatacgctttgagattaaagaaattgtcagatgaattaaaaggtgatttatccatcattatgagagcatacttggagaagccaagaacaaccgtcggctggaaaggtctaattaatgaccctgatgttaacaacactttcaacatcaacaagggtttgcaatccgctagacaattgtttgtcaacttgacaaatatcggtttgccaattggttctgaaatgcttgataccatttctcctcaatacttggctgatttggtctccttcggtgccattggtgccagaaccaccgaatctcaactgcacagagaattggcctccggtttgtctttcccagttggtttcaagaacggtaccgatggtaccttaaatgttgctgtggatgcttgtcaagccgctgctcattctcaccatttcatgggtgttactaagcatggtgttgctgctatcaccactactaagggtaacgaacactgcttcgttattctaagaggtggtaaaaagggtaccaactacgacgctaagtccgttgcagaagctaaggctcaattgcctgccggttccaacggtctaatgattgactactctcacggtaactccaataaggatttcagaaaccaaccaaaggtcaatgacgttgtttgtgagcaaatcgctaacggtgaaaacgccattaccggtgtcatgattgaatcaaacatcaacgaaggtaaccaaggcatcccagccgaaggtaaagccggcttgaaatatggtgtttccatcactgatgcttgtataggttgggaaactactgaagacgtcttgaggaaattggctgctgctgtcagacaaagaagagaagttaacaagaaa

SEQ ID NO: 77 - protein, 256 aa, Saccharomyces cerevisiae
MDFTKPETVLNLQNIRDELVRMEDSIIFKFIERSHFATCPSVYEANHPGLEIPNFKGSFLDWALSNLEIAHSRIRRFESPDETPFFPDKIQKSFLPSINYPQILAPYAPEVNYNDKIKKVYIEKIIPLISKRDGDDKNNFGSVATRDIECLQSLSRRIHFGKFVAEAKFQSDIPLYTKLIKSKDVEGIMKNITNSAVEEKILERLTKKAEVYGVDPTNESGERRITPEYLVKIYKEIVIPITKEVEVEYLLRRLEE

SEQ ID NO: 78 - DNA, 768 bp, Saccharomyces cerevisiae
atggatttcacaaaaccagaaactgttttaaatctacaaaatattagagatgaattagttagaatggaggattcgatcatcttcaaatttattgagaggtcgcatttcgccacatgtccttcagtttatgaggcaaaccatccaggtttagaaattccgaattttaaaggatctttcttggattgggctctttcaaatcttgaaattgcgcattctcgcatcagaagattcgaatcacctgatgaaactcccttctttcctgacaagattcagaaatcattcttaccgagcattaactacccacaaattttggcgccttatgccccagaagttaattacaatgataaaataaaaaaagtttatattgaaaagattataccattaatttcgaaaagagatggtgatgataagaataacttcggttctgttgccactagagatatagaatgtttgcaaagcttgagtaggagaatccactttggcaagtttgttgctgaagccaagttccaatcggatatcccgctatacacaaagctgatcaaaagtaaagatgtcgaggggataatgaagaatatcaccaattctgccgttgaagaaaagattctagaaagattaactaagaaggctgaagtctatggtgtggaccctaccaacgagtcaggtgaaagaaggattactccagaatatttggtaaaaatttataaggaaattgttatacctatcactaaggaagttgaggtggaatacttgctaagaaggttggaagag

SEQ ID NO: 79 - protein, 439 aa, Bougainvillea glabra
MGSEGEQKMQHLTHVFMISFPGQGHINPLLRLGKRLASKGLLVTFSTTADFGHAIRSSNDAVSDQPVPVGNGFIRFEFIDDEWPETDPRRIDMDQYLPQYELVGRKKLPQMLGRLAQEGRPVSCLINNPFIPWVSDVAEQLGLPSAMLWVQSCACFLAYYYYHNKLVPFPDENTPHMDVEIPSLPLLKWDEMPTFLHPTTPYPFLRRAILSQYENLSKPFCILADTFYELEKEIVDHTNTLCPIRPVGPLFKDPKPEPGSGFARVRADPMRADQECIKWLDSKPARSVVYISFGTVVFLKQEQVDEIAGGLEAAGIPFLWVMKPPHPDAGKQPHHLPEGFLDRAGDNGKVVHFSPQEQVLAHPSVACFMTHCGWNSSMEALTSGVPVIAFPQWGDQVTDAKFLCDVFGVGIQLCKGEHDNRIISREEVDKCLKDATSGP

SEQ ID NO: 80 - protein, 495 aa, Bougainvillea glabra
MKMENQESTKSTPPLTAYFLPYLTPSHFMPVVQMAQLFASRGVHVTILITHHNSLLFKDSITELADRGFQIHIHLLDFPSKQVGLPEGIESLSIATTKELAGKVIRAFMMLQKPMEDAVRAARPNFIVSDMHCFWASDLAHELGIPCLLFHARPFVGLCAIDAIYRYKPYESVTSDEDPFLLPGLPDPLQMYRSEMPDWIRTPNPYTHFVNKIMEGDKKSYGALVFSFNEVEQAYADHYTKTLGRQAWAIGPLPLHCATALPLAQAKKDEEQSKNPCLEWLDKMERDSVLYVSFGSLSRFTSDQLFEIAMGLESAGHPFIWVVRKSEGTEPGWLPDGFKTRIREQNLGMLIEGWAPQVKILDHTSTGGFMTHCGGNSITESVTSGVPMITWPLSADHFYHRKLVTEVAKIGVDVGNKKWGTMIEATDELVGRERIEWAVKELMGGGDEEAEERRRRARELSVAAKKTVQPGGSSYDNLTHVIDEIRRFTLSAQQS